(19) United States
(12) Patent Application Publication
(10) Pub. No.: US 2026/0183268 A1
(43) Pub. Date: Jul. 2, 2026

(54) Heteroaryl Compounds and Their Use in Treating Medical Conditions

(71) Applicant: Alphina Therapeutics, Inc., New Haven, CT (US)

(72) Inventors: Jotham Wadsworth Coe, Niantic, CT (US); John F. Kadow, Wallingford, CT (US); Brian Thomas O'Neill, Haddam, CT (US); Mark Stephen Plummer, Westbrook, CT (US)

(73) Assignee: Alphina Therapeutics, Inc., New Haven, CT (US)

(21) Appl. No.: 19/433,321

(22) Filed: Dec. 26, 2025

Related U.S. Application Data
(60) Provisional application No. 63/739,336, filed on Dec. 27, 2024.
(60) Provisional application No. 63/802,970, filed on May 9, 2025.

(51) Int. Cl. A61K 31/4245; A61K 31/4196; A61K 31/427; A61K 31/437; A61K 31/454; C07D 401/14; C07D 403/12; C07D 405/14; C07D 413/12; C07D 417/12; C07D 471/04

(52) U.S. Cl. CPC A61K 31/4245; A61K 31/4196; A61K 31/427; A61K 31/437; A61K 31/454; C07D 401/14; C07D 403/12; C07D 405/14; C07D 413/12; C07D 417/12; C07D 471/04

(57) ABSTRACT

Provided herein are heteroaryl compounds, pharmaceutical compositions, and their use in the treatment of a disease or condition, such as a proliferative disorder, inflammatory disorder, autoimmune disorder, or metabolic disorder. In some embodiments, the heteroaryl compounds are represented by Formula I:

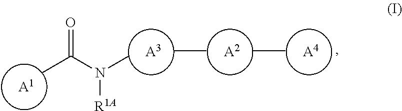

or a pharmaceutically acceptable salt thereof, wherein values for the variables (e.g., A¹, A², A³, A⁴, R^{1A}) are as described herein.

### CROSS-REFERENCE TO RELATED APPLICATIONS

This application claims the benefit of U.S. Provisional Application No. 63/739,336, filed on Dec. 27, 2024, and U.S. Provisional Application No. 63/802,970, filed on May 9, 2025. The entire teachings of these applications are incorporated herein by reference for any purpose.

### FIELD

Provided herein are heteroaryl compounds, pharmaceutical compositions, and their use in the treatment of a disease or condition, such as a proliferative disorder, inflammatory disorder, autoimmune disorder, or metabolic disorder.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. Moreover, new therapies that achieve an anti-cancer effect through a different mechanism present an opportunity to treat cancers more effectively and/or to treat cancers that have become resistant to currently available medicines.

Inflammatory disorders impact a substantial number of patients and often involve situations where the patient's biological response to a stimulus results in the immune system attacking the body's own cells or tissues. This can lead to abnormal inflammation and result in chronic pain, redness, swelling, stiffness, and/or damage to normal tissues. Current treatment options for these inflammatory disorders are not effective for all patients and/or can have substantial adverse side effects.

Nicotinamide adenine dinucleotide (NAD+) is a cofactor that plays an important role in many physiologically essential processes, such as metabolism, energy production, DNA repair, and signaling. Nicotinamide phosphoribosyltransferase (NAmPRTase or NAMPT) is essential for the biosynthesis of NAD+. In mammals, the rate-limiting step in NAD+biosynthesis is the salvage of NAM and conversion to nicotinamide mononucleotide (NMN) catalyzed by NAMPT. Thus, inhibition of NAMPT leads to depletion of NAD+ and a decrease in diseased cell proliferation and even diseased cell death. As a result, NAMPT inhibitors are useful for treating a variety of cancers, metabolic disorders, or inflammatory disorders.

The need exists for new therapeutic methods and compounds for treating diseases or conditions associated with NAMPT activity. The present disclosure addresses the foregoing needs and provides other related advantages.

### SUMMARY

Provided herein are heteroaryl compounds, pharmaceutical compositions, and their use in the treatment of a disease or condition, such as a proliferative disorder, inflammatory disorder, autoimmune disorder, or metabolic disorder. In particular, one aspect provides a collection of heteroaryl compounds, such as a compound represented by Formula I:

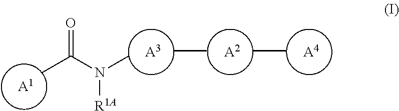

or a pharmaceutically acceptable salt thereof, where the variables are as defined in the detailed description. Further description of additional collections of heteroaryl compounds are described in the detailed description. The compounds may be part of a pharmaceutical composition comprising a pharmaceutically acceptable carrier.

Another aspect provides a collection of heteroaryl compounds, such as a compound represented by Formula II:

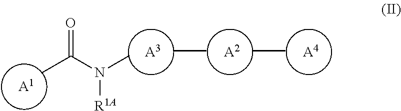

or a pharmaceutically acceptable salt thereof, where the variables are as defined in the detailed description. Further description of additional collections of heteroaryl compounds are described in the detailed description. The compounds may be part of a pharmaceutical composition comprising a pharmaceutically acceptable carrier.

Another aspect provides a method of treating a disease or condition mediated by NAMPT. The method comprises administering to a subject in need thereof a therapeutically effective amount of a compound described herein, such as a compound of Formula I, to treat the disease or condition, as further described in the detailed description.

Another aspect provides a method of inhibiting the activity of NAMPT. The method comprises contacting a NAMPT with an effective amount of a compound described herein, such as a compound of Formula I, to inhibit the activity of said NAMPT, as further described in the detailed description.

### DETAILED DESCRIPTION

Provided herein are heteroaryl compounds, pharmaceutical compositions, and their use in the treatment of a disease or condition, such as a proliferative disorder, inflammatory disorder, autoimmune disorder, or metabolic disorder. The practice of the present disclosure employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the disclosure are set forth below in sections; however, aspects of the disclosure described in one particular section are not to be limited to any particular section. Further, when a variable is not accompanied by a definition, the previous definition of the variable controls.

### Definitions

Compounds of the present disclosure include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

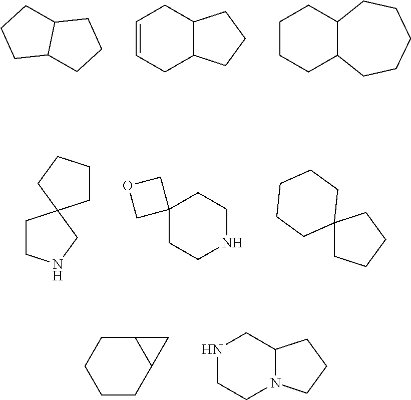

Exemplary bridged bicyclics include:

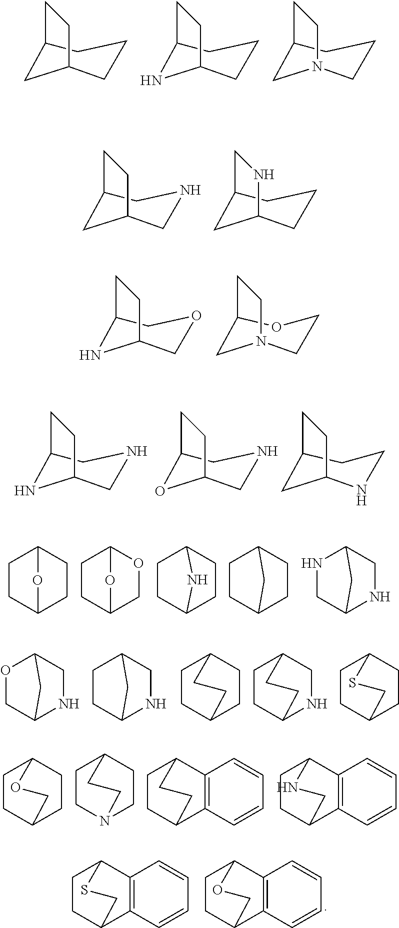

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

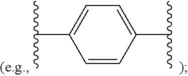 [SMILES: *c1ccc(*)cc1]

“phenylene” is a trivalent phenyl group when it has three groups attached to it

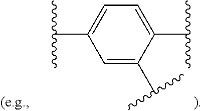 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 Tc electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —S(O)(NR^{o})R^{o}; —S(O)₂N═C(NR^{o}₂)₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂.

Each R^{o} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{o} selected from ═O and ═S; or each R^{o} is optionally substituted with a monovalent substituent independently selected from halogen, —(CH₂)₀₋₂R^{⋅}, -(haloR^{⋅}), —(CH₂)₀₋₂OH, —(CH₂)₀₋₂OR^{⋅}, —(CH₂)₀₋₂CH(OR^{⋅})₂; —O(haloR^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SH, —(CH₂)₀₋₂NH₂, —(CH₂)₀₋₂NHR^{⋅}, —(CH₂)_{0- 2}NR^{⋅}₂, —NO₂, —SiR^{o}3, —OSiR^{o}₃, —C(O)SR^{⋅}, —(C₁₋₄ straight or branched alkylene)C(O)OR^{⋅}, or —SSR^{⋅}.

Each R^{⋅} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{⋅} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆aliphatic, R* is optionally substituted with halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{⋅} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₃ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{⋅} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. The disclosure includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present disclosure may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the disclosure may, for example, be substantially free of other isomers, or may be admixed, for example, as racemates or with all other, or other selected, stereoisomers. For example, in some embodiments, an individual stereoisomer may have a stereochemical purity of at least or about: 50%, 60%, 70%, 80%, 90%, 95%, 98%, 99%, or 99.9%. Chiral center(s) in a compound of the present disclosure can have the S or R configuration as defined by the IUPAC 1974 Recommendations. Further, to the extent a compound described herein may exist as an atropisomer (e.g., substituted biaryls), all forms of such atropisomer are considered part of this disclosure.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane susbstituted with an oxo group is cyclopentanone.

The symbol “ [symbol] ” indicates a point of attachment.

When any substituent or variable occurs more than one time in any constituent or the compound of the disclosure, its definition on each occurrence is independent of its definition at every other occurrence, unless otherwise indicated.

One or more compounds of the disclosure may exist in unsolvated as well as solvated forms with pharmaceutically acceptable solvents such as water, ethanol, and the like, and it is intended that the disclosure embrace both solvated and unsolvated forms. “Solvate” means a physical association of a compound of this disclosure with one or more solvent molecules. This physical association involves varying degrees of ionic and covalent bonding, including hydrogen bonding. In certain instances, the solvate will be capable of isolation, for example, when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present disclosure. Such organisms preferably include, but are not limited to, mammals (e.g., murines, simians, equines, bovines, porcines, canines, felines, and the like), and most preferably include humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

In addition, when a compound of the disclosure contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the disclosure are pharmaceutically acceptable (i.e., non-toxic, physiologically acceptable) salts. Such salts of the compounds of the disclosure may be formed, for example, by reacting a compound of the disclosure with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified.

### I. Heteroaryl Compounds

One aspect of the disclosure provides heteroaryl compounds. The compounds may be used in the pharmaceutical compositions and therapeutic methods described herein. Exemplary compounds are described in the following sections, along with exemplary procedures for making the compounds.

One aspect provides a compound represented by Formula I:

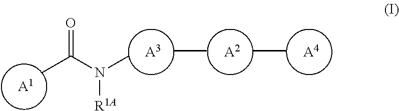

or a pharmaceutically acceptable salt thereof, wherein:
A¹ is

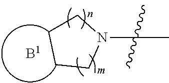

substituted with p occurrences of R², wherein B¹ is a 6-membered aryl ring or a 5-6 membered heteroaryl ring containing 1 or 2 heteroatoms independently selected from nitrogen and sulfur;
A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³;
A³ is phenylene substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1A} and R^{1B} each represent independently for each occurrence hydrogen or C₁₋₄ alkyl;
R² represents independently for each occurrence halo, C₁₋₄ haloalkyl, C₁₋₄ alkoxyl, C₁₋₄ alkyl, or C₁₋₄ hydroxyalkyl;
R³ represents independently for each occurrence halo or C₁₋₄ alkyl;
R⁴ represents independently for each occurrence C₁₋₆ hydroxyalkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ hydroxyhalo-alkyl, C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur);
R⁵ represents independently for each occurrence C₁₋₄ alkyl or —(C₀₋₃ alkylene)-(C₃₋₇ cycloalkyl);
m and n are independently 1 or 2;
p is 0, 1, 2, 3, or 4; and
x is 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, A¹ is

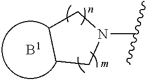

substituted with p occurrences of R², wherein B¹ is a 6-membered aryl ring or a 5-6 membered heteroaryl ring containing 1 or 2 heteroatoms independently selected from nitrogen and sulfur.

In certain embodiments, A¹ is

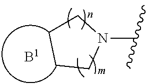

substituted with p occurrences of R², wherein B¹ is a 6-membered aryl ring or a 6-membered heteroaryl ring containing 1 or 2 heteroatoms that are nitrogen.

In certain embodiments, wherein A¹ is

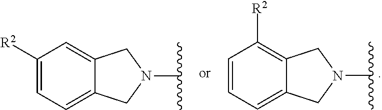

In certain embodiments, A¹ is

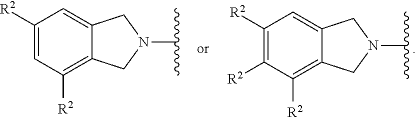

In certain embodiments, A¹ is

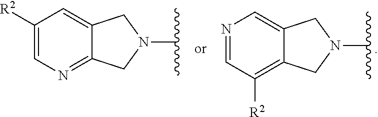

In certain embodiments, A¹ is

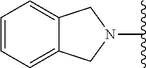 [SMILES: *N1Cc2ccccc2C1]

substituted with p occurrences of R². In certain embodiments, A¹ is

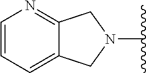 [SMILES: *N1Cc2cccnc2C1]

substituted with p occurrences of R².

In certain embodiments, A¹ is selected from the groups depicted in Table 1, below.

As generally defined above, A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³.

In certain embodiments, A² is a 5-8 membered saturated bridged bicyclic carbocyclylene, a 6-membered saturated or partially unsaturated monocyclic carbocyclylene, or a 6-membered saturated or partially unsaturated monocyclic heterocyclylene containing 1 heteroatom that is nitrogen; each of which is substituted with 0 or 1 occurrence of R³.

In certain embodiments, A² is an 8-membered saturated bridged bicyclic carbocyclylene substituted with 0 or 1 occurrence of R³. In certain embodiments, A² is

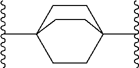 [SMILES: *C12CCC(*)(CC1)CC2]

substituted with 0 or 1 occurrence of R³.

In certain embodiments, A² is

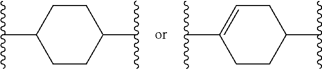 [SMILES: *C1CCC(*)CC1 ; *C1=CCC(*)CC1]

wherein the saturated carbon is attached to A⁴, and wherein

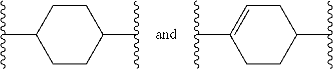 [SMILES: *C1CCC(*)CC1 ; *C1=CCC(*)CC1]

are substituted with 0 or 1 occurrence of R³. In certain embodiments, A² is

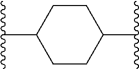 [SMILES: *C1CCC(*)CC1]

which is substituted with 0 or 1 occurrence of R³. In certain embodiments, A² is

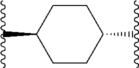 [SMILES: *[C@H]1CC[C@H](*)CC1]

which is substituted with 0 or 1 occurrence of R³. In certain embodiments, A² is

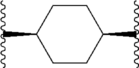 [SMILES: *[C@H]1CC[C@@H](*)CC1]

which is substituted with 0 or 1 occurrence of R³. In certain embodiments, A² contains 0 occurrences of R³.

In certain embodiments, A² is

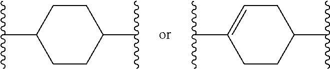 [SMILES: *C1CCC(*)CC1 ; *C1=CCC(*)CC1]

wherein the saturated carbon is attached to A⁴, wherein A² is substituted with 0 or 1 occurrence of R³. In certain embodiments, A² is substituted with 0 occurrences of R³.

In certain embodiments, A² is selected from the groups depicted in Table 1, below.

As generally defined above, A³ is phenylene substituted with 0 or 1 occurrence of R³. In certain embodiments, A³ is para-phenylene substituted with 0 or 1 occurrence of R³. In certain embodiments, A³ is selected from the groups depicted in Table 1, below.

As generally defined above, A⁴ is a 5-6 membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 2 or 3 heteroatoms independently selected from nitrogen, oxygen, and sulfur, wherein the heteroaryl is substituted with x occurrences of R⁴.

In certain embodiments, A⁴ is triazolyl, oxadiazolyl, thiadiazolyl, pyrazolyl, imidazolyl, oxazolyl, or thiazolyl, each of which is substituted with x occurrences of R⁴. In certain embodiments, A⁴ is triazolyl, oxadiazolyl, or thiadiazolyl, each of which is substituted with x occurrences of R⁴. In certain embodiments, A⁴ is pyrazolyl, imidazolyl, oxazolyl, or thiazolyl, each of which is substituted with x occurrences of R⁴. In certain embodiments, A⁴ is 1,2,3-triazolyl or 1,2,4-oxadiazolyl substituted with x occurrences of R⁴. In certain embodiments, A⁴ is a 6-membered heteroaryl containing 1, 2, 3, or 4 heteroatoms that are nitrogen, wherein the heteroaryl is substituted with x occurrences of R⁴. In certain embodiments, A⁴ is a 6-membered heteroaryl containing 1 or 2 heteroatoms that are nitrogen, wherein the heteroaryl is substituted with x occurrences of R⁴.

In certain embodiments, A⁴ is selected from the groups depicted in Table 1, below.

As generally defined above, R^{1A} and R^{1B} each represent independently for each occurrence hydrogen or C₁₋₄ alkyl.

In certain embodiments, R^{1B} is hydrogen.

In certain embodiments, R^{1A} is selected from the groups depicted in Table 1, below. In certain embodiments, R^{1B} is selected from the groups depicted in Table 1, below.

As generally defined above, R² represents independently for each occurrence halo, C₁₋₄ haloalkyl, C₁₋₄ alkoxyl, C₁₋₄ alkyl, or C₁₋₄ hydroxyalkyl. In certain embodiments, R² represents independently for each occurrence halo. In certain embodiments, R² is fluoro. In certain embodiments, R² is selected from the groups depicted in Table 1, below.

As generally defined above, R³ represents independently for each occurrence halo or C₁₋₄ alkyl. In certain embodiments, R³ is selected from the groups depicted in Table 1, below.

As generally defined above, R⁴ represents independently for each occurrence C₁₋₆ hydroxyalkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ hydroxyhalo-alkyl, C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur). In certain embodiments, R⁴ represents independently for each occurrence C₁₋₆ hydroxyalkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ hydroxyhaloalkyl, or C₁₋₆ haloalkyl.

In certain embodiments, R⁴ represents independently for each occurrence C₁₋₆ hydroxyalkyl. In certain embodiments, R⁴ represents independently for each occurrence C₁₋₆ alkyl. In certain embodiments, R⁴ represents independently for each occurrence C₁₋₆ alkylene substituted with 1 occurrence of —N(R^{1B})—C(O)R⁵ or —N(R^{1B})—SO₂R⁵.

In certain embodiments, R⁴ represents independently for each occurrence C₁₋₆ hydroxyhaloalkyl or C₁₋₆ haloalkyl. In certain embodiments, R⁴ represents independently for each occurrence —(C₀₋₃ alkylene)-(a monovalent 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent 5-7 membered ring containing 0 heteroatoms). In certain embodiments, R⁴ represents independently for each occurrence —(C₁₋₃ alkylene)-(a monovalent 5-7 membered ring containing 1 or 2 heteroatoms independently selected from nitrogen, oxygen, and sulfur).

In certain embodiments, R⁴ represents independently for each occurrence —(C₁₋₃ alkylene)-(a monovalent, saturated 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, saturated 5-7 membered ring containing 0 heteroatoms). In certain embodiments, R⁴ represents independently for each occurrence —(C₁₋₃ alkylene)-(a monovalent, saturated 5-7 membered ring containing 1 or 2 heteroatoms independently selected from nitrogen, oxygen, and sulfur). In certain embodiments, R⁴ represents independently for each occurrence —(C₁₋₃ alkylene)-(a monovalent, partially saturated 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, partially saturated 5-7 membered ring containing 0 heteroatoms). In certain embodiments, R⁴ represents independently for each occurrence —(C₁₋₃ alkylene)-(a monovalent, partially saturated 5-7 membered ring containing 1 or 2 heteroatoms independently selected from nitrogen, oxygen, and sulfur).

In certain embodiments, R⁴ represents independently for each occurrence —(C₁₋₃ alkylene)-(a monovalent, aromatic 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, aromatic 5-7 membered ring containing 0 heteroatoms). In certain embodiments, R⁴ represents independently for each occurrence —(C₁₋₃ alkylene)-(a monovalent, aromatic 5-7 membered ring containing 1 or 2 heteroatoms independently selected from nitrogen, oxygen, and sulfur).

In certain embodiments, R⁴ represents independently for each occurrence a monovalent 5-7 membered ring containing 0, 1, or 2 heteroatoms independently selected from nitrogen, oxygen, and sulfur. In certain embodiments, R⁴ represents independently for each occurrence a monovalent 5-7 membered ring containing 0 heteroatoms. In certain embodiments, R⁴ represents independently for each occurrence a monovalent 5-7 membered ring containing 1 or 2 heteroatoms independently selected from nitrogen, oxygen, and sulfur. In certain embodiments, R⁴ represents independently for each occurrence a monovalent, saturated 5-7 membered ring containing 0, 1, or 2 heteroatoms independently selected from nitrogen, oxygen, and sulfur. In certain embodiments, R⁴ represents independently for each occurrence a monovalent, saturated 5-7 membered ring containing 0 heteroatoms. In certain embodiments, R⁴ represents independently for each occurrence a monovalent, saturated 5-7 membered ring containing 1 or 2 heteroatoms independently selected from nitrogen, oxygen, and sulfur. In certain embodiments, R⁴ represents independently for each occurrence a monovalent, partially saturated 5-7 membered ring containing 0, 1, or 2 heteroatoms independently selected from nitrogen, oxygen, and sulfur. In certain embodiments, R⁴ represents independently for each occurrence a monovalent, partially saturated 5-7 membered ring containing 0 heteroatoms. In certain embodiments, R⁴ represents independently for each occurrence a monovalent, partially saturated 5-7 membered ring containing 1 or 2 heteroatoms independently selected from nitrogen, oxygen, and sulfur.

In certain embodiments, R⁴ represents independently for each occurrence a monovalent, aromatic 5-7 membered ring containing 0, 1, or 2 heteroatoms independently selected from nitrogen, oxygen, and sulfur. In certain embodiments, R⁴ represents independently for each occurrence a monovalent, aromatic 5-7 membered ring containing 0 heteroatoms. In certain embodiments, R⁴ represents independently for each occurrence a monovalent, aromatic 5-7 membered ring containing 1 or 2 heteroatoms independently selected from nitrogen, oxygen, and sulfur.

In certain embodiments, R⁴ represents independently for each occurrence

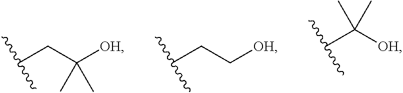

methyl, or

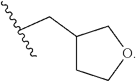 [SMILES: *CC1CCOC1]

In certain embodiments, R⁴ is selected from the groups depicted in Table 1, below.

As generally defined above, R⁵ represents independently for each occurrence C₁₋₄ alkyl or —(C₀₋₃ alkylene)-(C₃₋₇ cycloalkyl). In certain embodiments, R⁵ represents independently for each occurrence C₁₋₄ alkyl. In certain embodiments, R⁵ is selected from the groups depicted in Table 1, below.

As generally defined above, m and n are independently 1 or 2. In certain embodiments, m and n are 1. In certain embodiments, m is 1. In certain embodiments, m is 2. In certain embodiments, n is 1. In certain embodiments, n is 2.

As generally defined above, p is 0, 1, 2, 3, or 4. In certain embodiments, p is 1, 2, or 3. In certain embodiments, p is 0. In certain embodiments, p is 1. In certain embodiments, p is 2. In certain embodiments, p is 3. In certain embodiments, p is 4.

As generally defined above, x is 0, 1, or 2. In certain embodiments, x is 0. In certain embodiments, x is 1. In certain embodiments, x is 2.

In certain embodiments, the compound is represented by Formula I-A:

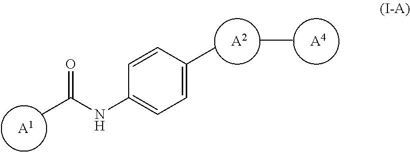

or a pharmaceutically acceptable salt thereof, wherein:
A¹ is

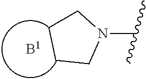 [SMILES: *N1CCCC1]

substituted with p occurrences of R², wherein B¹ is a 6-membered aryl ring or a 6-membered heteroaryl ring containing 1 or 2 heteroatoms that are nitrogen;
A² is a 5-8 membered saturated bridged bicyclic carbocyclylene or a 6-membered saturated or partially unsaturated monocyclic carbocyclylene; each of which is substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1B} represents independently for each occurrence hydrogen or C₁₋₄ alkyl;
R² represents independently for each occurrence halo or C₁₋₄ haloalkyl;
R³ is halo or C₁₋₄ alkyl;
R⁴ represents independently for each occurrence C₁₋₆ hydroxyalkyl, C₁₋₆ hydroxyhalo-alkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur);
R⁵ represents independently for each occurrence C₁₋₄ alkyl or —(C₀₋₃ alkylene)-(C₃₋₇ cycloalkyl);
p is 1 or 2; and
x is 0, 1, or 2.

The description above describes multiple embodiments relating to compounds of Formula I-A. The patent application specifically contemplates all combinations of the embodiments.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

As defined generally above, A¹ is

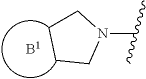 [SMILES: *N1CCCC1]

substituted with p occurrences of R², wherein B¹ is a 6-membered aryl ring or a 6-membered heteroaryl ring containing 1 or 2 heteroatoms that are nitrogen.

In certain embodiments, wherein A¹ is

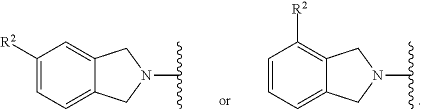

In certain embodiments, A¹ is

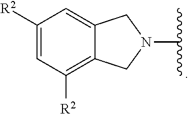

In certain embodiments, A¹ is

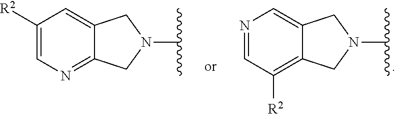

In certain embodiments, A¹ is

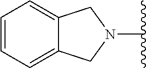 [SMILES: *N1Cc2ccccc2C1]

substituted with p occurrences of R². In certain embodiments, A¹ is

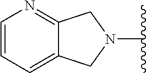 [SMILES: *N1Cc2cccnc2C1]

substituted with p occurrences of R².

In certain embodiments, A¹ is selected from the groups depicted in Table 1, below.

As generally defined above, A² is a 5-8 membered saturated bridged bicyclic carbocyclylene or a 6-membered saturated or partially unsaturated monocyclic carbocyclylene; each of which is substituted with 0 or 1 occurrence of R³.

In certain embodiments, A² is an 8-membered saturated bridged bicyclic carbocyclylene substituted with 0 or 1 occurrence of R³. In certain embodiments, A² is

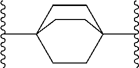 [SMILES: *C12CCC(*)(CC1)CC2]

substituted with 0 or 1 occurrence of R³.

In certain embodiments, A² is

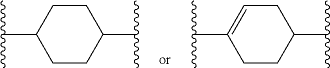 [SMILES: *C1CCC(*)CC1 ; *C1=CCC(*)CC1]

wherein the saturated carbon is attached to A⁴, wherein A² is substituted with 0 or 1 occurrence of R³. In certain embodiments, A² is

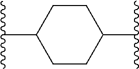 [SMILES: *C1CCC(*)CC1]

which is substituted with 0 or 1 occurrence of R³. In certain embodiments, A² is

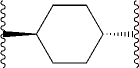 [SMILES: *[C@H]1CC[C@H](*)CC1]

which is substituted with 0 or 1 occurrence of R³. In certain embodiments, A² is

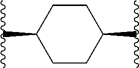 [SMILES: *[C@H]1CC[C@@H](*)CC1]

which is substituted with 0 or 1 occurrence of R³. In certain embodiments, A² contains 0 occurrences of R³.

In certain embodiments, A² is selected from the groups depicted in Table 1, below.

As generally defined above, A⁴ is a 5-6 membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 2 or 3 heteroatoms independently selected from nitrogen, oxygen, and sulfur, wherein the heteroaryl is substituted with x occurrences of R⁴.

In certain embodiments, A⁴ is triazolyl, oxadiazolyl, thiadiazolyl, pyrazolyl, imidazolyl, oxazolyl, or thiazolyl, each of which is substituted with x occurrences of R⁴. In certain embodiments, A⁴ is triazolyl, oxadiazolyl, or thiadiazolyl, each of which is substituted with x occurrences of R⁴. In certain embodiments, A⁴ is pyrazolyl, imidazolyl, oxazolyl, or thiazolyl, each of which is substituted with x occurrences of R⁴. In certain embodiments, A⁴ is 1,2,3-triazolyl or 1,2,4-oxadiazolyl substituted with x occurrences of R⁴. In certain embodiments, A⁴ is a 6-membered heteroaryl containing 1, 2, 3, or 4 heteroatoms that are nitrogen, wherein the heteroaryl is substituted with x occurrences of R⁴. In certain embodiments, A⁴ is a 6-membered heteroaryl containing 1 or 2 heteroatoms that are nitrogen, wherein the heteroaryl is substituted with x occurrences of R⁴.

In certain embodiments, A⁴ is selected from the groups depicted in Table 1, below.

As generally defined above, R^{1B} represents independently for each occurrence hydrogen or C₁₋₄ alkyl. In certain embodiments, R^{1B} is hydrogen. In certain embodiments, R^{1B} is selected from the groups depicted in Table 1, below.

As generally defined above, R² represents independently for each occurrence halo or C₁₋₄ haloalkyl. In certain embodiments, R² represents independently for each occurrence halo. In certain embodiments, R² is fluoro. In certain embodiments, R² is selected from the groups depicted in Table 1, below.

As generally defined above, R³ represents independently for each occurrence halo or C₁₋₄ alkyl. In certain embodiments, R³ is selected from the groups depicted in Table 1, below.

As generally defined above, R⁴ represents independently for each occurrence C₁₋₆ hydroxyalkyl, C₁₋₆ hydroxyhalo-alkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur). In certain embodiments, R⁴ represents independently for each occurrence C₁₋₆ hydroxyalkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ hydroxyhaloalkyl, or C₁₋₆ haloalkyl. In certain embodiments, R⁴ represents independently for each occurrence C₁₋₆ hydroxyalkyl. In certain embodiments, R⁴ represents independently for each occurrence C₁₋₆ alkyl. In certain embodiments, R⁴ represents independently for each occurrence C₁₋₆ alkylene substituted with 1 occurrence of —N(R^{1B})—C(O)R⁵ or —N(R^{1B})—SO₂R⁵.

In certain embodiments, R⁴ represents independently for each occurrence C₁₋₆ hydroxyhaloalkyl or C₁₋₆ haloalkyl. In certain embodiments, R⁴ represents independently for each occurrence —(C₀₋₃ alkylene)-(a monovalent 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent 5-7 membered ring containing 0 heteroatoms). In certain embodiments, R⁴ represents independently for each occurrence —(C₁ 3 alkylene)-(a monovalent 5-7 membered ring containing 1 or 2 heteroatoms independently selected from nitrogen, oxygen, and sulfur).

In certain embodiments, R⁴ represents independently for each occurrence —(C₁₋₃ alkylene)-(a monovalent, saturated 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, saturated 5-7 membered ring containing 0 heteroatoms). In certain embodiments, R⁴ represents independently for each occurrence —(C₁₋₃ alkylene)-(a monovalent, saturated 5-7 membered ring containing 1 or 2 heteroatoms independently selected from nitrogen, oxygen, and sulfur).

In certain embodiments, R⁴ represents independently for each occurrence —(C₁₋₃ alkylene)-(a monovalent, partially saturated 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, partially saturated 5-7 membered ring containing 0 heteroatoms). In certain embodiments, R⁴ represents independently for each occurrence —(C₁₋₃ alkylene)-(a monovalent, partially saturated 5-7 membered ring containing 1 or 2 heteroatoms independently selected from nitrogen, oxygen, and sulfur).

In certain embodiments, R⁴ represents independently for each occurrence —(C₁₋₃ alkylene)-(a monovalent, aromatic 5-7 membered ring containing 0, 1, or 2 heteroatoms independently selected from nitrogen, oxygen, and sulfur). In certain embodiments, R⁴ represents independently for each occurrence —(C₁₋₃ alkylene)-(a monovalent, aromatic 5-7 membered ring containing 0 heteroatoms). In certain embodiments, R⁴ represents independently for each occurrence —(C₁₋₃ alkylene)-(a monovalent, aromatic 5-7 membered ring containing 1 or 2 heteroatoms independently selected from nitrogen, oxygen, and sulfur). In certain embodiments, R⁴ represents independently for each occurrence a monovalent 5-7 membered ring containing 0, 1, or 2 heteroatoms independently selected from nitrogen, oxygen, and sulfur. In certain embodiments, R⁴ represents independently for each occurrence a monovalent 5-7 membered ring containing 0 heteroatoms. In certain embodiments, R⁴ represents independently for each occurrence a monovalent 5-7 membered ring containing 1 or 2 heteroatoms independently selected from nitrogen, oxygen, and sulfur.

In certain embodiments, R⁴ represents independently for each occurrence a monovalent, saturated 5-7 membered ring containing 0, 1, or 2 heteroatoms independently selected from nitrogen, oxygen, and sulfur. In certain embodiments, R⁴ represents independently for each occurrence a monovalent, saturated 5-7 membered ring containing 0 heteroatoms. In certain embodiments, R⁴ represents independently for each occurrence a monovalent, saturated 5-7 membered ring containing 1 or 2 heteroatoms independently selected from nitrogen, oxygen, and sulfur. In certain embodiments, R⁴ represents independently for each occurrence a monovalent, partially saturated 5-7 membered ring containing 0, 1, or 2 heteroatoms independently selected from nitrogen, oxygen, and sulfur. In certain embodiments, R⁴ represents independently for each occurrence a monovalent, partially saturated 5-7 membered ring containing 0 heteroatoms. In certain embodiments, R⁴ represents independently for each occurrence a monovalent, partially saturated 5-7 membered ring containing 1 or 2 heteroatoms independently selected from nitrogen, oxygen, and sulfur.

In certain embodiments, R⁴ represents independently for each occurrence a monovalent, aromatic 5-7 membered ring containing 0, 1, or 2 heteroatoms independently selected from nitrogen, oxygen, and sulfur. In certain embodiments, R⁴ represents independently for each occurrence a monovalent, aromatic 5-7 membered ring containing 0 heteroatoms. In certain embodiments, R⁴ represents independently for each occurrence a monovalent, aromatic 5-7 membered ring containing 1 or 2 heteroatoms independently selected from nitrogen, oxygen, and sulfur. In certain embodiments, R⁴ represents independently for each occurrence

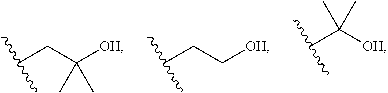

methyl, or

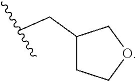 [SMILES: *CC1CCOC1]

In certain embodiments, R⁴ is selected from the groups depicted in Table 1, below.

As generally defined above, R⁵ represents independently for each occurrence C₁₋₄ alkyl or —(C₀₋₃ alkylene)-(C₃₋₇ cycloalkyl). In certain embodiments, R⁵ represents independently for each occurrence C₁₋₄ alkyl. In certain embodiments, R⁵ is selected from the groups depicted in Table 1, below.

As generally defined above, p is 1 or 2. In certain embodiments, p is 1. In certain embodiments, p is 2.

As generally defined above, x is 0, 1, or 2. In certain embodiments, x is 0. In certain embodiments, x is 1. In certain embodiments, x is 2.

### Exemplary Specific Compounds

In certain embodiments, the compound is a compound in Table 1 or a pharmaceutically acceptable salt thereof. In certain embodiments, the compound is a compound in Table 1. In certain embodiments, the compound is any one of compounds I-1 to I-17 in Table 1 or a pharmaceutically acceptable salt thereof. In certain embodiments, the compound is any one of compounds I-1 to I-17 in Table 1.

TABLE 1
Exemplary Compounds

| Compound | |
|---|---|
| No. | Chemical Structure |
| I-1 | 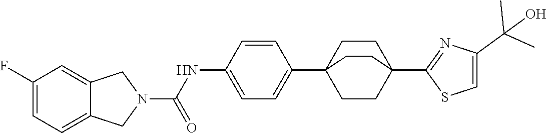 [SMILES: CC(C)(O)c1csc(C23CCC(c4ccc(NC(=O)N5Cc6ccc(F)cc6C5)cc4)(CC2)CC3)n1] |
| I-2 | 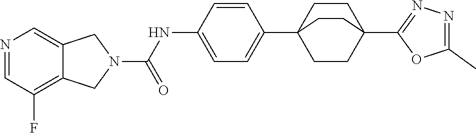 [SMILES: Cc1nnc(C23CCC(c4ccc(NC(=O)N5Cc6cncc(F)c6C5)cc4)(CC2)CC3)o1] |
| I-3 | 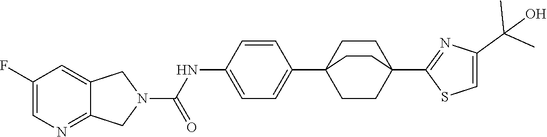 [SMILES: CC(C)(O)c1csc(C23CCC(c4ccc(NC(=O)N5Cc6cc(F)cnc6C5)cc4)(CC2)CC3)n1] |
| I-4 | 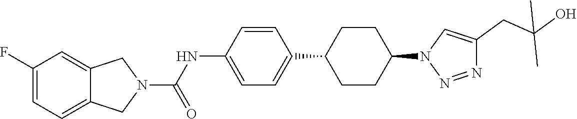 [SMILES: CC(C)(O)Cc1cn([C@H]2CC[C@H](c3ccc(NC(=O)N4Cc5ccc(F)cc5C4)cc3)CC2)nn1] |
| I-5 | 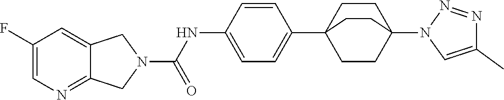 [SMILES: Cc1cn(C23CCC(c4ccc(NC(=O)N5Cc6cc(F)cnc6C5)cc4)(CC2)CC3)nn1] |
| I-6 | 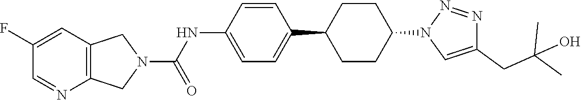 [SMILES: CC(C)(O)Cc1cn([C@H]2CC[C@H](c3ccc(NC(=O)N4Cc5cc(F)cnc5C4)cc3)CC2)nn1] |
| I-7 | 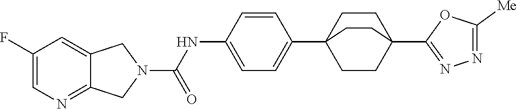 [SMILES: Cc1nnc(C23CCC(c4ccc(NC(=O)N5Cc6cc(F)cnc6C5)cc4)(CC2)CC3)o1] |
| I-8 | 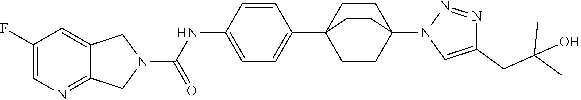 [SMILES: CC(C)(O)Cc1cn(C23CCC(c4ccc(NC(=O)N5Cc6cc(F)cnc6C5)cc4)(CC2)CC3)nn1] |
| I-9 | 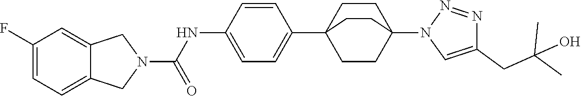 [SMILES: CC(C)(O)Cc1cn(C23CCC(c4ccc(NC(=O)N5Cc6ccc(F)cc6C5)cc4)(CC2)CC3)nn1] |
| I-10 | 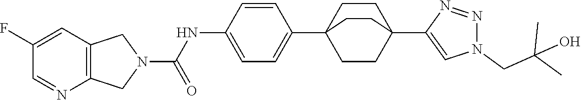 [SMILES: CC(C)(O)Cn1cc(C23CCC(c4ccc(NC(=O)N5Cc6cc(F)cnc6C5)cc4)(CC2)CC3)nn1] |
| I-11 | 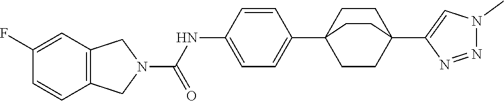 [SMILES: Cn1cc(C23CCC(c4ccc(NC(=O)N5Cc6ccc(F)cc6C5)cc4)(CC2)CC3)nn1] |
| I-12 | 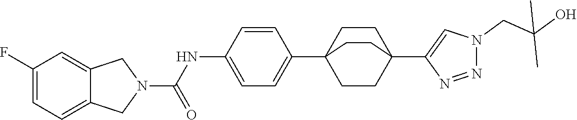 [SMILES: CC(C)(O)Cn1cc(C23CCC(c4ccc(NC(=O)N5Cc6ccc(F)cc6C5)cc4)(CC2)CC3)nn1] |
| I-13 | 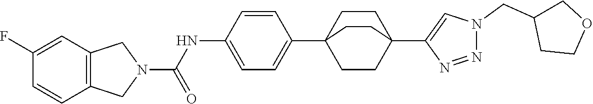 [SMILES: O=C(Nc1ccc(C23CCC(c4cn(CC5CCOC5)nn4)(CC2)CC3)cc1)N1Cc2ccc(F)cc2C1] |
| I-14 | 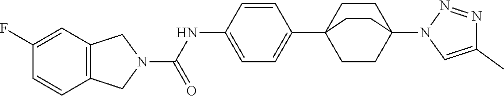 [SMILES: Cc1cn(C23CCC(c4ccc(NC(=O)N5Cc6ccc(F)cc6C5)cc4)(CC2)CC3)nn1] |
| I-15 | 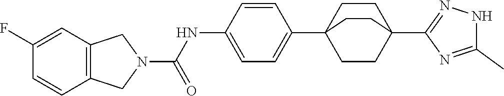 [SMILES: Cc1nc(C23CCC(c4ccc(NC(=O)N5Cc6ccc(F)cc6C5)cc4)(CC2)CC3)n[nH]1] |
| I-16 | 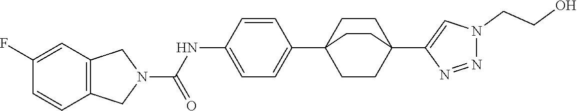 [SMILES: O=C(Nc1ccc(C23CCC(c4cn(CCO)nn4)(CC2)CC3)cc1)N1Cc2ccc(F)cc2C1] |
| I-17 | 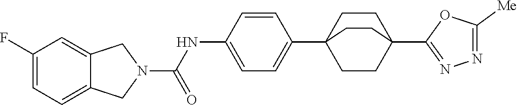 [SMILES: Cc1nnc(C23CCC(c4ccc(NC(=O)N5Cc6ccc(F)cc6C5)cc4)(CC2)CC3)o1] |
| I-18 | 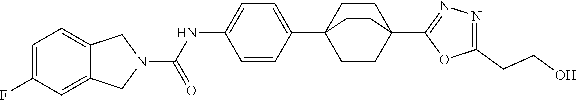 [SMILES: O=C(Nc1ccc(C23CCC(c4nnc(CCO)o4)(CC2)CC3)cc1)N1Cc2ccc(F)cc2C1] |
| I-19 | 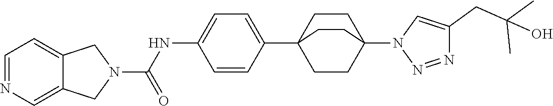 [SMILES: CC(C)(O)Cc1cn(C23CCC(c4ccc(NC(=O)N5Cc6ccncc6C5)cc4)(CC2)CC3)nn1] |
| I-20 | 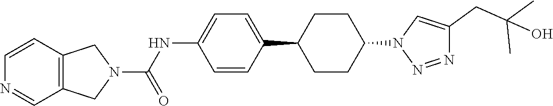 [SMILES: CC(C)(O)Cc1cn([C@H]2CC[C@H](c3ccc(NC(=O)N4Cc5ccncc5C4)cc3)CC2)nn1] |
| I-21 | 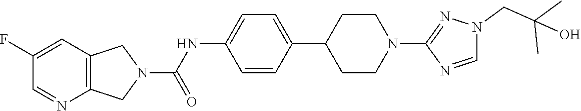 [SMILES: CC(C)(O)Cn1cnc(N2CCC(c3ccc(NC(=O)N4Cc5cc(F)cnc5C4)cc3)CC2)n1] |
| I-22 | 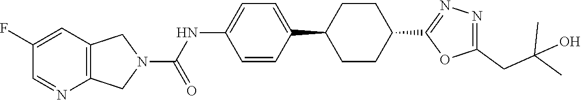 [SMILES: CC(C)(O)Cc1nnc([C@H]2CC[C@H](c3ccc(NC(=O)N4Cc5cc(F)cnc5C4)cc3)CC2)o1] |
| I-23 | 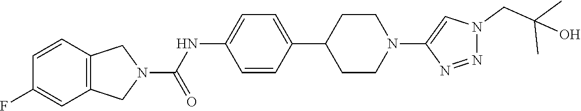 [SMILES: CC(C)(O)Cn1cc(N2CCC(c3ccc(NC(=O)N4Cc5ccc(F)cc5C4)cc3)CC2)nn1] |
| I-24 | 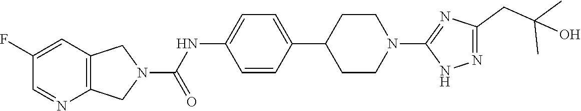 [SMILES: CC(C)(O)Cc1n[nH]c(N2CCC(c3ccc(NC(=O)N4Cc5cc(F)cnc5C4)cc3)CC2)n1] |
| I-25 | 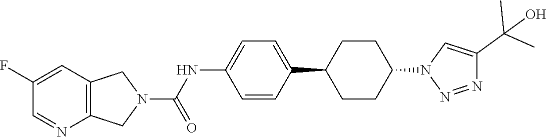 [SMILES: CC(C)(O)c1cn([C@H]2CC[C@H](c3ccc(NC(=O)N4Cc5cc(F)cnc5C4)cc3)CC2)nn1] |
| I-26 | 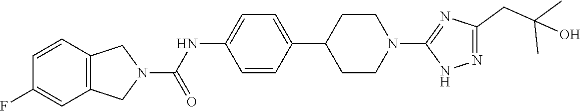 [SMILES: CC(C)(O)Cc1n[nH]c(N2CCC(c3ccc(NC(=O)N4Cc5ccc(F)cc5C4)cc3)CC2)n1] |
| I-27 | 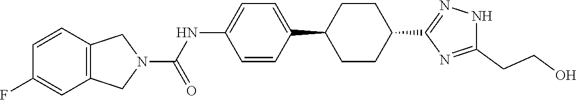 [SMILES: O=C(Nc1ccc([C@H]2CC[C@H](c3n[nH]c(CCO)n3)CC2)cc1)N1Cc2ccc(F)cc2C1] |
| I-28 | 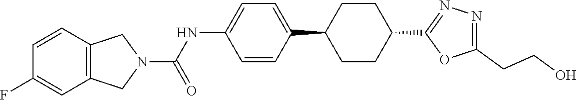 [SMILES: O=C(Nc1ccc([C@H]2CC[C@H](c3nnc(CCO)o3)CC2)cc1)N1Cc2ccc(F)cc2C1] |
| I-29 | 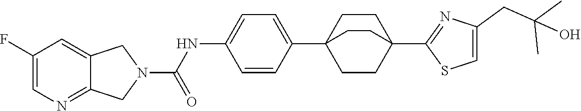 [SMILES: CC(C)(O)Cc1csc(C23CCC(c4ccc(NC(=O)N5Cc6cc(F)cnc6C5)cc4)(CC2)CC3)n1] |
| I-30 | 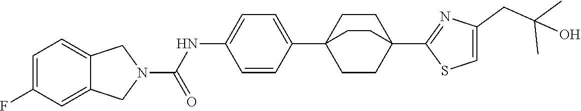 [SMILES: CC(C)(O)Cc1csc(C23CCC(c4ccc(NC(=O)N5Cc6ccc(F)cc6C5)cc4)(CC2)CC3)n1] |
| I-31 | 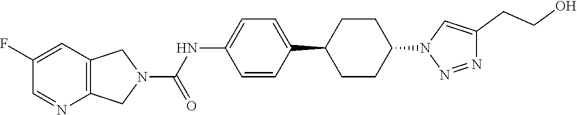 [SMILES: O=C(Nc1ccc([C@H]2CC[C@H](n3cc(CCO)nn3)CC2)cc1)N1Cc2cc(F)cnc2C1] |
| I-32 | 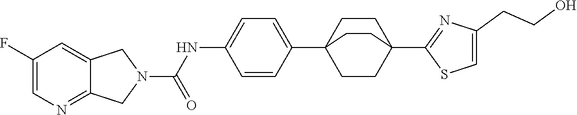 [SMILES: O=C(Nc1ccc(C23CCC(c4nc(CCO)cs4)(CC2)CC3)cc1)N1Cc2cc(F)cnc2C1] |
| I-33 | 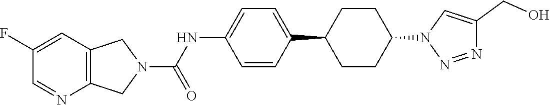 [SMILES: O=C(Nc1ccc([C@H]2CC[C@H](n3cc(CO)nn3)CC2)cc1)N1Cc2cc(F)cnc2C1] |
| I-34 | 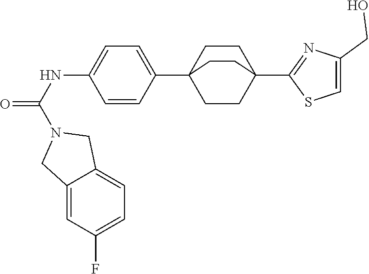 [SMILES: O=C(Nc1ccc(C23CCC(c4nc(CO)cs4)(CC2)CC3)cc1)N1Cc2ccc(F)cc2C1] |
| I-35 | 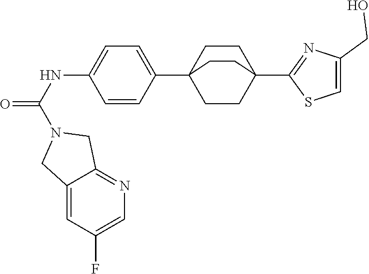 [SMILES: O=C(Nc1ccc(C23CCC(c4nc(CO)cs4)(CC2)CC3)cc1)N1Cc2cc(F)cnc2C1] |
| I-36 | 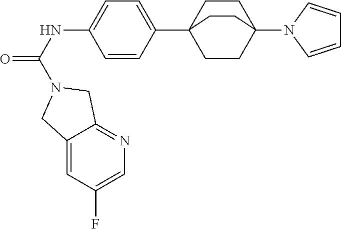 [SMILES: O=C(Nc1ccc(C23CCC(n4cccc4)(CC2)CC3)cc1)N1Cc2cc(F)cnc2C1] |
| I-37 | 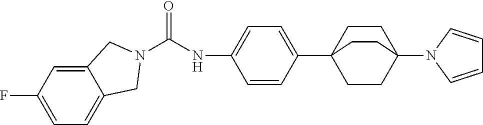 [SMILES: O=C(Nc1ccc(C23CCC(n4cccc4)(CC2)CC3)cc1)N1Cc2ccc(F)cc2C1] |
| I-38 | 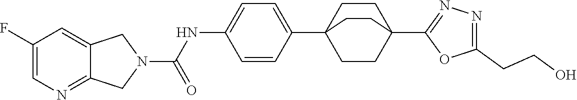 [SMILES: O=C(Nc1ccc(C23CCC(c4nnc(CCO)o4)(CC2)CC3)cc1)N1Cc2cc(F)cnc2C1] |
| I-39 | 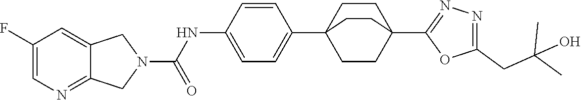 [SMILES: CC(C)(O)Cc1nnc(C23CCC(c4ccc(NC(=O)N5Cc6cc(F)cnc6C5)cc4)(CC2)CC3)o1] |
| I-40 | 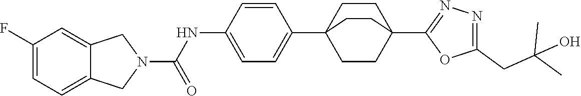 [SMILES: CC(C)(O)Cc1nnc(C23CCC(c4ccc(NC(=O)N5Cc6ccc(F)cc6C5)cc4)(CC2)CC3)o1] |

Another aspect provides a compound represented by Formula II:

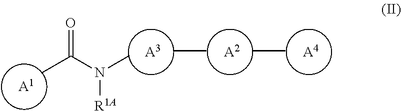

or a pharmaceutically acceptable salt thereof, wherein:
A¹ is

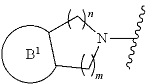

substituted with p occurrences of R², wherein B¹ is a 6-membered aryl ring or a 5-6 membered heteroaryl ring containing 1 or 2 heteroatoms independently selected from nitrogen and sulfur;
A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³;
A³ is phenylene substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1A} and R^{1B} each represent independently for each occurrence hydrogen or C₁₋₄ alkyl;
R² represents independently for each occurrence halo, C₁₋₄ haloalkyl, C₁₋₄ alkoxyl, C₁₋₄ alkyl, or C₁₋₄ hydroxyalkyl;
R³ represents independently for each occurrence halo or C₁₋₄ alkyl;
R⁴ represents independently for each occurrence —(C₀₋₆ alkylene)-CO₂R⁶; —(C₀₋₆ alkylene)-C(O)N(R^{1B})—(C₁₋₆ hydroxyalkyl), or —(C₀₋₄ alkylene)-COR⁵;
R⁵ represents independently for each occurrence C₁₋₄ alkyl or —(C₀₋₃ alkylene)-(C₃₋₇ cycloalkyl);
R⁶ represents independently for each occurrence hydrogen, C₁₋₆ alkyl or —(C₀₋₃ alkylene)-(C₃₋₇ cycloalkyl);
m and n are independently 1 or 2;
p is 0, 1, 2, 3, or 4; and
x is 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, A¹ is

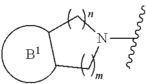

substituted with p occurrences of R², wherein B¹ is a 6-membered aryl ring or a 5-6 membered heteroaryl ring containing 1 or 2 heteroatoms independently selected from nitrogen and sulfur.

In certain embodiments, A¹ is

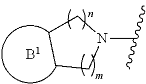

substituted with p occurrences of R², wherein B¹ is a 6-membered aryl ring or a 6-membered heteroaryl ring containing 1 or 2 heteroatoms that are nitrogen.

In certain embodiments, wherein A¹ is

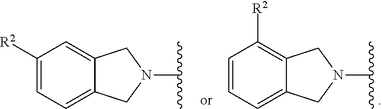

In certain embodiments, A¹ is

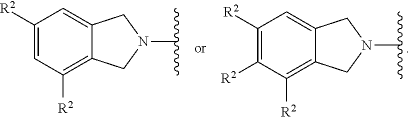

In certain embodiments, A¹ is

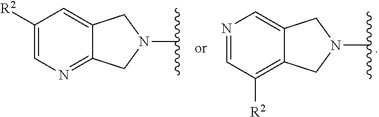

In certain embodiments A¹ is

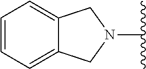 [SMILES: *N1Cc2ccccc2C1]

substituted with p occurrences of R². In certain embodiments, A¹ is

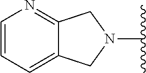 [SMILES: *N1Cc2cccnc2C1]

substituted with p occurrences of R².

In certain embodiments, A¹ is selected from the groups depicted in Table 2, below.

As generally defined above, A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³.

In certain embodiments, A² is a 5-8 membered saturated bridged bicyclic carbocyclylene, a 6-membered saturated or partially unsaturated monocyclic carbocyclylene, or a 6-membered saturated or partially unsaturated monocyclic heterocyclylene containing 1 heteroatom that is nitrogen; each of which is substituted with 0 or 1 occurrence of R³.

In certain embodiments, A² is an 8-membered saturated bridged bicyclic carbocyclylene substituted with 0 or 1 occurrence of R³. In certain embodiments, A² is

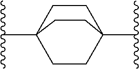 [SMILES: *C12CCC(*)(CC1)CC2]

substituted with 0 or 1 occurrence of R³.

In certain embodiments, A² is

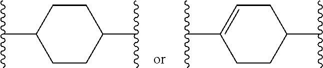 [SMILES: *C1CCC(*)CC1 ; *C1=CCC(*)CC1]

wherein the saturated carbon is attached to A⁴, and wherein

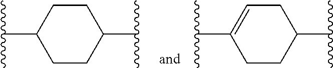 [SMILES: *C1CCC(*)CC1 ; *C1=CCC(*)CC1]

are substituted with 0 or 1 occurrence of R³. In certain embodiments, A² contains 0 occurrences of R³.

In certain embodiments, A² is

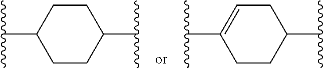 [SMILES: *C1CCC(*)CC1 ; *C1=CCC(*)CC1]

wherein the saturated carbon is attached to A⁴, wherein A² is substituted with 0 or 1 occurrence of R³. In certain embodiments, A² is

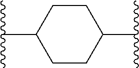 [SMILES: *C1CCC(*)CC1]

which is substituted with 0 or 1 occurrence of R³. In certain embodiments, A² is

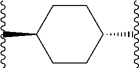 [SMILES: *[C@H]1CC[C@H](*)CC1]

which is substituted with 0 or 1 occurrence of R³. In certain embodiments, A² is

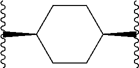 [SMILES: *[C@H]1CC[C@@H](*)CC1]

which is substituted with 0 or 1 occurrence of R³. In certain embodiments, A² is substituted with 0 occurrences of R³.

In certain embodiments, A² is selected from the groups depicted in Table 1, below.

As generally defined above, A³ is phenylene substituted with 0 or 1 occurrence of R³. In certain embodiments, A³ is para-phenylene substituted with 0 or 1 occurrence of R³. In certain embodiments, A³ is selected from the groups depicted in Table 2, below.

As generally defined above, A⁴ is a 5-6 membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 2 or 3 heteroatoms independently selected from nitrogen, oxygen, and sulfur, wherein the heteroaryl is substituted with x occurrences of R⁴.

In certain embodiments, A⁴ is triazolyl, oxadiazolyl, thiadiazolyl, pyrazolyl, imidazolyl, oxazolyl, or thiazolyl, each of which is substituted with x occurrences of R⁴. In certain embodiments, A⁴ is triazolyl, oxadiazolyl, or thiadiazolyl, each of which is substituted with x occurrences of R⁴. In certain embodiments, A⁴ is pyrazolyl, imidazolyl, oxazolyl, or thiazolyl, each of which is substituted with x occurrences of R⁴. In certain embodiments, A⁴ is 1,2,3-triazolyl; 1,2,4-oxadiazolyl; or pyrrolyl substituted with x occurrences of R⁴. In certain embodiments, A⁴ is a 6-membered heteroaryl containing 1, 2, 3, or 4 heteroatoms that are nitrogen, wherein the heteroaryl is substituted with x occurrences of R⁴. In certain embodiments, A⁴ is a 6-membered heteroaryl containing 1 or 2 heteroatoms that are nitrogen, wherein the heteroaryl is substituted with x occurrences of R⁴.

In certain embodiments, A⁴ is selected from the groups depicted in Table 2, below.

As generally defined above, R^{1A} and R^{1B} each represent independently for each occurrence hydrogen or C₁₋₄ alkyl.

In certain embodiments, R^{1B} is hydrogen.

In certain embodiments, R^{1A} is selected from the groups depicted in Table 1, below. In certain embodiments, R^{1B} is selected from the groups depicted in Table 1, below.

As generally defined above, R² represents independently for each occurrence halo, C₁₋₄ haloalkyl, C₁₋₄ alkoxyl, C₁₋₄ alkyl, or C₁₋₄ hydroxyalkyl. In certain embodiments, R² represents independently for each occurrence halo. In certain embodiments, R² is fluoro. In certain embodiments, R² is selected from the groups depicted in Table 2, below.

As generally defined above, R³ represents independently for each occurrence halo or C₁₋₄ alkyl. In certain embodiments, R³ is selected from the groups depicted in Table 2, below.

As generally defined above, R⁴ represents independently for each occurrence —(C₀₋₆ alkylene)-CO₂R⁶; —(C₀₋₆ alkylene)-C(O)N(RB)—(C₁₋₆ hydroxyalkyl), or —(C₀₋₄ alkylene)-COR⁵. In certain embodiments, R⁴ is —(C₀₋₆ alkylene)-CO₂R⁶. In certain embodiments, R⁴ is —(C₀₋₆ alkylene)-C(O)N(R^{1B})—(C₁₋₆ hydroxyalkyl). In certain embodiments, R⁴ is —(C₀₋₄ alkylene)-COR⁵. In certain embodiments, R⁴ is selected from the groups depicted in Table 2, below.

As generally defined above, R³ represents independently for each occurrence C₁₋₄ alkyl or —(C₀₋₃ alkylene)-(C₃₋₇ cycloalkyl). In certain embodiments, R⁵ represents independently for each occurrence C₁₋₄ alkyl. In certain embodiments, R⁵ is selected from the groups depicted in Table 2, below.

As generally defined above, R⁶ represents independently for each occurrence hydrogen, C₁₋₆ alkyl or —(C₀₋₃ alkylene)-(C₃₋₇ cycloalkyl). In certain embodiments, R⁶ is hydrogen. In certain embodiments, R⁶ is C₁₋₆ alkyl. In certain embodiments, R⁶ is —(C₀₋₃ alkylene)-(C₃₋₇ cycloalkyl). In certain embodiments, R⁶ is selected from the groups depicted in Table 2, below.

As generally defined above, m and n are independently 1 or 2. In certain embodiments, m and n are 1. In certain embodiments, m is 1. In certain embodiments, m is 2. In certain embodiments, n is 1. In certain embodiments, n is 2.

As generally defined above, p is 0, 1, 2, 3, or 4. In certain embodiments, p is 1, 2, or 3. In certain embodiments, p is 0. In certain embodiments, p is 1. In certain embodiments, p is 2. In certain embodiments, p is 3. In certain embodiments, p is 4.

As generally defined above, x is 0, 1, or 2. In certain embodiments, x is 0. In certain embodiments, x is 1. In certain embodiments, x is 2.

In certain embodiments, the compound is represented by Formula II-A:

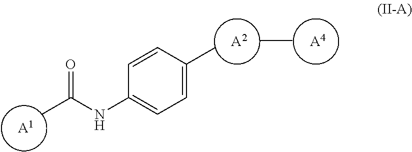

or a pharmaceutically acceptable salt thereof, wherein:
A¹ is

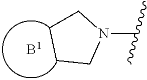 [SMILES: *N1CCCC1]

substituted with p occurrences of R², wherein B¹ is a 6-membered aryl ring or a 6-membered heteroaryl ring containing 1 or 2 heteroatoms that are nitrogen;
A² is a 5-8 membered saturated bridged bicyclic carbocyclylene or a 6-membered saturated or partially unsaturated monocyclic carbocyclylene; each of which is substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1B} represents independently for each occurrence hydrogen or C₁₋₄ alkyl;
R² represents independently for each occurrence halo or C₁₋₄ haloalkyl;
R³ is halo or C₁₋₄ alkyl;
R⁴ represents independently for each occurrence —(C₀₋₆ alkylene)-CO₂R⁶; —(C₀₋₆ alkylene)-C(O)N(R^{1B})—(C₁₋₆ hydroxyalkyl), or —(C₀₋₄ alkylene)-COR⁵;
R⁵ represents independently for each occurrence C₁₋₄ alkyl or —(C₀₋₃ alkylene)-(C₃₋₇ cycloalkyl);
R⁶ represents independently for each occurrence hydrogen, C₁₋₆ alkyl or —(C₀₋₃ alkylene)-(C₃₋₇ cycloalkyl);
p is 1 or 2; and
x is 0, 1, or 2.

The description above describes multiple embodiments relating to compounds of Formula II-A. The patent application specifically contemplates all combinations of the embodiments.

The definitions of variables in Formula II-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II-A.

As defined generally above, A¹ is

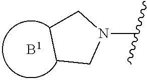 [SMILES: *N1CCCC1]

substituted with p occurrences of R², wherein B¹ is a 6-membered aryl ring or a 6-membered heteroaryl ring containing 1 or 2 heteroatoms that are nitrogen.

In certain embodiments, wherein A¹ is

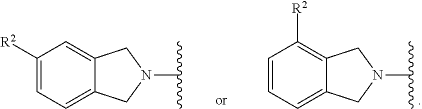

In certain embodiments, A¹ is

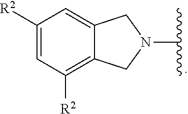

In certain embodiments, A¹ is

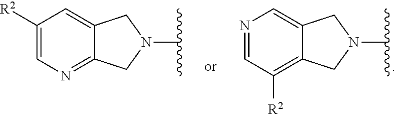

In certain embodiments, A¹ is

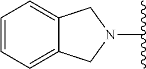 [SMILES: *N1Cc2ccccc2C1]

substituted with p occurrences of R². In certain embodiments, A¹ is

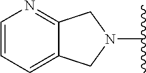 [SMILES: *N1Cc2cccnc2C1]

substituted with p occurrences of R².

In certain embodiments, A¹ is selected from the groups depicted in Table 2, below.

As generally defined above, A² is a 5-8 membered saturated bridged bicyclic carbocyclylene or a 6-membered saturated or partially unsaturated monocyclic carbocyclylene; each of which is substituted with 0 or 1 occurrence of R³.

In certain embodiments, A² is an 8-membered saturated bridged bicyclic carbocyclylene substituted with 0 or 1 occurrence of R³. In certain embodiments, A² is

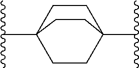 [SMILES: *C12CCC(*)(CC1)CC2]

substituted with 0 or 1 occurrence of R³.

In certain embodiments, A² is

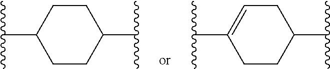 [SMILES: *C1CCC(*)CC1 ; *C1=CCC(*)CC1]

wherein the saturated carbon is attached to A⁴, wherein A² is substituted with 0 or 1 occurrence of R³. In certain embodiments, A² is

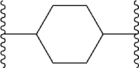 [SMILES: *C1CCC(*)CC1]

which is substituted with 0 or 1 occurrence of R³. In certain embodiments, A² is

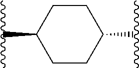 [SMILES: *[C@H]1CC[C@H](*)CC1]

which is substituted with 0 or 1 occurrence of R³. In certain embodiments, A² is

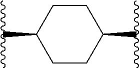 [SMILES: *[C@H]1CC[C@@H](*)CC1]

which is substituted with 0 or 1 occurrence of R³. In certain embodiments, A² is substituted with 0 occurrences of R³.

In certain embodiments, A² is selected from the groups depicted in Table 2, below.

As generally defined above, A⁴ is a 5-6 membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 1, 2, 3, or 4 heteroatoms independently selected from nitrogen, oxygen, and sulfur, wherein the heteroaryl is substituted with x occurrences of R⁴. In certain embodiments, A⁴ is a 5-membered heteroaryl containing 2 or 3 heteroatoms independently selected from nitrogen, oxygen, and sulfur, wherein the heteroaryl is substituted with x occurrences of R⁴.

In certain embodiments, A⁴ is triazolyl, oxadiazolyl, thiadiazolyl, pyrazolyl, imidazolyl, oxazolyl, or thiazolyl, each of which is substituted with x occurrences of R⁴. In certain embodiments, A⁴ is triazolyl, oxadiazolyl, or thiadiazolyl, each of which is substituted with x occurrences of R⁴. In certain embodiments, A⁴ is pyrazolyl, imidazolyl, oxazolyl, or thiazolyl, each of which is substituted with x occurrences of R⁴. In certain embodiments, A⁴ is 1,2,3-triazolyl; 1,2,4-oxadiazolyl; or pyrrolyl substituted with x occurrences of R⁴. In certain embodiments, A⁴ is a 6-membered heteroaryl containing 1, 2, 3, or 4 heteroatoms that are nitrogen, wherein the heteroaryl is substituted with x occurrences of R⁴. In certain embodiments, A⁴ is a 6-membered heteroaryl containing 1 or 2 heteroatoms that are nitrogen, wherein the heteroaryl is substituted with x occurrences of R⁴.

In certain embodiments, A⁴ is selected from the groups depicted in Table 2, below.

As generally defined above, R^{1B} represents independently for each occurrence hydrogen or C₁₋₄ alkyl. In certain embodiments, R^{1B} is hydrogen. In certain embodiments, R^{1B} is selected from the groups depicted in Table 2, below.

As generally defined above, R² represents independently for each occurrence halo or C₁₋₄ haloalkyl. In certain embodiments, R² represents independently for each occurrence halo. In certain embodiments, R² is fluoro. In certain embodiments, R² is selected from the groups depicted in Table 2, below.

As generally defined above, R³ represents independently for each occurrence halo or C₁₋₄ alkyl. In certain embodiments, R³ is selected from the groups depicted in Table 2, below.

As generally defined above, R⁴ represents independently for each occurrence —(C₀₋₆ alkylene)-CO₂R⁶; —(C₀₋₆ alkylene)-C(O)N(R^{1B})—(C₁₋₆ hydroxyalkyl), or —(C₀₋₄ alkylene)-COR⁵. In certain embodiments, R⁴ is —(C₀₋₆ alkylene)-CO₂R⁶. In certain embodiments, R⁴ is —(C₀₋₆ alkylene)-C(O)N(R^{1B})—(C₁₋₆ hydroxyalkyl). In certain embodiments, R⁴ is —(C₀₋₄ alkylene)-CO⁵R⁵. In certain embodiments, R⁴ is selected from the groups depicted in Table 2, below.

As generally defined above, R⁵ represents independently for each occurrence C₁₋₄ alkyl or —(C₀₋₃ alkylene)-(C₃₋₇ cycloalkyl). In certain embodiments, R⁵ represents independently for each occurrence C₁₋₄ alkyl. In certain embodiments, R⁵ is selected from the groups depicted in Table 1, below.

As generally defined above, R⁶ represents independently for each occurrence hydrogen, C₁₋₆ alkyl or —(C₀₋₃ alkylene)-(C₃₋₇ cycloalkyl). In certain embodiments, R⁶ is hydrogen. In certain embodiments, R⁶ is C₁₋₆ alkyl. In certain embodiments, R⁶ is —(C₀₋₃ alkylene)-(C₃₋₇ cycloalkyl). In certain embodiments, R⁶ is selected from the groups depicted in Table 2, below.

As generally defined above, p is 1 or 2. In certain embodiments, p is 1. In certain embodiments, p is 2.

As generally defined above, x is 0, 1, or 2. In certain embodiments, x is 0. In certain embodiments, x is 1. In certain embodiments, x is 2.

### Exemplary Specific Compounds

In certain embodiments, the compound is a compound in Table 2 or a pharmaceutically acceptable salt thereof. In certain embodiments, the compound is a compound in Table 2.

TABLE 2
Exemplary Compounds

| Compound | |
|---|---|
| No. | Chemical Structure |
| II-1 | 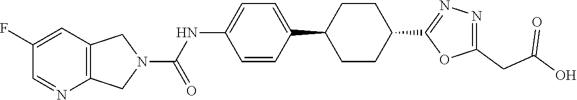 [SMILES: O=C(O)Cc1nnc([C@H]2CC[C@H](c3ccc(NC(=O)N4Cc5cc(F)cnc5C4)cc3)CC2)o1] |
| II-2 | 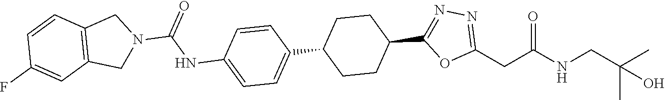 [SMILES: CC(C)(O)CNC(=O)Cc1nnc([C@H]2CC[C@H](c3ccc(NC(=O)N4Cc5ccc(F)cc5C4)cc3)CC2)o1] |
| II-3 | 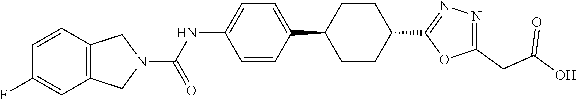 [SMILES: O=C(O)Cc1nnc([C@H]2CC[C@H](c3ccc(NC(=O)N4Cc5ccc(F)cc5C4)cc3)CC2)o1] |
| II-4 | 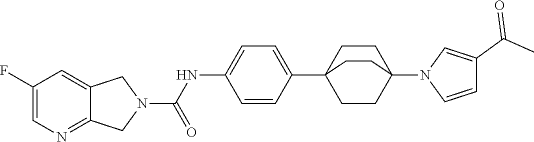 [SMILES: CC(=O)c1ccn(C23CCC(c4ccc(NC(=O)N5Cc6cc(F)cnc6C5)cc4)(CC2)CC3)c1] |
| II-5 | 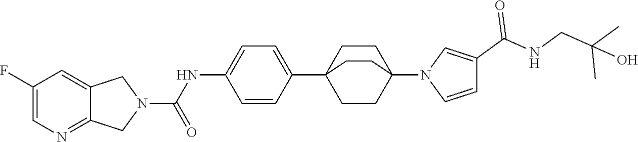 [SMILES: CC(C)(O)CNC(=O)c1ccn(C23CCC(c4ccc(NC(=O)N5Cc6cc(F)cnc6C5)cc4)(CC2)CC3)c1] |
| II-6 | 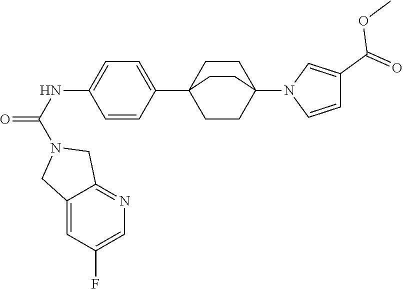 [SMILES: COC(=O)c1ccn(C23CCC(c4ccc(NC(=O)N5Cc6cc(F)cnc6C5)cc4)(CC2)CC3)c1] |

### Synthetic Methods

Methods for preparing compounds described herein are illustrated in the following synthetic Schemes. The Schemes are given for illustrative purposes, and are not intended to limit the scope or spirit of the disclosure. Starting materials shown in the Scheme can be obtained from commercial sources or can be prepared based on procedures described in the literature.

Scheme 1 illustrates a general method for preparing compound H. Protected aniline A is coupled with cyclic alkyl halide B (where X is an appropriate leaving group, such as bromide) using, for example, Friedel-Crafts Alkylation to afford compound C. Compound C is deprotected to afford the corresponding free aniline D. Free aniline D is reacted with an acyl chloride to form carbamate E. Carbamate E is then reacted with heteroaryl compound F to form substituted ester G. Substituted ester G is then elaborated to form compound H using methods known to one of skill in the art. Depending on the identity of A⁴, different methods may be used. For example, if A⁴ is a 1,2,3-triazole, substituted ester G may be converted to the corresponding alkyne (by, for example, reduction to the corresponding aldehyde, followed by Seyferth-Gilbert homologation) and reacted with a suitable azide by cycloaddition. Other conventional methodologies may also be employed for the conversion of substituted ester G into compound H. The modular synthetic route illustrated in Scheme 1 can be readily modified by one of skill in the art to alter the order of introducing the phenylene group, Ring A², Ring A⁴, and heterocyclic group F.

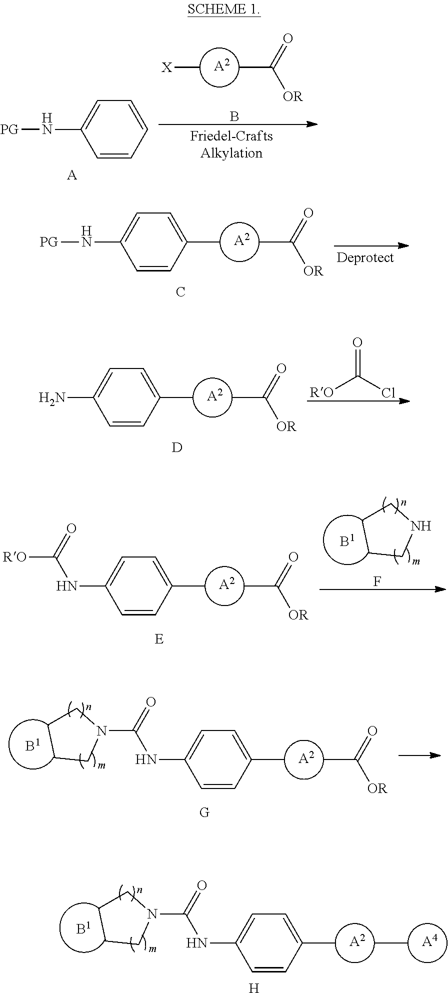

Scheme 2 illustrates a general method for preparing compound H. Transition-metal-mediated coupling of nitrobenzene A (where X is an appropriate leaving group, such as bromide, iodide, or sulfate) with coupling partner B (where [M] is an appropriate metal, such as a boronic acid, boronate ester, organotin, or zinc reagent) affords compound C. Compound C is reduced to afford the corresponding aniline D. Aniline D is reacted with an acyl chloride to form carbamate E. Carbamate E is then reacted with heteroaryl compound F to form compound G. Compound G is then elaborated to form compound H using methods known to one of skill in the art. Depending on the identity of A⁴, different methods may be used. For example, if A⁴ is a 1,2,3-triazole, compound G may be converted to the corresponding azide (by, for example, deprotection and reaction with TsN₃) and reacted with a suitable alkyne by cycloaddition. Other conventional methodologies may also be employed for the conversion of compound G into compound H. The modular synthetic route illustrated in Scheme 2 can be readily modified by one of skill in the art to alter the order of introducing the phenylene group, Ring A², Ring A⁴, and heterocyclic group F.

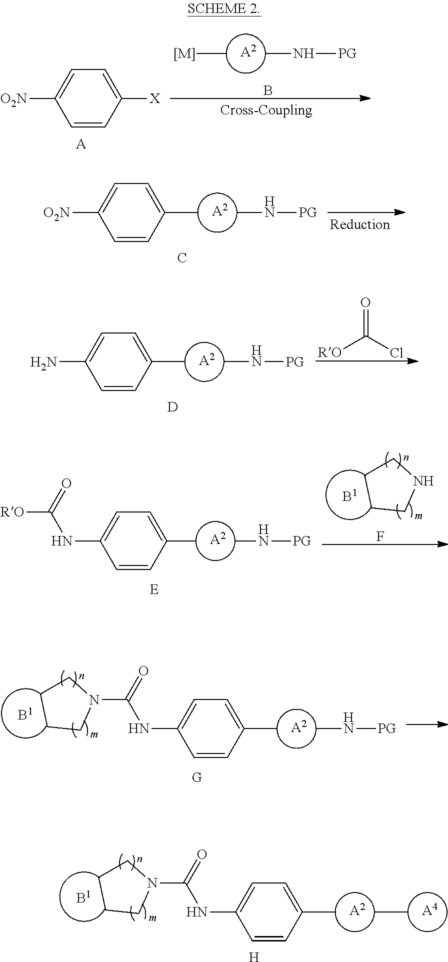

In Scheme 1 and 2, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in Schemes 1 and 2 can also be readily modified by one of skill in the art to provide additional compounds by conducting functional group transformations on the intermediate and final compounds. Such functional group transformations are well known in the art, as described in, for example, “Comprehensive Organic Synthesis” (B. M. Trost & I. Fleming, eds., 1991-1992).

### II. Therapeutic Applications

The heteroaryl compounds described herein, such as a compound of Formula I, or other compounds in Section I, provide therapeutic benefits to subjects suffering a disease or condition mediated by NAMPT. Description of exemplary therapeutic applications are provided herein below.

One aspect provides a method of treating a disease or condition mediated by NAMPT. The method comprises administering to a subject in need thereof a therapeutically effective amount of a compound described herein, such as a compound of Formula I, to treat the disease or condition.

Another aspect provides a method of treating a disease or condition described herein. The method comprises administering to a subject in need thereof a therapeutically effective amount of a compound described herein, such as a compound of Formula I, to treat the disease or condition.

Another aspect provides a method of inhibiting the activity of NAMPT. The method comprises contacting a NAMPT with an effective amount of a compound described herein, such as a compound of Formula I, to inhibit the activity of said NAMPT.

In certain embodiments, the disease or condition is selected from a proliferative disorder (e.g., cancer, neoplasia), inflammatory disorder (e.g., chronic inflammatory disorder, acute inflammatory disorder, auto-inflammatory disorder), autoimmune disorder, fibrotic disorder, metabolic disorder, cardiovascular disorder, cerebrovascular disorder, and myeloid cell-driven hyper-inflammatory response in a COVID-19 infection.

In certain embodiments, the disease or condition mediated by NAMPT is a NAPRT-negative cancer. In certain embodiments, the disease or condition mediated by NAMPT is psoriasis, dermatitis, vitiligo, ichthyosis, alopecia areata, epidermolysis bullosa, hidradenitis suppurativa, chronic obstructive pulmonary disease (COPD), rheumatoid arthritis, psoriatic arthritis, systemic lupus erythematosus, or kidney disease. In certain embodiments, the disease or condition mediated by NAMPT is inflammatory bowel disease.

In certain embodiments, the heteroaryl compounds described herein, such as a compound of Formula I, or other compounds in Section I, provide therapeutic benefits to subjects suffering from cancer (e.g., pancreatic cancer, melanoma, glioma, lung cancer, colon cancer, rectal cancer, breast cancer, cervical cancer, prostate cancer, gastric cancer, skin cancer, liver cancer, bile duct cancer, nervous system cancer, lymphoma, leukemia), intestinal or inflammatory bowel disease, diseases of overactive T-cells, psoriasis, neurodegenerative diseases, acute kidney disease, inflammation, stroke, acute infarct, inflammatory bowel disease, acute lung injury, rheumatoid arthritis, nephropathy, fibrosis, or sepsis.

Another aspect provides for the use of a compound described herein (such as a compound of Formula I, or other compounds in Section I) in the manufacture of a medicament. In certain embodiments, the medicament is for treating a disease or condition described herein, such as a proliferative disorder, inflammatory disorder, or metabolic disorder.

Another aspect provides for the use of a compound described herein (such as a compound of Formula I, or other compounds in Section I) for treating a disease or condition, such as a disease or condition described herein.

Another aspect provides a pharmaceutical composition comprising a compound described herein (such as a compound of Formula I, or other compounds in Section I) for treating a disease or condition, such as a disease or condition described herein.

In certain embodiments, the subject is a human. In certain embodiments, the subject is an adult human. In certain embodiments, the subject is a pediatric human. In certain embodiments, the subject is a geriatric human.

### Exemplary Diseases or Conditions

Exemplary diseases and disorders include but are not limited to those described herein. In certain embodiments, the disease or condition is a proliferative disorder (e.g., cancer, neoplasia), an inflammatory disorder (e.g., chronic inflammatory disorder, acute inflammatory disorder, auto-inflammatory disorder), an autoimmune disorder, a fibrotic disorder, a metabolic disorder, a cardiovascular disorder, a cerebrovascular disorder, or a myeloid cell-driven hyper-inflammatory response in COVID-19 infections. In certain embodiments, the proliferative disease is cancer. In certain embodiments, the proliferative disease is pancreatic cancer, melanoma, glioma, lung cancer, colon cancer, rectal cancer, breast cancer, cervical cancer, prostate cancer, gastric cancer, skin cancer, liver cancer, bile duct cancer, nervous system cancer, lymphoma, leukemia, or a combination thereof. In certain embodiments, the disease or condition is an inflammatory disorder (e.g., intestinal or inflammatory bowel disease, diseases of overactive T-cells, psoriasis, neurodegenerative diseases, acute kidney disease, inflammation, stroke, acute infarct, inflammatory bowel disease, acute lung injury, rheumatoid arthritis, nephropathy, fibrosis, or sepsis. In certain embodiments, the disease or condition is a NAPRT-negative cancer. In certain embodiments, the disease or condition is psoriasis, dermatitis, vitiligo, ichthyosis, alopecia areata, epidermolysis bullosa, hidradenitis suppurativa, chronic obstructive pulmonary disease (COPD), rheumatoid arthritis, psoriatic arthritis, systemic lupus erythematosus, or kidney disease. In certain embodiments, the disease or condition is inflammatory bowel disease.

Exemplary proliferative diseases include but are not limited to those described herein. In certain embodiments, the proliferative disease or condition to be treated or prevented using the compounds described herein is cancer.

Additional exemplary cancers include but are not limited to acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); Burkitt lymphoma; primary intraocular lymphoma; classic Hodgkin lymphoma; biphenotypic acute leukemia; T cell lymphoma; nasal-type T cell lymphoma; enteropathy-type T-cell lymphoma; subcutaneous panniculitis-like T-cell lymphoma; blastic NK-cell lymphoma; T-cell prolymphocytic leukemia, and NK-cell leukemia.

In certain embodiments, the cancer is a hematological malignancy. Exemplary hematological malignancies include but are not limited to leukemia, such as acute lymphoblastic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), acute non-lymphocytic leukemia (ANLL), acute promyelocytic leukemia (APL), and acute myelomonocytic leukemia (AMMoL); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; multiple myeloma (MM); heavy chain disease (e.g., alpha chain disease, gamma chain disease, mu chain disease), polycythemia vera, Wilm's tumor, and Ewing's sarcoma.

In certain embodiments, said disease or condition is a cancer. In certain embodiments, said disease or condition is an angiogenesis disorder. In certain embodiments, said disease or condition is a multiple myeloma. In certain embodiments, said disease or condition is a leukemia (e.g., acute lymphocytic leukemia, acute and chronic myelogenous leukemia, chronic lymphocytic leukemia, acute lymphoblastic leukemia, chronic myelomonocytic leukemia, or promyelocytic leukemia).

In certain embodiments, the cancer is a solid tumor, leukemia, or lymphoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, or thyroid cancer.

In certain embodiments, said disease or condition is a lymphoma (e.g., B-cell lymphoma, T-cell lymphoma, mantle cell lymphoma, diffuse large B cell lymphoma, hairy cell lymphoma, Burkitt's lymphoma, mast cell tumors, Hodgkin's disease or non-Hodgkin's disease). In certain embodiments, said disease or condition is myelodysplastic syndrome. In certain embodiments, said disease or condition is fibrosarcoma. In certain embodiments, said disease or condition is rhabdomyosarcoma. In certain embodiments, said disease or condition is astrocytoma. In certain embodiments, said disease or condition is neuroblastoma. In certain embodiments, said disease or condition is glioma and schwannomas. In certain embodiments, said disease or condition is melanoma. In certain embodiments, said disease or condition is seminoma. In certain embodiments, said disease or condition is teratocarcinoma. In certain embodiments, said disease or condition is osteosarcoma. In certain embodiments, said disease or condition is xenoderma pigmentosum. In certain embodiments, said disease or condition is keratoctanthoma. In certain embodiments, said disease or condition is thyroid follicular cancer. In certain embodiments, said disease or condition is Kaposi's sarcoma. In certain embodiments, said disease or condition is melanoma. In certain embodiments, said disease or condition is teratoma. In certain embodiments, said disease or condition is rhabdomyosarcoma. In certain embodiments, said disease or condition is a metastatic and bone disorder. In certain embodiments, said disease or condition is cancer of the bone. In certain embodiments, said disease or condition is cancer of the mouth/pharynx. In certain embodiments, said disease or condition is cancer of the esophagus. In certain embodiments, said disease or condition is cancer of the larynx. In certain embodiments, said disease or condition is cancer of the stomach. In certain embodiments, said disease or condition is cancer of the intestine. In certain embodiments, said disease or condition is cancer of the colon. In certain embodiments, said disease or condition is cancer of the rectum. In certain embodiments, said disease or condition is cancer of the lung (e.g., non-small cell lung cancer or small cell lung cancer). In certain embodiments, said disease or condition is cancer of the liver. In certain embodiments, said disease or condition is cancer of the pancreas. In certain embodiments, said disease or condition is cancer of the nerve. In certain embodiments, said disease or condition is cancer of the brain (e.g., glioma or glioblastoma multiforme). In certain embodiments, said disease or condition is cancer of the head and neck. In certain embodiments, said disease or condition is cancer of the throat. In certain embodiments, said disease or condition is cancer of the ovary. In certain embodiments, said disease or condition is cancer of the uterus. In certain embodiments, said disease or condition is cancer of the prostate. In certain embodiments, said disease or condition is cancer of the testis. In certain embodiments, said disease or condition is cancer of the bladder. In certain embodiments, said disease or condition is cancer of the kidney. In certain embodiments, said disease or condition is cancer of the breast. In certain embodiments, said disease or condition is cancer of the gall bladder. In certain embodiments, said disease or condition is cancer of the cervix. In certain embodiments, said disease or condition is cancer of the thyroid. In certain embodiments, said disease or condition is cancer of the prostate. In certain embodiments, said disease or condition is cancer of the skin (e.g., skin squamous cell carcinoma). In certain embodiments, said disease or condition is a solid tumor. In certain embodiments, said disease or condition is gastric cancer. In certain embodiments, said disease or condition is hepatocellular carcinoma. In certain embodiments, said disease or condition is a peripheral nerve sheath tumor. In certain embodiments, said disease or condition is pulmonary arterial hypertension.

In certain embodiments, the disease is a cancer associated with a viral infection. In certain embodiments, the disease is a cancer resulting from infection with an oncovirus. In certain embodiments, the oncovirus is hepatitis A, hepatitis B, hepatitis C, human T-lymphotropic virus (HTLV), human papillomavirus (HPV), Kaposi's sarcoma-associated herpesvirus (HHV-8), Merkel cell polyomavirus, or Epstein-Barr virus (EBV). In certain embodiments, the disease is human T-lymphotropic virus. In certain embodiments, the disease is Kaposi's sarcoma-associated herpesvirus. In certain embodiments, the disease is Epstein-Barr virus. Leukemias and lymphomas which may be associated with an oncoviral include: for HTLV, adult T-cell leukemia; for HHV-8, Castleman's disease and primary effusion lymphoma; and for EBV, Burkitt's lymphoma, Hodgkin's lymphoma, and post-transplant lymphoproliferative disease.

In certain embodiments, said disease or condition is an inflammatory disorder or allergic disorder. In certain embodiments, said disease or condition is an inflammatory disorder, such as autoimmune disorders, chronic inflammatory disorders, acute inflammatory disorders, auto-inflammatory disorders, fibrotic disorders, metabolic disorders, neoplasias, cardiovascular or cerebrovascular disorders, and myeloid cell-driven hyper-inflammatory response in COVID-19 infections. In certain embodiments, said disease or condition is an allergic disorder, such as asthma and allergic rhinitis.

In certain embodiments, said disease or condition is a disease or disorder of tissues and systemic disease [e.g., systemic lupus erythematosus (SLE); immune thrombocytopenic purpura (ITP); autoimmune hemolytic anemia (AHA); autoimmune neutropenia (AlN); Evans syndrome; proliferative and hyperproliferative diseases, such as cancer, atherosclerosis, rheumatoid arthritis, psoriasis, idiopathic pulmonary fibrosis, scleroderma, cirrhosis of the liver; and Acquired Immunodeficiency Syndrome (AIDS)]. In certain embodiments, said disease or condition is an immunologically-mediated disease, such as allograft rejection (e.g., rejection of transplanted organs or tissues). In certain embodiments, said disease or condition is a tissue injury (e.g., associated with organ transplant or revascularization procedures). In certain embodiments, said disease or condition is a disease or disorder of the respiratory tract (e.g., asthma). In certain embodiments, said disease or condition is allergic rhinitis. In certain embodiments, said disease or condition is a disease or disorder of the bone and joints (e.g., arthritis, rheumatoid arthritis). In certain embodiments, said disease or condition is a disease or disorder of the skin. In certain embodiments, said disease or condition is a disease or disorder of the gastrointestinal tract.

Exemplary diseases of the respiratory tract include but are not limited to those described herein. In certain embodiments, said disease or condition is a reversible obstructive airways disease, such as asthma (e.g., bronchial asthma, allergic asthma, intrinsic asthma, extrinsic asthma, and dust asthma). In certain embodiments, said disease or condition is chronic or inveterate asthma (e.g., late asthma airways hyper-responsiveness). In certain embodiments, said disease or condition is bronchitis. In certain embodiments, said disease or condition is a condition characterized by an inflammation of the nasal mucus membrane. In certain embodiments, said disease or condition is acute rhinitis. In certain embodiments, said disease or condition is allergic rhinitis. In certain embodiments, said disease or condition is atrophic rhinitis. In certain embodiments, said disease or condition is chronic rhinitis (e.g., rhinitis caseosa, hypertrophic rhinitis, rhinitis purulenta, rhinitis sicca, and rhinitis medicamentosa). In certain embodiments, said disease or condition is membranous rhinitis (e.g., croupous rhinitis, fibrinous rhinitis, pseudomembranous rhinitis, and scrofoulous rhinitis). In certain embodiments, said disease or condition is seasonal rhinitis [e.g., rhinitis nervosa (hay fever), vasomotor rhinitis, sarcoidosis, farmer's lung, and related diseases, such as fibroid lung and idiopathic interstitial pneumonia].

Exemplary diseases of the bone and joints include but are not limited to those described herein. In certain embodiments, said disease or condition includes pannus formation. In certain embodiments, said disease or condition does not include pannus formation. In certain embodiments, said disease or condition is rheumatoid arthritis. In certain embodiments, said disease or condition is seronegative spondyloarthropathis (e.g., ankylosing spondylitis, psoratic arthritis, and Reiter's disease). In certain embodiments, said disease or condition is Behcet's disease. In certain embodiments, said disease or condition is Sjogren's syndrome. In certain embodiments, said disease or condition is systemic sclerosis.

Exemplary diseases and disorders of the skin include but are not limited to those described herein. In certain embodiments, said disease or condition is psoriasis. In certain embodiments, said disease or condition is systemic sclerosis. In certain embodiments, said disease or condition is dermatitis. In certain embodiments, said disease or condition is atopical dermatitis. In certain embodiments, said disease or condition is contact dermatitis. In certain embodiments, said disease or condition is eczematous dermatitis. In certain embodiments, said disease or condition is seborrhoetic dermatitis. In certain embodiments, said disease or condition is Lichen planus. In certain embodiments, said disease or condition is Pemphigus. In certain embodiments, said disease or condition is bullous Pemphigus. In certain embodiments, said disease or condition is epidermolysis bullosa. In certain embodiments, said disease or condition is urticaria. In certain embodiments, said disease or condition is angiodermas. In certain embodiments, said disease or condition is vasculitides. In certain embodiments, said disease or condition is erythemas. In certain embodiments, said disease or condition is cutaneous eosinophilias. In certain embodiments, said disease or condition is uveitis. In certain embodiments, said disease or condition is alopecia. In certain embodiments, said disease or condition is areata. In certain embodiments, said disease or condition is alopecia areata. In certain embodiments, said disease or condition is vernal conjunctivitis.

Exemplary diseases and disorders of the gastrointestinal tract include but are not limited to those described herein. In certain embodiments, said disease or condition is Coeliac disease. In certain embodiments, said disease or condition is proctitis. In certain embodiments, said disease or condition is eosinophilic gastro-enteritis. In certain embodiments, said disease or condition is mastocytosis. In certain embodiments, said disease or condition is pancreatitis. In certain embodiments, said disease or condition is Crohn's disease. In certain embodiments, said disease or condition is ulcerative colitis. In certain embodiments, said disease or condition is a food-related allergy having effects remote from the gut (e.g., migraine, rhinitis, and eczema).

Exemplary diseases and disorders of other tissues and systemic disease include but are not limited to those described herein. In certain embodiments, said disease or condition is multiple sclerosis. In certain embodiments, said disease or condition is atherosclerosis. In certain embodiments, said disease or condition is acquired immunodeficiency syndrome (AIDS). In certain embodiments, said disease or condition is lupus. In certain embodiments, said disease or condition is lupus erythematosus. In certain embodiments, said disease or condition is systemic lupus erythematosus. In certain embodiments, said disease or condition is Hashimoto's thyroiditis. In certain embodiments, said disease or condition is myasthenia gravis. In certain embodiments, said disease or condition is type I diabetes. In certain embodiments, said disease or condition is nephrotic syndrome. In certain embodiments, said disease or condition is eosinophilia fasciitis. In certain embodiments, said disease or condition is hyper IgE syndrome. In certain embodiments, said disease or condition is lepromatous leprosy. In certain embodiments, said disease or condition is sezary syndrome. In certain embodiments, said disease or condition is idiopathic thrombocytopenia purpura. In certain embodiments, said disease or condition is restenosis following angioplasty. In certain embodiments, said disease or condition is a tumor (e.g., leukemia, lymphomas). In certain embodiments, said disease or condition is atherosclerosis.

Exemplary allograft rejections include but are not limited to those described herein. In certain embodiments, said disease or condition is acute chronic allograft rejection (e.g., following transplantation of kidney, heart, liver, lung, bone marrow, skin, or cornea). In certain embodiments, said disease or condition is chronic allograft rejection (e.g., following transplantation of kidney, heart, liver, lung, bone marrow, skin, or cornea). In certain embodiments, said disease or condition is chronic graft-versus-host disease.

In certain embodiments, said disease or condition is an acute inflammatory disorder. In certain embodiments, said disease or condition is an auto-inflammatory disorder. In certain embodiments, said disease or condition is a fibrotic disorder. In certain embodiments, said disease or condition is a metabolic disorder. In certain embodiments, said disease or condition is a neoplasia. In certain embodiments, said disease or condition is a cardiovascular or cerebrovascular disorder. In certain embodiments, said disease or condition is a myeloid cell-driven hyper-inflammatory response in COVID-19 infections.

In certain embodiments, said disease or condition is an autoimmune disorder. In certain embodiments, said disease or condition is a chronic inflammatory disorder. In certain embodiments, said disease or condition is an acute inflammatory disorder. In certain embodiments, said disease or condition is an auto-inflammatory disorder. In certain embodiments, said disease or condition is a combination of one, two, or all three of a chronic inflammatory disorder, an acute inflammatory disorder, and an auto-inflammatory disorder.

Exemplary autoimmune and/or inflammatory and/or auto-inflammatory disorders include but are not limited to those described herein. In certain embodiments, said disease or condition is an inflammatory bowel disease (e.g., ulcerative colitis or Crohn's disease). In certain embodiments, said disease or condition is multiple sclerosis. In certain embodiments, said disease or condition is psoriasis. In certain embodiments, said disease or condition is arthritis. In certain embodiments, said disease or condition is rheumatoid arthritis. In certain embodiments, said disease or condition is osteoarthritis. In certain embodiments, said disease or condition is juvenile arthritis. In certain embodiments, said disease or condition is psoriatic arthritis. In certain embodiments, said disease or condition is reactive arthritis. In certain embodiments, said disease or condition is ankylosing spondylitis. In certain embodiments, said disease or condition is cryopyrin-associated periodic syndromes. In certain embodiments, said disease or condition is Muckle-Wells syndrome. In certain embodiments, said disease or condition is familial cold auto-inflammatory syndrome. In certain embodiments, said disease or condition is neonatal-onset multisystem inflammatory disease. In certain embodiments, said disease or condition is TNF receptor-associated periodic syndrome. In certain embodiments, said disease or condition is acute and chronic pancreatitis. In certain embodiments, said disease or condition is atherosclerosis. In certain embodiments, said disease or condition is gout. In certain embodiments, said disease or condition is a fibrotic disorder (e.g., hepatic fibrosis or idiopathic pulmonary fibrosis). In certain embodiments, said disease or condition is nephropathy. In certain embodiments, said disease or condition is sarcoidosis. In certain embodiments, said disease or condition is scleroderma. In certain embodiments, said disease or condition is anaphylaxis. In certain embodiments, said disease or condition is diabetes (e.g., diabetes mellitus type 1 or diabetes mellitus type 2). In certain embodiments, said disease or condition is diabetic retinopathy. In certain embodiments, said disease or condition is Still's disease. In certain embodiments, said disease or condition is vasculitis. In certain embodiments, said disease or condition is sarcoidosis. In certain embodiments, said disease or condition is pulmonary inflammation. In certain embodiments, said disease or condition is respiratory failure. In certain embodiments, said disease or condition is acute respiratory distress syndrome. In certain embodiments, said disease or condition is chronic eosinophilic pneumonia. In certain embodiments, said disease or condition is wet and dry age-related macular degeneration. In certain embodiments, said disease or condition is autoimmune hemolytic syndromes. In certain embodiments, said disease or condition is autoimmune and inflammatory hepatitis. In certain embodiments, said disease or condition is autoimmune neuropathy. In certain embodiments, said disease or condition is autoimmune ovarian failure. In certain embodiments, said disease or condition is autoimmune orchitis. In certain embodiments, said disease or condition is autoimmune thrombocytopenia. In certain embodiments, said disease or condition is silicone implant-associated autoimmune disease. In certain embodiments, said disease or condition is Sjogren's syndrome. In certain embodiments, said disease or condition is familial Mediterranean fever. In certain embodiments, said disease or condition is systemic lupus erythematosus. In certain embodiments, said disease or condition is vasculitis syndromes (e.g., temporal, Takayasu's and giant cell arteritis, Behcet's disease or Wegener's granulomatosis). In certain embodiments, said disease or condition is vitiligo. In certain embodiments, said disease or condition is ichthyosis. In certain embodiments, said disease or condition is secondary hematologic manifestation of autoimmune diseases (e.g., anemias). In certain embodiments, said disease or condition is drug-induced autoimmunity. In certain embodiments, said disease or condition is Hashimoto's thyroiditis. In certain embodiments, said disease or condition is hypophysitis. In certain embodiments, said disease or condition is idiopathic thrombocytic pupura. In certain embodiments, said disease or condition is metal-induced autoimmunity. In certain embodiments, said disease or condition is myasthenia gravis. In certain embodiments, said disease or condition is pemphigus. In certain embodiments, said disease or condition is autoimmune deafness (e.g., Meniere's disease). In certain embodiments, said disease or condition is Goodpasture's syndrome. In certain embodiments, said disease or condition is Graves' disease. In certain embodiments, said disease or condition is an HW-related autoimmune syndromes. In certain embodiments, said disease or condition is Guillain-Barre disease. In certain embodiments, said disease or condition is Addison's disease. In certain embodiments, said disease or condition is anti-phospholipid syndrome. In certain embodiments, said disease or condition is asthma. In certain embodiments, said disease or condition is atopic dermatitis. In certain embodiments, said disease or condition is Celiac disease. In certain embodiments, said disease or condition is Cushing's syndrome. In certain embodiments, said disease or condition is dermatomyositis. In certain embodiments, said disease or condition is idiopathic adrenal atrophy. In certain embodiments, said disease or condition is idiopathic thrombocytopenia. In certain embodiments, said disease or condition is Kawasaki syndrome. In certain embodiments, said disease or condition is Lambert-Eaton Syndrome. In certain embodiments, said disease or condition is pernicious anemia. In certain embodiments, said disease or condition is pollinosis. In certain embodiments, said disease or condition is polyarteritis nodosa. In certain embodiments, said disease or condition is primary biliary cirrhosis. In certain embodiments, said disease or condition is primary sclerosing cholangitis. In certain embodiments, said disease or condition is Raynaud's disease. In certain embodiments, said disease or condition is Raynaud's phenomenon. In certain embodiments, said disease or condition is Reiter's Syndrome. In certain embodiments, said disease or condition is relapsing polychondritis. In certain embodiments, said disease or condition is Schmidt's syndrome. In certain embodiments, said disease or condition is thyrotoxidosis. In certain embodiments, said disease or condition is sepsis. In certain embodiments, said disease or condition is septic shock. In certain embodiments, said disease or condition is endotoxic shock. In certain embodiments, said disease or condition is exotoxin-induced toxic shock. In certain embodiments, said disease or condition is gram negative sepsis. In certain embodiments, said disease or condition is toxic shock syndrome. In certain embodiments, said disease or condition is glomerulonephritis. In certain embodiments, said disease or condition is peritonitis. In certain embodiments, said disease or condition is interstitial cystitis. In certain embodiments, said disease or condition is hyperoxia-induced inflammations. In certain embodiments, said disease or condition is chronic obstructive pulmonary disease (COPD). In certain embodiments, said disease or condition is emphysema. In certain embodiments, said disease or condition is nasal inflammation. In certain embodiments, said disease or condition is vasculitis. In certain embodiments, said disease or condition is graft vs. host reaction (e.g., graft vs. host disease). In certain embodiments, said disease or condition is allograft rejections (e.g., acute allograft rejection or chronic allograft rejection). In certain embodiments, said disease or condition is early transplantation rejection (e.g., acute allograft rejection). In certain embodiments, said disease or condition is reperfusion injury. In certain embodiments, said disease or condition is pain (e.g., acute pain, chronic pain, neuropathic pain, or fibromyalgia). In certain embodiments, said disease or condition is a chronic infection. In certain embodiments, said disease or condition is meningitis. In certain embodiments, said disease or condition is encephalitis. In certain embodiments, said disease or condition is myocarditis. In certain embodiments, said disease or condition is gingivitis. In certain embodiments, said disease or condition is post-surgical trauma. In certain embodiments, said disease or condition is tissue injury. In certain embodiments, said disease or condition is traumatic brain injury. In certain embodiments, said disease or condition is enterocolitis. In certain embodiments, said disease or condition is sinusitis. In certain embodiments, said disease or condition is uveitis. In certain embodiments, said disease or condition is ocular inflammation. In certain embodiments, said disease or condition is optic neuritis. In certain embodiments, said disease or condition is gastric ulcers. In certain embodiments, said disease or condition is esophagitis. In certain embodiments, said disease or condition is peritonitis. In certain embodiments, said disease or condition is periodontitis. In certain embodiments, said disease or condition is dermatomyositis. In certain embodiments, said disease or condition is gastritis. In certain embodiments, said disease or condition is myositis. In certain embodiments, said disease or condition is polymyalgia. In certain embodiments, said disease or condition is pneumonia. In certain embodiments, said disease or condition is bronchitis. In certain embodiments, the disease or condition is endometriosis. In certain embodiments, the disease or condition is necrotizing vasculitis. In certain embodiments, the disease or condition is lymphadenitis. In certain embodiments, the disease or condition is peri-arteritis nodosa. In certain embodiments, the disease or condition is anti-phospholipid antibody syndrome. In certain embodiments, the disease or condition is pemphigus vulgaris. In certain embodiments, the disease or condition is Lyme disease. In certain embodiments, the disease or condition is cardiomyopathy. In certain embodiments, the disease or condition is rheumatic fever. In certain embodiments, the disease or condition is a blistering disorder. In certain embodiments, the disease or condition is an antibody-mediated vasculitis syndrome. In certain embodiments, the disease or condition is an immune-complex vasculitide. In certain embodiments, the disease or condition is oedema. In certain embodiments, the disease or condition is embolism. In certain embodiments, the disease or condition is fibrosis. In certain embodiments, the disease or condition is silicosis. In certain embodiments, the disease or condition is BENTA disease. In certain embodiments, the disease or condition is berylliosis.

Exemplary fibrotic disorders include but are not limited to those described herein. In certain embodiments, said disease or condition is systemic sclerosis/scleroderma. In certain embodiments, said disease or condition is lupus nephritis. In certain embodiments, said disease or condition is connective tissue disease. In certain embodiments, said disease or condition is wound healing. In certain embodiments, said disease or condition is surgical scarring. In certain embodiments, said disease or condition is spinal cord injury. In certain embodiments, said disease or condition is CNS scarring. In certain embodiments, said disease or condition is acute lung injury. In certain embodiments, said disease or condition is pulmonary fibrosis (e.g., idiopathic pulmonary fibrosis or cystic fibrosis). In certain embodiments, said disease or condition is chronic obstructive pulmonary disease. In certain embodiments, said disease or condition is adult respiratory distress syndrome. In certain embodiments, said disease or condition is acute lung injury. In certain embodiments, said disease or condition is drug-induced lung injury. In certain embodiments, said disease or condition is glomerulonephritis. In certain embodiments, said disease or condition is kidney disease. In certain embodiments, said disease or condition is chronic kidney disease (e.g., diabetic nephropathy). In certain embodiments, said disease or condition is hypertension-induced nephropathy. In certain embodiments, said disease or condition is alimentary track or gastrointestinal fibrosis. In certain embodiments, said disease or condition is renal fibrosis. In certain embodiments, said disease or condition is hepatic or biliary fibrosis. In certain embodiments, said disease or condition is liver fibrosis (e.g., nonalcoholic steatohepatitis, hepatitis C, or hepatocellular carcinoma). In certain embodiments, said disease or condition is cirrhosis (e.g., primary biliary cirrhosis or cirrhosis due to fatty liver disease, such as alcoholic and nonalcoholic steatosis). In certain embodiments, said disease or condition is radiation-induced fibrosis (e.g., head and neck, gastrointestinal or pulmonary). In certain embodiments, said disease or condition is primary sclerosing cholangitis. In certain embodiments, said disease or condition is restenosis. In certain embodiments, said disease or condition is cardiac fibrosis (e.g., endomyocardial fibrosis or atrial fibrosis). In certain embodiments, said disease or condition is ophthalmic scarring. In certain embodiments, said disease or condition is fibrosclerosis. In certain embodiments, said disease or condition is a fibrotic cancer. In certain embodiments, said disease or condition is fibroids. In certain embodiments, said disease or condition is fibroma. In certain embodiments, said disease or condition is a fibroadenoma. In certain embodiments, said disease or condition is a fibrosarcoma. In certain embodiments, said disease or condition is transplant arteriopathy. In certain embodiments, said disease or condition is keloid. In certain embodiments, said disease or condition is mediastinal fibrosis. In certain embodiments, said disease or condition is myelofibrosis. In certain embodiments, said disease or condition is retroperitoneal fibrosis. In certain embodiments, said disease or condition is progressive massive fibrosis. In certain embodiments, said disease or condition is nephrogenic systemic fibrosis.

Exemplary metabolic disorders include but are not limited to those described herein. In certain embodiments, said disease or condition is obesity. In certain embodiments, said disease or condition is steroid-resistance. In certain embodiments, said disease or condition is glucose intolerance. In certain embodiments, said disease or condition is metabolic syndrome.

Exemplary cardiovascular or cerebrovascular disorders include but are not limited to those described herein. In certain embodiments, said disease or condition is atherosclerosis. In certain embodiments, said disease or condition is restenosis of an atherosclerotic coronary artery. In certain embodiments, said disease or condition is acute coronary syndrome. In certain embodiments, said disease or condition is myocardial infarction. In certain embodiments, said disease or condition is cardiac-allograft vasculopathy. In certain embodiments, said disease or condition is stroke. In certain embodiments, said disease or condition is a central nervous system disorder with an inflammatory or apoptotic component. In certain embodiments, said disease or condition is Alzheimer's disease. In certain embodiments, said disease or condition is Parkinson's disease. In certain embodiments, said disease or condition is Huntington's disease. In certain embodiments, said disease or condition is amyotrophic lateral sclerosis. In certain embodiments, said disease or condition is spinal cord injury. In certain embodiments, said disease or condition is neuronal ischemia. In certain embodiments, said disease or condition is peripheral neuropathy.

In certain embodiments, said disease or condition is a disease or disorder associated with a coronavirus (e.g., SARS-CoV-2). In certain embodiments, said coronavirus is SARS-CoV-2. In certain embodiments, the disease or disorder associated with SARS-CoV-2 is COVID-19.

In certain embodiments, the disease or condition is a rheumatic disease. In certain embodiments, the disease or condition is an inflammatory arthropathy. In certain embodiments, the disease or condition is rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile Idiopathic Arthritis (JIA), juvenile psoriatic arthritis, juvenile rheumatoid arthritis, systemic onset juvenile rheumatoid arthritis, giant cell arteritis, secondary osteoarthritis from an inflammatory disease.

In certain embodiments, the disease or condition is a connective tissue disease. In certain embodiments, the disease or condition is lupus, systemic lupus erythematosus, juvenile systemic lupus erythematosus, nephritis, Sjogren's syndrome, scleroderma (systemic sclerosis), Raynaud's phenomenon, juvenile scleroderma, polymyositis, dermatomyositis, polymyositis-dermatomyositis, polymyalgia rheumatica, a mixed connective tissue disease, sarcoidosis, fibromyalgia, vasculitis microscopic polyangiitis, vasculitis, eosinophilic granulomatosis with polyangiitis (formerly known as Churg-Strauss Syndrome), granulomatosis with polyangiitis (formerly known as Wegener's granulomatosis), polyarteritis nodosa, Henoch-Schönlein purpura, idiopathic thrombocytopenic thrombotic purpura, juvenile vasculitis, polyarteritis nodossa (also known as panarteritis nodosa, periarteritis nodosa Kussmaul disease, Kussmaul-Maier disease or PAN), serum sickness, myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, primary biliary sclerosis, autoimmune hepatitis, chronic aggressive hepatitis, nonalcoholic hepatic steatosis, sclerosing cholangitis, membranous glomerulopathy, polymyositis, myositis, atherosclerosis, autoimmune hemolytic anemia, autoimmune orchitis, Goodpasture's disease,

In certain embodiments, the disease or condition is a neurodegenerative disease or neuroinflammatory disease. In certain embodiments, the disease or condition is multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder.

In certain embodiments, the disease or condition is an inflammatory bowel disease (IBD). In certain embodiments, the disease or condition is Crohn's disease, ulcerative colitis, Celiac Sprue, Celiac disease, proctitis, eosinophilic gastroenteritis, autoimmune atrophic gastritis of pernicious anemia, or mastocytosis.

In certain embodiments, the disease or condition is a skin autoimmune disorder. In certain embodiments, the disease or condition is psoriasis. In certain embodiments, the disease or condition is eczema. In certain embodiments, the disease or condition is plaque psoriasis, Guttate psoriasis, psoriatic epidermal hyperplasia, inverse psoriasis, pustular psoriasis, erythrodermic psoriasis, atopic dermatitis, eczema dermatitis, dermatitis, rosacea, pruritus, alopecia areata, vitiligo, epidermal hyperplasia, juvenile dermatomyositis, dermatomyositis, or hidradenitis suppurativa. In certain embodiments, said disease or condition is hidradenitis suppurativa.

In certain embodiments, the disease or condition is an organ or cell transplant rejection. In certain embodiments, the disease or condition is graft-versus-host disease. In certain embodiments, the disease or condition is chronic graft-versus-host disease, acute graft-versus-host disease, or organ or cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In certain embodiments, the disease or condition is an autoimmune disease of the eye. In certain embodiments, the disease or condition is Graves' disease, noninfectious uveitis, dry eye syndrome, sympathetic ophthalmia, Cogan's syndrome, keratoconjunctivitis, vernal conjunctivitis, uveitis (e.g., uveitis associated with Behcet's disease and lens-induced uveitis), keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, or ocular neovascularization

In certain embodiments, the disease or condition is an ocular manifestation of an autoimmune disease.

In certain embodiments, the disease or condition is a respiratory disease. In certain embodiments, the disease or condition is asthma, chronic obstructive pulmonary disease, or acute respiratory disease.

In certain embodiments, the disease or condition is diabetes. In certain embodiments, the disease or condition is Type I diabetes mellitus, Type II diabetes mellitus, or juvenile onset diabetes.

In certain embodiments, the disease or condition is thyroid cancer, duodenal, neuroendocrine carcinoma (NEC), uterine cancer, small cell lung cancer (SCLC), hepatocellular carcinoma, mesothelioma, breast cancer, sarcoma, ovarian cancer, renal cell carcinoma, rectal cancer, head and neck cancer, prostate cancer, pancreatic cancer, melanoma, colorectal cancer, cervix cancer, non-small cell lung cancer (NSCLC), cholangiocarcinoma, or endometrial cancer.

In certain embodiments, the disease or condition is an autoimmune disease or atherosclerosis.

In certain embodiments, the disease or condition is ovarian cancer, breast cancer, uterine cancer, colon cancer, cervical cancer, lung cancer, prostate cancer, skin cancer, bladder cancer, pancreatic cancer, leukemia, lymphoma, Hodgkin's disease, viral infections, Human Immunodeficiency Virus, hepatitis virus, herpes virus, herpes simplex, inflammatory disorders, irritable bowel syndrome, inflammatory bowel disease, rheumatoid arthritis, asthma, chronic obstructive pulmonary disease, osteoarthritis, osteoporosis, dermatitis, atopic dermatitis, psoriasis, systemic lupus erythematosus, multiple Sclerosis, psoriatic arthritis, ankylosing spondylitis, graft-versus-host disease, Alzheimer's disease, cerebrovascular accident, atherosclerosis, diabetes, glomerulonephritis, metabolic Syndrome, non-Small cell lung cancer, Small cell lung cancer, multiple myeloma, leukemias, lymphomas, squamous cell cancers, kidney cancer, urethral and bladder cancers, cancers of head and neck, cancers of the brain and central nervous system (CNS).

In certain embodiments, the disease or condition is cancer, an autoimmune disease, viral disease, fungal disease, neurological/neurodegenerative disorder, arthritis, inflammation, anti-proliferative (e.g., ocular retinopathy), neuronal, alopecia and cardiovascular disease. In certain embodiments, the disease or condition is a carcinoma, including that of the bladder, breast, colon, kidney, liver, lung, including Small cell lung cancer, non-small cell lung cancer, head and neck, esophagus, gallbladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoietic tumors of myeloid lineage, including acute and chronic myelogenous leukemias, myelodysplastic syndrome and pro myelocytic leukemia; tumors of mesenchymal origin, including fibrosarcoma and rhabdomyosarcoma; tumors of the central and peripheral nervous system, including astrocytoma, neuroblastoma, glioma and Schwannomas; and other tumors, including melanoma, seminoma, teratocarcinoma, osteosarcoma, Xeroderma pigmentosum, keratocanthoma, thyroid follicular cancer and Kaposi's sarcoma.

Without being bound by theory, there are multiple instances where decreasing NAD levels through NAMPT inhibition or in general have been associated with positive outcome in treating disease. For example, it has previously been shown that activated T-cells require increased NAD levels, and are more sensitive to NAMPTi than quiescent cells (Bruzzone et al. (2009) in PLoS ONE 4(11): e7897). Therefore NAMPT inhibitors show promise in immunosuppression of auto-immune disease, decreasing hyperactivation of the immune system in IBD (Gerner et al. in Gut 2018; 67:1813-1823), Psoriasis (Arroyo et al. in Int. J. Mol. Sci. 2023, 24(9), 7992), vitiligo (Becatti et al. in J Cell Mol Med. (2014) vol. 18(3): 514-529) and alopecia among others (Mercurio et al. in Int. J. Mol. Sci. 2021, 22(13), 6860; Martinez-Morcillo et al. (2021) in PLoS Biol 19(11): e3001455).

NAMPT expression has also been positively correlated with markers of inflammation, and NAMPTi decreases the expression of these same inflammation markers (Gerner et al. in Gut 2018; 67:1813-1823). Multiple mouse models of lung injury have demonstrated a significant benefit to treatment with NAMPT inhibitors (Geng-Chin Wu et al. in Respiratory Research 2017; 18(1):71; Moreno-Vinasco et al. in Am J Respir Cell Mol Biol. 2014 August; 51(2): 223-228). NAMPT is also found to be upregulated in COPD and negatively correlated with lung function (Huang Y, Niu Y, Wang X, Li X, He Y and Liu X (2024) in Identification of novel biomarkers related to neutrophilic inflammation in COPD. Front. Immunol.).

NAMPT knockdown has been shown to reduce atherosclerosis by promoting cholesterol efflux and macrophage RCT through the PPARa-LXRa-ABCA1/G1 pathway both in vitro and in vivo (Li et al. in Scientific Reports volume 6, Article number: 26746 (2016)).

In graft-versus-host disease (GVHD), NAMPT is highly expressed in both serum and T-cells. NAMPT inhibition strongly attenuates symptoms of GVHD in in vivo models and depletes NAD levels in activated T-cells (Gerner R R et al. in Leukemia 2020; 34(7):1885-1897). In rheumatoid arthritis, miR-23b is associated with RA where it is downregulated. It has been demonstrated to target NAMPT so NAMPT inhibitors are understood to provide a benefit in treating rheumatoid arthritis (Lv, R., Yu, J. & Sun, Q. in Future Oncol. (Lond. Engl.) 16(10), 541-458; and Liu, X. et al. in Gene 712(143911), 143911).

### Medical Uses

Another aspect provides for the use of a compound described herein (such as a compound of Formula I, or other compounds in Section I) in the manufacture of a medicament. In certain embodiments, the medicament is for treating a disorder described herein, such as cancer.

Another aspect provides for the use of a compound described herein (such as a compound of Formula I, or other compounds in Section I) for treating a medical disorder, such as a medical disorder described herein, such as cancer.

Compounds described herein may also be used as a component in an antibody-drug conjugate. For example, compounds described herein may be conjugated to an antibody to form an antibody-drug conjugate. The antibody-drug conjugate may be used to treat disorder and conditions described herein.

### III. Combination Therapy

Another aspect provides for combination therapy. Heteroaryl compounds described herein (e.g., a compound of Formula I, or other compounds in Section I) or their pharmaceutically acceptable salts may be used in combination with additional therapeutic agents to treat diseases or conditions, such as an inflammatory disorder.

Accordingly, in some embodiments, provided herein is a method of treating a disclosed disease or condition comprising administering to a patient in need thereof an effective amount of a compound disclosed herein and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents.

One or more other therapeutic agents may be administered separately from a compound or composition of the disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the disclosure may be administered simultaneously, sequentially or within a period of time from one another.

In certain embodiments, the compounds of the disclosure can be administered with one or more of an additional therapeutic agent, sequentially or concurrently, either by the same route or by different routes of administration. When administered sequentially, the time between administrations is selected to benefit, among others, the therapeutic efficacy and/or safety of the combination treatment. In certain embodiments, the compound of the disclosure can be administered first followed by a second therapeutic agent, or alternatively, the second therapeutic agent administered first followed by the compound of the disclosure. In certain embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the additional therapeutic agent, by the same or different routes, or administered in a single composition by the same route. In certain embodiments, the compound of the disclosure is prepared as a first pharmaceutical composition, and the additional therapeutic agent prepared as a second pharmaceutical composition, where the first pharmaceutical composition and the additional pharmaceutical composition are administered simultaneously, sequentially, or separately. In certain embodiments, the amount and frequency of administration of the additional therapeutic agent can used standard dosages and standard administration frequencies used for the particular therapeutic agent. See, e.g., Physicians' Desk Reference, 70th Ed., PDR Network, 2015; incorporated herein by reference.

In certain embodiments, the additional therapeutic agent is a leukotriene inhibitor, non-steroidal anti-inflammatory drug (NSAID), steroid, tyrosine kinase inhibitor, receptor kinase inhibitor, modulator of nuclear receptor family of transcription factor, HSP90 inhibitor, adenosine receptor (A₂A) agonist, disease modifying antirheumatic drugs (DMARDS), phosphodiesterase (PDE) inhibitor, neutrophil elastase inhibitor, modulator of Axl kinase, an anti-cancer agent, anti-allergic agent, anti-nausea agent (or anti-emetic), pain reliever, cytoprotective agent, anticoagulant, antiplatelet agent and dual antiplatelet therapy, angiotensin-converting enzyme (ACE) inhibitor, angiotensin II receptor blocker, angiotensin receptor-neprilysin inhibitor, beta blocker, combined alpha- and beta-blocker, calcium channel blocker, cholesterol-lowering medication (e.g., statins, nicotinic acids, cholesterol absorption inhibitors, combination statin and cholesterol absorption inhibitors), digitalis preparation, diuretic, vasodilator, antiarrhythmic agent (e.g., Class Ia, Class Ib, Class Ic, Class II, Class III, Class IV, Class V), or a combination thereof. In certain embodiments, the additional therapeutic agent is an anti-cancer agent, an analgesic, an anti-inflammatory agent, or a combination thereof.

In certain embodiments, the additional therapeutic agent is a leukotriene inhibitor. Examples of leukotriene inhibitors considered for use in combination therapies of the disclosure include but are not limited to montelukast, zafirlukast, pranlukast, zileuton, or combinations thereof.

In certain embodiments, the additional therapeutic agent is a NSAID. Examples of NSAIDs considered for use in combination therapies of the disclosure include but are not limited to acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib, or combinations thereof.

In certain embodiments, the additional therapeutic agent is a steroid. Examples of steroids considered for use in combination therapies of the disclosure include but are not limited to prednisone, prednisolone, methylprednisone, triacmcinolone, betamethasone, dexamethasone, and prodrugs thereof.

In certain embodiments, the additional therapeutic agent is a tyrosine kinase inhibitor. Examples of tyrosine kinase inhibitors considered for use in combination therapies of the disclosure include but are not limited to inhibitors of the following kinases, including, among others: JAK, Syk, JNK/SAPK, MAPK, PI-3K, and/or Ripk2. In certain embodiments, the tyrosine kinase inhibitor is ruxolitinib, tofacitinib, oclactinib, filgotinib, ganotinib, lestaurtinib, momelotinib, pacritinib, upadacitinib, peficitinib, fedratinib, bentamapimod, D-JNKI-1 (XG-102, AM-111), ponatinib, WEHI-345, OD36, GSK583, idelalisib, copanlisib, taselisib, duvelisib, alpelisib, umbralisib, dactolisib, CUDC-907, entospletinib, fostamatinib, or combinations thereof.

In certain embodiments, the additional therapeutic agent is a receptor kinase inhibitor, including among others, an inhibitor of EGFR or HER2. Examples of receptor kinase inhibitors considered for use in combination therapies of the disclosure include but are not limited to gefitinib, erlotinib, neratinib, lapatinib, cetuximab, panitumumab, vandetanib, necitumumab, osimertinib, trastuzumab, neratinib, lapatinib, pertuzumab, or combinations thereof.

In certain embodiments, the additional therapeutic agent is a modulator of nuclear receptor family of transcription factors, including, among others, an inhibitor of PPAR, RXR, FXR, or LXR. In certain embodiments, the inhibitor is pioglitazone, bexarotene, obeticholic acid, ursodeoxycholic acid, fexaramine, hypocholamide, or combinations thereof.

In certain embodiments, the additional therapeutic agent is an HSP90 inhibitor. Examples of HSP90 inhibitors considered for use in combination therapies of the disclosure include but are not limited to ganetespib, 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010, or combinations thereof.

In certain embodiments, the additional therapeutic agent is an adenosine receptor 2A (A₂A) agonist. Examples of adenosine receptor agonists considered for use in combination therapies of the disclosure include but are not limited to those disclosed in U.S. Pat. No. 9,067,963, which is incorporated herein by reference. In certain embodiments, the adenosine receptor agonist is LNC-3050, LNC-3015, LNC-3047, LNC-3052, or combinations thereof.

In certain embodiments, the additional therapeutic agent is selected from disease modifying antirheumatic drugs (DMARDS). Examples of DMARDS considered for use in combination therapies of the disclosure include but are not limited to tocilizumab, certolizumab, etanercept, adalimumab, anakinra, abatacept, infliximab, rituximab, golimumab, uteskinumab, or combinations thereof.

In certain embodiments, the additional therapeutic agent is a phosphodiesterase (PDE) inhibitor. Examples of phosphodiesterase inhibitor considered for use in combination therapies of the disclosure include but are not limited to apremilast, crisaborole, piclimilast, drotaverine, ibudulast, roflumilast, sildenafil, tadalafil, vardenafil, or combinations thereof.

In certain embodiments, the additional therapeutic agent is a neutrophil elastase inhibitor. Examples of neutrophil elastase inhibitors considered for use in combination therapies of the disclosure include but are not limited to sivelestat.

In certain embodiments, the additional therapeutic agent is a modulator of Axl kinase. Examples of modulators of Axl kinase considered for use in combination therapies of the disclosure include but are not limited to bemcentinib (BGB324 or R428), TP-0903, LY2801653, amuvatinib (MP-470), bosutinib (SKI-606), MGCD 265, ASP2215, cabozantinib (XL184), foretinib (GSK1363089/XL880), and SGI-7079. In certain embodiments, the modulator of Axl kinase is a monoclonal antibody targeting AXL (e.g., YW327.6S2) or an AXL decoy receptor (e.g., GL2I.T), or glesatinib, merestinib, or a dual Flt3-Axl inhibitor such as gilteritinib.

In certain embodiments, the additional therapeutic agent is an anti-cancer agent or chemo-therapeutic agent. Examples of anti-cancer agents considered for use in combination therapies of the disclosure include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960, Cea-Vac, BrevaRex AR54, IMC-1C11, GlioMab-H, ING-1, anti-LCG MAbs, MT-103, KSB-303, Therex, KW2871, anti-HMI.24, Anti-PTHrP, 2C4 antibody, SGN-30, TRAIL-RI MAb, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MYLERAN®), busulfan injection (BUSULFEX®), capecitabine (XELODA®), N4-pentoxycarbonyl-5-deoxy-5-fluorocytidine, carboplatin (PARAPLATIN®), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, gemcitabine (difluorodeoxycitidine), hydroxyurea (HYDREA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MYLOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present disclosure are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON® A), ipilimumab, trametinib (e.g., MEKINIST®), peginterferon alfa-2b (e.g., PEGINTRON®, SYLATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOL®, PLATINOL-AQ®), doxorubicin hydrochloride liposome (DOXIL®, DOX-SL®, EVACET®, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL®, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (IRESSA®), and gemcitabine hydrochloride (GEMZAR®).

For the treatment of pancreatic cancer, the additional therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATE®, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In certain embodiments, the additional therapeutic agent is a PARP inhibitor, a dihydrofolate reductase inhibitor (e.g., pemetrexed), or a BCL-2 inhibitor (e.g., ventoclax).

In certain embodiments, the additional therapeutic agent is Temozolomide, Venetoclax, Niraparib, 5-Fluoruracil, Paclitaxel, Gemcitabine, Olaparib, Pemetrexed, Bortezomib, Etoposide, TRAIL, Ibrutinib, Vorinostat, FX-11, Rituxumab, Cisplatin, Indoximod, or a pharmaceutically acceptable salt thereof. In certain embodiments, the additional therapeutic agent is anti-PD1 agent, such as an anti-PD1 antibody.

In certain embodiments, the additional therapeutic agent is a DNA interactive agent (such as cisplatin or doxorubicin)); a taxane (e.g. taxotere, taxol); a topoisomerase II inhibitor (such as etoposide); a topoisomerase I inhibitor (such as irinotecan (or CPT-11), camptostar, or topotecan); a tubulin interacting agent (such as paclitaxel, docetaxel or the epothilones); a hormonal agent (such as tamoxifen); a thymidilate synthase inhibitor (such as 5-fluorouracil or 5-FU); an anti-metabolite (such as methoxtrexate); an alkylating agent (such as temozolomide, cyclophosphamide); a farnesyl protein transferase inhibitor (such as, SARASAR™ (4-2-4-(11R)-3,10-dibromo-8-chloro-6,11-dihydro-5H-benzo[5-6cyclohepta 1.2-bipyridin-11-yl-1-piperidinyl-2-oxoethyl-1-piperidine-carboxamide, or SCH 66336), tipifarnib (Zarnestra® or R115777 from Janssen Pharmaceuticals), L778,123 (a farnesyl protein transferase inhibitor from Merck & Company), BMS 214662 (a farnesyl protein transferase inhibitor from Bristol-Myers Squibb Pharmaceuticals, Princeton, N.J.); signal transduction inhibitors (such as, IressaR, TarcevaR (EGFR kinase inhibitors), antibodies to EGFR (e.g., C225), GLEEVECR (C-ablkinase inhibitor from Novartis Pharmaceuticals); an interferon such as, for example, Intron® (from Merck & Company), Peg-Intron® (from Merck & Company); a hormonal therapy combination; or an aromatase combination; ara C, adriamycin, cytoxan, and gemcitabine. Other anti-cancer (also known as anti-neoplastic) agents include but are not limited to Uracil mustard, Chlormethine, Ifosfamide, Melphalan, Chlorambucil, Pipobroman, Triethylenemelamine, Triethylenethiophosphoramine, BuSulfan, Carmustine, Lomustine, Streptozocin, Dacarbazine, FloXuri dine, Cytarabine, 6-Mercaptopurine, 6-Thioguanine, Fludarabine phosphate, oxaliplatin, leucovirin, oxaliplatin (ELOXATINR), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mito mycin-C. L-Asparaginase, Teniposide 17C-Ethinylestradiol. Diethylstilbestrol, Testosterone, Prednisone, Fluoxymesterone, Dromostanolone propionate, Testolactone, Megestro lacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levami, Navelbene, Anastrazole, Letrazole, Capecitabine, Reloxafine, Droloxafine, Hexamethylmelamine, Avastin, herceptin, Bexxar, Velcade®, Zevalin, Trisenox, Xeloda, Vinorelbine, Porfimer, Erbitux, Liposomal, Thiotepa, Altre tamine, Melphalan, Trastuzumab, Lerozole, Fulvestrant, Exemestane, Ifosfomide, Rituximab, C225, and Campath, 5-fluorouracil and leucovorin, with or without a 5-HT, receptor inhibitor (e.g., dolansetron, granisetron, ondansetron).

In certain embodiments, the additional therapeutic agent is an inhibitor of DNA Polymerase Theta (PolO) activity such as molecules described in WO 2023/233295 and WO 2024/121290.

In certain embodiments, the additional therapeutic agent is a Pol Theta inhibitor and a PARP inhibitor.

In some instances, patients may experience allergic reactions to the compounds of the present disclosure and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, HYDROCORT Acetate® and LANACORT®), prednisolone (e.g., DELTA-Cortel®, ORAPRED®, PEDIAPRED® and PRELONE®), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the compound of the present disclosure and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®. dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TYLENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In certain embodiments, the second therapeutic agent is an anticoagulant. Examples of anticoagulants considered for use in combination therapies of the disclosure include but are not limited to apixaban, dabigatran, edoxaban, heparin, rivaroxaban, warfarin, or combinations thereof.

In certain embodiments, the second therapeutic agent is an antiplatelet agent and dual antiplatelet therapy. Examples of antiplatelet agents and dual antiplatelet therapy considered for use in combination therapies of the disclosure include but are not limited to aspirin, clopidogrel, dipyridamole, prasugrel, ticagrelor, or combinations thereof.

In certain embodiments, the second therapeutic agent is an angiotensin-converting enzyme (ACE) inhibitor. Examples of angiotensin-converting enzyme (ACE) inhibitors considered for use in combination therapies of the invention include but are not limited to benazepril, captopril, enalapril, fosinopril, lisinopril, moexipril, perindopril, quinapril, ramipril, trandolapril, or combinations thereof.

In certain embodiments, the second therapeutic agent is an angiotensin II receptor blocker. Examples of angiotensin II receptor blockers considered for use in combination therapies of the disclosure include but are not limited to azilsartan, candesartan, eprosartan, irbesartan, losartan, olmesartan, telmisartan, valsartan, or combinations thereof.

In certain embodiments, the second therapeutic agent is an angiotensin receptor-neprilysin inhibitor. Examples of angiotensin receptor-neprilysin inhibitors considered for use in combination therapies of the disclosure include but are not limited to sacubitril/valsartan.

In certain embodiments, the second therapeutic agent is a beta blocker. Examples of beta blockers considered for use in combination therapies of the disclosure include but are not limited to acebutolol, atenolol, betaxolol, bisoprolol/hydrochlorothiazide, bisoprolol, metoprolol, nadolol, propranolol, sotalol, or combinations thereof.

In certain embodiments, the second therapeutic agent is a combined alpha- and beta-blocker. Examples of combined alpha- and beta-blockers considered for use in combination therapies of the disclosure include but are not limited to carvedilol, labetalol hydrochloride, or combinations thereof.

In certain embodiments, the second therapeutic agent is a calcium channel blocker. Examples of calcium channel blockers considered for use in combination therapies of the disclosure include but are not limited to amlodipine, diltiazem, felodipine, nifedipine, nimodipine, nisoldipine, verapamil, or combinations thereof.

In certain embodiments, the second therapeutic agent is a cholesterol-lowering medication. Examples of cholesterol-lowering medications considered for use in combination therapies of the disclosure include but are not limited to statins (e.g., atorvastatin, fluvastatin, lovastatin, pitavastatin, pravastatin, rosuvastatin, simvastatin), nicotinic acids (e.g., niacin), cholesterol absorption inhibitors (e.g., ezetimibe), combination statin and cholesterol absorption inhibitors (e.g., ezetimibe/simvastatin), or combinations thereof.

In certain embodiments, the second therapeutic agent is a digitalis preparation. Examples of digitalis preparations considered for use in combination therapies of the disclosure include but are not limited to digoxin.

In certain embodiments, the second therapeutic agent is a diuretic. Examples of diuretics considered for use in combination therapies of the disclosure include but are not limited to acetazolamide, amiloride, bumetanide, chlorothiazide, chlorthalidone, furosemide, hydro-chlorothiazide, indapamide, metalozone, spironolactone, torsemide, or combinations thereof.

In certain embodiments, the second therapeutic agent is a vasodilator. Examples of vasodilators considered for use in combination therapies of the disclosure include but are not limited to isosorbide dinitrate, isosorbide mononitrate, hydralazine, nitroglycerin, minoxidil, or combinations thereof.

In certain embodiments, the second therapeutic agent is an antiarrhythmic agent. Examples of antiarrhythmic agent considered for use in combination therapies of the disclosure include but are not limited to Class Ia antiarrhythmic agents (e.g., quinidine, ajmaline, procain amide, dispyramide, sparteine), Class Ib antiarrhythmic agents (e.g., lidocaine, phenytoin, mexiletine, tocainide), Class Ic antiarrhythmic agents (e.g., encainide, flecainide, propafenone, moricizine), Class II antiarrhythmic agents (e.g., carvedilol, propranolol, esmolol, timolol, metoprolol, atenolol, bisoprolol, nebivolol), Class III antiarrhythmic agents (e.g., amiodarone, sotalol, ibutilide, dofetilide, dronedarone, E-4031, vemakalant), Class IV antiarrhythmic agents (e.g., verapamil, diltiazem; Class V, such as adenosine, digoxin, magnesium sulfate), or combinations thereof.

In certain embodiments, the second therapeutic agent is an agent used to treat a metabolic disorder. Examples of metabolic disorder agents considered for use in combination therapies of the disclosure include but are not limited to statins (e.g., atorvastatin, simvastatin, rosuvastatin), cholesterol absorption inhibitors (e.g., ezetimibe), insulin-sensitizing agents (e.g., rosiglitazone, metformin), GLP-1 receptor agonists (e.g., exenatide), DPP-4 inhibitors (e.g., sitagliptin), thiazolidinediones (e.g., pioglitazone), or combinations thereof.

In yet another aspect, a compound of the present disclosure may be used in combination with known therapeutic processes, for example, with the administration of hormones or in radiation therapy. In certain instances, a compound of the present disclosure may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the compound described herein (e.g., a compound of Formula I, or other compounds in Section I) and the additional therapeutic agent(s) are administered in doses commonly employed when such agents are used as monotherapy for treating the disease or condition. In other embodiments, the compound described herein (e.g., a compound of Formula I, or other compounds in Section I) and the additional therapeutic agent(s) are administered in doses lower than the doses commonly employed when such agents are used as monotherapy for treating the disease or condition. In certain embodiments, the compound described herein (e.g., a compound of Formula I, or other compounds in Section I) and the additional therapeutic agent(s) are present in the same composition, which is suitable for oral administration.

In certain embodiments, the compound described herein (e.g., a compound of Formula I, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### IV. Pharmaceutical Compositions and Dosing Considerations

As indicated above, the disclosure provides pharmaceutical compositions. In some embodiments, the pharmaceutical compositions comprise a therapeutically effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the disclosure provides a pharmaceutical composition comprising a compound described herein (e.g., a compound of Formula I, or other compounds in Section I) and a pharmaceutically acceptable carrier.

The phrase “therapeutically effective amount” as used herein means that amount of a compound, material, or composition comprising a compound of the present disclosure which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound of the present disclosure. In certain embodiments, an aforementioned formulation renders orally bioavailable a compound of the present disclosure.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present disclosure may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Unless otherwise stated, “administering” or “administration” of an agent (such as a compound of the disclosure) encompasses the delivery to a subject of the referenced agent or a pharmaceutically acceptable salt and/or solvate thereof, as well as delivery of a prodrug or antibody-drug conjugate of the referenced agent, using any suitable formulation or route of administration, e.g., as described herein.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The disclosure further provides a unit dosage form (such as a tablet or capsule) comprising a heteroaryl described herein in a therapeutically effective amount for the treatment of a medical disorder described herein.

### V. Medical Kits

Another aspect of this disclosure is a kit comprising a therapeutically effective amount of a compound described herein (e.g., a compound of Formula I, or other compounds in Section I), a pharmaceutically acceptable carrier, vehicle or diluent, and optionally at least one additional therapeutic agent listed above. In certain embodiments, the kit further comprises instructions, such as instructions for treating a disease described herein.

### VI. Examples

The disclosure now being generally described, will be more readily understood by reference to the following examples, which are included merely for purposes of illustration of certain aspects and embodiments of the present disclosure, and is not intended to limit the disclosure.

### General Procedure A: Preparation of Phenyl Carbamates:

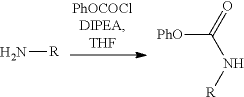

To a stirred solution of amine (1 equiv) and DIPEA (2.5 equiv) in THF (concentration of amine in THF=˜0.18 M) was added phenyl chloroformate (1.2 equiv) at 0° C. After the addition, the reaction mixture was stirred at 0° C. for 30 min and rt for 2 hours. The reaction mixture was poured into ice cold water. The precipitate was filtered and dried.

General Procedure B: Preparation of Ureas from Phenyl Carbamates:

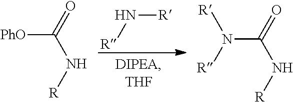

Amine (1.1 equiv) was added to a solution of phenyl carbamate (1.0 equiv) and DIPEA (5 equiv) in THF (concentration of phenyl carbamate in THF=˜0.13 M) at ambient temperature. The reaction mixture was stirred at 70° C. for 16 hours. The volatiles were removed, and the reaction mixture was poured in ice cold water, stirred for 10 min, and the precipitate was filtered and dried.

### LCMS Methods Used in the Preparation of Chemical Compounds

Method F: CORTECS UPLC C18 (3×30) mm, 1.6 μm; flow rate: 0.85 mL/min; mobile phase A: 0.05% formic acid in water; mobile phase B: 0.05% formic acid in ACN; injection volume: 2 μL; column temp.: 45° C.; gradient program (time/% B): 0.0/3, 0.1/3, 1.4/97, 2.0/97, 2.05/3, 2.5/3.

Method G: X-select CSH 18 (3×50 mm×2.5 mm); mobile phase: A: 0.05% TFA in H₂O; B: 0.05% TFA in ACN; injection volume: 2 μL; flow rate: 1 mL/min; column temperature: 40° C.; gradient (time/% B): 0.0/2, 0.3/2, 2/98, 2.8/98, 3.0/2, 3.7/2.

Method I: X-Select CSH C18, (50 mm*3.0 mm, 2.5 μm); mobile phase A: 0.05% formic acid in water; mobile phase B: 0.05% formic acid in acetonitrile; flow rate: 1.0 mL/min; column temperature: 40° C.; gradient (time/B %): 0.01/2, 0.3/2, 2.0/98, 2.8/98, 3.0/2, 3.7/2.

Method J: Column: X Bridge C18 4.6*50 mm*3.5 um; mobile phase A: 10 mM ammonium acetate; B: 100% ACN; gradient (time/% B) 0.0/10, 0.5/10, 4.0/90, 8.0/90, 8.1/10, 10.0/10; flow rate: 0.6 mL/min.

### HPLC Methods Used in the Preparation of Chemical Compounds

Method Z: Column: X Bridge C18 4.6*250 mm*5.0 μm; mobile phase: solvent A: 10 mM ammonium acetate in water; solvent B: CH₃CN; flow rate: 1.2 mL/min; diluent: methanol (100%).

### Example 1—Synthesis of 4-5-FLUORO-N-(4-(4-(5-METHYL-1,3,4-OXADIAZOL-2-YL) BICYCLO [2.2.2]OCTAN-1-YL) PHENYL) ISOINDOLINE-2-CARBOXAMIDE (I-17)

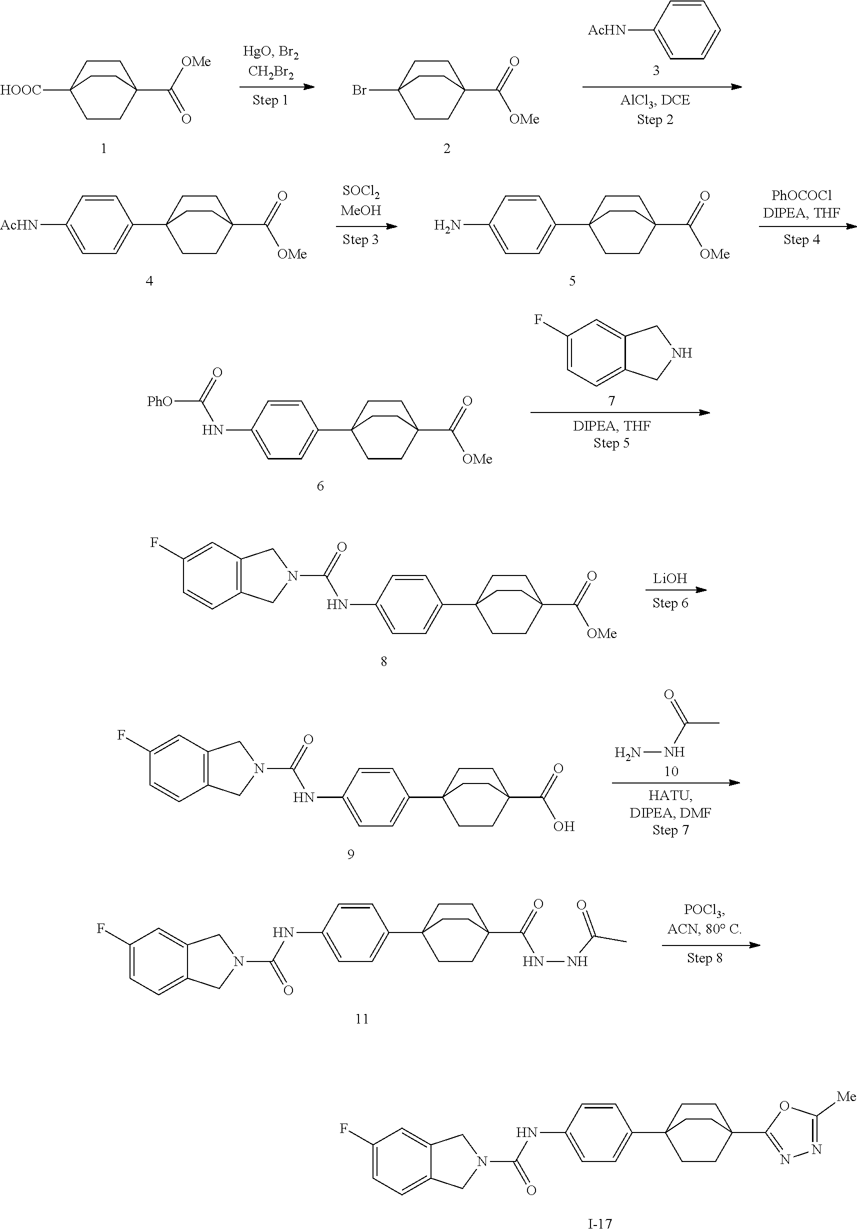

Step 1: Synthesis of methyl 4-bromobicyclo[2.2.2]octane-1-carboxylate (2): To a stirred solution of 1 (10.0 g, 47.16 mmol) in dibromomethane (300 mL) was added mercury oxide (10.18 g, 47.16 mmol) under a nitrogen atmosphere. Bromine (3.13 mL, 61.32) in dibromomethane (10 mL) was added slowly at 80° C. and maintained for 3 hours. The progress of the reaction was monitored by TLC. After complete consumption of the starting material, the reaction mixture was cooled to rt and then filtered through a pad of CELITE®. The filtrate was concentrated under reduced pressure to obtain the crude product. The residue was purified by column chromatography (eluting with 5% EtOAc-hexanes) which afforded 8.0 g of 2 (69% yield) as an off-white solid. ¹H NMR (300 MHz, DMSO-d₆) δ 3.57 (d, J=5.7 Hz, 3H), 2.30-2.11 (m, 6H), 2.04-1.81 (m, 6H).

Step 2: Synthesis of methyl 4-(4-acetamidophenyl)bicyclo[2.2.2]octane-1-carboxylate (4): To a stirred solution of 2 (5.8 g, 23.5 mmol) and 3 (3.2 g, 23.5 mmol) in 1,2-dichloroethane (230 mL) was added aluminum chloride (14.0 g, 105.7 mmol) in portions at 0° C. Then the reaction mixture was stirred at rt for 16 hours. After completion of the reaction, the reaction mixture was slowly poured into ice cold water. The aqueous layer was extracted with DCM (2×50 mL). The combined organic extracts were washed with water, brine, dried over sodium sulphate, filtered, and concentrated under reduced pressure to obtain the crude product. The crude product was purified by column chromatography (eluting with 25% EtOAc-hexanes), which afforded 3.5 g of 4 (49% yield) as a pale-yellow solid. ¹H NMR (300 MHz, DMSO-d₆) δ 9.85 (s, 1H), 7.55-7.39 (m, 2H), 7.28-7.11 (m, 2H), 3.34 (s, 3H), 2.01 (d, J=3.7 Hz, 3H), 1.83-1.75 (m, 12H). LC-MS (Method J): [M+H]⁺=302.2.

Step 3: Synthesis of methyl 4-(4-aminophenyl)bicyclo[2.2.2]octane-1-carboxylate (5): To a stirred solution of 4 (3.5 g, 11.6 mmol) in MeOH (75 mL) was added thionyl chloride (1.52 mL, 11.6 mmol) dropwise at rt, then the reaction mixture was heated to 70° C. for 8 hours. After completion of the reaction, the reaction mixture was cooled to rt and the volatiles were removed under reduced pressure. The reaction mixture was diluted with water, basified with a saturated aqueous solution of NaHCO₃ and extracted with EtOAc (2×50 mL). The combined organic layer was dried over sodium sulfate, filtered and concentrated under reduced pressure to obtain the crude product. The product was purified by column chromatography (eluting with 15% EtOAc-Hexanes), which afforded 1.5 g of 5 (42%) as a pale-yellow solid. ¹H NMR (300 MHz, DMSO-d₆) δ 6.98 (dd, J=17.0, 8.2 Hz, 2H), 6.61-6.48 (m, 2H), 4.88 (s, 2H), 3.39 (s, 3H), 1.99-1.64 (m, 12H).

Step 4: Synthesis of methyl 4-(4-((phenoxycarbonyl) amino)phenyl) bicyclo[2.2.2]octane-1-carboxylate (6): 6 was prepared according to General Procedure A using methyl 4-(4-aminophenyl)bicyclo [2.2.2]octane-1-carboxylate (5) (1.5 g, 5.0 mmol). 1.8 g of 6 (69% yield) was obtained as an off-white solid. ¹H NMR (300 MHz, DMSO-d₆) δ 10.12 (s, 1H), 7.55-7.34 (m, 4H), 7.33-7.10 (m, 5H), 3.33 (s, 3H), 1.99-1.64 (d, 12H). LC-MS (Method J): [M+H]+=380.2.

Step 5: Preparation of methyl 4-(4-(5-fluoroisoindoline-2-carboxamido)phenyl) bicyclo[2.2.2]octane-1-carboxylate (8): 8 was prepared according to General Procedure B using methyl 4-(4-((phenoxycarbonyl)amino) phenyl) bicyclo[2.2.2]octane-1-carboxylate (6) (1.8 g, 4.74 mmol). 1.0 g of 8 (50% yield) was obtained as an off-white solid. ¹H NMR (300 MHz, DMSO-d₆) δ 8.29 (s, 1H), 7.54-7.33 (m, 3H), 7.33-7.05 (m, 4H), 4.73 (d, J=8.2 Hz, 4H), 3.60 (s, 3H), 1.99-1.64 (m, 12H). LC-MS (Method J): [M+H]⁺=423.2.

Step 6: Synthesis of 4-(4-(5-fluoro-2,3-dihydro-1H-indene-2-carboxamido) phenyl)bicyclo[2.2.2]octane-1-carboxylic acid (9): To a stirred solution of 8 (1.0 g, 2.36 mmol) in a mixture of THF:MeOH:H₂O (30 mL; 1:1:1) was added lithium hydroxide monohydrate (0.59 g, 14.18 mmol) at rt and stirred for 24 hours. After completion of the reaction, the volatiles were removed under reduced pressure and the reaction mixture was acidified with 1.0 N HCl. The precipitate was filtered and dried to obtain 820 mg of 9 (85% yield) as an off-white solid. ¹H NMR (300 MHz, DMSO-d₆) δ 12.03 (s, 1H), 8.28 (s, 1H), 7.56-7.29 (m, 3H), 7.29-6.98 (m, 4H), 4.72 (d, J=8.1 Hz, 4H), 1.78 (s, 12H). LC-MS (Method J): [M+H]⁺=409.2.

Step 7: Synthesis of N-(4-(4-(2-acetylhydrazine-1-carbonyl) bicyclo [2.2.2]octan-1-yl) phenyl)-5-fluoro isoindoline-2-carboxamide (11): To a stirred solution of 9 (1.0 g, 2.45 mmol) in DMF (10.0 mL) was added 10 (0.19 g, 2.60 mmol) followed by addition of DIPEA (1.2 mL, 7.35 mmol) and HATU (1.12 g, 3.18 mmol) at 0° C. The reaction mixture was stirred at RT for 16 hours. After completion of the reaction, the reaction mixture was poured into ice cold water. The precipitate was filtered, washed with water (25 mL), and dried to obtain 700 mg of 11 (61.9% yield) as an off-white solid. ¹H NMR (300 MHz, DMSO-d₆) δ 9.57 (d, J=1.4 Hz, 1H), 9.25 (d, J=1.2 Hz, 1H), 8.28 (s, 1H), 7.48-7.34 (m, 3H), 7.26-7.08 (m, 4H), 4.74 (s, 2H), 4.71 (s, 2H), 1.83 (s, 3H), 1.81-1.76 (m, 12H).

Step 8: Synthesis of 5-fluoro-N-(4-(4-(5-methyl-1,3,4-oxadiazol-2-yl) bicyclo [2.2.2]octan-1-yl) phenyl) isoindoline-2-carboxamide (I-17): To a stirred solution of 11 (250 mg, 0.537 mmol) in ACN (10 mL) in a sealed tube was added POCl₃ (0.1 mL, 1.61 mmol) at RT. The reaction mixture was heated to 80° C. for 2 hours. After completion of the reaction, the reaction mixture was cooled to 0° C. and quenched with a saturated solution of NaHCO₃ and extracted with EtOAc (2×50 mL). The combined organic layer was dried over anhydrous Na₂SO₄, filtered and concentrated under reduced pressure to obtain the crude product. The crude product was purified by column chromatography (eluent: 0-5% MeOH in DCM) to afford 35 mg of I-17 (14% yield) as an off-white solid. ¹H NMR (300 MHz, DMSO-d₆) δ 8.30 (s, 1H), 7.46 (d, J=8.8 Hz, 2H), 7.42-7.35 (m, 1H), 7.27-7.20 (m, 3H), 7.18-7.10 (m, 1H), 4.74 (s, 2H), 4.71 (s, 2H), 2.46 (s, 3H), 2.02-1.82 (m, 12H). LCMS (Method J): 447.32 (M+H), purity: 98.50% by AUC, ret. time: 5.27 min, UV: 245 nm. HPLC (Method Z): purity: 95.03% by AUC, ret. time: 14.92 min, UV: 250 nm.

### Example 2—Synthesis of 5-FLUORO-N-(4-(4-(1-(2-HYDROXYETHYL)-1H-1,2,3-TRIAZOL-4-YL) BICYCLO [2.2.2]OCTAN-1-YL) PHENYL) ISOINDOLINE-2-CARBOXAMIDE (I-16)

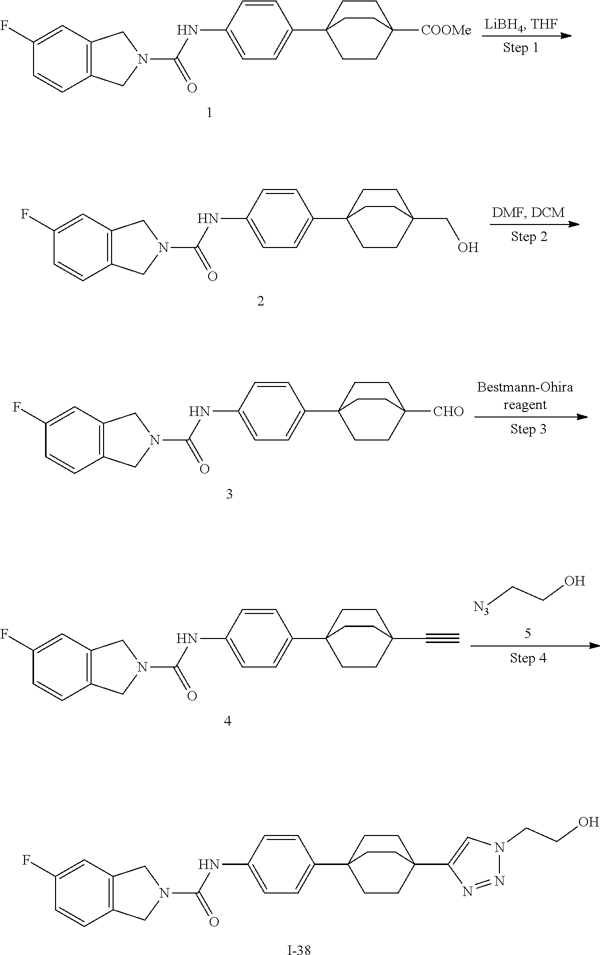

Step 1: Synthesis of 5-fluoro-N-(4-(4-(hydroxymethyl) bicyclo [2.2.2]octan-1-yl) phenyl) isoindoline-2-carboxamide (2): To a stirred solution of 1 (2.3 g, 5.43 mmol) in THF (100 mL) was added LiBH₄ (16.3 mL, 32.6 mmol, 2.0 M in THF) at 0° C. The reaction mixture was stirred at rt for 16 hours. After completion of the reaction, the reaction mixture was cooled to 0° C. and quenched with a saturated solution of NH₄Cl (50 mL) and extracted with EtOAc (3×50 mL). The combined organic extracts were dried over anhydrous Na₂SO₄, filtered and concentrated under reduced pressure to afford 1.5 g of 2 (71% yield) as an off-white solid. ¹H NMR (300 MHz, DMSO-d₆) δ 8.27 (s, 1H), 7.46-7.35 (m, 3H), 7.26-7.09 (m, 4H), 4.73 (s, 2H), 4.71 (s, 2H), 4.36 (t, J=5.5 Hz, 1H), 3.07 (d, J=5.5 Hz, 2H), 1.79-1.66 (m, 6H), 1.50-1.38 (m, 6H). LCMS (Method J): 395.29 (M+H), purity: 99.64% by AUC, ret. time: 5.22 min, UV: 240 nm.

Step 2: Synthesis of 5-fluoro-N-(4-(4-formylbicyclo [2.2.2]octan-1-yl) phenyl) isoindoline-2-carboxamide (3): To a stirred solution of 2 (500 mg, 1.26 mmol) in DCM (10 mL) was added a solution of Dess Martin periodinane (807 mg, 1.90 mmol) at 0° C. The reaction mixture was stirred at RT for 2 hours. After completion of the reaction, the reaction mixture was quenched with a saturated NHCO₃ solution and extracted with DCM (2×50 mL). The combined organic extracts were dried over anhydrous Na₂SO₄, filtered and concentrated under reduced pressure to afford 160 mg of 3 (32.17%) as an off-white solid. ¹H NMR (300 MHz, DMSO-d₆) δ 9.47 (s, 1H), 8.29 (s, 1H), 7.45 (d, J=8.8 Hz, 2H), 7.41-7.35 (m, 1H), 7.29-7.18 (m, 3H), 7.18-7.09 (m, 1H), 4.74 (s, 2H), 4.71 (s, 2H), 1.84-1.63 (m, 12H). LCMS (Method J): 393.28 (M+H), purity: 71.47% by AUC, ret. time: 5.72 min, UV: 240 nm.

Step 3: Synthesis of N-(4-(4-ethynylbicyclo [2.2.2]octan-1-yl) phenyl)-5-fluoroisoindoline-2-carboxamide (4): To a stirred solution of 3 (160 mg, 0.40 mmol) in MeOH (10 mL) was added Bestmann-Ohira reagent (0.063 mL, 0.48 mmol), followed by addition of K₂CO₃ at 0° C. The reaction mixture was stirred at RT for 2 hours. After completion of the reaction, the volatiles were removed under reduced pressure to obtain the crude product. The crude product was dissolved in EtOAc (50 mL) and washed with water (50 mL). The organic layer was dried over anhydrous Na₂SO₄, filtered and concentrated under reduced pressure to afford 120 mg of 4 (75.94%) as an off-white solid. ¹H NMR (300 MHz, DMSO-d₆) δ 8.28 (s, 1H), 7.46-7.35 (m, 3H), 7.26-7.09 (m, 4H), 4.73 (s, 2H), 4.71 (s, 2H), 2.91 (s, 1H), 1.87-1.68 (m, 12H). LCMS (Method J): 389.24 (M+H), purity: 93.55% by AUC, ret. time: 6.12 min, UV: 240 nm.

Step 4: Synthesis of 5-fluoro-N-(4-(4-(1-(2-hydroxyethyl)-1H-1,2,3-triazol-4-yl) bicyclo [2.2.2]octan-1-yl) phenyl) isoindoline-2-carboxamide (I-16): To a stirred solution of 4 (120 mg, 0.309 mmol) in IPA/DCM (2.5 mL, 1:3) and water (0.5 mL) was added sodium ascorbate (6.1 mg, 0.03 mmol), CuSO₄.5H₂O (3.0 mg, 0.012 mmol), and 5 (40 mg, 0.463 mmol) at RT. The reaction mixture was stirred at RT for 16 hours. The reaction mixture was monitored by TLC and LCMS. After completion of the reaction, the reaction mixture was diluted with water (25 mL) and extracted with 10% MeOH-DCM (2×50 mL). The organic layer was dried over anhydrous Na₂SO₄, filtered and concentrated under reduced pressure to afford crude product. The crude product was triturated with n-hexane to afford 120 mg of I-16 (82%) as an off-white solid. ¹H NMR (300 MHz, DMSO-d₆) δ 8.29 (s, 1H), 7.78 (s, 1H), 7.45 (d, J=8.7 Hz, 2H), 7.42-7.35 (m, 1H), 7.28-7.19 (m, 3H), 7.18-7.09 (m, 1H), 5.02 (t, J=5.3 Hz, 1H), 4.74 (s, 2H), 4.71 (s, 2H), 4.33 (t, J=5.5 Hz, 2H), 3.77 (q, J=5.3 Hz, 2H), 1.95-1.80 (m, 12H). LCMS (Method J): 476.49 (M+H), purity: 98.63% by AUC, ret. time: 5.00 min, UV: 240 nm. HPLC (Method Z): purity: 98.00% by AUC, ret. time: 14.08 min, UV: 250 nm.

### Example 3—Synthesis of 5-FLUORO-N-(4-(4-(5-METHYL-1H-1,2,4-TRIAZOL-3-YL)BICYCLO[2.2.2]OCTAN-1-YL)PHENYL)ISOINDOLINE-2-CARBOXAMIDE (I-15)

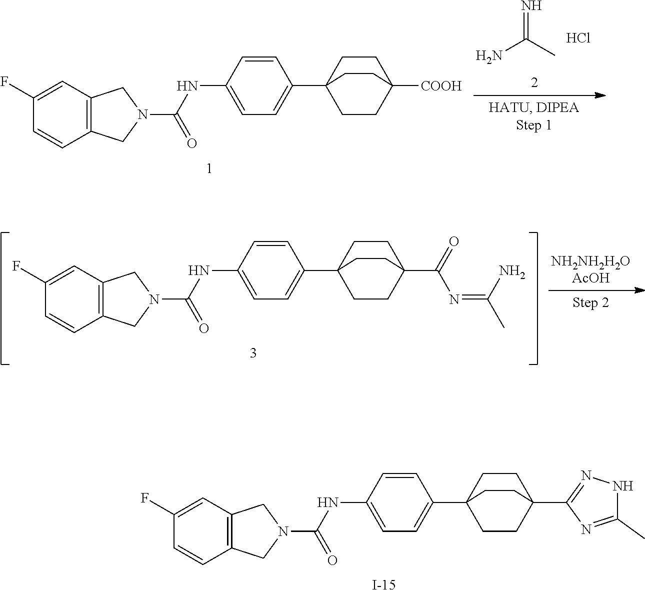

Step 1: Synthesis of (Z)—N-(4-(4-((1-aminoethylidene) carbamoyl) bicyclo [2.2.2]octan-1-yl) phenyl)-5-fluoroisoindoline-2-carboxamide (3): To a stirred solution of 1 (250 mg, 0.61 mmol) in DMF (5.0 mL) was added 2 (69 mg, 0.73 mmol), followed by addition of DIPEA (0.5 mL, 3.05 mmol) and HATU (695 mg, 1.83 mmol) at 0° C. The reaction mixture was stirred at RT for 12 hours. After completion of the reaction, the reaction mixture was poured into ice cold water and the precipitate was filtered, washed with water, and dried to obtain 250 mg of crude 3 as an off-white solid. The crude product was used in the next step without further purification.

Step 2: Synthesis of 5-fluoro-N-(4-(4-(5-methyl-1H-1,2,4-triazol-3-yl) bicyclo [2.2.2]octan-1-yl) phenyl) isoindoline-2-carboxamide (I-15): A solution of 3 (250 mg, 0.61 mmol) in AcOH (413 mg, 5.9 mmol) was stirred at 0° C. The reaction mixture was stirred at RT for 30 min and then NH₂NH₂·H₂O (48 mg, 1.18 mmol) was added. The reaction mixture was stirred at RT for 1 hour. The reaction mixture was then heated to 80° C. for 1.5 hours. After completion of the reaction, the reaction mixture was poured into ice cold water and the precipitate was filtered, washed with water, and dried to obtain crude product. The crude product was purified by neutral alumina column chromatography (eluent: 2-10% IPA in DCM) which afforded 58 mg of I-15 (22% yield) as an off-white solid. ¹H NMR (300 MHz, DMSO-d₆) δ 8.28 (s, 1H), 7.49-7.35 (m, 3H), 7.27-7.08 (m, 4H), 4.74 (s, 2H), 4.71 (s, 2H), 2.29 (s, 3H), 1.96-1.71 (m, 12H). LCMS (Method J): 446.51 (M+H), purity: 97.23% by AUC, ret. time: 4.88 min, UV: 270 nm. HPLC (Method Z): purity: 96.61% by AUC, ret. time: 13.88 min, UV: 260 nm.

### Example 4—Synthesis of 5-FLUORO-N-(4-(4-(4-(2-HYDROXY-2-METHYL PROPYL)-1H-1,2,3-TRIAZOL-1-YL)BICYCLO[2.2.2]OCTAN-1-YL)PHENYL) ISOINDOLINE-2-CARBOXAMIDE (I-9)

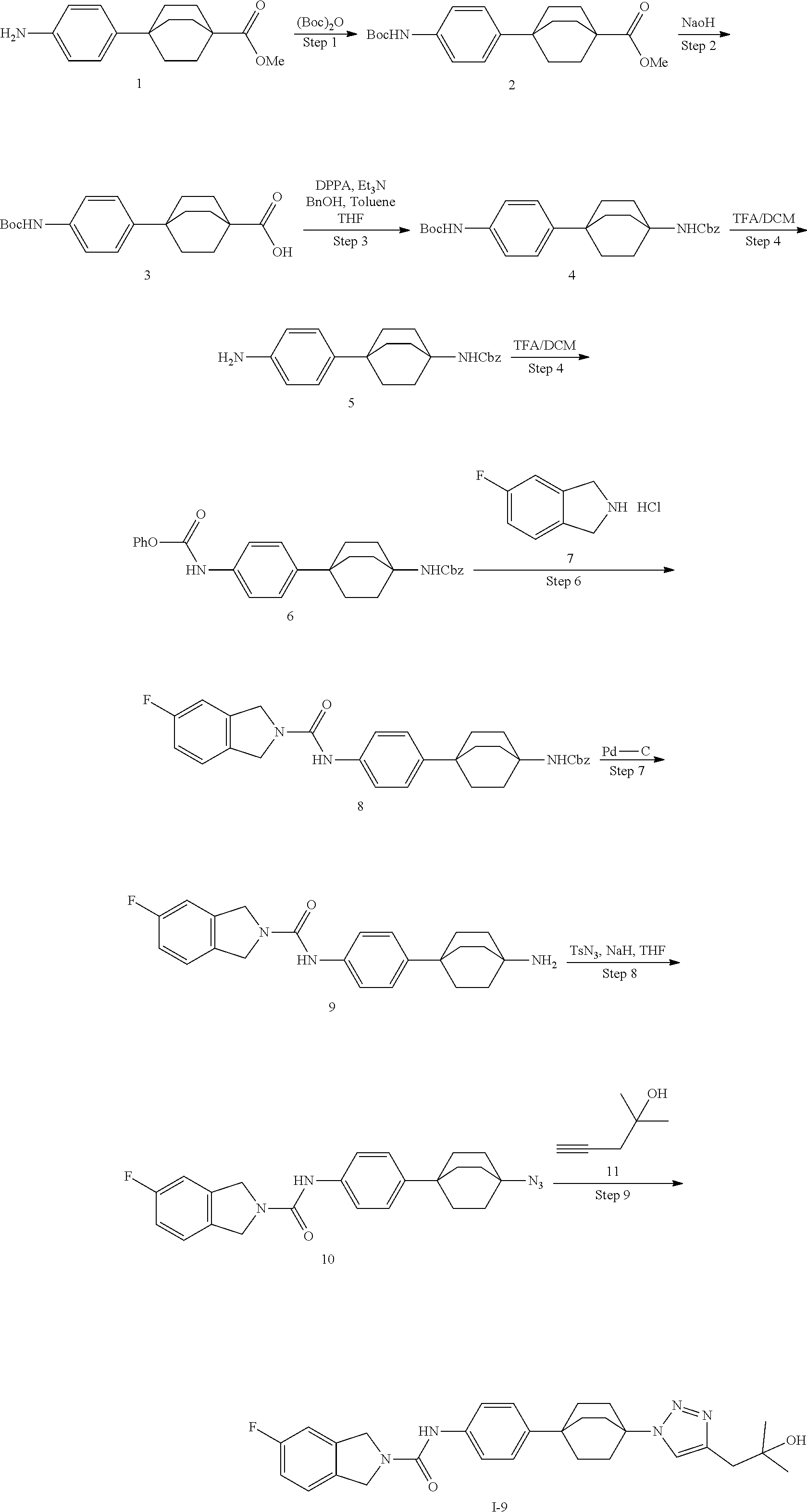

Step 1: Synthesis of methyl 4-(4-((tert-butoxy carbonyl) amino) phenyl) bicyclo [2.2.2]octane-1-carboxylate (2): To a stirred solution of 1 (540 mg, 2.08 mmol) in a mixture of 1,4-dioxane:water (40 mL 1:1) was added NaHCO₃ (870 mg, 10.60 mmol), followed by Boc-anhydride (1.36 g, 6.24 mmol) at 0° C. under N₂ atmosphere. The reaction mixture was stirred at RT for 4 hours. After completion of the reaction, the reaction mixture was diluted with water (10 mL) and extracted with EtOAc (2×20 mL). The combined organic layer was dried over Na₂SO₄ and concentrated under reduced pressure to afford 740 mg of 2 (98.93% yield) as a brown solid. ¹H NMR (300 MHz, DMSO-d₆) δ 9.21 (s, 1H), 7.33 (d, J=8.7 Hz, 2H), 7.18 (d, J=8.8 Hz, 2H), 3.59 (s, 3H), 1.86-1.68 (m, 12H), 1.46 (s, 9H).

Step 2: Synthesis of 4-(4-((tert-butoxy carbonyl) amino) phenyl) bicyclo [2.2.2]octane-1-carboxylic acid (3): To a stirred solution of 2 (7.0 g, 19.49 mmol) in a mixture of THF:MeOH:H₂O (375 mL, 2:2:1) was added sodium hydroxide powder (7.01 g, 175.48 mmol) over 20 minutes at 0° C. and the reaction mixture was stirred at RT for 24 hours. After completion of the reaction, the volatiles were removed under reduced pressure. The reaction mixture was then diluted with ice cold water, acidified (to pH ˜2) with sodium bisulfate, and the precipitate was filtered and dissolved in 30% MeOH-EtOAc (300 mL). The mixture was dried over Na₂SO₄ and concentrated under reduced pressure to afford 6.5 g of 3 (97.01% yield) as a brown solid. ¹H NMR (300 MHz, DMSO-d₆) δ 11.2 (s, 1H), 9.20 (s, 1H), 7.33 (d, J=8.8 Hz, 2H), 7.17 (d, J=8.8 Hz, 2H), 1.81-1.69 (m, 12H), 1.46 (s, 9H).

Step 3: Synthesis of tert-butyl (4-(4-(((benzyloxy)carbonyl) amino) bicyclo [2.2.2]octan-1-yl) phenyl) carbamate (4): To a stirred solution of 3 (3.0 g, 8.69 mmol) in a mixture of THF:toluene (100 mL; 2:1) was added molecular sieves (3.0 g), triethylamine (3.51 g, 34.78 mmol), and DPPA (5.73 g, 20.86 mmol) at RT, and the reaction mixture was stirred at RT for 4 hours in a sealed tube. After completion of the reaction, benzyl alcohol (1.87 g, 17.39 mmol) was added at RT and the reaction mixture was stirred at 100° C. for 16 hours. After completion of the reaction, the reaction mixture was diluted with water (30 mL) and extracted with EtOAc (3×100 mL). The combined organic extracts were dried over Na₂SO₄ and concentrated under reduced pressure to afford the crude product. The product was purified by column chromatography (eluent-O-30% ethyl acetate in hexanes) which afforded 2.5 g of 4 (64.1% yield) as an off-white solid. ¹H NMR (300 MHz, DMSO-d₆) δ 9.19 (s, 1H), 7.39-7.28 (m, 7H), 7.17 (d, J=8.8 Hz, 2H), 6.99 (s, 1H), 4.96 (s, 2H), 1.91-1.73 (m, 12H), 1.45 (s, 9H).

Step 4: Synthesis of benzyl (4-(4-aminophenyl) bicyclo [2.2.2]octan-1-yl) carbamate (5): To a stirred solution of 4 (3.2 g, 7.11 mmol) in DCM (100 mL) was added TFA (12.16 g, 106.65 mmol) at 0° C. under N₂ atmosphere. Then the reaction mixture was stirred at 50° C. for 30 min and then the reaction mixture was stirred at RT for 2 hours. After completion of the reaction, the reaction mixture was basified (pH ˜8-9) with a saturated NaHCO₃ solution (30 mL). The reaction mixture was extracted with DCM (2×50 mL). The combined organic layer was dried over Na₂SO₄ and concentrated under reduced pressure to afford the 2.0 g of 5 (80.64%) as an off-white solid. The crude product was directly used in the next step without further purification. ¹H NMR (300 MHz, DMSO-d₆) δ 7.40-7.26 (m, 5H), 6.94 (d, J=8.6 Hz, 2H), 6.46 (d, J=8.6 Hz, 2H), 5.02-4.70 (m, 3H), 1.89-1.68 (m, 12H). LC-MS (Method J): [M+H]⁺=351.36 (M+H), purity: 90.03% by AUC, ret. time: 5.50 min, UV: 260 nm.

Step 5: Synthesis of benzyl (4-(4-((phenoxycarbonyl)amino) phenyl) bicyclo [2.2.2]octan-1-yl) carbamate (6): To a stirred solution of 5 (19.0 g, 68.76 mmol) and DIPEA (56.29 mL, 343.82 mmol) in THF (400 mL) was added phenyl chloroformate (13.22 g, 103.14 mmol) at 0° C., and then the mixture was stirred at RT for 2 hours. After completion of the reaction, the reaction mixture was diluted with water (200 mL) and extracted with EtOAc (3×350 mL). The combined organic extracts were dried over Na₂SO₄ and concentrated under reduced pressure to afford 17 g of 6 (92.30% yield) as a white solid. ¹H NMR (300 MHz, DMSO-d₆) δ 10.11 (s, 1H), 7.49-7.29 (m, 9H), 7.29-7.10 (m, 5H), 7.00 (s, 1H), 4.96 (s, 2H), 1.93-1.75 (m, 12H).

Step 6: Synthesis of benzyl (4-(4-(5-fluoroisoindoline-2-carboxamido) phenyl) bicyclo [2.2.2]octan-1-yl) carbamate (8): To a stirred solution of 6 (2.0 g, 4.25 mmol) and triethylamine (1.68 g, 63.82 mmol) in THF (40 mL) was added 7 (1.1 g, 6.38 mmol) at ambient temperature. Then the reaction mixture was stirred at 55° C. for 16 hours. After completion of the reaction, the reaction mixture was poured into ice cold water and stirred for 10 min. The precipitate was washed with MTBE, filtered, and dried to obtain 1.93 g of 8 (88% yield) as an off-white solid. ¹H NMR (300 MHz, DMSO-d₆) δ 8.27 (s, 1H), 7.46-7.27 (m, 8H), 7.26-7.09 (m, 4H), 7.00 (s, 1H), 4.97 (s, 2H), 4.73 (s, 2H), 4.71 (s, 2H), 1.93-1.74 (m, 12H).

Step 7: Synthesis of N-(4-(4-aminobicyclo[2.2.2]octan-1-yl)phenyl)-5-fluoroisoindoline-2-carboxamide hydrobromide (9): To a stirred solution of 8 (1.93 g, 3.762 mmol) in a mixture of THF:ethanol (60 mL, 1:1) was added Pd/C (570 mg 50% wet) at RT under H₂ atmosphere. Then the reaction mixture was stirred at RT for 16 hours. After completion of the reaction, the reaction mixture was filtered through a CELITE® pad and the CELITE® was washed with 50% MeOH in DCM (200 mL). The filtrate was concentrated under reduced pressure to afford 1.3 g of 9 (91% yield) as a white solid. ¹H NMR (300 MHz, DMSO-d₆) δ 8.32-8.20 (m, 1H), 7.46-7.35 (m, 3H), 7.26-7.08 (m, 4H), 4.73 (s, 2H), 4.71 (s, 2H), 1.87-1.72 (m, 6H), 1.59-1.47 (m, 6H).

Step 8: Synthesis of N-(4-(4-azidobicyclo[2.2.2]octan-1-yl)phenyl)-5-fluoroisoindoline-2-carboxamide (10): To a stirred solution of NaH (205 mg, 8.57 mmol) in THF (5 mL) was added a solution of 9 (500 mg, 1.32 mmol) in THF (5 mL) at 0° C., followed by the addition of TsN₃ (395 mg, 2.0 mmol) in THF (5 mL). The reaction was stirred at RT for 24 hours. After completion of reaction, the reaction was quenched with a saturated NH₄Cl solution and extracted with EtOAc. The separated organic layer was dried over anhydrous Na₂SO₄, filtered, and concentrated under reduced pressure to obtain the crude product. The crude product was purified by flash chromatography and eluted with 0-5% EtOAc in hexane to afford 350 mg (65% yield) of 10 as an off-white solid. ¹H NMR (300 MHz, DMSO-d6) δ 8.29 (s, 1H), 7.44 (d, J=8.8 Hz, 3H), 7.29-7.09 (m, 4H), 4.74 (s, 2H), 4.71 (s, 2H), 1.94-1.85 (m, 6H), 1.85-1.74 (m, 6H). LCMS (Method J): 406.43 (M+H), purity: 99.74% by AUC, ret. time: 6.25 min, UV: 245 nm.

Step 9: Synthesis of 5-fluoro-N-(4-(4-(4-(2-hydroxy-2-methylpropyl)-1H-1,2,3-triazol-1-yl) bicyclo [2.2.2]octan-1-yl) phenyl) isoindoline-2-carboxamide (I-9): To a stirred solution of 10 (230 mg, 0.57 mmol) in THF:water (6 mL; 1:1) in a sealed tube was added copper sulfate pentahydrate (28.3 mg, 0.11 mmol), 11 (166 mg, 1.70 mmol) and sodium ascorbate (22.4 mg, 0.11 mmol). The reaction was heated to 45° C. for 16 hours. After the completion of the reaction, the reaction was cooled to RT and diluted with water. The reaction mixture was stirred for 2 hours and the obtained solid was filtered and dried under reduced pressure to afford 60 mg (21% yield) of I-9 as an off-white solid. ¹H NMR (300 MHz, DMSO-d₆) δ 8.31 (s, 1H), 7.87 (s, 1H), 7.47 (d, J=8.8 Hz, 2H), 7.39 (dd, J=5.2, 8.4 Hz, 1H), 7.29-7.21 (m, 3H), 7.19-7.10 (m, 1H), 4.75 (s, 2H), 4.72 (s, 2H), 4.40 (s, 1H), 2.70 (s, 2H), 2.25-2.12 (m, 6H), 2.05-1.96 (m, 6H), 1.07 (s, 6H). LCMS (Method J): 504.43 (M+H), purity: 98.90% by AUC, ret. time: 5.20 min, UV: 245 nm. HPLC (Method Z): purity: 98.60% by AUC, ret. time: 14.52 min, UV: 245 nm.

### Example 5—Synthesis of 3-FLUORO-N-(4-(4-(4-(2-HYDROXY-2-METHYL PROPYL)-1H-1,2,3-TRIAZOL-1-YL)BICYCLO[2.2.2]OCTAN-1-YL)PHENYL)-5,7-DIHYDRO-6H-PYRROLO[3,4-B]PYRIDINE-6-CARBOXAMIDE (I-8)

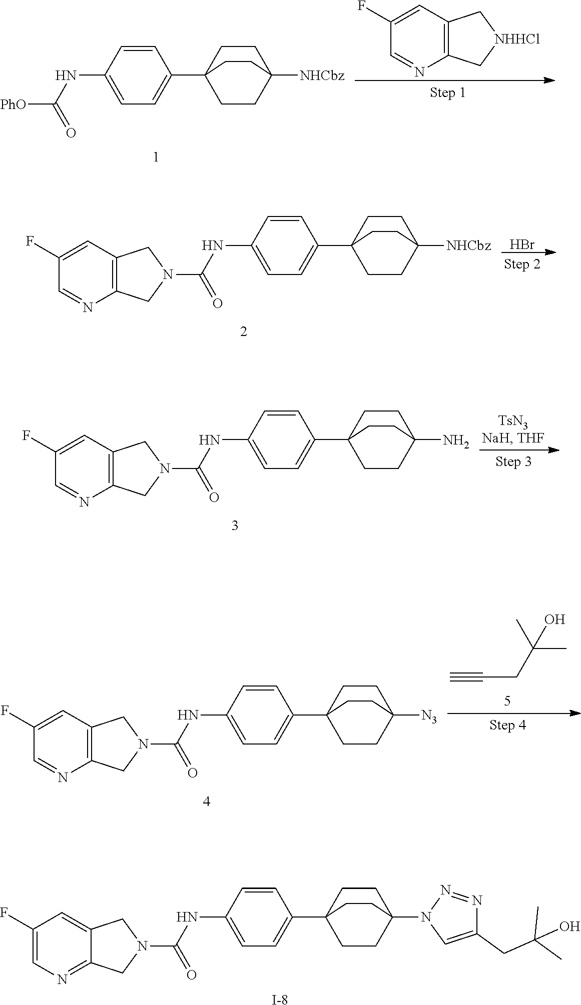

Step 1: Synthesis of benzyl (4-(4-(3-fluoro-6,7-dihydro-5H-pyrrolo [3,4-b]pyridine-6-carboxamido) phenyl) bicyclo [2.2.2]octan-1-yl) carbamate (2): 2 was prepared using General Procedure B using 1 (2.49 g, 7.94 mmol). The product was obtained after solid filtration, which afforded 2.5 g of 2 (91% yield) as a white solid. ¹H NMR (300 MHz, DMSO-d₆) δ 8.48 (s, J=1.1 Hz, 1H), 8.35 (s, 1H), 7.80 (dd, J=2.7, 9.1 Hz, 1H), 7.47-7.41 (m, 2H), 7.39-7.30 (m, 5H), 7.21 (d, J=9.0 Hz, 2H), 7.02 (s, 1H), 4.97 (s, 2H), 4.77 (s, 2H), 4.73 (s, 2H), 1.98-1.74 (m, 12H). LCMS (Method J): 515.44 (M+H), purity: 98.59% by AUC, ret. time: 5.70 min, UV: 240 nm.

Step 2: Synthesis of N-(4-(4-aminobicyclo [2.2.2]octan-1-yl) phenyl)-3-fluoro-5,7-dihydro-6H-pyrrolo [3,4-b]pyridine-6-carboxamide (3): To a stirred solution of 2 (1.8 g, 3.50 mmol) in acetic acid (10.0 mL) was added HBr (25.0 mL, 33% in acetic acid) at 0° C., then the mixture was stirred at RT for 16 hours. After completion of the reaction, the volatiles were removed under reduced pressure. The resulting mixture was triturated with MTBE (3×50 mL). The obtained solid was filtered and dried to obtain 1.30 g of 3 (97% yield) as an off-white solid. ¹H NMR (300 MHz, DMSO-d₆) δ 8.48 (s, 1H), 8.34 (s, 1H), 7.80 (dd, J=2.5, 8.9 Hz, 1H), 7.43 (d, J=8.6 Hz, 2H), 7.20 (d, J=8.8 Hz, 2H), 4.77 (s, 2H), 4.72 (s, 2H), 1.88-1.72 (m, 6H), 1.61-1.34 (m, 6H). LCMS (Method J): 381.42 (M+H), purity: 96.9% by AUC, ret. time: 4.05 min, UV: 240 nm.

Step 3: Synthesis of N-(4-(4-azidobicyclo [2.2.2]octan-1-yl) phenyl)-3-fluoro-5,7-dihydro-6H-pyrrolo [3,4-b]pyridine-6-carboxamide (4): To a stirred solution of NaH in THF (20 mL) was added 3 (500 mg, 1.31 mmol) and tosyl azide (1.04 g, 5.26 mmol) at 0° C. under a N₂ atmosphere. The reaction mixture was stirred at 65° C. for 32 hours. Tosyl azide (1.04 g, 5.26 mmol) was added, and then the reaction mixture was stirred at 65° C. for another 64 hours. The reaction mixture was monitored by TLC and LCMS. After completion of the reaction, the reaction mixture was diluted with water (50 mL) and extracted with EtOAc (3×100 mL). The combined organic extracts were dried over Na₂SO₄ and concentrated under reduced pressure to afford the crude product. The crude product was purified by column chromatography (eluent: 0-80% ethyl acetate in hexanes) to afford 100 mg of 4 (19% yield) as an off-white solid. ¹H NMR (300 MHz, DMSO-d₆) δ 8.48 (s, 1H), 8.36 (s, 1H), 7.80 (t, J=1.0 Hz, 1H), 7.49-7.43 (m, 2H), 7.26-7.18 (m, 2H), 4.77 (s, 2H), 4.73 (s, 2H), 2.08-1.56 (m, 12H). LCMS (Method J): 381.42 (M+H), purity: 96.9% by AUC, ret. time: 4.05 min, UV: 240 nm.

Step 4: Synthesis of 3-fluoro-N-(4-(4-(4-(2-hydroxy-2-methylpropyl)-1H-1,2,3-triazol-1-yl) bicyclo [2.2.2]octan-1-yl) phenyl)-5,7-dihydro-6H-pyrrolo [3,4-b]pyridine-6-carboxamide (I-8): To a stirred solution of 5 (22 mg, 0.22 mmol) in tert-BuOH (3 mL) and water (3 mL) was added sodium ascorbate (14.5 mg, 0.073 mmol), CuSO₄.5H₂O (18.3 mg, 0.073 mmol), and 4 (60 mg, 0.147) at RT. The reaction mixture was stirred at 100° C. for 1 hour in a microwave. After completion of the reaction, the reaction mixture was diluted with water (50 mL) and stirred for 2 hours at RT. The precipitate was filtered and washed with n-pentane (50 mL) to afford 25 mg of title compound I-8 (34%) as an off-white solid. ¹H NMR (300 MHz, DMSO-d₆) δ 8.49 (s, 1H), 8.38 (s, 1H), 7.92 (s, 1H), 7.81 (t, J=8.3 Hz, 1H), 7.49 (d, J=8.6 Hz, 2H), 7.28 (d, J=8.6 Hz, 2H), 4.78 (s, 2H), 4.73 (s, 2H), 4.45 (s, 1H), 2.69 (s, 2H), 2.29-2.10 (m, 6H), 2.08-1.90 (m, 6H), 1.09 (s, 6H). LCMS (Method J): 505.12 (M+H), purity: 95.15% by AUC, ret. time: 4.75 min, UV: 240 nm. HPLC (Method Z): purity: 93.82% by AUC, ret. time: 13.18 min, UV: 240 nm.

### Example 6—Synthesis of 5-FLUORO-N-(4-(4-(4-METHYL-1H-1,2,3-TRIAZOL-1-YL)BICYCLO[2.2.2]OCTAN-1-YL)PHENYL)ISOINDOLINE-2-CARBOXAMIDE (I-14)

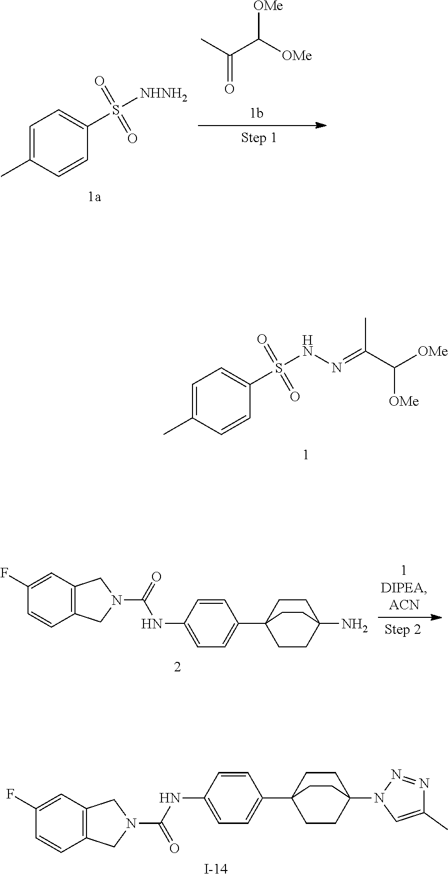

Step 1: Synthesis of (E)-N′-(1,1-dimethoxypropan-2-ylidene)-4-methylbenzene sulfonohydrazide (1): To a stirred solution of 1a (1.0 g, 5.37 mmol) in a DMSO (5 mL) was added 1b (634 mg 5.37 mmol) at RT under N₂, then the reaction mixture was stirred at RT for 1 hour. After completion of the reaction, the reaction mixture was quenched with ice cold water and the precipitate was filtered and washed with water (20 mL) to afford the crude product. The crude product was triturated with n-heptane to afford 1.0 g of 1 (65% yield) as an off-white solid. ¹H NMR (300 MHz, DMSO-d₆) δ 10.46 (s, 1H), 7.78-7.68 (m, 2H), 7.39 (d, J=8.0 Hz, 2H), 4.36 (s, 1H), 3.14 (s, 6H), 2.37 (s, 3H), 1.69 (s, 3H).

Step 2: Synthesis of 5-fluoro-N-(4-(4-(4-methyl-1H-1,2,3-triazol-1-yl) bicyclo [2.2.2]octan-1-yl) phenyl) isoindoline-2-carboxamide (I-14): To a stirred solution of 2 (100 mg, 0.26 mmol) in ACN (5 mL) was added DIPEA (0.27 mL, 1.56 mmol) and 1 (95.9 mg 0.31 mmol) at RT under N₂, then the reaction mixture was stirred at 80° C. for 6 hours. After completion of the reaction, the reaction mixture was quenched with ice cold water and the precipitate was filtered and washed with EtOAc (10 mL) to afford the crude product. The crude product was purified by column chromatography (eluent: 0-7% MeOH in DCM) to afford 22 mg of I-14 (19% yield) as an off-white solid. ¹H NMR (300 MHz, DMSO-d₆) δ 8.32 (s, 1H), 7.92 (s, 1H), 7.47 (d, J=8.7 Hz, 2H), 7.44-7.33 (m, 1H), 7.30-7.10 (m, 4H), 4.74 (s, 2H), 4.70 (s, 2H), 2.22 (s, 3H), 2.21-2.14 (m, 6H), 2.03-1.94 (m, 6H). LC-MS (Method J): [M+H]⁺=446.31 (M+H), purity: 98.64% by AUC, ret. time: 5.42 min, UV: 260 nm. HPLC (Method Z): purity: 97.87% by AUC, ret. time: 15.02, UV: 240 nm.

### Example 7—Synthesis of 5-FLUORO-N-(4-(4-(1-METHYL-1H-1,2,3-TRIAZOL-4-YL)BICYCLO[2.2.2]OCTAN-1-YL)PHENYL)ISOINDOLINE-2-CARBOXAMIDE (I-11) AND 5-FLUORO-N-(4-(4-(1-((TETRAHYDROFURAN-3-YL)METHYL)-1H-1,2,3-TRIAZOL-4-YL)BICYCLO[2.2.2]OCTAN-1-YL)PHENYL)ISOINDOLINE-2-CARBOXAMIDE (I-13)

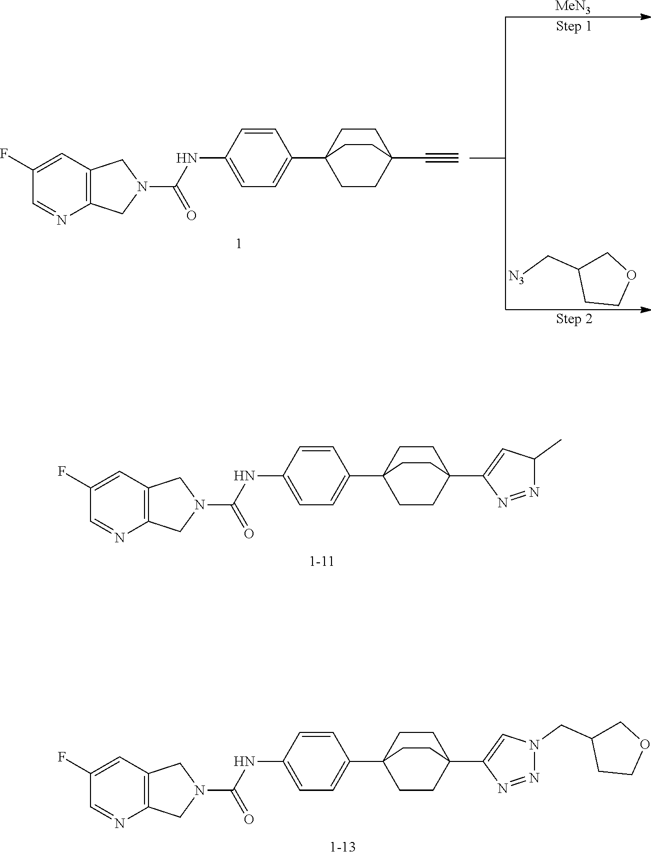

Step 1: Synthesis of 5-fluoro-N-(4-(4-(1-methyl-1H-1,2,3-triazol-4-yl) bicyclo [2.2.2]octan-1-yl) phenyl) isoindoline-2-carboxamide (I-11): To a stirred solution of 1 (40 mg, 0.10 mmol) in IPA/DCM (3.0 mL, 1:3) and water (0.6 mL) was added sodium ascorbate (1.98 mg, 0.01 mmol), CuSO₄.5H₂O (0.99 mg, 0.004 mmol) and methyl azide (8.8 mg, 0.15 mmol) at RT. The reaction mixture was stirred at RT for 2 hours in a sealed tube. After completion of the reaction, the reaction mixture was diluted with water (25 mL) and extracted with DCM (2×30 mL). The organic layer was dried over anhydrous Na₂SO₄, filtered and concentrated under reduced pressure to afford the crude product. The crude product was purified by column chromatography (eluent: 0-5% MeOH in DCM) and triturated with n-hexane to afford 25 mg of I-11 (55%) as an off-white solid. ¹H NMR (300 MHz, DMSO-d₆) δ 8.29 (s, 1H), 7.79 (s, 1H), 7.45 (d, J=8.7 Hz, 2H), 7.38 (dd, J=5.3, 8.3 Hz, 1H), 7.24 (d, J=8.6 Hz, 3H), 7.18-7.10 (m, 1H), 4.74 (s, 2H), 4.71 (s, 2H), 3.98 (s, 3H), 1.87 (s, 12H). LCMS (Method J): 446.31 (M+H), purity: 99.01% by AUC, ret. time: 5.23 min, UV: 240 nm. HPLC (Method Z): purity: 98.54% by AUC, ret. time: 14.81 min, UV: 250 nm.

Step 2: Synthesis of 5-fluoro-N-(4-(4-(1-((tetrahydrofuran-3-yl) methyl)-1H-1,2,3-triazol-4-yl) bicyclo [2.2.2]octan-1-yl) phenyl) isoindoline-2-carboxamide (I-13): I-13 was prepared using the procedure of Step 1 above, using 1 (100 mg, 0.25 mmol). The product was purified by column chromatography (eluent: 0-50% EtOAc in hexane), followed by trituration with n-pentane to afford 25 mg of I-13 (19%) as an off-white solid. ¹H NMR (300 MHz, DMSO-d₆) δ 8.29 (s, 1H), 7.88 (s, 1H), 7.48-7.38 (m, 3H), 7.24 (d, J=8.7 Hz, 3H), 7.18-7.11 (m, 1H), 4.74 (s, 2H), 4.72 (s, 2H), 4.29 (d, J=7.4 Hz, 2H), 3.79-3.60 (m, 3H), 3.45 (dd, J=5.5, 8.7 Hz, 1H), 2.71-2.63 (m, 1H), 1.88 (s, 13H), 1.64-1.55 (m, 1H). LCMS (Method J): 516.35 (M+H), purity: 98.64% by AUC, ret. time: 5.28 min, UV: 240 nm. HPLC (Method Z): purity: 99.51% by AUC, ret. time: 14.93 min, UV: 250 nm.

### Example 8—Synthesis of 3-FLUORO-N-(4-(4-(5-METHYL-1,3,4-OXADIAZOL-2-YL)BICYCLO[2.2.2]OCTAN-1-YL)PHENYL)-5,7-DIHYDRO-6H-PYRROLO[3,4-B]PYRIDINE-6-CARBOXAMIDE (I-7)

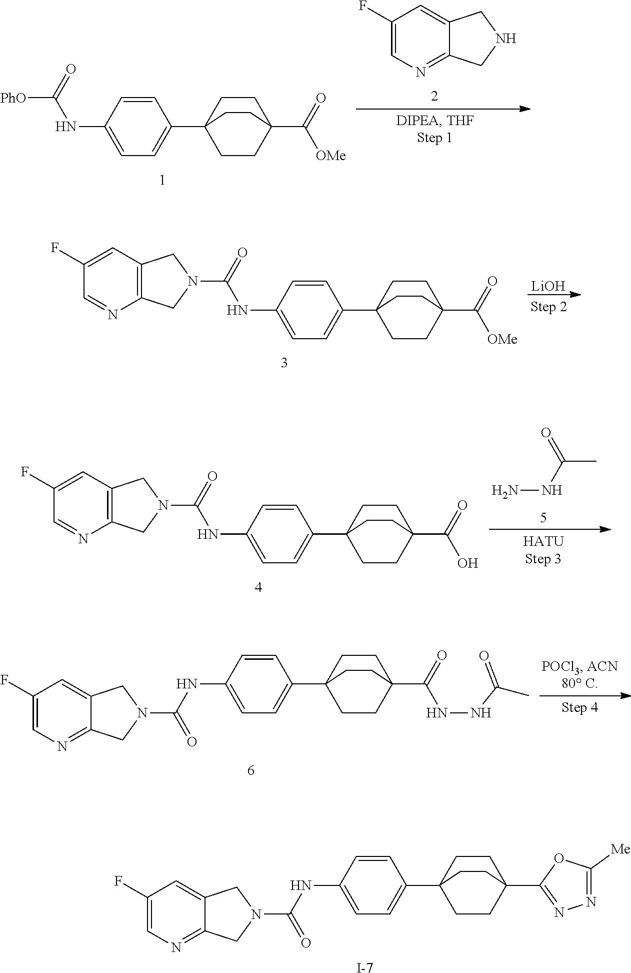

Step 1: Synthesis of methyl 4-(4-(3-fluoro-6,7-dihydro-5H-pyrrolo[3,4-b]pyridine-6-carboxamido) phenyl) bicyclo [2.2.2]octane-1-carboxylate (3): To 1 (250 mg, 0.659 mmol) in THF (10 mL) was added 2 (167 mg, 0.791 mmol) and DIPEA (1.3 mL, 9.88 mmol) at ambient temperature. The reaction mixture was stirred at 60° C. for 16 hours. After completion of the reaction, the reaction mixture was cooled to RT and the volatiles were removed under reduced pressure. The residue was stirred in a mixture of MTBE (10 mL) and water (10 mL) for 2 hours, and the precipitate was filtered and dried to obtain 200 mg of 3 (74% yield) as a white solid. ¹H NMR (300 MHz, DMSO-d₆) δ 8.50-8.46 (m, 1H), 8.34 (s, 1H), 7.83-7.76 (m, 1H), 7.45 (d, J=8.8 Hz, 2H), 7.21 (d, J=8.6 Hz, 2H), 4.77 (s, 2H), 4.72 (s, 2H), 3.59 (s, 3H), 1.88-1.72 (m, 12H).

Step 2: Synthesis of 4-(4-(3-fluoro-6,7-dihydro-5H-pyrrolo[3,4-b]pyridine-6-carboxamido) phenyl) bicyclo [2.2.2]octane-1-carboxylic acid (4): To a stirred solution of 3 (200 mg, 0.47 mmol) in a mixture of THF:MeOH:H₂O (24 mL, 5:5:1) was added lithium hydroxide (102.1 mg, 4.25 mmol) at 0° C. and the reaction mixture was stirred at RT for 48 hours. The reaction mixture was monitored by TLC and LCMS. After completion of the reaction, the reaction mixture was diluted with ice cold water and acidified (to pH ˜2) using 2.0 N HCl. The resulting precipitate was filtered and dried to obtain 190 mg of 4 (98% yield) as an off-white solid. ¹H NMR (300 MHz, DMSO-d₆) δ 12.03 (s, 1H), 8.49-8.46 (m, 1H), 8.34 (s, 1H), 7.79 (dd, J=2.3, 9.1 Hz, 1H), 7.45 (d, J=8.7 Hz, 2H), 7.21 (d, J=8.7 Hz, 2H), 4.77 (s, 2H), 4.72 (s, 2H), 1.85-1.70 (m, 12H).

Step 3: Synthesis of N-(4-(4-(2-acetylhydrazine-1-carbonyl) bicyclo [2.2.2]octan-1-yl) phenyl)-3-fluoro-5,7-dihydro-6H-pyrrolo[3,4-b]pyridine-6-carboxamide (6): To a stirred solution of 4 (300 mg, 0.73 mmol) in DMF (5.0 mL) was added 5 (59.7 mg, 0.80 mmol), followed by the addition of DIPEA (0.39 mL, 2.19 mmol) and HATU (362 mg, 0.95 mmol) at 0° C. The reaction mixture was stirred at RT for 16 hours. After completion of the reaction, the reaction mixture was poured into ice cold water and the precipitate was filtered, washed with water (25 mL), and dried to obtain 310 mg of 6 (90% yield) as an off-white solid. ¹H NMR (300 MHz, DMSO-d₆) δ 9.58 (s, 1H), 9.25 (s, 1H), 8.49 (s, 1H), 8.35 (s, 1H), 7.80 (dd, J=2.5, 8.9 Hz, 1H), 7.55-7.37 (m, J=8.6 Hz, 2H), 7.36-7.08 (m, 3H), 4.77 (s, 2H), 4.73 (s, 2H), 1.84 (s, 3H), 1.82-1.71 (m, 12H). LCMS (Method J): 466.33 (M+H), purity: 96.39% by AUC, ret. time: 4.12 min, UV: 240 nm.

Step 4: Synthesis of 3-fluoro-N-(4-(4-(5-methyl-1,3,4-oxadiazol-2-yl) bicyclo [2.2.2]octan-1-yl) phenyl)-5,7-dihydro-6H-pyrrolo[3,4-b]pyridine-6-carboxamide (I-7): To a stirred solution of 6 (100 mg, 0.537 mmol) in ACN (5 mL) was added POCl₃ (0.059 mL, 0.64 mmol) at RT. The reaction mixture was heated to 80° C. for 3 hours. After completion of the reaction, the reaction mixture was cooled to 0° C., quenched with a saturated NaHCO₃ solution and extracted with EtOAc (2×50 mL). The combined organic layer was dried over anhydrous Na₂SO₄, filtered, and concentrated under reduced pressure to obtain the crude product. The crude product was purified by column chromatography (eluent: 0-5% MeOH in DCM) to afford 22 mg of I-7 (22.91% yield) as an off-white solid. ¹H NMR (300 MHz, DMSO-d₆) δ 8.49 (d, J=1.1 Hz, 1H), 8.37 (s, 1H), 7.80 (dd, J=2.8, 9.1 Hz, 1H), 7.48 (d, J=8.8 Hz, 2H), 7.25 (d, J=8.6 Hz, 2H), 4.78 (s, 2H), 4.73 (s, 2H), 2.47 (s, 3H), 2.02-1.86 (m, 12H). LCMS (Method J): 448.06 (M+H), purity: 97.90% by AUC, ret. time: 5.03 min, UV: 240 nm. HPLC (Method Z): purity: 96.48% by AUC, ret. time: 13.75 min, UV: 240 nm.

### Example 9—Synthesis of 3-FLUORO-N-(4-((1R,4R)-4-(4-(2-HYDROXY-2-METHYLPROPYL)-1H-1,2,3-TRIAZOL-1-YL) CYCLOHEXYL) PHENYL)-5,7-DIHYDRO-6H-PYRROLO[3,4-B]PYRIDINE-6-CARBOXAMIDE (I-6)

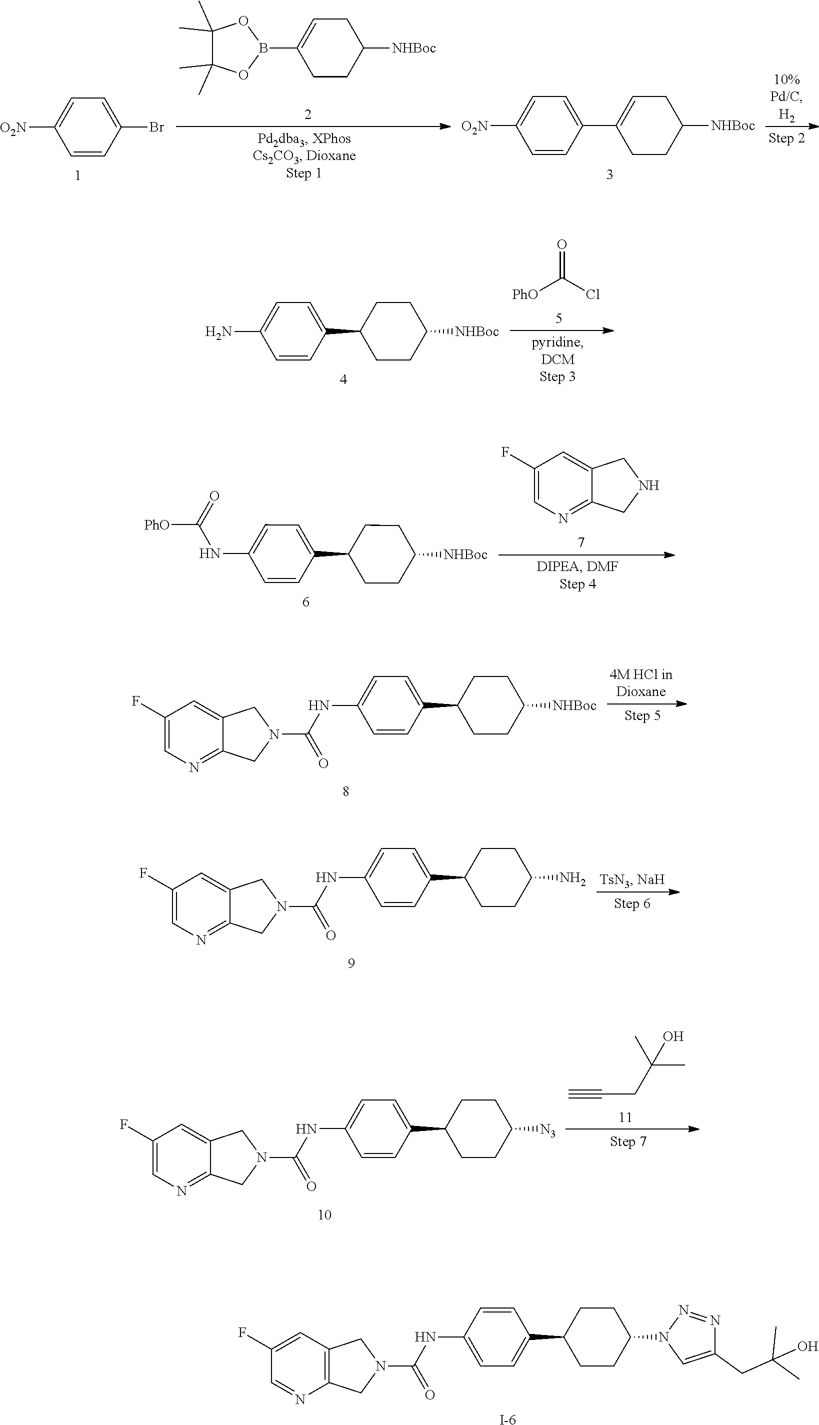

Step 1: Synthesis of tert-butyl (4′-nitro-2,3,4,5-tetrahydro-[1,1′-biphenyl]-4-yl)carbamate (3): A mixture of 1 (5 g, 24.75 mmol), 2 (10 g, 30.94 mmol), and Cs₂CO₃ (24 g, 73.65 mmol) in 1,4-dioxane:H₂O (1:1, 100 mL) was degassed with argon for 5 minutes. Pd₂(dba)₃ (1.3 g, 1.4 mmol) and X-phos (1.2 g, 2.5 mmol) were added to the above mixture at RT. The reaction mixture was stirred for 15 minutes while degassing with argon, and then the reaction mixture was heated to 90° C. and stirred for 16 hours. After completion of the reaction (monitored by TLC), the reaction mixture was filtered through a CELITE® pad and washed with EtOAc. The organic layer was diluted with water and extracted with EtOAc. The combined organic layers were washed with brine, dried over anhydrous Na₂SO₄, and concentrated under reduced pressure. The crude product was triturated with n-heptane, filtered and dried under reduced pressure to afford 3 (4.2 g, 13 mmol, 53% yield) as an off-white solid. The crude compound was used in the next step without further purification. LC-MS (Method I): ret. time: 2.30 min, mass calculated for chemical formula C₁₇H₂₂N₂O₄: 318.37; found: 262.9 [(M-Isobutene)+H].

Step 2: Synthesis of tert-butyl (4-(4-aminophenyl)cyclohexyl)carbamate (4): To a stirred solution of 3 (4.2 g, 13 mmol) in MeOH (40 mL) was added 10% palladium on carbon (50% wet, 2.0 g) and the mixture was stirred at RT for 16 hours under hydrogen gas at 150 psi in an autoclave. After completion of the reaction (monitored by TLC), the reaction mixture was filtered through a CELITE® pad and washed with EtOAc. The combined organic layers were concentrated under reduced pressure to give a gummy mass. The crude residue was triturated with diethyl ether, and the solid was filtered and dried under reduced pressure to give 4 (trans-isomer, 1.0 g, off-white solid). The other isomer that was found in the mother liquor (filtrate) and was concentrated to give the cis-isomer (3.0 g, as a solid). The trans isomer was used for the next step. LC-MS (Method I): ret. time: 1.98 min, mass calculated for chemical formula C₁₇H₂₆N₂O₂: 290.41; Found: 235.0 [M-Isobutene+H]⁺.

Step 3: Synthesis of phenyl (4-((1r,4r)-4-((tert-butoxycarbonyl)amino) cyclohexyl) phenyl)carbamate (6): To a stirred solution of 4 (1.0 g, 3.44 mmol) in DCM (30 mL) was added pyridine (0.56 mL, 6.88 mmol) and 5 (0.647 g, 4.13 mmol) at 0° C. The mixture was stirred at RT for 2 hours under nitrogen atmosphere. After completion of the reaction (monitored by TLC), the reaction mixture was diluted with water and extracted with DCM. The combined organic layers were washed with brine, dried over anhydrous Na₂SO₄, and concentrated under reduced pressure. The crude compound was triturated with diethyl ether, filtered and dried under reduced pressure to give 6 (1.2 g, 2.9 mmol, 85% yield) as an off-white solid. LC-MS (Method I): ret. time: 2.28 min, mass calculated for chemical formula C₂₄H₃₀N₂O₄: 410.51; found: 354.9 [(M-isobutene)+H]*.

Step 4: Synthesis of tert-butyl ((1r,4r)-4-(4-(3-fluoro-6,7-dihydro-5H-pyrrolo [3,4-b]pyridine-6-carboxamido)phenyl)cyclohexyl)carbamate (8): To a stirred solution of 6 (0.55 g, 1.34 mmol) and 7 (0.292 g, 1.608 mmol) in DMF (10 mL) was added DIPEA (1.17 mL, 6.69 mmol) at RT and then the mixture was refluxed at 80° C. for 3-4 hours under nitrogen atmosphere. After completion of the reaction (monitored by TLC), the reaction mixture was diluted with water and extracted with DCM. The combined organic layers were washed with water, followed by brine, dried over anhydrous Na₂SO₄, and concentrated under reduced pressure to afford 8 (0.55 g, 1.21 mmol, 90.32% yield) as an off-white solid. LC-MS (Method I): ret. time: 1.32 min, mass calculated for chemical formula C₂₅H₃₁FN₄O₃: 454.55; Found: 455.61 [M+H]⁺.

Step 5: Synthesis of N-(4-((1r,4r)-4-aminocyclohexyl)phenyl)-3-fluoro-5,7-dihydro-6H-pyrrolo[3,4-b]pyridine-6-carboxamide hydrochloride (9): To a stirred solution of 8 (0.55 g, 1.21 mmol) in DCM (10 mL) was added 4 M HCl in 1,4-dioxane (5 mL) at 0° C. and the mixture was stirred at RT for 2-3 hours. After completion of the reaction (monitored by TLC), volatiles were removed under reduced pressure. The residue was then stirred in diethyl ether (10 mL) and then with pentane (5 mL×2), filtered and the solid compound was dried under reduced pressure to afford 9 (0.45 g, crude) as an off-white solid. The resulting crude product was used in the next step without further purification. LC-MS (Method I): ret. time: 1.28 min, mass calculated for chemical formula C₂₀H₂₃FN₄O: 354.4; Found: 355.1 [M+H]⁺.

Step 6: Synthesis of N-(4-((1r,4r)-4-azidocyclohexyl)phenyl)-3-fluoro-5,7-dihydro-6H-pyrrolo[3,4-b]pyridine-6-carboxamide (10): To a stirred solution of NaH (54 mg, 2.22 mmol) in THF (10 mL) was added 9 (130 mg, 0.37 mmol) and tosyl azide (146 mg, 0.74 mmol) at 0° C. under N₂ atmosphere. The reaction mixture was stirred at 65° C. for 32 hours. Tosylazide (146 mg, 0.74 mmol) was added and then the reaction mixture was stirred at 65° C. for another 64 hours. The reaction mixture was monitored by TLC and LCMS. After completion of the reaction, the reaction mixture was diluted with water (25 mL) and extracted with EtOAc (3×50 mL). The combined organic extracts were dried over Na₂SO₄ and concentrated under reduced pressure to afford the crude product (65 mg), which was used in the next step without further purification.

Step 7: Synthesis of 3-fluoro-N-(4-((1r,4r)-4-(4-(2-hydroxy-2-methylpropyl)-1H-1,2,3-triazol-1-yl) cyclohexyl)phenyl)-5,7-dihydro-6H-pyrrolo[3,4-b]pyridine-6-carboxamide (I-6): To a stirred solution of 10 (65 mg, 0.147 mmol) in tert-BuOH (3 mL) and water (3 mL) was added 11 (50 mg, 0.44 mmol), sodium ascorbate (14.5 mg, 0.073 mmol) and CuSO₄.5H₂O (18.3 mg, 0.073 mmol) at RT. The reaction mixture was stirred at 50° C. for 16 hours. The reaction mixture was monitored by TLC and LCMS. After completion of the reaction, the reaction mixture was diluted with water (20 mL) and stirred at RT for 2 hours. The precipitate was filtered and washed with n-pentane (50 mL) to afford 30 mg of I-6 (17% yield over two steps) as an off-white solid. ¹H NMR (300 MHz, DMSO-d₆) δ 8.52-8.46 (m, 1H), 8.37 (s, 1H), 7.95-7.89 (m, 1H), 7.80 (dd, J=2.0, 8.8 Hz, 1H), 7.48 (d, J=8.6 Hz, 2H), 7.17 (d, J=8.6 Hz, 2H), 4.78 (s, 2H), 4.73 (s, 2H), 4.63-4.46 (m, 2H), 2.75-2.70 (m, 3H), 2.21-2.11 (m, 2H), 1.94 (d, J=11.9 Hz, 4H), 1.72-1.62 (m, 2H), 1.13-1.06 (m, 6H). LCMS (Method J): 479.05 (M+H), purity: 97.16% by AUC, ret. time: 4.45 min, UV: 240 nm. HPLC (Method Z): purity: 92.75% by AUC, ret. time: 12.62 min, UV: 245 nm.

### Example 10—Synthesis of 3-FLUORO-N-(4-(4-(4-METHYL-1H-1,2,3-TRIAZOL-1-YL)BICYCLO[2.2.2]OCTAN-1-YL)PHENYL)-5,7-DIHYDRO-6H-PYRROLO[3,4-B]PYRIDINE-6-CARBOXAMIDE (I-5)

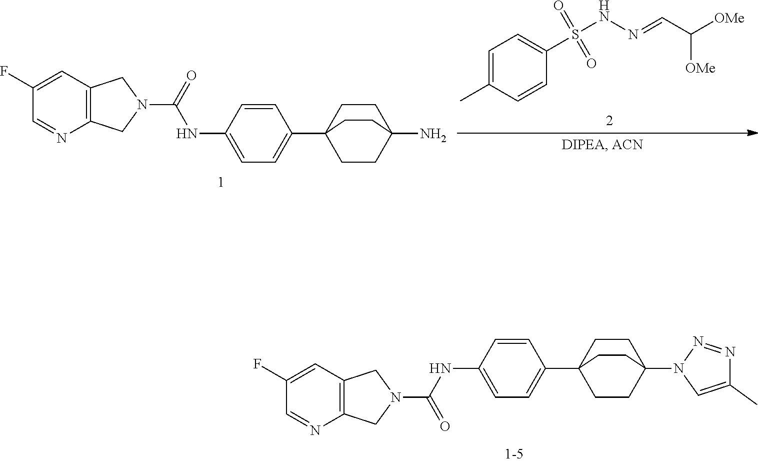 [SMILES: NC12CCC(c3ccc(NC(=O)N4Cc5cc(F)cnc5C4)cc3)(CC1)CC2 ; COC(/C=N/NS(=O)(=O)c1ccc(C)cc1)OC ; Cc1cn(C23CCC(c4ccc(NC(=O)N5Cc6cc(F)cnc6C5)cc4)(CC2)CC3)nn1]

To a stirred solution of 1 (200 mg, 0.52 mmol) in acetonitrile (5 mL) was added DIPEA (0.54 mL, 3.12 mmol) and 2 (192 mg 0.62 mmol) at RT under N₂. The reaction mixture was stirred at 80° C. for 6 hours. After completion of the reaction, the reaction mixture was quenched with ice cold water and the precipitate was filtered and washed with EtOAc (10 mL) to afford the crude product. The product was purified by column chromatography (eluent: 5-10% MeOH-DCM) to afford 80 mg of I-5 (34% yield) as an off-white solid. ¹H NMR (300 MHz, DMSO-d₆) δ 8.48 (d, J=1.4 Hz, 1H), 8.37 (s, 1H), 7.91 (d, J=0.7 Hz, 1H), 7.80 (dd, J=2.7, 9.0 Hz, 1H), 7.48 (d, J=8.8 Hz, 2H), 7.27 (d, J=8.8 Hz, 2H), 4.77 (s, 2H), 4.73 (s, 2H), 2.22 (d, J=0.6 Hz, 3H), 2.21-2.13 (m, 6H), 2.04-1.95 (m, 6H). LC-MS (Method J): [M+H]⁺=447.32 (M+H), purity: 98.63% by AUC, ret. time: 4.73 min, UV: 240 nm. HPLC (Method Z): purity: 93.72% by AUC, ret. time: 13.45 min, UV: 245 nm.

### Example 11—Synthesis of 3-FLUORO-N-(4-(4-(1-(2-HYDROXY-2-METHYL PROPYL)-1H-1,2,3-TRIAZOL-4-YL)BICYCLO[2.2.2]OCTAN-1-YL)PHENYL)-5,7-DIHYDRO-6H-PYRROLO[3,4-B]PYRIDINE-6-CARBOXAMIDE (I-10)

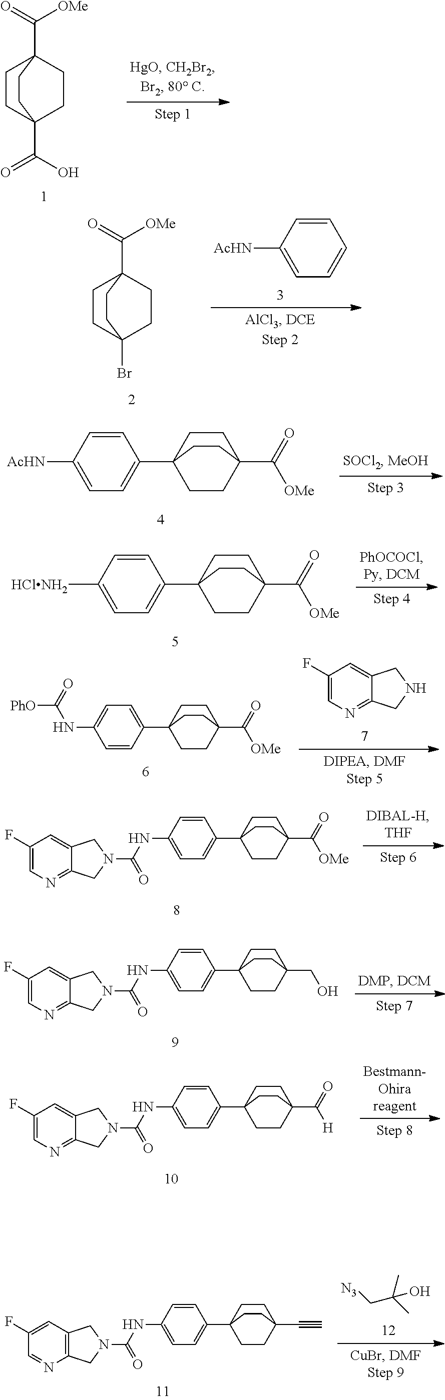

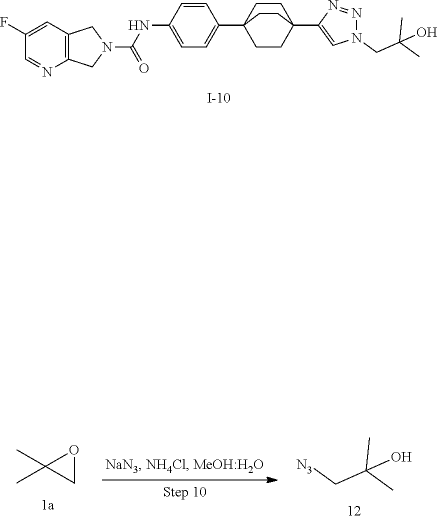

Step 1: Synthesis of methyl 4-bromobicyclo[2.2.2]octane-1-carboxylate (2): To a stirred solution of 1 (5.0 g, 23.55 mmol) in 1,2-dibromoethane (15 mL) was added mercuric oxide (5.15 g, 23.55 mmol) at 0° C. The temperature was raised to 80° C. and bromine (1.58 mL, 30.62 mmol) in 1,2-dibromoethane (5 mL) was added. The reaction mixture was stirred at 80° C. for 3 hours. After completion of the reaction (monitored by TLC), the reaction mixture was filtered through a CELITE® pad and the pad was washed with EtOAc. The organic layer was concentrated under reduced pressure. The resulting crude compound was purified by CombiFlash (Nexgen-300) chromatography (24 g silica gel cartridge) using 5% EtOAc in n-heptane as the eluent to afford 2 (5.5 g, 22 mmol, 94% yield) as an off-white semi-solid. ¹H NMR (400 MHz, DMSO-d₆) δ 3.69-3.56 (s, 3H), 2.56-2.25 (m, 6H), 1.89-1.82 (m, 6H).

Step 2: Synthesis of methyl 4-(4-acetamidophenyl)bicyclo[2.2.2]octane-1-carboxylate (4): To stirred solution of 2 (5.50 g, 20.23 mmol) and 3 (3.0 g, 22 mmol) in 1,2-DCE (50 mL) was added aluminum chloride (12 g, 89 mmol) portion wise at 0° C. The mixture was then stirred at RT for 16 hours. After completion of the reaction (monitored by TLC), the reaction mixture was slowly poured in ice cold water. The aqueous layer was extracted with DCM. The combined organic extracts were washed with brine, dried over sodium sulfate, filtered, and concentrated under reduced pressure. The obtained crude compound was purified by CombiFlash (Nexgen-300) chromatography (24 g silica gel cartridge) using 25% EtOAc in n-heptane as the eluent to afford 4 (1.0 g, 3.23 mmol, 16.0% yield) as a white solid. LC-MS (Method G): ret. time: 2.19 min, mass calculated for chemical formula: C₁₈H₂₃NO₃: 301.39; found: 302.50 [M+H]⁺.

Step 3: Synthesis of methyl 4-(4-aminophenyl)bicyclo[2.2.2]octane-1-carboxylate (5): To stirred solution of 4 (1.0 g, 3.32 mmol) in MeOH (15 mL) was added SOCl₂ (0.24 mL, 3.32 mmol) dropwise at RT. Then the reaction mixture was heated to 80° C. for 8 hours. After completion of the reaction (monitored by TLC), the reaction mixture was cooled to RT. The volatiles were removed under reduced pressure. The crude residue was diluted with water, basified with a saturated NaHCO₃ solution, and extracted with EtOAc. The combined organic layer was dried over sodium sulfate, filtered and concentrated under reduced pressure to afford 5 (0.8 g, 3 mmol, 90% yield) as an off-white solid. LC-MS (Method F): ret. time: 0.99 min, mass calculated for chemical formula: C₁₆H₂₁NO₂: 259.3; found: 260.42 [M+H]⁺.

Step 4: Synthesis of methyl 4-(4-((phenoxycarbonyl)amino) phenyl) bicyclo[2.2.2]octane-1-carboxylate (6): To a stirred solution of 5 (0.8 g, 3 mmol) in DCM (20 mL) was added pyridine (1.0 mL, 10 mmol), phenyl chloroformate (0.7 g, 5 mmol), and DMAP (40 mg) at 0° C. The mixture was then stirred at RT for 2 hours under nitrogen atmosphere. After completion of the reaction (monitored by TLC), the reaction mixture was diluted with water and extracted with 5% MeOH in DCM. The combined organic layers were washed with water followed by brine, dried over anhydrous Na₂SO₄, and concentrated under reduced pressure. The resulting crude compound was triturated with diethyl ether and n-pentane, filtered, and dried under reduced pressure to afford the 6 (1.1 g, 2.6 mmol, 80% yield) as an off-white solid. LC-MS (Method F): ret. time: 1.51 min, mass calculated for chemical formula: C₂₃H₂₅NO₄: 379.46; found: 380.50 [M+H]⁺.

Step 5: Synthesis of methyl 4-(4-(3-fluoro-6,7-dihydro-5H-pyrrolo[3,4-b]pyridine-6-carboxamido)phenyl)bicyclo[2.2.2]octane-1-carboxylate (8): To a stirred solution of 6 (1.0 g, 2.63 mmol) in DMF (15 mL) was added 7 (0.48 g, 2.76 mmol) and DIPEA (2.76 mL, 15.81 mmol) at RT, and the mixture was stirred at 90° C. for 6 hours. After completion of the reaction (monitored by TLC), the reaction mixture was quenched with ice cold water and extracted with EtOAc. The combined organic layers were washed with water, followed by brine, dried over anhydrous Na₂SO₄, and concentrated under reduced pressure. The crude product was triturated with diethyl ether and n-pentane, filtered and dried under reduced pressure to afford 8 (0.6 g, 1.41 mmol, 50% yield) as an off-white solid. LCMS (Method F): ret. time: 1.29 min, mass calculated for chemical formula: C₂₄H₂₆FN₃O₃: 423.49; found: 424.53 [M+H]⁺.

Step 6: Synthesis of 3-fluoro-N-(4-(4-(hydroxymethyl)bicyclo[2.2.2]octan-1-yl)phenyl)-5,7-dihydro-6H-pyrrolo[3,4-b]pyridine-6-carboxamide (9): To a stirred solution of 8 (0.5 g, 1.18 mmol) in THF (10 mL) was added DIBAL-H (1.0 mol/L) in toluene (3.54 mL, 3.54 mmol) at 0° C. The reaction mixture stirred at RT for 16 hours. After completion of the reaction (monitored by TLC), the reaction mixture was poured in ice cold water and extracted with EtOAc. The combined organic layers were dried over anhydrous sodium sulfate, filtered and concentrated under reduced pressure. The crude semi-solid was triturated/washed with diethyl ether (7 mL) and n-pentane (5 mL), filtered and concentrated under reduced pressure to afford 9 (0.45 g, 1.14 mmol, 33.66% yield) as an off-white solid. LCMS (Method F): ret. time: 1.13 min, mass calculated for chemical formula: C₂₃H₂₆FN₃O₂: 395.48; found: 396.51 [M+H]⁺.

Step 7: Synthesis of 3-fluoro-N-(4-(4-formylbicyclo[2.2.2]octan-1-yl)phenyl)-5,7-dihydro-6H-pyrrolo[3,4-b]pyridine-6-carboxamide (10): To a stirred solution of 9 (0.45 g, 1.14 mmol) in dry DCM (25 mL) was added in 4 portions of Dess-Martin periodinane (0.72 g, 1.71 mmol) at 0° C. and the reaction was stirred at RT for 8 hours. After completion of the reaction, the reaction mixture was poured into ice cold water and extracted with DCM. The combined organic layer was dried over anhydrous sodium sulfate, filtered and concentrated under reduced pressure to afford the semi solid crude product. The crude product was triturated with n-pentane (5 mL), filtered and concentrated under reduced pressure to afford 10 (0.38 g, 0.96 mmol, 12.52% yield) as an off-white solid. LCMS (Method F): ret. time: 1.25 min, mass calculated for chemical formula: C₂₃H₂₄FN₃O₂: 393.46; found: 394.47 [M+H]⁺.

Step 8: Synthesis of N-(4-(4-ethynylbicyclo[2.2.2]octan-1-yl)phenyl)-3-fluoro-5,7-dihydro-6H-pyrrolo[3,4-b]pyridine-6-carboxamide (11): A two-neck round bottom flask was charged with 10 (0.3 g, 0.76 mmol) and dry potassium carbonate (0.316 g, 2.29 mmol). The flask was degassed by evacuating and backfilling with argon for a total of three cycles. The reaction flask was placed under a slight positive pressure of argon. Dry MeOH (20 mL) was added under argon atmosphere. To the mixture was added dimethyl (1-diazo-2-oxopropyl)phosphonate (0.179 g, 0.915 mmol) dropwise at 0° C. The mixture was then stirred at RT for 2 h. Progress of the reaction was monitored by TLC. After completion of the reaction, the reaction mixture was poured into ice cold water and extracted with EtOAc. The combined organic layer was dried with anhydrous Na₂SO₄, filtered and concentrated under pressure to afford a brown sticky semi solid crude product. The crude product was purified by combi-flash column chromatography and eluted with 35% of EtOAc in heptanes to afford 11 (0.25 g) as an off-white solid. LCMS (Method F): ret. time: 1.38 min, mass calculated for chemical formula: C₂₄H₂₄FN₃O: 389.47; found: 390.52 [M+H]⁺.

Step 9: Synthesis of 3-fluoro-N-(4-(4-(1-(2-hydroxy-2-methylpropyl)-1H-1,2,3-triazol-4-yl)bicyclo[2.2.2]octan-1-yl)phenyl)-5,7-dihydro-6H-pyrrolo[3,4-b]pyridine-6-carboxamide (I-10): A screw type vial was charged with 11 (0.04 g, 0.103 mmol) and 12 (0.023 g, 0.2054 mmol) in dry DMF (3 mL) which was degassed with argon gas for 10 min. CuBr (5.8 mg, 00.04108 mmol) was added. The vial was sealed and the mixture was stirred at 40° C. for 4 hours. After completion of the reaction, the reaction mixture was poured in to ice cold water (3 mL). The precipitated solid was filtered and dried under vacuum and then washed with diethyl ether (2×2 mL) and n-pentane (4 mL) to afford the crude product as an off-white solid. The crude product was purified by prep-HPLC purification to afford I-10 (11.9 mg) as a white solid. ¹H NMR (400 MHz, DMSO-d₆) δ: 8.48 (br s, 1H), 8.35 (s, 1H), 7.80 (dd, J=2.4, 9.0 Hz, 1H), 7.68 (s, 1H), 7.50-7.43 (m, 2H), 7.26 (d, J=8.8 Hz, 2H), 4.81 (s, 1H), 4.76 (br d, J=19.6 Hz, 4H), 4.21 (s, 2H), 1.95-1.83 (m, 12H), 1.06 (s, 6H). LC-MS (Method F): ret. time: 1.12 min, 99.93%; mass calculated for chemical formula C₂₈H₃₃FN₆O₂: 504.61; Found: 505.67 [M+H]⁺. HPLC: ret. time: 7.312 min, 99.53%; method: column: X-Select C18 (4.6×150) mm 5.0 μm; mobile phase A: 0.1% FA in (ACN:H₂O) (5:95, v/v); mobile phase B: acetonitrile (100%); column temperature: 30° C., flow rate: 1.0 mL/min, gradient: 0.00/5, 1.00/5, 8.00/100, 12.00/100, 14.00/5, 18.00/5; diluent: ACN:water (80:20). Prep-HPLC purification method: column: X-SELECT C18 (30*250 mm); mobile phase A: 0.1% formic acid in water, mobile phase B: acetonitrile, flow rate: 25 ml/min; loading (mg/injection): 20, gradient (time/% B): 0/20, 2/20, 10/30, 20/60, 25/100; sample diluent: ACN+water+THF.

Step 10: Synthesis of 1-azido-2-methylpropan-2-ol (12): To a stirred solution of 2,2-dimethyloxirane (0.25 g, 3.5 mmol) and sodium azide (0.57 g, 8.7 mmol) in MeOH (6.92 mL) was added a solution of ammonium chloride (0.37 g, 6.9 mmol) in water (0.765 mL). The reaction mixture was stirred at room temperature for 36 hours. Progress of the reaction was monitored by TLC. After completion of the reaction, the reaction solution was concentrated to 0.4 mL by rotary evaporation at 50° C. The mixture was diluted with 2 ml of water, and then extracted with EtOAc (3×20 ml). The organic layer was collected and dried over Na₂SO₄, filtered and concentrated under reduced pressure to afford 12 (0.15 g, 1.3 mmol, 38% yield) as a colorless oil. ¹H NMR (401 MHz, DMSO-d₆) δ: 4.75-4.73 (m, 1H), 3.10 (s, 2H), 1.11 (s, 6H). ¹³C NMR (101 MHz, DMSO-d₆) δ: 69.98, 60.96, 26.89.

### Example 12—Synthesis of 5-FLUORO-N-(4-((1R,4R)-4-(4-(2-HYDROXY-2-METHYLPROPYL)-1H-1,2,3-TRIAZOL-1-YL)CYCLOHEXYL) PHENYL) ISOINDOLINE-2-CARBOXAMIDE (I-4)

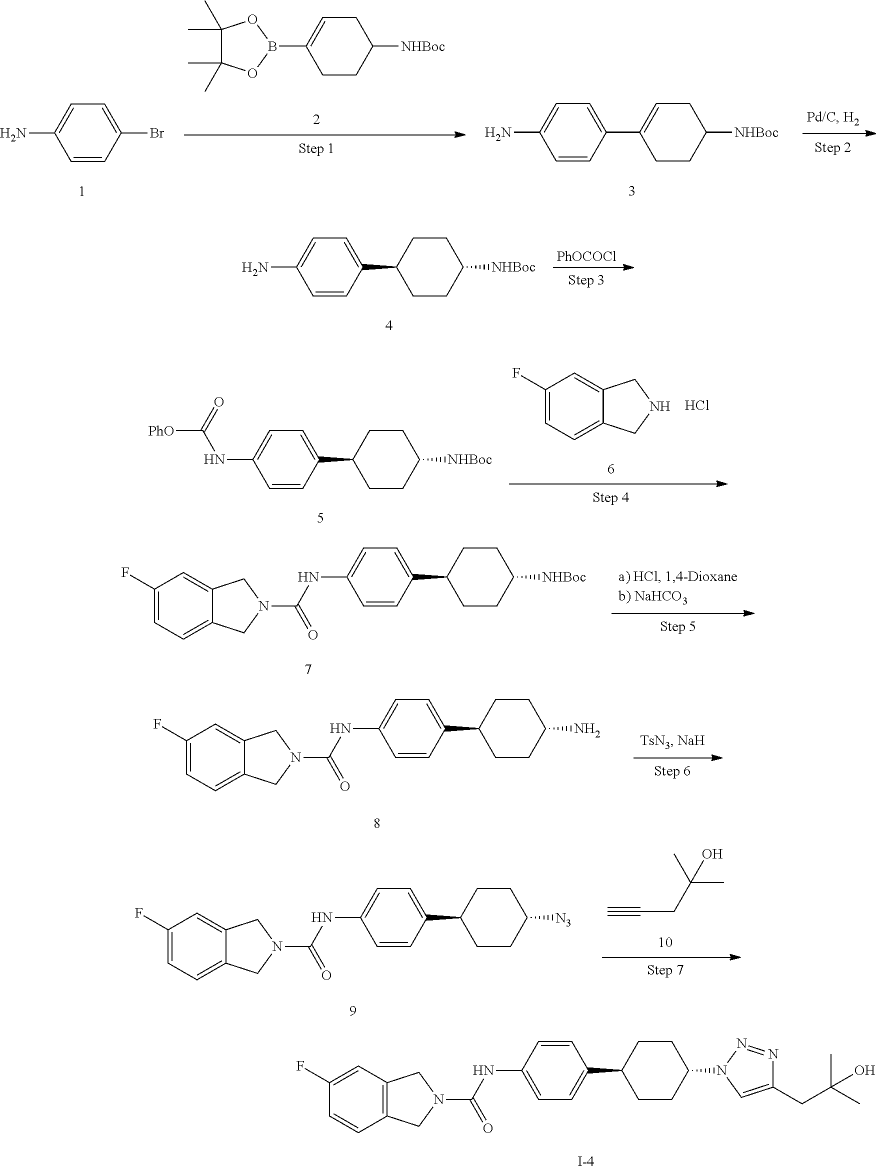

Step 1: Synthesis of tert-butyl (4′-amino-2,3,4,5-tetrahydro-[1,1′-biphenyl]-4-yl) carbamate (3): To a stirred solution of 2 (2.0 g, 6.19 mmol) and 1 (1.05 g, 6.19 mmol) in 1,4-dioxane/water (40 mL) was added cesium carbonate (2.0 g, 12.38 mmol). The reaction mixture was purged with argon for 10 min, then Pd₂(dba)₃ (28 mg, 0.03 mmol) was added and the mixture was purged with argon for another 5 min. XPhos (0.91 g, 0.62 mmol) was added at rt. The reaction mixture was stirred at 90° C. for 6 hours. After completion of the reaction, the reaction mixture was cooled to rt, filtered through a pad of CELITE®, diluted with ice-cold water (100 mL) and extracted with ethyl acetate (3×80 mL). The combined organic extracts were washed with brine solution (2×50 mL), dried over anhydrous Na₂SO₄ and concentrated under reduced pressure to afford the crude product. The product was purified by combi-flash chromatography (eluent-0-10% ethyl acetate in hexanes) which afforded 1.2 g of 3 (70% yield) as an off-white solid. ¹H NMR (300 MHz, DMSO-d₆) δ 7.08 (d, J=8.6 Hz, 2H), 6.79 (d, J=7.5 Hz, 1H), 6.53-6.45 (m, J=8.6 Hz, 2H), 5.79 (s, 1H), 5.04 (s, 2H), 3.54-3.38 (m, 1H), 2.42-2.25 (m, 3H), 2.08-1.81 (m, 2H), 1.58-1.43 (m, 1H), 1.42-1.33 (m, 9H), 1.09-1.03 (m, 1H). LC-MS (Method J): [M+H]⁺=289.11 (M+H), purity: 97.9% by AUC, ret. time: 5.47 min, UV: 260 nm.

Step 2: Synthesis of tert-butyl ((1r,4r)-4-(4-aminophenyl) cyclohexyl) carbamate (4): To a solution of 3 (1.2 g, 4.16 mmol) in MeOH (20 mL) was added Pd—C(150 mg, 10%) at rt, and then the mixture was stirred under hydrogen atmosphere for 12 hours. After complete consumption of the starting material, the reaction mixture was filtered through a pad of CELITE®, and the pad was washed with MeOH. The combined filtrate was concentrated under reduced pressure to obtain the crude product. The crude product was taken in 2% EtOAc-hexanes and stirred at 70° C. for 3 hours and the mixture was filtered and dried to afford 250 mg of 4 (20% yield) as an off-white solid. ¹H NMR (300 MHz, DMSO-d6) δ 6.85 (d, J=8.3 Hz, 2H), 6.73 (d, J=8.0 Hz, 1H), 6.46 (d, J=8.4 Hz, 2H), 4.79 (s, 2H), 3.29-3.14 (m, 1H), 2.29-2.15 (m, 1H), 1.82 (bd, J=11.2 Hz, 2H), 1.70 (d, J=12.4 Hz, 2H), 1.45-1.17 (m, 13H). LC-MS (Method J): [M+H]⁺=291.17 (M+H), purity: 98.77% by AUC, ret. time: 5.52 min, UV: 260 nm.

Step 3: Synthesis of phenyl (4-((1r,4r)-4-((tert-butoxycarbonyl) amino) cyclohexyl) phenyl) carbamate (5): To a stirred solution of 4 (200 mg, 0.69 mmol) and DIPEA (0.31 mL, 1.73 mmol) in THF (10.0 mL) was added phenyl chloroformate (0.09 mL, 0.76 mmol) slowly at 0° C. The reaction mixture was stirred at 0° C. for 30 min and rt for 2 hours. The reaction mixture was monitored by TLC and LCMS. After completion of the reaction, the reaction mixture was poured into ice-cold water (50 mL) and extracted with EtOAc (2×50 mL). The combined organic extracts were dried with Na₂SO₄, filtered and concentrated to give the crude product. The crude product was triturated with n-pentane (30 mL) to afford 300 mg of 5 (96% yield) as an off-white solid. ¹H NMR (300 MHz, DMSO-d₆) δ 10.12 (s, 1H), 7.47-7.35 (m, 4H), 7.29-7.14 (m, 5H), 6.81-6.70 (m, 1H), 3.28-3.10 (m, 1H), 2.38 (s, 1H), 1.88-1.73 (m, 3H), 1.49-1.32 (m, 11H), 1.30-1.22 (m, 3H). LC-MS (Method J): [M+H]⁺=411.15 (M+H), purity: 96% by AUC, ret. time: 6.17 min, UV: 260 nm.

Step 4: Synthesis of tert-butyl ((1r,4r)-4-(4-(5-fluoroisoindoline-2-carboxamido) phenyl) cyclohexyl) carbamate (7): To a stirred solution of 5 (283 mg, 0.691 mmol) in THF (25 mL) was added triethylamine (1.4 mL, 10.37 mmol) and 6 (180 mg, 1.04 mmol) at rt. The reaction mixture was stirred at 60° C. for 16 hours. The reaction mixture was monitored by TLC and LCMS. After completion of the reaction, the reaction mixture was cooled to rt, then the reaction mixture was poured into ice-cold water (50 mL) and extracted with EtOAc (2×50 mL). The combined organic extracts were dried with Na₂SO₄, filtered and concentrated to give the crude product. The crude compound was triturated with n-pentane (30 mL) to afford 300 mg of 7 (95% yield) as a pale-brown solid. ¹H NMR (300 MHz, DMSO-d₆) δ 8.27 (s, 1H), 7.46-7.36 (m, 3H), 7.26-7.19 (m, 1H), 7.19-7.05 (m, 3H), 6.76 (d, J=7.8 Hz, 1H), 4.74 (s, 2H), 4.71 (s, 2H), 3.28-3.19 (m, 1H), 2.40-2.26 (m, 1H), 1.92-1.68 (m, 4H), 1.52-1.18 (m, 13H). LC-MS (Method J): [M+H]⁺=454.09 (M+H), purity: 98% by AUC, ret. time: 5.97 min, UV: 260 nm.

Step 5: Synthesis of N-(4-((1r,4r)-4-aminocyclohexyl) phenyl)-5-fluoroisoindoline-2-carboxamide (8): To a stirred solution of 7 (300 mg, 0.662 mmol) in DCM (3.0 mL) was added HCl (3.0 mL; 4.0 N in 1,4-dioxane) at 0° C., and the mixture was then stirred at rt for 2 hours. The reaction was monitored by TLC and LCMS. After completion of the reaction, the volatiles removed under reduced pressure. The reaction mixture was then basified with aqueous NaHCO₃ and the precipitate was filtered and dried to afford 230 mg of 8 (98% yield) as an off-white solid. ¹H NMR (300 MHz, DMSO-d₆) δ 8.32 (s, 1H), 7.48-7.40 (m, 3H), 7.20-7.09 (m, 4H), 4.74 (s, 2H), 4.71 (s, 2H), 2.68-2.53 (m, 1H), 2.46-2.23 (m, 2H), 2.06-1.77 (m, 5H), 1.52-1.40 (m, 4H). LC-MS (Method J): [M+H]⁺=354.07 (M+H), purity: 87% by AUC, ret. time: 4.42 min, UV: 260 nm.

Step 6: Synthesis of N-(4-((1r,4r)-4-azidocyclohexyl) phenyl)-5-fluoroisoindoline-2-carboxamide (9): To stirred solution of dry NaH (167 mg, 6.99 mmol) in THF (15 mL) was added 8 (190 mg, 0.54 mmol) and tosyl azide (424 mg, 2.15 mmol) under N₂, and the reaction mixture was stirred for 16 hours at rt, and the reaction was monitored by TLC and LCMS. Tosyl azide (212 mg, 1.07 mmol) was added at rt and stirred for 16 hours at 40° C. The reaction was monitored by TLC and LCMS. After completion of the reaction, the reaction mixture was poured into ice-cold water and extracted with EtOAc (2×50 mL). The combined organic layer was dried over anhydrous Na₂SO₄, and concentrated under reduced pressure to afford the crude product. The crude product was purified by combi-flash chromatography (eluent: 0-40% ethyl acetate in hexanes) which afforded 90 mg of 9 (44% yield) as an off-white solid. ¹H NMR (300 MHz, DMSO-d6) δ 8.29 (s, 1H), 7.44 (d, J=8.6 Hz, 2H), 7.40-7.34 (m, 1H), 7.29-7.20 (m, 1H), 7.18-7.08 (m, 3H), 4.74 (s, 2H), 4.71 (s, 2H), 3.53-3.42 (m, 1H), 2.37 (s, 1H), 2.01 (dd, J=5.8, 10.9 Hz, 2H), 1.89-1.80 (m, 2H), 1.60-1.32 (m, 4H). LC-MS (Method J): [M+H]⁺=380.02 (M+H), purity: 97.97% by AUC, ret. time: 6.07 min, UV: 260 nm.

Step 7: Synthesis of 5-fluoro-N-(4-((1r,4r)-4-(4-(2-hydroxy-2-methylpropyl)-1H-1,2,3-triazol-1-yl) cyclohexyl)phenyl)isoindoline-2-carboxamide (I-4): To a stirred solution of 9 (90 mg, 0.24 mmol) in t-BuOH/H₂O (5.0 mL, 1:1) was added sodium ascorbate (23.5 mg, 0.12 mmol), CuSO₄.5H₂O (29.5 mg, 0.12 mmol) and 10 (69.8 mg, 0.71 mmol) at rt. The reaction mixture was stirred at 60° C. for 1 hour under microwave irradiation. The reaction mixture was monitored by TLC and LCMS. After completion of the reaction, the reaction mixture was diluted with water (50 mL), the precipitate was filtered and dried, the crude product was triturated with n-pentane to afford 40 mg of I-4 (35%) as a pale-yellow solid. ¹H NMR (300 MHz, DMSO-d6) δ 8.31 (s, 1H), 7.90 (s, 1H), 7.47 (d, J=8.5 Hz, 2H), 7.39 (dd, J=5.2, 8.4 Hz, 1H), 7.23 (dd, J=2.0, 9.0 Hz, 1H), 7.20-7.11 (m, 3H), 4.75 (s, 2H), 4.72 (s, 2H), 4.60-4.35 (m, 2H), 2.71 (s, 2H), 2.63-2.56 (m, 1H), 2.16 (d, J=9.2 Hz, 2H), 2.01-1.87 (m, 4H), 1.77-1.60 (m, 2H), 1.08 (s, 6H). LCMS: 478.15 (M+H), purity: 95.87% by AUC, ret. time: 5.00 min, UV: 240 nm. HPLC (Method Z): purity: 95.04 by AUC, ret. time: 13.99 min, UV: 250 nm.

### Example 13—Synthesis of 3-FLUORO-N-(4-(4-(4-(2-HYDROXYPROPAN-2-YL)THIAZOL-2-YL)BICYCLO[2.2.2]OCTAN-1-YL)PHENYL)-5,7-DIHYDRO-6H-PYRROLO[3,4-B]PYRIDINE-6-CARBOXAMIDE (I-3)

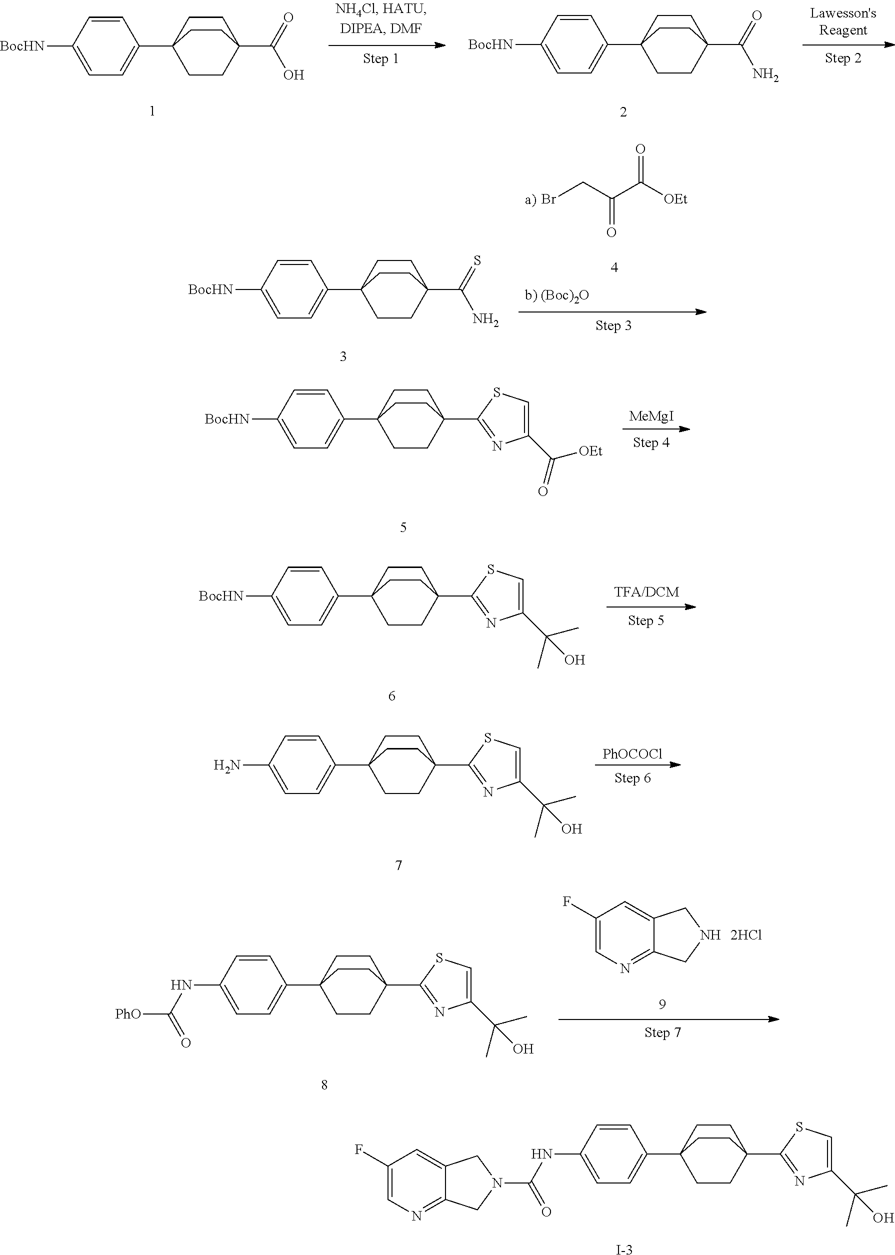

Step 1: Synthesis of tert-butyl (4-(4-carbamoylbicyclo [2.2.2]octan-1-yl) phenyl) carbamate (2): To a stirred solution of 1 (2.0 g, 5.78 mmol) in DMF (10.0 mL) was added NH₄Cl (0.93 g, 17.34 mmol) followed by addition of DIPEA (4.73 mL, 28.9 mmol) and HATU (4.39 g, 11.56 mmol) at 0° C. The reaction mixture was stirred at rt for 16 hours. The reaction mixture was monitored by TLC and LCMS. After completion of the reaction, the reaction mixture was poured into ice-cold water and the precipitate was filtered, washed with water (50 mL), and dried to obtain 1.3 g of 2 (65% yield) as an off-white solid. ¹H NMR (300 MHz, DMSO-d6) δ 9.20 (s, 1H), 7.33 (d, J=8.7 Hz, 2H), 7.22-7.14 (m, 2H), 6.96 (s, 1H), 6.73 (s, 1H), 1.73 (s, 12H), 1.46 (s, 9H). LCMS (Method J): 345.08 (M+H), purity: 99.96% by AUC, ret. time: 5.17 min, UV: 240 nm.

Step 2: Synthesis of tert-butyl (4-(4-carbamothioylbicyclo [2.2.2]octan-1-yl) phenyl) carbamate (3): To a stirred solution of 2 (1.3 g, 3.77 mmol) in THF (10.0 mL) was added Lawesson's reagent (1.06 g, 2.64 mmol) at rt. The reaction mixture was stirred at rt for 3 hours. The reaction mixture was monitored by TLC and LCMS. After completion of the reaction, the reaction mixture was poured into ice-cold water and the precipitate was filtered, washed with water (50 mL), and dried to obtain 1.3 g of 3 (95% yield) as an off-white solid. ¹H NMR (300 MHz, DMSO-d₆) δ 9.48 (s, 1H), 9.21 (s, 1H), 8.67 (s, 1H), 7.33 (d, J=8.6 Hz, 2H), 7.19 (d, J=8.8 Hz, 2H), 1.92-1.70 (m, 12H), 1.46 (s, 9H). LCMS (Method J): 361.03 (M+H), purity: 97.9% by AUC, ret. time: 5.75 min, UV: 240 nm.

Step 3: Synthesis of ethyl 2-(4-(4-((tert-butoxycarbonyl) amino) phenyl) bicyclo [2.2.2]octan-1-yl) thiazole-4-carboxylate (5): To a stirred solution of 3 (200 mg, 0.55 mmol) in THF (5.0 mL) was added 4 (118 mg, 0.61 mmol) at rt. The reaction mixture was stirred at 50° C. for 16 hours. After 16 hours, LCMS analysis of the crude mixture suggested the presence of 5 and Boc-deprotected 5 with complete consumption of the starting material. The reaction mixture was poured into ice cold water, and the resulting precipitate was filtered and dried to afford the crude compound. The crude compound was taken into DCM (5.0 mL). To this mixture, Et₃N (1.0 eq) and Boc-anhydride (0.5 eq) were added at RT and the reaction mixture was stirred for 16 hours. After complete consumption of the starting material, the reaction mixture was diluted with water (20 mL) and extracted with DCM (20 mL). The combined organic layer was washed with brine, dried over Na₂SO₄, and concentrated under reduced pressure to afford the crude compound. The crude compound was purified by column chromatography (eluent: 0-10% ethyl acetate in hexanes), which afforded 210 mg of 5 (83% yield) as an off-white solid. ¹H NMR (300 MHz, DMSO-d₆) δ 9.24 (s, 1H), 8.41 (s, 1H), 7.40-7.32 (m, 2H), 7.29-7.16 (m, J=8.8 Hz, 2H), 4.29 (q, J=7.1 Hz, 2H), 2.06-1.94 (m, 6H), 1.91-1.81 (m, 6H), 1.46 (s, 9H), 1.30 (t, J=7.1 Hz, 3H). LCMS (Method J): 457.10 (M+H), purity: 92.78% by AUC, ret. time: 6.78 min, UV: 240 nm.

Step 4: Synthesis of tert-butyl (4-(4-(4-(2-hydroxypropan-2-yl) thiazol-2-yl) bicyclo [2.2.2]octan-1-yl) phenyl) carbamate (6): To a stirred solution of 5 (210 mg, 0.46 mmol) in THF (10 mL) was added methyl magnesium iodide (1.53 mL, 4.6 mmol, 3.0 M in DEE) at 0° C. and the mixture was stirred for 1 hour. The reaction mixture was monitored by TLC and LCMS. After completion of the reaction, the reaction mixture was quenched with a saturated solution of NH₄Cl and diluted with ethyl acetate (2×25 mL). The combined organic extracts were washed with brine, dried over Na₂SO₄ and concentrated under reduced pressure to afford the crude product. The crude product was purified by column chromatography (eluent: 0-50% ethyl acetate in hexanes) which afforded 200 mg of 6 (98% yield) as an off-white solid. ¹H NMR (300 MHz, DMSO-d₆) δ 9.23 (s, 1H), 7.39-7.31 (m, 2H), 7.25-7.19 (m, J=8.8 Hz, 2H), 7.17 (s, 1H), 5.03 (s, 1H), 2.01-1.93 (m, 6H), 1.90-1.82 (m, 6H), 1.46 (s, 9H), 1.42 (s, 6H). LCMS: 443.05 (M+H), purity: 99.42% by AUC, ret. time: 6.53 min, UV: 240 nm.

Step 5: Synthesis of 2-(2-(4-(4-aminophenyl) bicyclo [2.2.2]octan-1-yl) thiazol-4-yl) propan-2-ol (7): To a stirred solution of 6 (200 mg, 0.45 mmol) in DCM (10 mL) was added TFA (0.069 mL, 0.9 mmol) at 0° C. under N₂ atmosphere. Then the reaction mixture was stirred at rt for 2 hours. The reaction mixture was monitored by TLC and LCMS. After completion of the reaction, the volatiles were removed under reduced pressure. The reaction mixture was diluted with water and extracted with diethyl ether (2×30 mL). The aqueous layer was basified to (pH ˜8-9) using a saturated NaHCO₃ solution (30 mL). The aqueous layer was extracted with DCM (2×30 mL). The combined organic layer was dried over Na₂SO₄ and concentrated under reduced pressure to afford the crude product. The crude product was triturated with n-heptane to afford 150 mg of 7 (97% yield) as a brown solid. ¹H NMR (300 MHz, DMSO-d₆) δ 7.17 (s, 1H), 7.02-6.95 (m, 2H), 6.49 (d, J=8.5 Hz, 2H), 5.03 (s, 1H), 4.82 (s, 2H), 1.99-1.92 (m, 6H), 1.84-1.78 (m, 6H), 1.42 (s, 6H). LC-MS (Method J): [M+H]⁺=343.05 (M+H), purity: 81.82% by AUC, ret. time: 5.48 min, UV: 260 nm.

Step 6: Synthesis of phenyl (4-(4-(4-(2-hydroxypropan-2-yl) thiazol-2-yl) bicyclo [2.2.2]octan-1-yl) phenyl) carbamate (8): To a stirred solution of 7 (150 mg, 0.44 mmol) and triethylamine (0.18 mL, 1.31 mmol) in THF (5.0 mL) was added phenyl chloroformate (0.06 mL, 0.53 mmol) slowly at 0° C. The reaction mixture was then stirred at 0° C. for 30 min and at rt for 4 hours. The reaction mixture was poured into ice-cold water and the precipitate was filtered and dried to obtain the crude product. The crude product was triturated with n-heptane to give 150 mg of 8 (99% yield) as an off-white solid. ¹H NMR (300 MHz, DMSO-d₆) δ 10.19-9.97 (m, 1H), 7.46-7.38 (m, 4H), 7.35-7.19 (m, 5H), 7.18 (s, 1H), 5.04 (s, 1H), 2.02-1.94 (m, 6H), 1.89 (d, J=9.3 Hz, 6H), 1.42 (s, 6H). LC-MS (Method J): [M+H]⁺=463.07 (M+H), purity: 82.01% by AUC, ret. time: 6.23 min, UV: 260 nm.

Step 7: Synthesis of 3-fluoro-N-(4-(4-(4-(2-hydroxypropan-2-yl) thiazol-2-yl) bicyclo [2.2.2]octan-1-yl) phenyl)-5,7-dihydro-6H-pyrrolo [3,4-b]pyridine-6-carboxamide (I-3): 8 (110 mg, 0.24 mmol) was added to a mixture of 9 (75 mg, 0.36 mmol) and DIPEA (0.2 mL, 1.19 mmol) in THF (5 mL) at ambient temperature. The reaction mixture was stirred at 60° C. for 16 hours. After completion of the reaction, the reaction mixture was poured into ice-cold water and stirred for 3 hours. The precipitate was filtered and washed with MTBE (500 mL) to afford 45 mg of I-3 (33% yield) as an off-white solid. H NMR (300 MHz, DMSO-d₆) δ 8.48 (d, J=1.3 Hz, 1H), 8.36 (s, 1H), 7.80 (dd, J=2.6, 9.0 Hz, 1H), 7.47 (d, J=8.8 Hz, 2H), 7.25 (d, J=8.8 Hz, 2H), 7.18 (s, 1H), 5.04 (s, 1H), 4.77 (s, 2H), 4.73 (s, 2H), 2.03-1.94 (m, 6H), 1.93-1.85 (m, 6H), 1.43 (s, 6H). LCMS (Method J): 507.01 (M+H), purity: 97.95% by AUC, ret. time: 5.43 min, UV: 240 nm. HPLC (Method Z): purity: 95.12% by AUC, ret. time: 15.05 min, UV: 250 nm.

### Example 14—Synthesis of 5-FLUORO-N-(4-(4-(4-(2-HYDROXYPROPAN-2-YL)THIAZOL-2-YL)BICYCLO[2.2.2]OCTAN-1-YL)PHENYL)ISOINDOLINE-2-CARBOXAMIDE (I-1)

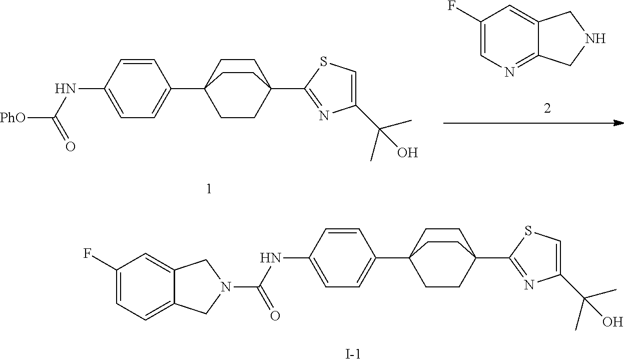 [SMILES: CC(C)(O)c1csc(C23CCC(c4ccc(NC(=O)Oc5ccccc5)cc4)(CC2)CC3)n1 ; Fc1cnc2c(c1)CNC2 ; CC(C)(O)c1csc(C23CCC(c4ccc(NC(=O)N5Cc6ccc(F)cc6C5)cc4)(CC2)CC3)n1 ; [I-]]

2 (44 mg, 0.32 mmol) was added to a solution of 1 (100 mg, 0.22 mmol) and DIPEA (0.17 mL, 1.08 mmol) in THF (5 mL) at ambient temperature. The reaction mixture was stirred at 60° C. for 16 hours. After completion of the reaction, the reaction mixture was poured into ice-cold water and stirred for 3 hours. The precipitate was filtered and washed with MTBE (500 mL) to afford 45 mg of I-1 (41% yield) as an off-white solid. ¹H NMR (300 MHz, DMSO-d₆) δ 8.30 (s, 1H), 7.50-7.43 (m, 2H), 7.42-7.35 (m, 1H), 7.31-7.08 (m, 5H), 5.04 (s, 1H), 4.74 (bs, 2H), 4.73-4.68 (m, 2H), 2.03-1.93 (m, 6H), 1.93-1.85 (m, 6H), 1.43 (s, 6H). LCMS (Method J): 506.13 (M+H), purity: 99.71% by AUC, ret. time: 5.95 min, UV: 240 nm. HPLC (Method Z): purity: 95.55% by AUC, ret. time: 16.27 min, UV: 250 nm.

### Example 15—Synthesis of 7-FLUORO-N-(4-(4-(5-METHYL-1,3,4-OXADIAZOL-2-YL)BICYCLO[2.2.2]OCTAN-1-YL)PHENYL)-1,3-DIHYDRO-2H-PYRROLO[3,4-C]PYRIDINE-2-CARBOXAMIDE (I-2)

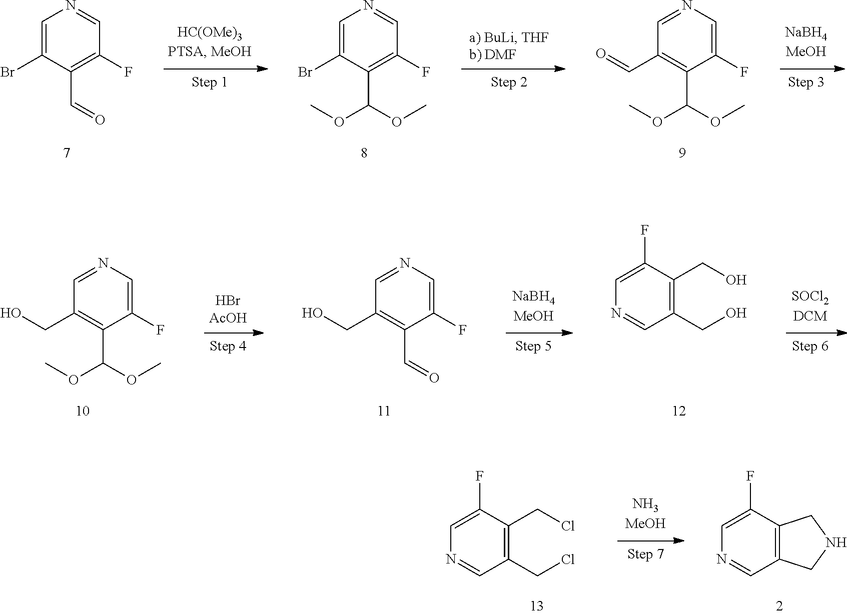

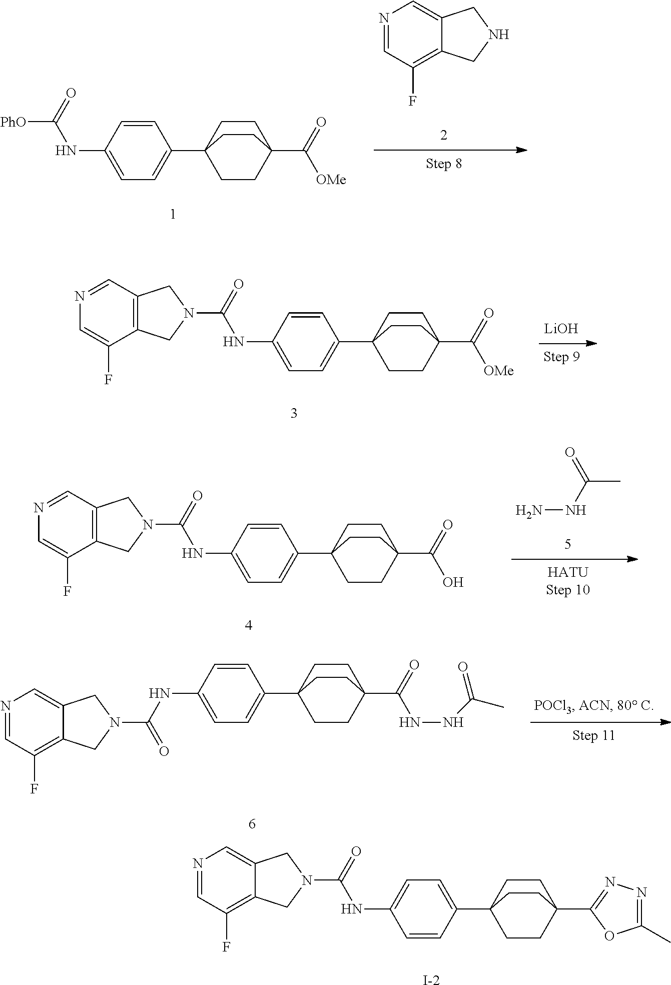

Step 1: Synthesis of 3-bromo-4-(dimethoxymethyl)-5-fluoropyridine (8): To a stirred solution of 7 (10.0 g, 49.01 mmol) in MeOH (100 mL) was added trimethyl orthoformate (20.8 g, 196.07 mmol) and PTSA (0.844 g, 4.90 mmol) at 0° C. under N₂. The reaction mixture was stirred at 80° C. for 3 hours. The reaction mixture was monitored by TLC and LCMS. After completion of the reaction, the volatiles were removed under reduced pressure and the reaction mixture was diluted with water (200 mL) and extracted with EtOAc (2×150 mL). The combined organic layer was dried over Na₂SO₄ and concentrated under reduced pressure to afford the crude product. The crude product was used in the next step without purification. ¹H NMR (300 MHz, DMSO-d₆) δ 8.67 (s, 1H), 8.62 (d, J=1.9 Hz, 1H), 5.63 (d, J=0.9 Hz, 1H), 3.40 (s, 6H). LCMS (Method J): 251.86 (M+H), purity: 98.65% by AUC, ret. time: 4.87 min, UV: 240 nm.

Step 2: Synthesis of 4-(dimethoxymethyl)-5-fluoronicotinaldehyde (9): To stirred solution of 8 (5.8 g, 23.2 mmol) in toluene (80 mL) was added n-BuLi (13.92 mL, 34.8 mmol, 2.5 M in n-hexanes) dropwise at −78° C. under N₂, and the mixture was stirred for 10 min. DMF (3.38 g, 46.4 mmol) was then added dropwise at −78° C. and the mixture was stirred for 1 hour. The reaction was monitored by TLC and LCMS. After completion of the reaction, the reaction mixture was quenched with a saturated solution of NH₄Cl and extracted with EtOAc (2×100 mL). The combined organic layer was dried over anhydrous Na₂SO₄ and concentrated under reduced pressure to afford 10.4 g of 9 (crude) as brown color liquid. ¹H NMR (300 MHz, CHLOROFORM-d) δ 10.64 (s, 1H), 8.92 (s, 1H), 8.65 (d, J=1.3 Hz, 1H), 5.71 (s, 1H), 3.52 (s, 6H).

Step 3: Synthesis of (4-(dimethoxymethyl)-5-fluoropyridin-3-yl) methanol (10): To a stirred solution of 9 (10.0 g, 50.25 mmol) in MeOH (100 mL) was added NaBH₄ (3.79 g, 100.5 mmol) at 0° C. under N₂, then the mixture was stirred at rt for 2 hours. The reaction mixture was monitored by TLC and LCMS. After completion of the reaction, the reaction mixture was quenched with water (100 mL), basified with saturated Na₂CO₃, and extracted with ethyl acetate (2×80 mL). The combined organic extracts were washed with brine, dried over Na₂SO₄ and concentrated under reduced pressure to afford the crude product. The product was purified by column chromatography (eluent: 0-20% ethyl acetate in hexanes) which afforded 6.0 g of 10 (59% yield) as an off-white solid. ¹H NMR (300 MHz, CHLOROFORM-d) δ 8.51-8.44 (m, 2H), 5.66 (s, 1H), 4.84 (s, 2H), 3.54 (s, 6H), 3.18-3.03 (m, 1H). LCMS (Method J): 202.04 (M+H), purity: 98.51% by AUC, ret. time: 3.65 min, UV: 240 nm.

Step 4: Synthesis of 3-fluoro-5-(hydroxymethyl) isonicotinaldehyde (11): To a stirred solution of 10 (6.0 g, 29.85 mmol) in AcOH (42 mL) was added HBr (12.07 g, 149.25 mmol, 33% in AcOH) at 0° C. under N₂, and then the mixture was stirred at rt for 1 hour. The reaction mixture was monitored by TLC and LCMS. After completion of the reaction, the reaction mixture was quenched with water (60 mL) and basified with Na₂CO₃. The mixture was extracted with ethyl acetate (2×80 mL) and the combined organic extracts were washed with brine, dried over Na₂SO₄ and concentrated under reduced pressure to afford 3.6 g of 11 (78% yield) as brown color liquid. ¹H NMR (300 MHz, DMSO-d₆) δ 8.52-8.47 (m, 2H), 7.25 (d, J=7.7 Hz, 1H), 6.54 (dd, J=2.1, 7.8 Hz, 1H), 5.22-5.13 (m, 1H), 5.05-4.99 (m, 1H). LCMS (Method J): 156.02 (M+H), purity: 92.62% by AUC, ret. time: 1.67 min, UV: 240 nm.

Step 5: Synthesis of (5-fluoropyridine-3,4-diyl)dimethanol (12): To a stirred solution of 11 (3.6 g, 23.22 mmol) in MeOH (50 mL) was added NaBH₄ (1.75 g, 46.45 mmol) at 0° C., and then the mixture stirred at rt for 2 hours. The reaction mixture was monitored by TLC and LCMS. After completion of the reaction, the reaction mixture was quenched with water (40 mL) and basified with Na₂CO₃. The mixture was then extracted with ethyl acetate (2×80 mL), and the combined organic extracts were washed with brine, dried over Na₂SO₄ and concentrated under reduced pressure to obtain 3.6 g of 12 (99% yield) as brown color liquid. ¹H NMR (300 MHz, DMSO-d₆) δ 8.47-8.41 (m, 2H), 5.39 (t, J=5.5 Hz, 1H), 5.30 (t, J=5.7 Hz, 1H), 4.70 (d, J=5.5 Hz, 2H), 4.56 (dd, J=1.4, 5.7 Hz, 2H). LCMS (Method J): 58.03 (M+H), purity: 85.71% by AUC, ret. time: 1.37 min, UV: 240 nm.

Step 6: Synthesis of 3,4-bis(chloromethyl)-5-fluoropyridine (13): To a stirred solution of 12 (3.6 g, 22.92 mmol) in DCM (50 mL) was added SOCl₂ (24.7 mL, 343.94 mmol) at 0° C. under N₂, and then the mixture was stirred at 40° C. for 16 hours. The reaction mixture was monitored by TLC and LCMS. After completion of the reaction, the volatiles were removed under reduced pressure. The reaction mixture was quenched with a saturated solution NaHCO₃ and extracted with ethyl acetate (2×80 mL). The combined organic extracts were washed with brine, dried over Na₂SO₄ and concentrated under reduced pressure to afford the crude product. The crude product was purified by column chromatography (eluent: 0-15% ethyl acetate in hexanes) which afforded 2.3 g of 13 (52% yield) as a pale-yellow solid. ¹H NMR (300 MHz, DMSO-d₆) δ 8.66 (d, J=1.1 Hz, 1H), 8.60 (s, 1H), 5.00 (s, 2H), 4.90 (d, J=0.9 Hz, 2H). LCMS (Method J): 195.88 (M+H), purity: 98.89% by AUC, ret. time: 5.03 min, UV: 240 nm.

Step 7: Synthesis of 7-fluoro-2,3-dihydro-1H-pyrrolo[3,4-c]pyridine (2): To a stirred solution of 13 (2.3 g, 11.85 mmol) in MeOH (23 mL) in a sealed tube was added NH₃ in MeOH (70.0 mL) under N₂, and the mixture was stirred at 50° C. for 1.5 hours. The reaction mixture was monitored by TLC and LCMS. After completion of the reaction, the volatiles were removed under reduced pressure. The crude product was triturated with MTBE (2×30 mL) to afford 1.0 g of 2 (62% yield) as gummy brown color solid. ¹H NMR (300 MHz, DMSO-d₆) δ 8.39-8.37 (m, 2H), 4.89 (s, 4H), 3.27 (d, J=4.3 Hz, 1H). LCMS (Method J): 139.05 (M+H), purity: 63.47% by AUC, ret. time: 1.25 min, UV: 240 nm.

Step 8: Synthesis of methyl 4-(4-(7-fluoro-2,3-dihydro-1H-pyrrolo [3,4-c]pyridine-2-carboxamido) phenyl) bicyclo [2.2.2]octane-1-carboxylate (3): 1 (465 mg, 1.22 mmol) was added to a solution of 2 (304 mg, 2.2 mmol) and triethylamine (0.86 mL, 6.13 mmol) in THF (10 mL) at ambient temperature. The reaction mixture was stirred at 60° C. for 16 hours. After completion of the reaction, the reaction mixture was poured into ice-cold water and extracted with EtOAc (2×50 mL). The combined organic extracts were washed with brine, dried over Na₂SO₄ and concentrated under reduced pressure to afford the crude product. The product was purified by column chromatography (eluent: 0-50% ethyl acetate in hexanes) which afforded 380 mg of 3 (73% yield) as an off-white solid. ¹H NMR (300 MHz, DMSO-d₆) δ 8.50-8.47 (m, 2H), 8.41 (s, 1H), 7.49-7.40 (m, 2H), 7.21 (d, J=8.8 Hz, 2H), 4.88 (s, 2H), 4.85 (s, 2H), 3.59 (s, 3H), 1.80 (d, J=3.7 Hz, 12H). LCMS (Method J): 424.10 (M+H), purity: 94.62% by AUC, ret. time: 5.32 min, UV: 240 nm.

Step 9: Synthesis of 4-(4-(7-fluoro-2,3-dihydro-1H-pyrrolo [3,4-c]pyridine-2-carboxamido) phenyl) bicyclo [2.2.2]octane-1-carboxylic acid (4): LiOHH₂O (198 mg, 4.73 mmol) was added to a stirred solution of 3 (400 mg, 0.95 mmol) in THF, MeOH and water (1:1:1, 30 mL) at RT. The mixture was then stirred at 50° C. for 6 hours. After completion of the reaction, the volatiles were removed under reduced pressure. The mixture was then acidified (pH ˜2-3) with 1.0 N HCl. The precipitate was filtered and dried to obtain 342 mg of 4 (88% yield) as an off-white solid. ¹H NMR (300 MHz, DMSO-d₆) δ 12.04 (s, 1H), 8.53-8.46 (m, 2H), 8.41 (s, 1H), 7.45 (d, J=8.8 Hz, 2H), 7.21 (d, J=8.8 Hz, 2H), 4.87 (d, J=7.7 Hz, 4H), 1.78 (s, 12H). LCMS (Method J): 410.01 (M+H), purity: 99.52% by AUC, ret. time: 4.02 min, UV: 240 nm.

Step 10: Synthesis of N-(4-(4-(2-acetylhydrazine-1-carbonyl) bicyclo [2.2.2]octan-1-yl) phenyl)-7-fluoro-1,3-dihydro-2H-pyrrolo [3,4-c]pyridine-2-carboxamide (6): To a stirred solution of 4 (160 mg, 0.39 mmol) in DMF (5.0 mL) was added 5 (31 mg, 0.43 mmol), followed by addition of DIPEA (151 mg, 1.17 mmol) and HATU (2296 mg, 0.78 mmol) at 0° C. The reaction mixture was stirred at rt for 16 hours. The reaction mixture was monitored by TLC and LCMS. After completion of the reaction, the reaction mixture was poured into ice-cold water, extracted with EtOAc (2×50 mL), dried over Na₂SO₄ and concentrated under reduced pressure to afford the crude product. The crude product was purified by column chromatography (eluent: 0-5% ethyl MeOH in DCM) which afforded 135 mg of 6 (74% yield) as an off-white solid. ¹H NMR (300 MHz, DMSO-d₆) δ 9.59-9.54 (m, 1H), 9.24 (s, 1H), 8.52-8.37 (m, 3H), 7.45 (d, J=8.7 Hz, 2H), 7.22 (d, J=8.6 Hz, 2H), 4.88 (s, 2H), 4.85 (s, 2H), 1.86-1.74 (m, 15H). LCMS (Method J): 466.05 (M+H), purity: 88.94% by AUC, ret. time: 4.10 min, UV: 240 nm.

Step 11: Synthesis of 7-fluoro-N-(4-(4-(5-methyl-1,3,4-oxadiazol-2-yl) bicyclo [2.2.2]octan-1-yl) phenyl)-1,3-dihydro-2H-pyrrolo [3,4-c]pyridine-2-carboxamide (I-2): To a stirred solution of 6 (120 mg, 0.39 mmol) in toluene (10.0 mL) was added POCl₃ (0.5 mL) at 0° C. The reaction mixture was stirred at 80° C. for 3 hours. The reaction mixture was monitored by TLC and LCMS. After completion of the reaction, the reaction mixture was poured into ice-cold water, basified with Na₂CO₃, extracted with EtOAc (2×50 mL), dried over Na₂SO₄ and concentrated under reduced pressure to afford the crude product. The crude product was purified by column chromatography (eluent: 0-5% MeOH in DCM) which afforded 40 mg of I-2 (35% yield) as an off-white solid. ¹H NMR (300 MHz, DMSO-d₆) δ 8.51-8.47 (m, 2H), 8.43 (s, 1H), 7.47 (d, J=8.9 Hz, 2H), 7.29-7.22 (m, 2H), 4.89 (s, 2H), 4.86 (s, 2H), 2.46 (s, 3H), 2.03-1.81 (m, 12H). LCMS (Method J): 448.06 (M+H), purity: 99.16% by AUC, ret. time: 4.73 min, UV: 240 nm. HPLC: purity: 96.28% by AUC, ret. time: 13.20 min, UV: 250 nm.

### Example 16—Synthesis of 5-FLUORO-N-(4-(4-(1-(2-HYDROXY-2-METHYLPROPYL)-1H-1,2,3-TRIAZOL-4-YL)BICYCLO[2.2.2]OCTAN-1-YL)PHENYL)ISOINDOLINE-2-CARBOXAMIDE (I-12)

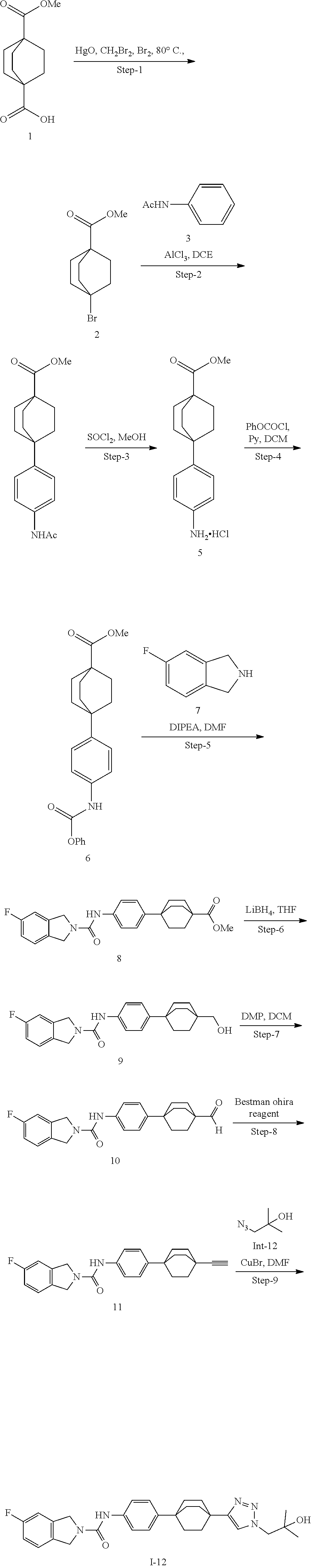

Step 1: Synthesis of methyl 4-bromobicyclo[2.2.2]octane-1-carboxylate (2): To the stirred solution of compound 1 (5.0 g, 23.55 mmol) in 1,2-dibromoethane (15 mL) was added Mercuric Oxide (5.15 g, 23.55 mmol) at 0° C. Reaction mixture temperature was raised to 80° C. and added Bromine (1.58 mL, 30.62 mmol) in 1,2-dibromoethane (5 mL). The reaction mixture was stirred at 80° C. for 3 hours. after completion of the reaction (monitored by TLC), reaction mas was filtered through the CELITE® pad washed with EtOAc. The organic layer was concentrated under reduced pressure. Obtained crude compound was purified by CombiFlash (Nexgen-300) chromatography 24 g cartridge silica gel, by using 5% EtOAc in n-heptane as an eluent to afford compound 2 (5.5 g, 22 mmol, 94% Yield) as an off-white semi-solid. LC-MS: MS calcd. for chemical formula: C₁₀H₁₅BrO₂: 247.13; found: NA M+H]⁺. ¹H NMR (400 MHz, DMSO-d₆) δ: 3.69-3.56 (s, 3H), 2.56-2.25 (m, 6H), 1.89-1.82 (m, 6H).

Step 2: Synthesis of methyl 4-(4-acetamidophenyl)bicyclo[2.2.2]octane-1-carboxylate (4): To stirred solution of compound 2 (5.50 g, 20.23 mmol) and compound 3 (3.0 g, 22 mmol) in 1,2-DCE (50 mL) was added Aluminum Chloride (12 g, 89 mmol) portion wise at 0° C.; then stirred at RT for 16 hours. after completion of the reaction (monitored by TLC), reaction mixture was slowly poured in ice cold water. The aqueous layer was extracted with DCM. The combined organic extracts were washed with brine, dried over sodium sulphate, filtered, and concentrated under reduced pressure. Obtained crude compound was purified by CombiFlash (Nexgen-300) chromatography 24 g cartridge silica gel, by using 25% EtOAc in n-heptane as an eluent to afford compound 4 (1.0 g, 3.23 mmol, 16.0% Yield) as a white solid. LC-MS (Method G): Ret. Time: 2.19 min, MS calcd. for chemical formula: C₁₈H₂₃NO₃: 301.39; found: 302.50 [M+H]⁺.

Step 3: Synthesis of methyl 4-(4-aminophenyl)bicyclo[2.2.2]octane-1-carboxylate (5): To stirred solution of compound 4 (1.0 g, 3.32 mmol) in MeOH (15 mL) was added SOCl₂ (0.24 mL, 3.32 mmol) drop-wise at RT. then the reaction mixture was heated to 80° C. for 8 hours. after completion of the reaction (monitored by TLC), reaction mixture was cooled to RT; volatiles were concentrated under reduced pressure. The crude residue was diluted with water, basified with saturated NaHCO₃ sol and extracted with EtOAc. The combined organic layer was dried over sodium sulfate; filtered and concentrated under reduced pressure as an eluent to afford crude compound 5 (0.8 g, 3 mmol, 90% yield) as an off-white solid. LC-MS (Method F): Ret. Time: 0.99 min, MS calcd. for chemical formula: C₁₆H₂₁NO₂: 259.3; found: 260.42 [M+H]⁺.

Step 4: Synthesis of methyl 4-(4-((phenoxycarbonyl)amino)phenyl) bicyclo[2.2.2]octane-1-carboxylate (6): To a stirred solution of compound 5 (0.8 g, 3 mmol) in DCM (20 mL) were added pyridine (1.0 mL, 10 mmol), Phenyl Chloroformate (0.7 g, 5 mmol), DMAP (40 mg) at 0° C. then stirred at RT for 2 hours under nitrogen atmosphere. After completion of the reaction (monitored by TLC), the reaction mixture was diluted with water and extracted with 5% MeOH in DCM. The combined organic layers were washed with water followed by brine, dried over anhydrous Na₂SO₄, and concentrated under reduced pressure. Obtained crude compound was triturated with diethyl ether, n-pentane, filtered, dried under reduced pressure to afford the title compound 6 (1.1 g, 2.6 mmol, 80% yield) as an off-white solid. LC-MS (Method F): Ret. Time: 1.51 min, MS calcd. for chemical formula: C₂₃H₂₅NO₄: 379.46; found: 380.50 [M+H]⁺.

Step 5: Synthesis of methyl 4-(4-(5-fluoroisoindoline-2-carboxamido)phenyl) bicyclo[2.2.2]octane-1-carboxylate (8): To a stirred solution of compound 6 (0.5 g, 1.32 mmol) in DMF (10 mL) were added compound 7 (0.26 g, 1.516 mmol) and DIPEA (1.38 mL, 7.91 mmol) at RT and stirred at 90° C. for 6 hours. After completion of the reaction (monitored by TLC), the reaction mixture was quenched with ice cold water and extracted with EtOAc. The combined organic layers were washed with water followed by brine, dried over anhydrous Na₂SO₄, and concentrated under reduced pressure. The crude triturated with diethyl ether and n-pentane, filtered and dried under reduced pressure to afford the title compound 8 (0.52 g, 1.23 mmol, 86.32% yield) as an off-white solid. LCMS (Method F): Ret. Time: 1.46 min, MS calcd. for chemical formula: C₂₅H₂₇FN₂O₃: 422.50; found: 423.51 [M+H]⁺.

Step 6: Synthesis of 5-fluoro-N-(4-(4-(hydroxymethyl)bicyclo[2.2.2]octan-1-yl)phenyl) isoindoline-2-carboxamide (9): To a stirred solution of compound 8 (0.52 g, 1.23 mmol) in THF (12 mL), was added Lithium borohydride (4.0 mol/L) in THF (0.92 mL, 3.69 mmol) at 0° C. The reaction mixture stirred RT at for 8 hours. After completion of the reaction (monitored by TLC), reaction mixture was poured in ice-cool water (23 mL)& neutralized with aqueous 1N HCl at 5° C. (pH ˜7), extracted with EtOAc. The combined organic layers were dried over anhydrous sodium sulphate, filtered and concentrated under reduced pressure. Obtained crude semi-solid was triturated with washed diethyl ether (5 mL)& with n-pentane (10 mL), filtered and concentrated under reduced pressure to afford compound 9 (0.46 g, 1.16 mmol, 83.91% Yield) as an off-white solid. LCMS: (Method F) Ret. Time: 1.29 min, MS calcd. for chemical formula: C₂₄H₂₇FN₂O₂: 394.49; found: 395.53 [M+H]⁺.

Step 7: Synthesis of 5-fluoro-N-(4-(4-formylbicyclo[2.2.2]octan-1-yl)phenyl)isoindoline-2-carboxamide (10): To a stirred solution of compound 9 (0.46 g, 1.16 mmol) in dry DCM (40 mL), to this added in 4 portions of Dess-Martin periodinane (2.5 equiv., 2.915 mmol), at 0° C. the reaction was allowed to stirred at RT for 8 hours. Progress of the reaction was monitored by TLC. After completion, The reaction mass was poured in to ice-cool water, extracted with DCM. The combined organic layer was dried over with anhydrous sodium sulphate, filtered and concentrated under rota evaporator to give semi solid crude. The above crude compound was purified by combi-flash column chromatography and eluted with 25% EtOAc in heptanes to afford compound 10 (0.35 g, 0.87 mmol, 74.95% yield) as an Off-white solid. LCMS (Method F): Ret. Time: 1.39 min, MS calcd. for chemical formula: C₂₄H₂₅FN₂O₂: 392.47; found: 393.53 [M+H]⁺.

Step 8: Synthesis of N-(4-(4-ethynylbicyclo[2.2.2]octan-1-yl)phenyl)-5-fluoroisoindoline-2-carboxamide (11): To a two-necked RBF was charged with compound 10 (0.45 g, 1.14 mmol) and dry Potassium carbonate (0.349 g, 2.52 mmol) degassed the solid by evacuating and backfilling with argon for a total of three cycles; afterwards place the reaction flask under a slight positive pressure of argon charge dry MeOH (10 mL) under argon gas atmosphere to this added drop-wise of Dimethyl (1-Diazo-2-Oxopropyl)Phosphonate (1.2 equiv., 1.376 mmol) at 0° C., then allowed to stirred at RT for 2 h. Progress of the reaction was monitored by TLC. After completion, the reaction mass was poured into ice-cool water and extracted with EtOAc, the combined organic layers were dried over with anhydrous Na₂SO₄, then filtered and concentrated under pressure to give brown sticky semi solid crude. The crude compound was purified by combi-flash column chromatography and eluted with 18-20% of EtOAc in heptanes to afford compound 10 (0.35 g, 0.90 mmol, 78.58% Yield) as an off-white solid. ¹H NMR (400 MHz, DMSO-d₆) δ: 8.27 (s, 1H), 7.44 (d, J=8.8 Hz, 2H), 7.39 (br dd, J=5.1, 8.4 Hz, 1H), 7.28-7.11 (m, 4H), 4.73 (br d, J=11.9 Hz, 4H), 2.90 (s, 1H), 1.78 (q, J=9.3 Hz, 12H).

Step 9: Synthesis of 5-fluoro-N-(4-(4-(1-(2-hydroxy-2-methylpropyl)-1H-1,2,3-triazol-4-yl)bicyclo[2.2.2]octan-1-yl)phenyl)isoindoline-2-carboxamide: To a screw type vial was charged compound 11 (0.15 g, 0.386 mmol) and compound Int-12 (0.058 g, 0.502 mmol) in dry DMF (3.5 mL) degassed with argon gas for 10 min. then added CuBr (5.5 mg, 0.038 mmol). The vial was sealed and allowed to stirred at 40° C. for 4 hours. Progress of the reaction was monitored by TLC. After completion, the reaction mass was poured in to ice-cool water (7 mL), the solid precipitated were filtered and dried over under vacuum, then washed with diethyl ether (2×2 mL) and n-pentane (4 mL) to give crude as an off-white solid. The crude compound was purified by prep-HPLC purification to afford the title compound (30.00 mg, 0.059 mmol, 15.42% Yield) as an white solid. ¹H NMR (400 MHz, DMSO-d₆) δ: 8.31-8.24 (m, 1H), 7.68 (s, 1H), 7.46 (d, J=8.8 Hz, 2H), 7.39 (dd, J=5.3, 8.4 Hz, 1H), 7.28-7.21 (m, 3H), 7.19-7.12 (m, 1H), 4.80 (s, 1H), 4.74 (d, J=11.6 Hz, 4H), 4.21 (s, 2H), 1.96-1.82 (m, 12H), 1.06 (s, 6H). LC-MS (Method F): Ret time: 1.25 min, 99.95%; MS calcd. for chemical formula C₂₉H₃₄FN₅O₂: 503.62; Found: 504.69 [M+H]⁺. HPLC: Ret time: 8.055 min, 99.83%; Method: column: X-Select C18 (4.6×150) mm 5.0 μm Mobile Phase A: 0.1% FA in (ACN:H₂O) (5:95)V/V, Mobile Phase B: Acetonitrile (100%), Colum Temperature: 30° C., Flow Rate: 1.0 mL/min, Gradient: 0.00/5, 1.00/5, 8.00/100, 12.00/100, 14.00/5, 18.00/5, Diluent: ACN:Water (80:20). Prep-HPLC purification method: Column Name: X-SELECT C18 (30*250 mm), Mobile Phase A: 0.1% formic acid in Water, Mobile Phase B: Acetonitrile, Flow rate: 25 ml/min, Instrument List: AMC-PREP-19, Loading (mg/injection): 50, Gradient (Time/% B): 0/25, 2/25, 25/100, Sample Diluent: ACN+WATER.

Step-10: Synthesis of 1-azido-2-methylpropan-2-ol (int-12): To a stirred solution of 2,2-dimethyloxirane (0.25 g, 3.5 mmol) and Sodium Azide (0.57 g, 8.7 mmol) in MeOH (6.92 mL) was added a solution of Ammonium Chloride (0.37 g, 6.9 mmol) in Water (0.765 mL). The reaction mixture was stirred at room temperature for 36 hours. Progress of the reaction was monitored by TLC. After completion, the reaction solution was concentrated to 0.4 ml by rotary evaporation under 50° C. The mixture was diluted with 2 ml water, and then extracted with EtOAc (3×20 ml). The organic layer was collected and dried with Na₂SO₄. Then filtered and concentrated under rotary evaporator to compound Int-12 (0.15 g, 1.3 mmol, 38% Yield) as a colorless oil. ¹H NMR (401 MHz, DMSO-d₆) δ: 4.75-4.73 (m, 1H), 3.10 (s, 2H), 1.11 (s, 6H). ¹³C NMR (101 MHz, DMSO-d₆) δ: 69.98, 60.96, 26.89.

### Example 17—Synthesis of 5-fluoro-N-(4-((1r,4r)-4-(5-(2-hydroxyethyl)-1,3,4-oxadiazol-2-yl)cyclohexyl) phenyl)isoindoline-2-carboxamide (I-28)

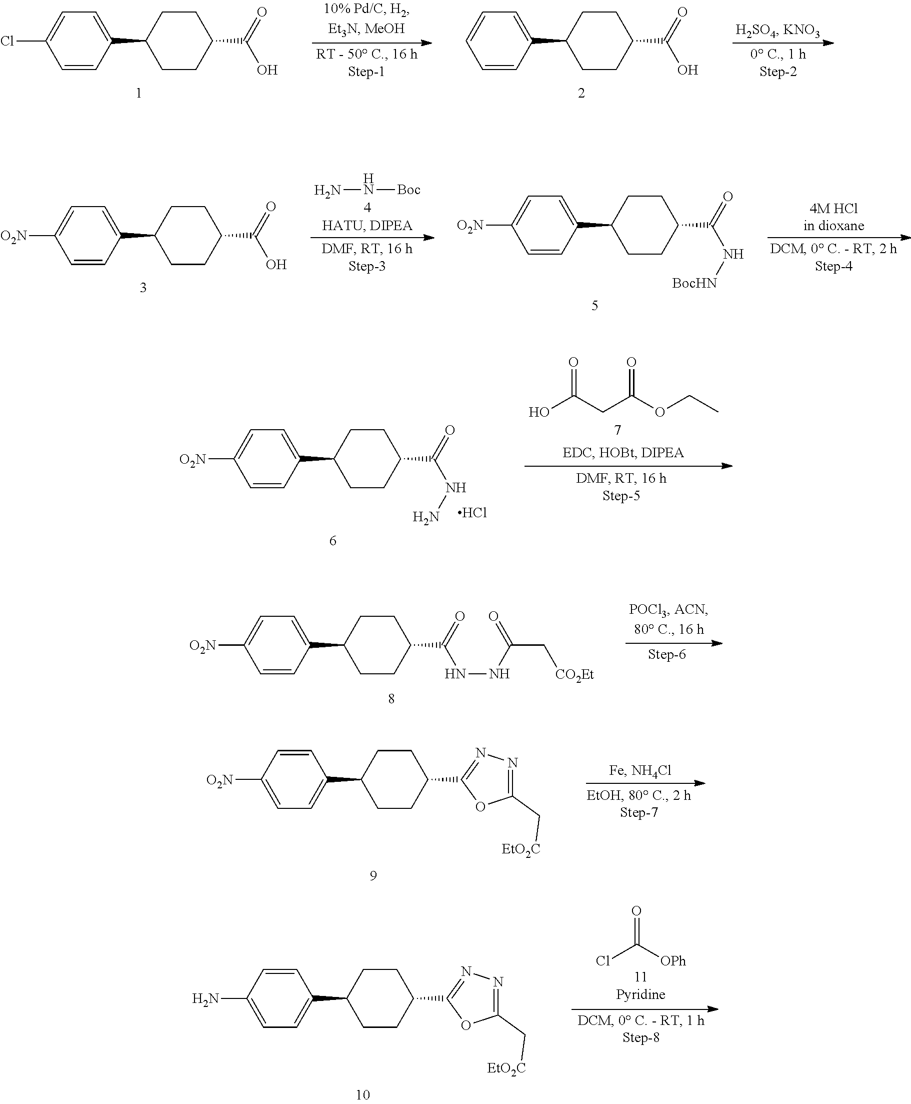

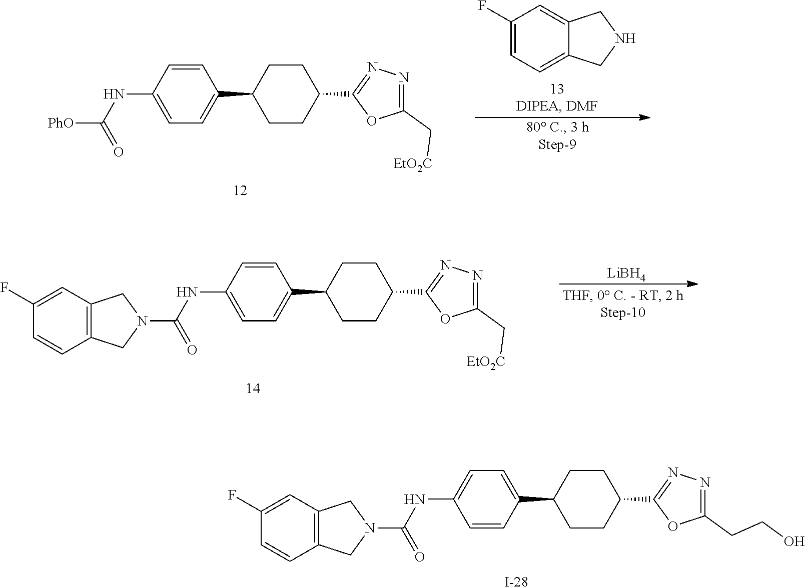

Step-1: Synthesis of 4-phenylcyclohexanecarboxylic acid (2): To a stirred solution of compound 1 (30 g, 15.7 mmol) in methanol (300 mL) was added TEA (18 mL, 128 mmol) and Pd—C (50% wet, 9 g) at room temperature. The reaction mixture was stirred for 16 hours at 50° C. under H₂. After completion of the reaction (monitored by TLC), the reaction mixture was diluted with methanol and filtered through a pad of CELITE®. The filtrate was concentrated under reduced pressure to give the crude product, which was triturated with n-heptane and dried to afford the title compound 2 (24 g, 117.6 mmol, 93.38% yield) as a white solid. LC-MS: Ret. Time: 2.00 min. MS calcd. for chemical formula C₁₃H₁₆O₂, 204.3; Found: 205.3[M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-2: Synthesis of 4-(4-nitrophenyl) cyclohexane carboxylic acid (3): To a stirred solution of KNO₃ (1.75 g, 17.13 mmol) in H₂SO₄ (100 mL) was added compound 2 (10 g, 48.95 mmol) at 0° C. The reaction mixture was stirred at room temperature for 1 hour. After completion of the reaction (monitored by TLC), ice-cooled water was added to the reaction mixture to form a precipitate, which was filtered and dried to afford the title compound 3 (4 g, 15.24 mmol, 31.14% yield) as a white solid. LC-MS: Ret. Time: 1.14 min. MS calcd. for chemical formula: C₁₃H₁₅NO₄: 249.3; Found: 232(M−18)⁺. Method: FA BEH 4.2 MINS, Column: Acuity UPLC BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 40° C. Gradient Program Time/B: 0.0/2, 0.3/2, 2.0/98, 3.5/98, 3.6/2, 4.2/2.

Step-3: Synthesis of tert-butyl 2-((1r,4r)-4-(4-nitrophenyl)cyclohexane-1-carbonyl) hydrazine-1-carboxylate (5): To a stirred solution of compound 3 (3.6 g, 14 mmol) in DMF (36 mL) was added HATU (8.5 g, 22 mmol) and DIPEA (7.6 mL, 43 mmol) followed by compound 4 (2.3 g, 17 mmol) at room temperature. This reaction mixture was stirred at room temperature for 16 hours under nitrogen atmosphere. After completion of the reaction (monitored by TLC), ice-cooled water was added to the reaction mixture, and a solid precipitated. The solid was filtered and dried to afford the title compound 5 (3.5 g, 9.631 mmol, 66.79% yield) as an off-white solid. LC-MS: Ret. Time: 1.17 min. MS calcd. for chemical formula C₁₈H₂₅N₃O₅: 363.4; Found: 264 [M−10]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-4: Synthesis of 4-(4-nitrophenyl) cyclohexane carbohydrazide (6): To a stirred solution of compound 5 (3.5 g, 9.631 mmol) in DCM (35 mL) was added HCl (4 M) in 1, 4-dioxane (5 mL) at 0° C. The mixture was stirred at room temperature for 2 hours. After completion of the reaction (monitored by TLC), the solvent was evaporated to give a residue, which was washed with sodium bicarbonate solution and extracted with ethyl acetate. The combined organic layer was dried over anhydrous sodium sulphate, and the solvent was removed under reduced pressure to afford the desired compound 6 (2.0 g, 7.595 mmol, 54% yield) as an off-white solid. LC-MS: Ret. Time: 0.92 min. MS calcd. for chemical formula: C₁₃H₁₇N₃O₃: 263.3; Found: 264.21[M+H]⁺. Method: TFA BEH 2.5 MIN, Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-5: Synthesis of ethyl 3-[2-[4-(4-nitrophenyl)cyclohexanecarbonyl]hydrazino]-3-oxo-propanoate (8): To a stirred solution of compound 6 (2.0 g, 5.393 mmol) in DMF (5 mL) was added EDC.HCl (2.17 g, 11.393 mmol), HOBt (1.5 g, 11.393 mmol), and DIPEA (1.6 mL, 15.18 mmol) followed by compound 7 (1.2 g, 9.115 mmol) at room temperature. The reaction mixture was stirred at room temperature for 16 hours under nitrogen atmosphere. After completion of the reaction (monitored by TLC), ice-cooled water was added to the reaction mixture and a solid precipitated. The solid was filtered and dried to afford the title compound 8 (2.2 g, 5.829 mmol, 76.92% yield) as an off-white solid. LC-MS: Ret. Time: 1.07 min. MS calcd. for chemical formula: C₁₈H₂₃N₃O₆ 377.4; Found: 378.31 [M+H]⁺; Method: TFA BEH 2.5 MIN. Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-6: Synthesis of ethyl 2-[5-[4-(4-nitrophenyl)cyclohexyl]-1,3,4-oxadiazol-2-yl]acetate (9): To a stirred solution of compound 8 (2 g, 5.285 mmol) in ACN (20 mL) was added POCl₃ (1.4 mL, 15.856 mmol) at 0° C. The reaction mixture refluxed at 80° C. for 16 hours. After completion of the reaction (monitored by TLC), the mixture was allowed to cool to room temperature, washed with sodium bicarbonate solution, and extracted with ethyl acetate. The combined organic layer was dried over anhydrous sodium sulphate, and the solvent was evaporated under reduced pressure to give the crude product, which was purified by flash column chromatography (100-200 silica, 50% ethyl acetate-hexane eluent) to afford the desired compound 9 (1.2 g, 2.3 mmol, 63.15% yield) as an off-white sold. LC-MS: Ret. Time: 1.28 min. MS calcd. for chemical formula: C₁₈H₂₁N₃O₅ 359.4; Found: 360.29 [M+H]⁺. Method: TFA BEH 2.5 MIN, Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-7: Synthesis of ethyl 2-[5-[4-(4-aminophenyl)cyclohexyl]-1,3,4-oxadiazol-2-yl]acetate (10): To a stirred solution of compound 9 (0.7 g, 1.948 mmol), ethanol (10 mL), and water (2 mL) was added Fe (0.89 g, 13.63 mmol) and ammonium chloride (0.729 g, 13.63 mmol) at room temperature. The reaction mixture was stirred for 2 hours at 80° C. After completion of the reaction (monitored by TLC), the reaction mixture was diluted with methanol and filtered through a pad of CELITE®. The filtrate was concentrated under reduced pressure to afford the title compound 10 (0.4 g, 0.432 mmol, 22.44% yield) as a white solid. LC-MS: Ret. Time: 0.86 min. MS calcd. for chemical formula: C₁₈H₂₃N₃O₃ 329.4; Found: 330.29 [M+H]⁺. Method: FA BEH 2.5 min, Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-8: Synthesis of ethyl 2-[5-[4-[4-(phenoxycarbonylamino)phenyl]cyclohexyl]-1,3,4-oxadiazol-2-yl]acetate (12): To a stirred solution of compound 10 (0.4 g, 1.121 mmol) in DCM (10 mL) were added pyridine (0.46 mL, 3.636 mmol) and compound 11 (0.2 mL, 1.45 mmol) at 0° C. The mixture was then stirred at room temperature for 1 hour under nitrogen atmosphere. After completion of the reaction (monitored by TLC), the mixture was diluted with water and extracted with EtOAc. The combined organic layers were concentrated under reduced pressure to give the crude product, which was purified by flash column chromatography (100-200 silica, 5% methanol-DCM as eluent) to afford the title compound 12 (0.6 g, 0.760 mmol, 47.69% yield) as a white solid. LC-MS: Ret. Time: 1.32 min. MS calcd. for chemical formula: C₂₅H₂₇N₃O₅ 449.5; Found: 450.27 [M+H]⁺. Method: FA BEH 2.5 min. Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-9: Synthesis of ethyl 2-[5-[4-[4-[(5-fluoroisoindoline-2-carbonyl)amino]phenyl]cyclohexyl]-1,3,4-oxadiazol-2-yl]acetate (14): To a stirred solution of compound 12 (0.5 g, 0.344 mmol) in DMF (5 mL) were added DIPEA (0.58 mL, 1.112 mmol) and compound 13 (0.183 g, 1.335 mmol) at 0° C. The mixture was then stirred at 80° C. for 3 hours under nitrogen atmosphere. After completion of the reaction (monitored by TLC), the reaction mixture was cooled to room temperature, ice-cooled water was added to the reaction mixture, and a solid was precipitated. The solid was filtered and dried to give the crude product, which was purified by Flash column chromatography (230-400 silica, 5% methanol-DCM as eluent) to afford the title compound 14 (0.4 g, 0.657 mmol 59.14% yield) as a white solid. LC-MS: Ret. Time: 1.27 min. MS calcd. for chemical formula: C₂₇H₂₉FN₄O₄ 492.5; Found: 493.33 [M+H]⁺. Method: TFA BEH 2.5 MIN, Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-10: Synthesis of 5-fluoro-{N}-[4-[4-[5-(2-hydroxyethyl)-1,3,4-oxadiazol-2-yl]cyclohexyl]phenyl]isoindoline-2-carboxamide (I-28): To a stirred solution of compound 14 (0.1 g, 0.203 mmol) in THF (10 mL) was added LiBH₄ (0.2 ml, 0.406 mmol) at 0° C. The mixture was stirred at room temperature for 2 hours under nitrogen atmosphere. After completion of the reaction (monitored by TLC), the mixture was cooled to 0° C., quenched with ammonium chloride solution, washed with water, and extracted with ethyl acetate. The combined organic layer was dried with anhydrous sodium sulphate, and the solvent was evaporated under reduced pressure to give a crude product, which was purified by Prep-HPLC to afford the title compound I-28 (0.011 g, 0.0241 mmol 11.91% yield) as a white solid. Purification method: Column: Xtimate C18 (250×21.2) mm, 5 μm. Mobile phase A: 0.1% FA in water. Mobile phase B: 100% ACN. Flow rate: 25 mL/min. Instrument ID: PREP-19. Gradient (Time/% B): 0/40, 2/40, 15/65. LC-MS: Ret. Time: 2.09 min. Purity: 99.83%. MS calcd. for chemical formula: C₂₅H₂₇FN₄O₃ 450.55; Found: 451.34 [M+H]⁺. LC-MS Method: A BEH 4.2 MINS, Column: Acquity UPLC BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 40° C. Gradient Program Time/B: 0.0/2, 0.3/2, 2.0/98, 3.5/98, 3.6/2, 4.2/2. HPLC Method: Ret. Time: 7.509 min. Purity: 99.46%. Column: X-Select CSH C18 5 μm, (4.6×150) mm. Mobile Phase A: 0.05% FA in water and acetonitrile (95:5). Mobile Phase B: 100% acetonitrile. Colum Temperature: 30° C. Flow rate: 10 mL/min. Gradient: 0.0/5, 1.0/5, 8.0/95, 12.0/95, 14.0/5, 18.0/5. Diluent: ACN:Water (80:20). Instrument ID: AMC-HPLC-32. ¹H NMR (400 MHz, DMSO-d₆): δ 8.30 (s, 1H), 7.46 (d, J=8.6 Hz, 2H), 7.41-7.36 (m, 1H), 7.26-7.20 (m, 1H), 7.14 (br d, J=8.6 Hz, 3H), 4.95-4.87 (m, 1H), 4.73 (br d, J=11.6 Hz, 4H), 3.80-3.71 (m, 2H), 2.95 (t, J=6.3 Hz, 3H), 2.57-2.53 (m, 1H), 2.19-2.11 (m, 2H), 1.93-1.84 (m, 2H), 1.70-1.52 (m, 4H).

### Example 18—Synthesis of 5-fluoro-N-(4-(1-(3-(2-hydroxy-2-methylpropyl)-1H-1,2,4-triazol-5-yl)piperidin-4-yl) phenyl)isoindoline-2-carboxamide (I-26)

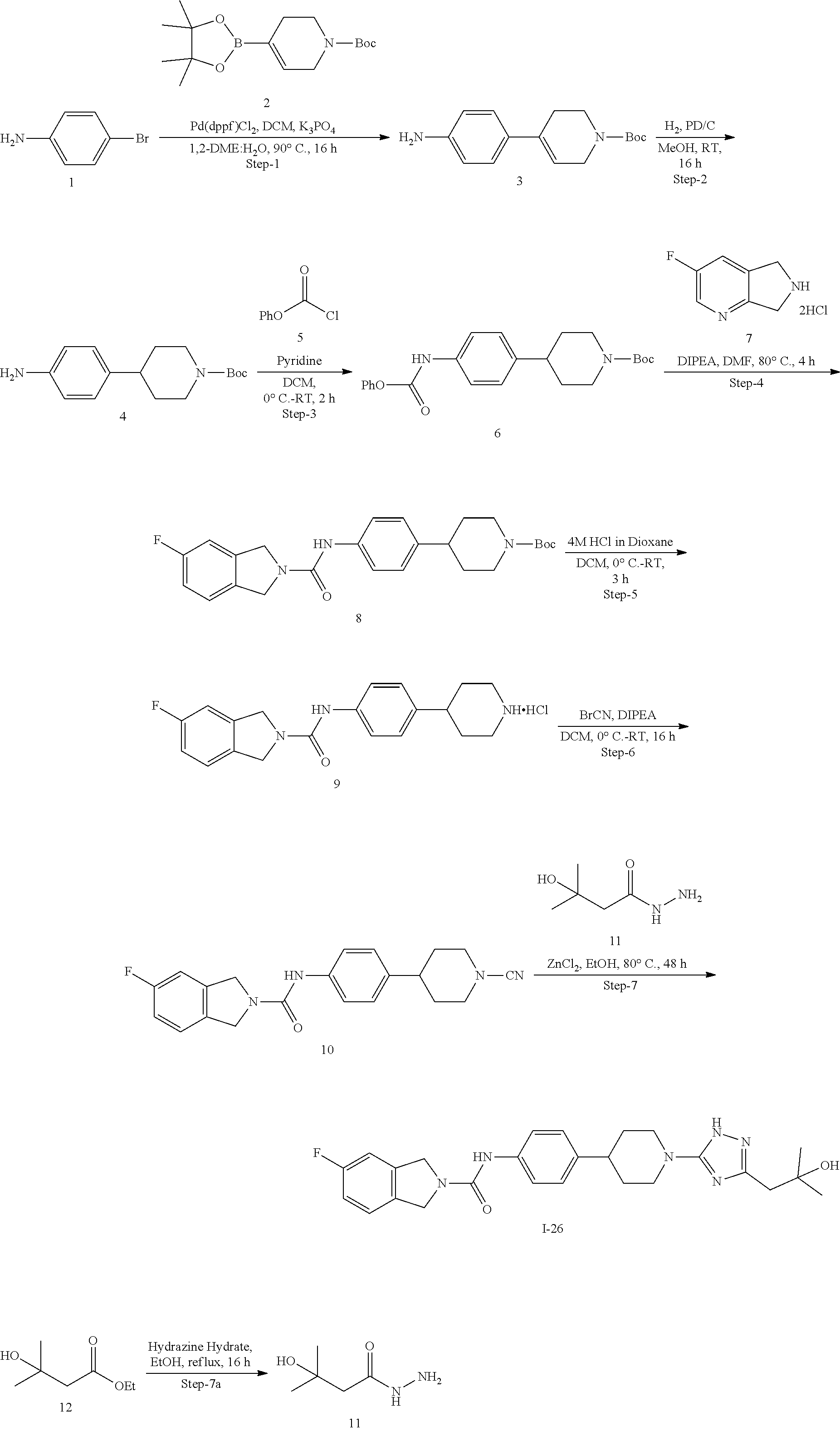

Step-1: Synthesis of tert-butyl 4-(4-aminophenyl)-3,6-dihydropyridine-1(2H)-carboxylate (3): A mixture of compound 1 (10 g, 56.38 mmol), compound 2 (20 g, 64.68 mmol), and K₃PO₄ (40 g, 184.7 mmol) in 1,2-DME:H₂O (10:2, 120 mL) was degassed with argon for 5 minutes. Pd(dppf)Cl₂. DCM (2.3 g, 2.8 mmol) was added to the mixture at room temperature. The reaction mixture was stirred for 15 minutes under degassing with argon, and then the reaction mixture was heated at 90° C. and stirred for 16 hours. After completion of the reaction (monitored by TLC), the reaction mixture was filtered through a pad of CELITE® and washed with EtOAc. The organic layer was diluted with water and extracted with EtOAc. The combined organic layers were washed with brine, dried over anhydrous Na₂SO₄, and concentrated under reduced pressure to afford the crude compound (20 g), which was purified in a combiflash column (30% EtOAc in heptane) to afford the title compound 3 (15.95 g, 54.66 mmol, 94% yield) as a brown solid. LC-MS: Ret. Time: 1.83 min. Purity: 85.72%. MS calcd. for chemical formula: C₁₆H₂₂N₂O₂: 274.4; Found: 275.1 [M+H]⁺. Method: Column: X-Select CSH C18 (50 mm×3.0 mm, 2.5p). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-2: Synthesis of tert-butyl 4-(4-aminophenyl)piperidine-1-carboxylate (4): To a stirred solution of compound 3 (15 g, 54.66 mmol) in MeOH (150 mL) was added 10% palladium on carbon (50% wet, 6.0 g). The mixture was stirred at room temperature for 16 hours under hydrogen gas (150 psi) in an autoclave. After completion of the reaction (monitored by TLC), the reaction mixture was filtered through a CELITE® pad and washed with EtOAc. The combined organic layers were concentrated under reduced pressure to afford the title compound 4 (14 g, 50.65 mmol, 92.66% yield) as an off-white solid. The crude compound was used directly in the next step without purification. LC-MS: Ret. Time: 1.70 min. Purity: 64.64%. MS calcd. for chemical formula: C₁₆H₂₄N₂O₂: 276.4; Found: 221.0 [(M-Isobutene)+H]⁺. Method: Column: X-Select CSH C18 (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-3: Synthesis of tert-butyl 4-(4-((phenoxycarbonyl)amino)phenyl)piperidine-1-carboxylate (6): To a stirred solution of compound 4 (12.00 g, 41.04 mmol) in DCM (120 mL) was added pyridine (6.53 g, 82.08 mmol) and compound 5 (6.4 g, 41.04 mmol) at 0° C. The mixture was stirred at room temperature for 2 hours under nitrogen atmosphere. After completion of the reaction (monitored by TLC), the reaction mixture was diluted with water and extracted with DCM. The combined organic layers were washed with brine, dried over anhydrous Na₂SO₄, and concentrated under reduced pressure. The crude compound was triturated with diethyl ether, filtered, and dried under reduced pressure to afford the title compound 6 (11.0 g, 27.74 mmol, 67.60% yield) as a pale-brown solid. LC-MS: Ret. Time: 2.64 min. Purity: 85.28%. MS calcd. for chemical formula: C₂₃H₂₈N₂O₄: 396.49; Found: 340.9 [(M-Isobutene)+H]⁺. Method: Column: Acquity UPLC BEH C18 (2.1×50) mm, 1.7 μm. Flow rate: 0.5 mL/min. Mobile Phase A: 0.05% FA in water. Mobile Phase B: 0.05% FA in acetonitrile. Column temperature: 40° C. Gradient Program Time/B: 0.0/2, 0.3/2, 2.0/98, 3.5/98, 3.6/2, 4.2/2.

Step-4: Synthesis of tert-butyl 4-(4-(5-fluoroisoindoline-2-carboxamido)phenyl) piperidine-1-carboxylate (8): To a stirred solution of compound 6 (3.8 g, 9.6 mmol) and compound 7 (1.5 g, 8.6 mmol) in DMF (38 mL) was added DIPEA (5.0 mL, 29 mmol) at room temperature. The mixture was refluxed at 80° C. for 4 hours under nitrogen atmosphere. After completion of the reaction (monitored by TLC), the reaction mixture was quenched with ice-cold water to form a precipitate. The solid was filtered and washed with diethyl ether and n-pentane and dried under reduced pressure to afford the title compound 8 (4.10 g, 9.3 mmol, 97% yield) as a brown solid. LC-MS: Ret. Time: 2.21 min. Purity: 76.93%. MS calcd. for chemical formula: C₂₅H₃₀FN₃O₃: 439.5; Found: 384.3 [M-Isobutene)+H]⁺. Method: Column: X-Select CSH C18 (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-5: Synthesis of 5-fluoro-N-(4-(piperidin-4-yl)phenyl)isoindoline-2-carboxamide hydrochloride (9): To a stirred solution of compound 8 (4.0 g, 9.101 mmol) in DCM (40 mL) was added HCl (4 M) in 1,4-dioxane (20 mL) at 0° C. The mixture was stirred at room temperature for 3 hours. After completion of the reaction (monitored by TLC), volatiles were removed under reduced pressure. The residue was stirred in diethyl ether (50 mL) and then with pentane (50 mL×2). The solid was filtered and dried under reduced pressure to afford the title compound 9 (3.0 g, 8.839 mmol, 97.12%) as a pale-brown solid. LC-MS: Ret. Time: 1.44 min. Purity: 81.60%. MS calcd. for chemical formula: C₂₀H₂₃ClFN₃O: 339.4; Found: 340.4 [M+H]⁺. Method: Column: X-Select CSH C18 (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-6: Synthesis of N-(4-(1-cyanopiperidin-4-yl)phenyl)-5-fluoroisoindoline-2-carboxamide (10): To a stirred solution of compound 9 (0.250 g, 0.7366 mmol) in DCM (10 mL) was added DIPEA (0.283 mL, 1.621 mmol) and cyanogen bromide (0.3346 g, 1.273 mmol) at 0° C. The reaction mixture was stirred at room temperature for 16 hours. After completion of the reaction (monitored by TLC), the reaction mixture was diluted with water and extracted with DCM. The combined organic layers were washed with brine, dried over anhydrous Na₂SO₄, and concentrated under reduced pressure to afford the crude compound (0.250 g), which was purified by combiflash column (5% MeOH in DCM) to afford the title compound 10 (0.180 g, 0.4940 mmol, 67% yield) as a pale-brown solid. LC-MS: Ret. Time: 1.97 min. Purity: 90.10%. MS calcd. for chemical formula: C₂₁H₂₁FN₄O: 364.4; Found: 365.3 [M+H]⁺. Method: Column: X-Select CSH C18 (50 mm×3.0 mm, 2.5 μm). Mobile Phase A: 0.05% formic acid in water. Mobile Phase B: 0.05%; formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-7: Synthesis of 5-fluoro-N-(4-(1-(3-(2-hydroxy-2-methylpropyl)-1H-1,2,4-triazol-5-yl)piperidin-4-yl)phenyl)isoindoline-2-carboxamide (I-26): To the stirred solution of compound 11 (0.110 g, 0.8323 mmol) and compound 10 (0.182 g, 0.4993 mmol) in EtOH (10 mL) was added zinc chloride (1.0 mol/L in THF, 0.42 mL, 0.4161 mmol) at room temperature. The reaction mixture was heated at 80° C. and stirred for 48 hours. After completion of the reaction (monitored by TLC), the reaction mixture was cooled to room temperature and concentrated under reduced pressure to give the crude compound (0.300 g), which was purified using PREP-HPLC to afford the desired compound (I-26) (0.060 g, 0.1216 mmol, 14.6% yield) as an off-white solid. PREP-HPLC Method: Column: Xtimate C18 (250×30) mm, 5 μm. Mobile Phase A: 0.1% FA in water. Mobile Phase B: 100% ACN. Flow rate: 22 mL/min. Instrument ID: PREP-19; Gradient (Time/% B): 0/10, 3/10, 8/35, 22/60, 23/100. Solvents used for dilution: acetonitrile+water+THF. Actual Quantity Purified: 800 mg. Volume of fraction: 400 mL. Number of Injections: 15. Loading: 53 mg/inj acetonitrile: 8 mg/inj crude. Purity: 17%. Fraction purity: 95%. LCMS: Ret. Time: 1.05 min. Purity: 97.29%. MS calcd. for chemical formula: C₂₆H₃₁FN₆O₂: 478.6; Found: 479.3 [M+H]⁺. LCMS Method: Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3. HPLC: Ret. Time: 6.025 min. Purity: 95.36%. HPLC Method: Column: X-Select CSH C18 5 μm, (4.6×150) mm. Mobile Phase A: 0.05% FA in water and acetonitrile (95:5). Mobile Phase B: 100% Acetonitrile. Colum Temperature: 30° C. Flow Rate: 1.0 mL/min. Gradient: 0.0/5, 1.0/5, 8.0/95, 12.0/95, 14.0/5, 18.0/5. Diluent: acetonitrile:water (80:20). Instrument ID: AMC-HPLC-32. ¹H NMR (400 MHz, DMSO-d₆): δ 12.64-12.19 (m, 1H), 8.30 (s, 1H), 7.46 (d, J=8.5 Hz, 2H), 7.38 (dd, J=8.3, 5.3 Hz, 1H), 7.26-7.20 (m, 1H), 7.13 (d, J=8.6 Hz, 3H), 4.73 (br d, J=11.8 Hz, 4H), 4.66-4.59 (m, 1H), 4.09-3.87 (m, 2H), 2.98-2.74 (m, 2H), 2.73-2.60 (m, 3H), 1.86-1.74 (m, 2H), 1.72-1.58 (m, 2H), 1.13 (s, 6H).

Step-8: Synthesis of 3-hydroxy-3-methylbutanehydrazide (11): To a stirred solution of compound 12 (0.25 g, 1.7102 mmol) in MeOH (5 mL) was added hydrazine (0.53 mL, 13.682 mmol) at room temperature. The reaction mixture was heated at 70° C. and stirred for 16 hours. After completion of the reaction (monitored by TLC), the reaction mixture was cooled to room temperature and concentrated under reduced pressure to give the crude product (0.300 g), which was triturated with DCM (2×10 mL) and dried under reduced pressure to afford the title compound 11 (0.200 g, 1.5133 mmol, 88.49% yield) as an off-white solid. LCMS: MS calcd. for formula: C₅H₁₂N₂O₂: 132.16; Found: Not observed [M+H]⁺. ¹H NMR (400 MHz, DMSO-d₆): δ 8.97 (br s, 1H), 4.43-3.99 (m, 2H), 3.71-3.21 (m, 2H), 2.14 (s, 2H), 1.13 (s, 6H).

### Example 19—Synthesis of 3-fluoro-N-(4-(1-(3-(2-hydroxy-2-methylpropyl)-1H-1,2,4-triazol-5-yl)piperidin-4-yl)phenyl)-5,7-dihydro-6H-pyrrolo[3,4-b]pyridine-6-carboxamidecarboxamidecarboxamide (I-24)

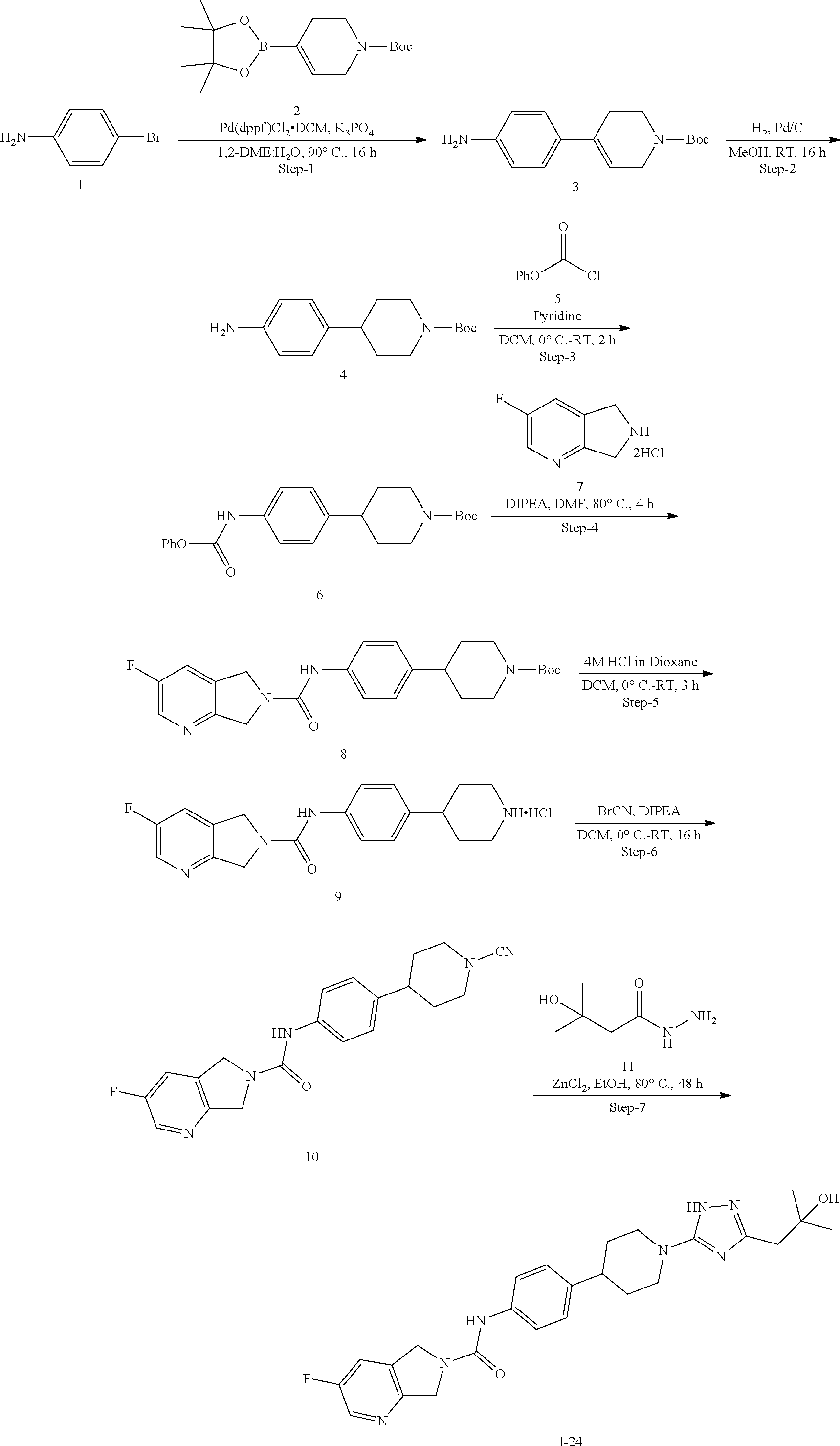

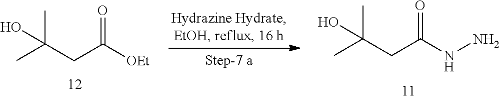

Step-1: Synthesis of tert-butyl 4-(4-aminophenyl)-3,6-dihydropyridine-1(2H)-carboxylate (3): A mixture of compound 1 (10 g, 56.38 mmol), compound 2 (20 g, 64.68 mmol), and K₃PO₄ (40 g, 184.7 mmol) in 1,2-DME:H₂O (10:2, 120 mL) was degassed with argon for 5 minutes. Pd(dppf)Cl₂. DCM (2.3 g, 2.8 mmol) was added to the mixture at room temperature. The reaction mixture was stirred for 15 minutes under degassing with Argon, and then the reaction mixture was stirred at 90° C. for 16 hours. After completion of the reaction (monitored by TLC), the reaction mixture was filtered through a CELITE® pad and washed with EtOAc. The organic layer was diluted with water and extracted with ethyl acetate. The combined organic layers were washed with water followed by brine, dried over anhydrous Na₂SO₄, and concentrated under reduced pressure. The crude compound was purified by CombiFlash (Nexgen-300) chromatography 12 g cartridge silica gel using 20-35% EtOAc in n-heptane as an eluent to afford the title compound 3 (11 g, 40.09 mmol, 69% yield) as a brown solid. LC-MS: Ret. Time: 1.29 min. Purity: 85.72%. MS calcd. for chemical formula C₁₆H₂₂N₂O₂, 274.36; Found: 275.41 [M+H]⁺. Method F: Column: CORTECS UPLC C18 (3×30) mm, 1.6 μm. Flow rate: 0.85 mL/min. Mobile Phase A: 0.05% formic acid in water. Mobile Phase B: 0.05% formic acid in ACN. Injection volume: 2 μL. Column Temp.: 45° C. Gradient Program % Time/B: 0.0/3, 0.1/3, 1.4/97, 2.0/97, 2.05/3, 2.5/3.

Step-2: Synthesis of tert-butyl 4-(4-aminophenyl)piperidine-1-carboxylate (4): To a stirred solution of compound 3 (5.6 g, 20.04 mmol) in MeOH (50 mL) was added 10% palladium on carbon (50% wet, 2.77 g). The mixture was stirred at room temperature for 16 hours under hydrogen atmosphere (150 psi) in an autoclave. After completion of the reaction (monitored by TLC), the reaction mixture was filtered through a CELITE® pad and washed with EtOAc. The organic layer was concentrated under reduced pressure to afford the crude title compound 4 (5.5 g, 20 mmol, 99% yield) as an off-white solid. LC-MS: Ret. Time: 1.119 min. Purity: 64.64%. MS calcd. for chemical formula: C₁₆H₂₄N₂O₂: 276.38; Found: 221.44 [(M-Isobutene)+H]⁺. Method G: X-select CSH 18 (3×50 mm×2.5 mm). Mobile phase A: 0.05% TFA in H₂O. Mobile Phase B: 0.05% TFA in ACN. Injection volume: 2 μL. Flow rate: 1 mL/min. Column temperature: 40° C. Gradient program: 0% B, % Time/B: 0.0/2, 0.3/2, 2/98, 2.8/98, 3.0/2, 3.7/2.

Step-3: Synthesis of tert-butyl 4-(4-((phenoxycarbonyl)amino)phenyl)piperidine-1-carboxylate (6): To a stirred solution of compound 4 (5.5 g, 20 mmol) in DCM (40 mL) were added pyridine (3.2 mL, 40 mmol), compound 5 (3.5 g, 22 mmol), and DMAP (0.25 g, 2.0 mmol) at 0° C. The mixture was stirred at room temperature for 2 hours under nitrogen atmosphere. After completion of the reaction (monitored by TLC), the reaction mixture was diluted with water and extracted with DCM. The combined organic layers were washed with water followed by brine, dried over anhydrous Na₂SO₄, and concentrated under reduced pressure. The crude compound was triturated with diethyl ether and n-pentane. The solid was filtered and dried under reduced pressure to afford the title compound 6 (5.0 g, 12.61 mmol, 87.14% yield) as a pale-brown solid. The crude compound was used directly in the next step without purification. LC-MS: Ret. Time: 3.01 min. Purity: 85.28%. MS calcd. for chemical formula: C₂₃H₂₈N₂O₄: 396.49; Found: 340.9 [(M-Isobutene)+H]⁺. Method I: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-4: Synthesis of tert-butyl 4-(4-(3-fluoro-6,7-dihydro-5H-pyrrolo[3,4-b]pyridine-6-carboxamido)phenyl)piperidine-1-carboxylate (8): To a stirred solution of compound 6 (1.4 g, 3.53 mmol) and compound 7 (0.90 g, 3.18 mmol) in DMF (14 mL) was added DIPEA (1.85 mL, 10.6 mmol) at room temperature. The mixture was refluxed at 80° C. for 6 hours under nitrogen atmosphere. After completion of the reaction (monitored by TLC), the reaction mixture was quenched with ice-cold water. The solid thus precipitated was filtered, washed with diethyl ether and n-pentane, and evaporated under reduced pressure to afford the title compound 8 (1.4 g, 3.2 mmol, 90% yield) as a brown solid. LCMS: Ret. Time: 2.26 min. Purity: 84.44%. MS calcd. for chemical formula: C₂₄H₂₉FN₄O₃: 440.22; Found: 439.0 [(M-Isobutene)-H]—. Method: column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-5: Synthesis of 3-fluoro-N-(4-(piperidin-4-yl)phenyl)-5,7-dihydro-6H-pyrrolo[3,4-b]pyridine-6-carboxamide hydrochloride (9): To a stirred solution of compound 8 (1.4 g, 3.2 mmol) in DCM (14 mL) was added HCl (4 M) in 1,4-dioxane (12 mL) at 0° C. The mixture was stirred at room temperature for 3 hours. After completion of the reaction (monitored by TLC), volatiles were removed under reduced pressure. The residue was stirred in diethyl ether (10 mL) and then with pentane (10 mL×2). The solid compound was filtered and dried under reduced pressure to afford the title compound 9 (0.80 g, 2 mmol, 70.0%) as a pale-brown solid. LC-MS: Ret. Time: 1.37 min. Purity: 74.0%. MS calcd. for chemical formula: C₁₉H₂₁FN₄O: 340.17; Found: 341.48 [M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-6: Synthesis of N-(4-(1-cyanopiperidin-4-yl)phenyl)-3-fluoro-5,7-dihydro-6H-pyrrolo[3,4-b]pyridine-6-carboxamide (10): To a stirred solution of compound 9 (0.200 g, 0.5875 mmol) in DCM (3 mL) was added DIPEA (0.22 mL, 1.293 mmol) and cyanogen bromide (0.076 g, 0.6463 mmol) at 0° C. The reaction mixture was stirred at room temperature for 16 hours. After completion of the reaction (monitored by TLC, LC), the reaction mixture was diluted with water and extracted with DCM. The combined organic layers were washed with brine, dried over anhydrous Na₂SO₄, and concentrated under reduced pressure to afford the crude compound 10 (0.250 g). Purification was done using combi flash column (5% MeOH in DCM) to afford the title compound 10 (0.150 g, 0.4105 mmol, 69.87% yield) as a pale-brown solid. LC-MS: Ret. Time: 1.86 min. Purity: 89.91%. MS calcd. for chemical formula: C₂₀H₂₀FN₅O 365.17; Found: 366.2 [M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-7: Synthesis of 3-fluoro-N-(4-(1-(3-(2-hydroxy-2-methylpropyl)-1H-1,2,4-triazol-5-yl)piperidin-4-yl)phenyl)-5,7-dihydro-6H-pyrrolo[3,4-b]pyridine-6-carboxamide (I-24): To a stirred solution of compound 11 (0.150 g, 1.135 mmol) and compound 10 (0.130 g, 0.355 mmol) in EtOH (10 mL) was added zinc chloride (1.0 mol/L in THF, 0.91 mL, 0.907 mmol) at room temperature. The reaction mixture was heated at 80° C. and stirred for 48 hours. After completion of the reaction (monitored by TLC, LC), the reaction mixture was cooled to room temperature and concentrated under reduced pressure to give the crude compound (0.300 g), which was purified using Prep-HPLC to afford the desired compound I-24 (0.025 g, 0.05421 mmol, 4.776% yield) as an off-white solid. Prep-HPLC Method: Column Xtimate C18 (250×30) mm, 5 μm. Mobile Phase A: 0.1% formic acid in water. Mobile Phase B: 100% acetonitrile. Flow rate: 22 mL/min. Instrument ID PREP-26 Gradient (Time/% B) 0/10, 3/10, 8/25, 22/30, 23/98. LCMS: Ret. Time: 2.30 min. Purity: 97.69%. MS calcd. for chemical formula: C₂₅H₃₀FN₇O₂: 479.24; Found: 480.4 [M+H]⁺. LCMS Method: Column: XbridgeC18 (4.6×50 mm, 3.5 μm). Mobile Phase: A: 10 mM NH₄HCO₃ in water. Mobile Phase: B: ACN (Gradient) T/B %: 0.0/2, 0.3/2, 2.5/98, 4.0/98, 4.2/2, 5/2. Flow rate: 1 mL/min. Column Temp.: 40° C. HPLC: Ret. Time: 4.17 min. Purity: 98.31%. HPLC Method: Column: X Bridge BEH C18, 2.5 μm, 100×3 mm. Mobile Phase A: 0.05% TFA in water. Mobile Phase B: 0.05% TFA in acetonitrile. Colum Temperature: 50° C. Flow Rate: 0.5 mL/min. Gradient: 0/5, 1/5, 6/90, 8.5/90, 8.8/5, 11/5. Instrument ID: AMC-UPLC-04. ¹H NMR (400 MHz, DMSO-d₆): δ 12.62-12.19 (m, 1H), 8.50-8.45 (m, 1H), 8.36 (s, 1H), 7.81-7.77 (m, 1H), 7.47 (d, J=8.6 Hz, 2H), 7.14 (d, J=8.5 Hz, 2H), 4.75 (br d, J=19.4 Hz, 4H), 4.66-4.58 (m, 1H), 4.09-3.85 (m, 2H), 3.01-2.61 (m, 5H), 1.84-1.73 (m, 2H), 1.71-1.58 (m, 2H), 1.13 (s, 6H).

Step-8: Synthesis of 3-hydroxy-3-methylbutanehydrazide (11): To a stirred solution of compound 11 (0.25 g, 1.7102 mmol) in MeOH (5 mL) was added hydrazine (0.53 mL, 13.682 mmol) at room temperature. The reaction mixture was heated at 70° C. and stirred for 16 hours. After completion of the reaction (monitored by TLC), the reaction mixture was cooled to room temperature and concentrated under reduced pressure to give the crude compound (0.300 g), which was triturated with DCM (2×10 mL) and dried under reduced pressure to afford the title compound 12 (0.150 g, 1.1350 mmol, 67.37% yield) as an off-white solid. LCMS: MS calcd. Formula: C₅H₁₂N₂O₂: 132.16; Found: Not observed [M+H]⁺. ¹H NMR (400 MHz, DMSO-d₆): δ 8.97 (br s, 1H), 4.43-3.99 (m, 2H), 3.71-3.21 (m, 2H), 2.14 (s, 2H), 1.13 (s, 6H).

### Example 20—Synthesis of 5-fluoro-N-(4-(4-(5-(2-hydroxyethyl)-1,3,4-oxadiazol-2-yl) bicyclo [2.2.2]octan-1-yl) phenyl) isoindoline-2-carboxamide (I-18)

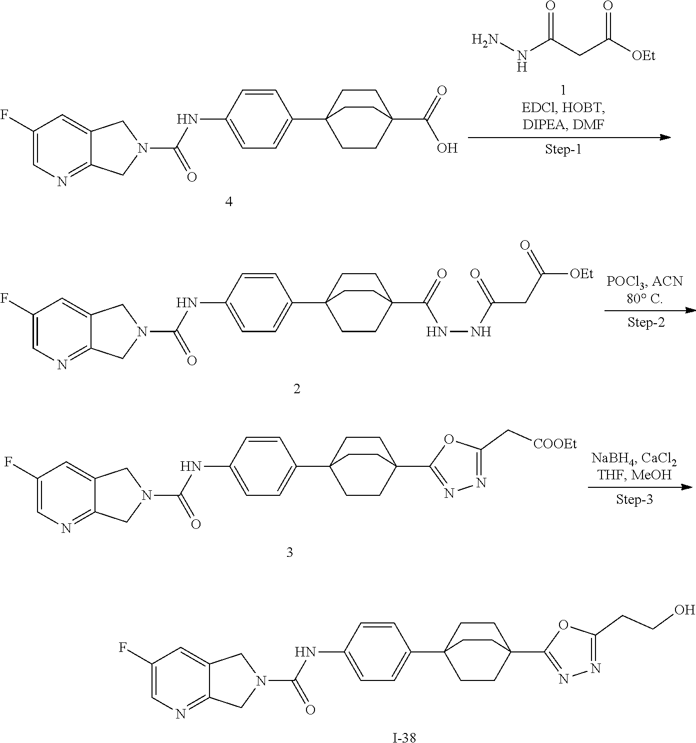

The following LCMS method was used: Column: X Bridge C18 4.6×50 mm×3.5 μm. Mobile Phase A: 10 mm ammonium acetate. Mobile B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of ethyl 3-(2-(4-(4-(5-fluoroisoindoline-2-carboxamido) phenyl) bicyclo [2.2.2]octane-1-carbonyl) hydrazineyl)-3-oxopropanoate (3): To a stirred solution of 1 (1.0 g, 2.45 mmol) in DMF (10 mL) was added 2 (429 mg, 2.94 mmol), EDCI (1.87 g, 9.80 mmol), HOBt (1.49 g, 9.80 mmol), and DIPEA (2.7 mL, 14.70 mmol) at room temperature. The mixture was stirred at room temperature for 16 hours. The progress of the reaction was monitored by TLC. After complete consumption of starting material, the reaction mixture was diluted with ice-cold water. The precipitate was filtered, washed with water, and dried to give a residue, which was triturated with n-pentane (20 mL) to obtain 3 (1.18 g, 90% yield) as an off-white solid. ¹H NMR (300 MHz, DMSO-d₆): δ 8.29 (s, 1H), 7.47-7.41 (m, 2H), 7.38 (dd, J=5.5, 8.3 Hz, 1H), 7.26-7.18 (m, 3H), 7.17-7.09 (m, 1H), 4.72 (d, J=8.1 Hz, 4H), 4.08 (q, J=7.1 Hz, 2H), 3.26 (s, 2H), 1.93-1.65 (m, 12H), 1.19 (t, J=7.1 Hz, 3H), 1.05-0.82 (m, 2H). LCMS: 537.09 (M+H). Purity: 82.36% by AUC. RT: 5.02. UV: 254 nm.

Step-2: Synthesis of ethyl 2-(5-(4-(4-(5-fluoroisoindoline-2-carboxamido) phenyl) bicyclo [2.2.2]octan-1-yl)-1,3,4-oxadiazol-2-yl) acetate (4): To a stirred solution of 3 (300 mg, 0.55 mmol) in ACN (10.0 mL) was added POCl₃ (0.16 mL, 1.67 mmol) slowly at 0° C. The reaction mixture was stirred at room temperature for 4 hours in a sealed tube and monitored by TLC and LCMS. After complete consumption of starting material, the volatiles were concentrated under reduced pressure to obtain a residue, which was diluted with water (20 mL) and basified (to pH ˜8-9) with saturated NaHCO₃ solution. The reaction mixture was extracted with EtOAc (2×80 mL). The combined organic layers were dried over Na₂SO₄ and concentrated under reduced pressure to afford the crude product, which was purified by column chromatography (eluent: 50% EtOAc in hexane), which afforded 4 (200 mg, 69% yield) as an off-white solid. ¹H NMR (300 MHz, DMSO-d₆): δ 8.30 (s, 1H), 7.47 (d, J=8.7 Hz, 2H), 7.39 (dd, J=5.2, 8.5 Hz, 1H), 7.27-7.20 (m, 3H), 7.18-7.09 (m, 1H), 4.74 (s, 2H), 4.73-4.68 (m, 2H), 4.18-4.10 (m, 4H), 2.02-1.93 (m, 6H), 1.93-1.82 (m, 6H), 1.19 (t, J=7.1 Hz, 3H). LCMS: 519.34 (M+H). Purity: 92.73% by AUC. RT: 5.58. UV: 254 nm.

Step-3: Synthesis of 5-fluoro-N-(4-(4-(5-(2-hydroxyethyl)-1,3,4-oxadiazol-2-yl) bicyclo [2.2.2]octan-1-yl) phenyl) isoindoline-2-carboxamide (I-18): To a stirred solution of 4 (150 mg, 0.28 mmol) in a mixture of THF:MeOH (2:1, 7.0 mL) was added CaCl₂ (127 mg, 1.15 mmol) followed by NaBH₄ (77 mg, 2.02 mmol) portion-wise at 0° C. The reaction mixture was stirred at room temperature for 1 hour and monitored by TLC and LCMS. After complete consumption of starting material, the reaction mixture was quenched with saturated NH₄Cl solution. The reaction mixture was extracted with EtOAc (2×50 mL). The combined organic layers were dried over Na₂SO₄ and concentrated under reduced pressure to afford the crude product, which was purified by column chromatography (eluent: 3% MeOH in DCM) to afford I-18 (60 mg, 44% yield) as an off-white solid. ¹H NMR (300 MHz, DMSO-d₆): δ 8.30 (s, 1H), 7.51-7.42 (m, 2H), 7.42-7.35 (m, 1H), 7.28-7.19 (m, 3H), 7.18-7.09 (m, 1H), 4.91 (t, J=5.5 Hz, 1H), 4.73 (d, J=8.0 Hz, 4H), 3.75 (q, J=6.3 Hz, 2H), 2.94 (t, J=6.3 Hz, 2H), 2.01-1.92 (m, 6H), 1.87 (d, J=8.3 Hz, 6H). LCMS: 477.07 (M+H), Purity—95.02% by AUC, RT—5.00, UV—245 nm. HPLC: Purity—95.17% by AUC, RT—13.85, UV—245 nm.

### Example 21—Synthesis of 3-fluoro-N-(4-((1r,4r)-4-(4-(2-hydroxypropan-2-yl)-1H-1,2,3-triazol-1-yl) cyclohexyl) phenyl)-5,7-dihydro-6H-pyrrolo [3,4-b]pyridine-6-carboxamide (I-25)

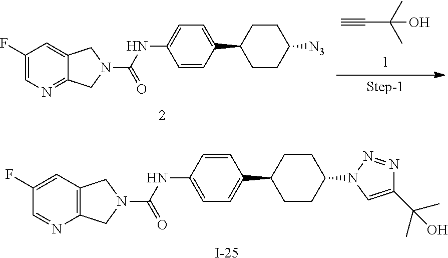

The following LCMS method was used: Column: X Bridge C18 4.6×50 mm×3.5 μm; Mobile Phase A: 10 mM ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

To a stirred solution of 2 (150 mg, 0.39 mmol) in a mixture of t-buOH:water (1:1, 5.0 mL) was added sodium ascorbate (39.07 mg, 0.19 mmol), CuSO₄.5H₂O (49 mg, 0.19 mmol), and 1 (132 mg, 1.58 mmol) at room temperature. The reaction mixture was heated in a microwave at 100° C. for 1 hour. The reaction mixture was monitored by TLC and LCMS. After complete consumption of starting material, the reaction mixture was diluted with water (50 mL) and extracted with EtOAc (2×50 mL). The organic layer was dried over anhydrous Na₂SO₄, filtered, and concentrated under reduced pressure to afford crude product, which was purified by column chromatography (eluent: 0-5% MeOH in DCM) followed by trituration with n-pentane to afford I-25 (50 mg, 27%) as an off-white solid. ¹H NMR (300 MHz, DMSO-d₆): δ 8.52-8.44 (m, 1H), 8.37 (s, 1H), 7.96 (s, 1H), 7.84-7.74 (m, 1H), 7.48 (d, J=8.4 Hz, 2H), 7.18 (d, J=8.4 Hz, 2H), 5.11-5.03 (m, 1H), 4.76 (d, J=13.8 Hz, 4H), 4.61-4.47 (m, 1H), 2.60 (s, 1H), 2.16 (d, J=9.4 Hz, 2H), 1.98-1.89 (m, 4H), 1.75-1.64 (m, 2H), 1.47 (s, 6H). LCMS: 465.33 (M+H). Purity: 98.81% by AUC. RT: 4.57. UV: 245 nm. HPLC: Purity: 94.88% by AUC. RT: 12.45. UV: 245 nm.

### Example 22—Synthesis of 3-fluoro-N-(4-(4-(4-(2-hydroxy-2-methylpropyl) thiazol-2-yl) bicyclo [2.2.2]octan-1-yl) phenyl)-5,7-dihydro-6H-pyrrolo [3,4-b]pyridine-6-carboxamide (I-29)

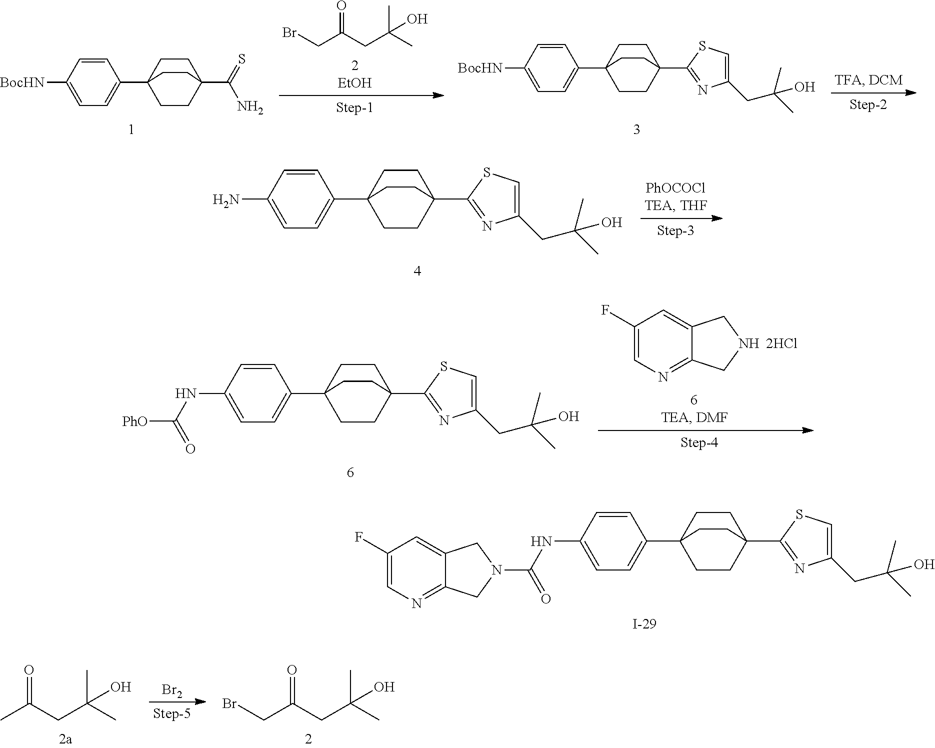

The following LCMS method was used: Column: X Bridge C18 4.6×50 mm×3.5 μm. Mobile Phase A: 10 mM ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of tert-butyl (4-(4-(4-(2-hydroxy-2-methylpropyl) thiazol-2-yl) bicyclo [2.2.2]octan-1-yl) phenyl) carbamate (3): To a stirred solution of 1 (200 mg, 0.55 mmol) in EtOH (10.0 mL) was added 2 (161 mg, 0.83 mmol) at room temperature. The reaction mixture was stirred at 50° C. for 16 hours. The reaction mixture was monitored by TLC and LCMS. After complete consumption of starting material, the reaction mixture was poured into ice-cold water. The precipitate was filtered, washed with water (50 mL), and dried to obtain 3 (210 mg, 83% yield) as an off-white solid. LCMS: 457.33 (M+H). Purity: 78.62% by AUC. RT: 6.78. UV: 245 nm.

Step-2: Synthesis of 1-(2-(4-(4-aminophenyl) bicyclo [2.2.2]octan-1-yl) thiazol-4-yl)-2-methylpropan-2-ol (4): To a stirred solution of 3 (300 mg, 0.66 mmol) in DCM (5 mL) was added TFA (225 mg, 1.97 mmol) at 0° C. under N₂ atmosphere. The reaction mixture was stirred at room temperature for 4 hours. The reaction mixture was monitored by TLC and LCMS. After complete consumption of starting material, the reaction mixture was diluted with water, basified to pH ˜8-9 with saturated NaHCO₃ solution (30 mL), and extracted with DCM (2×80 mL). The combined organic layer was dried over Na₂SO₄ and concentrated under reduced pressure to afford the crude product, which was triturated with n-pentane to give 4 (210 mg, 89% yield) as a light-brown solid. ¹H NMR (300 MHz, DMSO-d₆): δ=7.12 (s, 1H), 7.02-6.95 (m, 2H), 6.53-6.43 (m, 2H), 4.92-4.80 (m, 2H), 4.54-4.49 (m, 1H), 2.76 (s, 2H), 1.99-1.91 (m, 6H), 1.85-1.75 (m, 6H), 1.08 (s, 6H). LCMS: 357.37 (M+H). Purity: 97.57% by AUC. RT: 5.68. UV: 245 nm.

Step-3: Synthesis of phenyl (4-(4-(4-(2-hydroxy-2-methylpropyl) thiazol-2-yl) bicyclo [2.2.2]octan-1-yl) phenyl) carbamate (5): To a stirred solution of 4 (75 mg, 0.21 mmol) and triethyl amine (0.08 mL, 0.63 mmol) in THF (5.0 mL) was added phenyl chloroformate (0.03 mL, 0.25 mmol) at 0° C. After addition, the reaction mixture was stirred at 0° C. for 30 minutes and at room temperature for 3 hours. The reaction mixture was monitored by TLC and LCMS. After complete consumption of starting material, the reaction mixture was poured into ice-cold water (25 mL). The precipitate was filtered and dried to obtain the crude product, which was triturated with n-heptane to give 5 (72 mg, 72% yield) as an off-white solid. ¹H NMR (300 MHz, DMSO-d₆): δ=10.20-10.04 (m, 1H), 7.46-7.38 (m, 4H), 7.33-7.18 (m, 5H), 7.14 (s, 1H), 4.53-4.51 (m, 1H), 2.77 (s, 2H), 2.01-1.83 (m, 12H), 1.08 (s, 6H). LCMS: 477.06 (M+H). Purity: 96.31% by AUC. RT: 6.43. UV: 245 nm.

Step-4: Synthesis of 3-fluoro-N-(4-(4-(4-(2-hydroxy-2-methylpropyl) thiazol-2-yl) bicyclo [2.2.2]octan-1-yl) phenyl)-5,7-dihydro-6H-pyrrolo [3,4-b]pyridine-6-carboxamide (I-29): Compound 5 (100 mg, 0.21 mmol) was added to a solution of 6 (66.49 mg, 0.31 mmol) and TEA (0.13 mL, 1.05 mmol) in THF (5 mL) at ambient temperature. The reaction mixture was stirred at 60° C. for 16 hours. After complete consumption of starting material, the reaction mixture was poured in ice-cold water (25 mL) and stirred for 3 hours. The precipitate was filtered and washed with MTBE (50 mL) to afford I-29 (47 mg, 43% yield) as an off-white solid. ¹H NMR (300 MHz, DMSO-d₆): δ=8.48 (s, 1H), 8.36 (s, 1H), 7.79 (dd, J=2.4, 9.0 Hz, 1H), 7.47 (d, J=8.8 Hz, 2H), 7.25 (d, J=8.7 Hz, 2H), 7.14 (s, 1H), 4.75 (d, J=14.5 Hz, 4H), 4.52 (s, 1H), 2.77 (s, 2H), 2.06-1.81 (m, 12H), 1.08 (s, 6H). LCMS: 521.34 (M+H). Purity: 99.64% by AUC. RT: 5.55. UV: 245 nm. HPLC: Purity: 99.0% by AUC. RT: 15.19. UV: 250 nm.

Step-5: Synthesis of 1-bromo-4-hydroxy-4-methylpentan-2-one (2): To a stirred solution of 2a (5.0 g, 43.10 mmol) in MeOH (30.0 mL) was added Br₂ (2.14 mL, 42.24 mmol) at 0° C. The reaction mixture was stirred at room temperature for 6 hours and monitored by TLC. After completion of the reaction, the reaction mixture was quenched slowly with Na₂S₂O₃ solution at 0° C. and extracted with EtOAc (2×150 mL). The combined organic extracts were dried over Na₂SO₄ and concentrated under reduced pressure to afford the crude product, which was purified by column chromatography (eluent: 10% EtOAc in hexane) to obtain 2 (3 g, 36% yield) as a pale-yellow liquid. ¹H NMR (300 MHz, DMSO-d₆): δ=4.77 (s, 1H), 4.39 (s, 2H), 2.64 (s, 2H), 1.15 (s, 6H).

### Example 23—Synthesis of 5-fluoro-N-(4-(4-(4-(2-hydroxy-2-methylpropyl) thiazol-2-yl) bicyclo [2.2.2]octan-1-yl) phenyl) isoindoline-2-carboxamide (I-30)

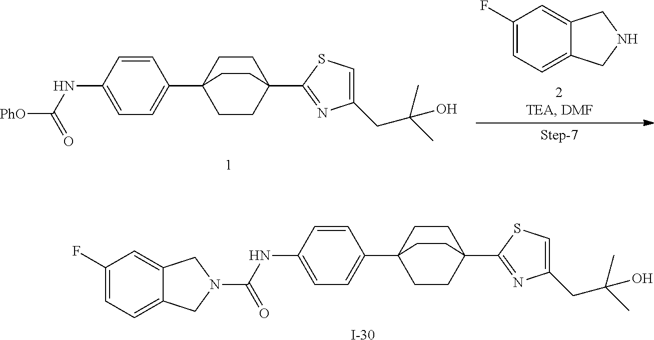

The following LCMS method was used: Column: X Bridge C18 4.6×50 mm×3.5 μm. Mobile Phase A: 10 mm ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Compound 1 (100 mg, 0.21 mmol) was added to a solution of 2 (66.17 mg, 0.31 mmol) and TEA (0.14 mL, 1.05 mmol) in THF (5 mL) at ambient temperature. The reaction mixture was stirred at 60° C. for 16 hours. After complete consumption of starting material, the reaction mixture was poured into ice-cold water (25 mL) and stirred for 3 hours. The precipitate was filtered and washed with MTBE (50 mL) to afford I-30 (30 mg, 27% yield) as an off-white solid. ¹H NMR (300 MHz, DMSO-d₆): δ-8.30 (s, 1H), 7.46 (d, J=8.8 Hz, 2H), 7.42-7.35 (m, 1H), 7.24 (d, J=8.8 Hz, 3H), 7.18-7.09 (m, 2H), 4.73 (d, J=8.3 Hz, 4H), 4.52 (s, 1H), 2.77 (s, 2H), 2.03-1.84 (m, 12H), 1.08 (s, 6H). LCMS: 520.37 (M+H). Purity: 99.06% by AUC. RT: 6.10. UV: 245 nm. HPLC: Purity: 98.46% by AUC. RT: 16.59. UV: 250 nm.

### Example 24—Synthesis of N-(4-(4-(3-acetyl-1H-pyrrol-1-yl) bicyclo [2.2.2]octan-1-yl) phenyl)-3-fluoro-5,7-dihydro-6H-pyrrolo [3,4-b]pyridine-6-carboxamide (II-4)

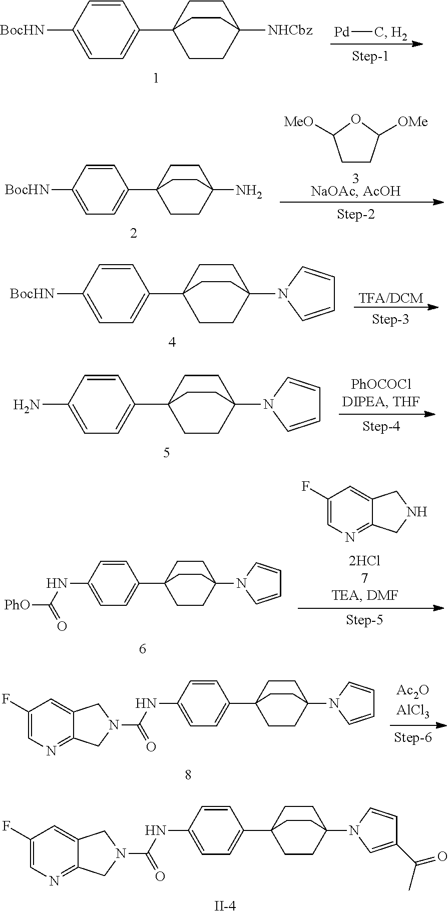

The following LCMS method was used: Column: X Bridge C18 4.6×50 mm×3.5 μm. Mobile Phase A: 10 mm ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of tert-butyl (4-(4-aminobicyclo [2.2.2]octan-1-yl) phenyl) carbamate (2): A stirred solution of 1 (2.5 g, 5.55 mmol) in ethanol:THF (2:1, 260 mL) was added Pd/C (600 mg, 50% wet) at room temperature under H₂ atmosphere. The reaction mixture was stirred at room temperature for 4 hours. The reaction mixture was monitored by TLC and LCMS. After completion of the reaction, the reaction mixture was filtered through a CELITE® pad and washed with 10% MeOH in DCM (200 mL). The filtrate was concentrated under reduced pressure to obtain the crude product, which was washed with n-pentane to obtain 2 (1.57 g, 90% yield) as an off-white solid. LCMS: 317.06 (M+H). Purity: 77.97% by AUC. RT: 4.60. UV: 240 nm.

Step-2: Synthesis of tert-butyl (4-(4-(1H-pyrrol-1-yl) bicyclo [2.2.2]octan-1-yl) phenyl) carbamate (4): To a stirred solution of 2 (100 mg, 0.32 mmol) in AcOH (0.5 mL) was added NaOAc (115 mg, 0.63 mmol) followed by 3 (41 mg, 0.32 mmol) at room temperature. The reaction mixture was stirred at 60° C. for 16 hours and monitored by TLC. After completion of the reaction, the reaction mixture was quenched slowly with NaHCO₃ solution at 0° C., basified to pH ˜8 to 9, and extracted with EtOAc (2×50 mL). The combined organic extracts were dried over Na₂SO₄ and concentrated under reduced pressure to afford the crude product, which was purified by column chromatography (eluent: 20% EtOAc in hexane) to give 4 (30 mg, 26% yield) as an off-white solid. ¹H NMR (300 MHz, DMSO-d₆): δ=9.23 (s, 1H), 7.43-7.28 (m, 2H), 7.28-7.18 (m, J=8.7 Hz, 2H), 6.89 (t, J=2.2 Hz, 2H), 6.03-5.92 (m, 2H), 2.08-1.87 (m, 12H), 1.46 (s, 9H). LCMS: 367.34 (M+H). Purity: 85.29% by AUC. RT: 6.88. UV: 240 nm.

Step-3: Synthesis of 4-(4-(1H-pyrrol-1-yl) bicyclo [2.2.2]octan-1-yl) aniline (5): To a stirred solution of 4 (300 mg, 0.82 mmol) in DCM (5 mL) was added TFA (959 mg, 8.2 mmol) at 0° C. under N₂ atmosphere. The reaction mixture was stirred at room temperature for 4 hours and monitored by TLC and LCMS. After complete consumption of starting material, the reaction mixture was diluted with water and basified to pH ˜8-9 by using saturated NaHCO₃ solution (30 mL). The reaction mixture was extracted with DCM (2×50 mL). The combined organic layer was dried over Na₂SO₄ and concentrated under reduced pressure to afford the crude product, which was triturated with n-pentane to give 5 (210 mg, crude) as a light-brown solid. ¹H NMR (300 MHz, DMSO-d₆): δ=7.03-6.95 (m, 2H), 6.94-6.81 (m, 3H), 6.55-6.42 (m, 2H), 5.98 (t, J=2.1 Hz, 1H), 4.83 (s, 2H), 2.09-1.75 (m, 12H).

Step-4: Synthesis of phenyl (4-(4-(1H-pyrrol-1-yl) bicyclo [2.2.2]octan-1-yl) phenyl) carbamate (6): To a stirred solution of 5 (350 mg, 1.31 mmol) and DIPEA (0.6 mL, 3.28 mmol) in THF (5.0 mL) was added phenyl chloroformate (245 mg, 0.25 mmol) slowly at 0° C. After addition, the reaction was stirred at 0° C. for 30 minutes and at room temperature for 2 hours. The reaction mixture was monitored by TLC and LCMS. After complete consumption of starting material, the reaction mixture was poured into ice-cold water (50 mL) and extracted with EtOAc (2×50 mL). The combined organic layer was dried over Na₂SO₄ and concentrated under reduced pressure to afford the crude product, which was triturated with n-pentane give 6 (285 mg, 76% yield) as an off-white solid. ¹H NMR (300 MHz, DMSO-d₆): δ=10.15 (s, 1H), 7.48-7.12 (m, 11H), 6.90 (t, J=2.2 Hz, 1H), 5.99 (t, J=2.2 Hz, 1H), 2.14-1.81 (m, 12H). LCMS: 387.33 (M+H). Purity: 76.04% by AUC. RT: 6.65. UV: 240 nm.

Step-5: Synthesis of N-(4-(4-(1H-pyrrol-1-yl) bicyclo [2.2.2]octan-1-yl) phenyl)-3-fluoro-5,7-dihydro-6H-pyrrolo [3,4-b]pyridine-6-carboxamide (8): Compound 6 (247 mg, 1.17 mmol) was added to a solution of 2 (380 mg, 0.78 mmol) and TEA (151 mg, 11.70 mmol) in THF (10 mL) at ambient temperature. The reaction mixture was stirred at 60° C. for 16 hours. After complete consumption of starting material, the reaction mixture was poured into ice-cold water (30 mL) and stirred for 1 hour. The precipitate was filtered and washed with n-pentane (20 mL) to afford 8 (244 mg, 90% yield) as an off-white solid. LCMS: 431.13 (M+H). Purity: 68.62% by AUC. RT: 5.83. UV: 240 nm.

Step-6: Synthesis of N-(4-(4-(3-acetyl-1H-pyrrol-1-yl) bicyclo [2.2.2]octan-1-yl) phenyl)-3-fluoro-5,7-dihydro-6H-pyrrolo [3,4-b]pyridine-6-carboxamide (II-4): To a stirred solution of 8 (300 mg, 0.70 mmol) in DCE (20.0 mL) was added Ac₂O (113 mg, 1.12 mmol) and AlCl₃ (370 mg, 2.78 mmol) slowly at 0° C. After addition, the reaction mixture was stirred at room temperature under N₂ atmosphere for 16 hours. The reaction mixture was monitored by TLC and LCMS. After complete consumption of starting material, the reaction mixture was poured into ice-cold water (50 mL) and extracted with EtOAc (2×50 mL). The combined organic extracts were dried over Na₂SO₄, filtered, and concentrated to give the crude product, which was purified by column chromatography (eluent: 30% EtOAc in hexane) to give II-4 (110 mg, 29% yield) as an off-white solid. ¹H NMR (300 MHz, DMSO-d₆): δ=8.51-8.45 (m, 1H), 8.37 (s, 1H), 7.80 (dd, J=2.0, 9.1 Hz, 1H), 7.73-7.68 (m, 1H), 7.48 (d, J=8.7 Hz, 2H), 7.26 (d, J=8.8 Hz, 2H), 7.07-7.01 (m, 1H), 6.46-6.41 (m, 1H), 4.81-4.75 (m, 4H), 2.30 (s, 3H), 2.14-1.91 (m, 12H). LCMS: 473.27 (M+H). Purity: 96.28% by AUC. RT: 5.10. UV: 240 nm. HPLC: Purity: 91.37% by AUC. RT: 14.0. UV: 250 nm.

### Example 25—Synthesis of 3-fluoro-N-(4-(4-(3-((2-hydroxy-2-methylpropyl) carbamoyl)-1H-pyrrol-1-yl) bicyclo [2.2.2]octan-1-yl) phenyl)-5,7-dihydro-6H-pyrrolo [3,4-b]pyridine-6-carboxamide (II-5)

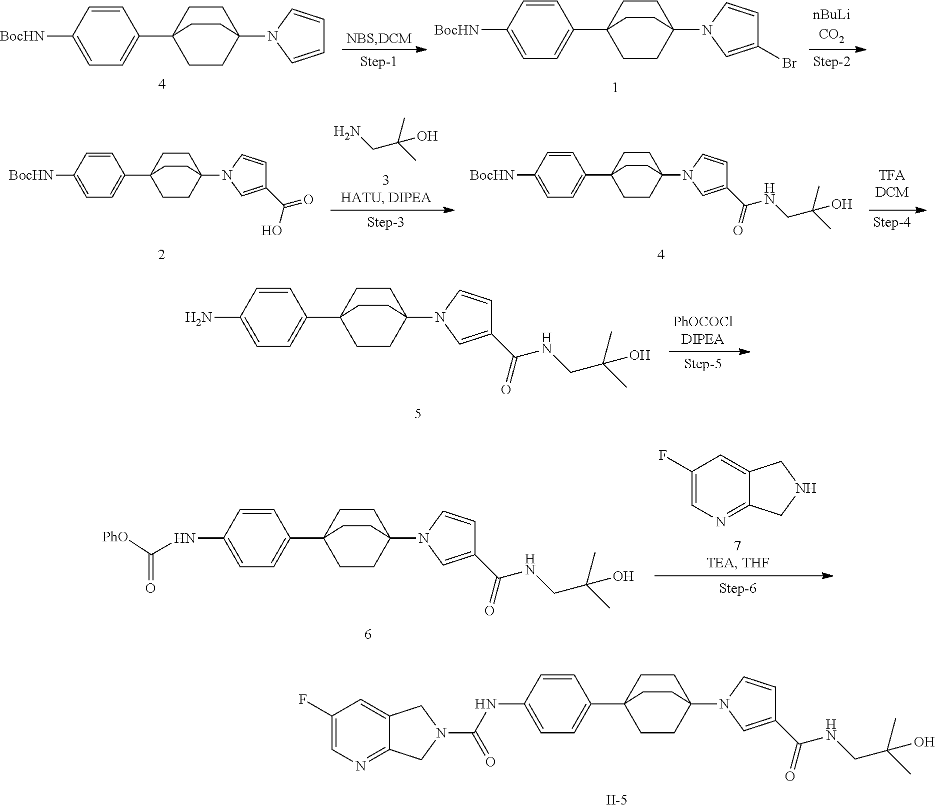

The following LCMS method was used: Column: X Bridge C18 4.6×50 mm×3.5 μm. Mobile Phase A: 10 mm ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of tert-butyl (4-(4-(3-bromo-1H-pyrrol-1-yl) bicyclo [2.2.2]octan-1-yl) phenyl) carbamate (1): To a stirred solution of 4 (1.2 g, 3.278 mmol) in DCM (40.0 mL) was added NBS (700 mg, 3.61 mmol) at −78° C. The reaction mixture was stirred at room temperature for 3 hours and monitored by TLC and LCMS. The reaction mixture was quenched with water and extracted with DCM (2×150 mL). The combined organic extracts were dried over Na₂SO₄ and concentrated under reduced pressure to afford the crude product, which was triturated with n-pentane to give 1 (1.2 g, 82% yield) as a pale-yellow liquid. LCMS: 446.98 (M+2H). Purity: 68.86% by AUC. RT: 7.25. UV: 240 nm.

Step-2: Synthesis of 1-(4-(4-((tert-butoxycarbonyl) amino) phenyl) bicyclo [2.2.2]octan-1-yl)-1H-pyrrole-3-carboxylic acid (2): To a stirred solution of 1 (500 mg, 1.10 mmol) in THF (20 mL) was added n-BuLi (0.67 mL, 1.70 mmol, 2.5 M in n-hexanes) at −78° C. The mixture was maintained for 30 minutes, and CO₂ gas was passed through the mixture using dry ice at −78° C. for 30 minutes. The mixture was stirred for 30 minutes at room temperature and monitored by TLC and LCMS. The reaction mixture was quenched with saturated NH₄Cl and extracted with EtOAc (3×50 mL). The combined organic extracts were dried over Na₂SO₄ and concentrated to give a crude product, which was acidified (using 1.0 N HCl) to pH 2-3 and extracted with EtOAc (3×50 mL). The combined organic extracts were dried over Na₂SO₄, concentrated, and dried to give 2 (50 mg, 11% yield) as an off-white solid. ¹H NMR (300 MHz, DMSO-d₆): δ=11.77 (s, 1H), 9.24 (s, 1H), 7.50-7.41 (m, 1H), 7.36 (d, J=8.6 Hz, 2H), 7.23 (d, J=8.7 Hz, 2H), 6.99 (t, J=2.6 Hz, 1H), 6.38 (dd, J=1.7, 2.8 Hz, 1H), 2.11-1.89 (m, 12H), 1.46 (s, 9H). LCMS: 411.07 (M+H). Purity: 82.46% by AUC. RT: 5.20. UV: 240 nm.

Step-3: Synthesis of tert-butyl (4-(4-(3-((2-hydroxy-2-methylpropyl) carbamoyl)-1H-pyrrol-1-yl) bicyclo [2.2.2]octan-1-yl) phenyl) carbamate (4): To a stirred solution of 2 (300 mg, 0.73 mmol) in DMF (10 mL) was added DIPEA (031 mL, 1.83 mmol) followed by HATU (417 mg, 1.09 mmol) and 3 (325 mg, 3.66 mmol) at room temperature. The reaction mixture was stirred at room temperature for 16 hours and monitored by TLC and LCMS. After complete consumption of starting material, the reaction mixture was poured into ice-cold water (25 mL). The precipitate was filtered and dried to obtain 4 (300 mg, 85% yield) as a brown solid. ¹H NMR (300 MHz, DMSO-d₆): δ=9.24 (s, 1H), 7.60-7.49 (m, 2H), 7.40-7.32 (m, 2H), 7.23 (d, J=8.7 Hz, 2H), 6.94 (t, J=2.6 Hz, 1H), 6.47 (dd, J=1.6, 2.7 Hz, 1H), 4.61 (s, 1H), 3.15 (d, J=6.1 Hz, 2H), 2.08-1.91 (m, 12H), 1.46 (s, 9H), 1.06 (s, 6H). LCMS: 482.19 (M+H), Purity—90.91% by AUC, RT—5.52, UV-240 nm.

Step-4: Synthesis of 1-(4-(4-aminophenyl) bicyclo [2.2.2]octan-1-yl)-N-(2-hydroxy-2-methylpropyl)-1H-pyrrole-3-carboxamide (5): To a stirred solution of 4 (300 mg, 0.623 mmol) in DCM (15.0 mL) was added TFA (1.5 mL) slowly at 0° C. The reaction mixture was stirred at room temperature for 3 hours and monitored by TLC and LCMS. After complete consumption of starting material, the reaction mixture was quenched with NaHCO₃ and extracted with DCM (3×50 mL). The combined organic extracts were dried over Na₂SO₄ and concentrated to give crude product, which was triturated with MTBE (10 mL) and n-pentane (10 mL) to obtain 5 (170 mg, 71% yield) as a brown solid.

¹H NMR (300 MHz, DMSO-d₆): δ=7.59-7.48 (m, 2H), 7.00 (d, J=8.6 Hz, 2H), 6.93 (t, J=2.6 Hz, 1H), 6.52-6.44 (m, 3H), 4.87-4.79 (m, 2H), 4.61 (s, 1H), 3.15 (d, J=6.1 Hz, 2H), 2.05-1.86 (m, 12H), 1.06 (s, 6H). LCMS: 382.16 (M+H). Purity: 92.59% by AUC. RT: 4.60. UV: 240 nm.

Step-5: Synthesis of phenyl (4-(4-(3-((2-hydroxy-2-methylpropyl) carbamoyl)-1H-pyrrol-1-yl) bicyclo [2.2.2]octan-1-yl) phenyl) carbamate (6): To a stirred solution of 5 (200 mg, 0.52 mmol) and DIPEA (0.30 mL, 1.31 mmol) in THF (10.0 mL) was added phenyl chloroformate (0.12 mL, 0.63 mmol) slowly at 0° C. After addition, the reaction mixture was stirred at 0° C. for 30 minutes and at room temperature for 2 hours. The reaction mixture was monitored by TLC and LCMS. After complete consumption of starting material, the reaction mixture was poured into ice-cold water (20 mL). The precipitate was filtered and dried to give crude product, which was triturated with n-pentane to give 6 (150 mg, 57% yield) as an off-white solid. ¹H NMR (300 MHz, DMSO-d₆): δ=10.22-10.09 (m, 1H), 7.57 (t, J=6.2 Hz, 1H), 7.53 (t, J=2.0 Hz, 1H), 7.47-7.38 (m, 4H), 7.35-7.29 (m, 2H), 7.27 (td, J=1.4, 7.4 Hz, 1H), 7.24-7.18 (m, 2H), 6.97-6.92 (m, 1H), 6.49-6.45 (m, 1H), 4.61 (s, 1H), 3.19-3.11 (m, 2H), 2.10-1.90 (m, 12H), 1.07 (s, 6H). LCMS: 502.10 (M+H). Purity: 96.54% by AUC. RT: 5.38. UV: 240 nm.

Step-6: Synthesis of 3-fluoro-N-(4-(4-(3-((2-hydroxy-2-methylpropyl) carbamoyl)-1H-pyrrol-1-yl) bicyclo [2.2.2]octan-1-yl) phenyl)-5,7-dihydro-6H-pyrrolo [3,4-b]pyridine-6-carboxamide (II-5): To a stirred solution of 6 (100 mg, 0.199 mmol) in DMF (5 mL) was added triethyl amine (0.43 mL, 2.98 mmol) and 7 (63 mg, 0.30 mmol) at room temperature. The reaction mixture was stirred at 60° C. for 16 hours and monitored by TLC and LCMS. After complete consumption of starting material, the reaction mixture was cooled to room temperature and poured into ice-cold water (50 mL). The precipitate thus obtained was filtered to give crude product, which was triturated with ACN (10 mL) to obtain II-5 (60 mg, 55% yield) as a light-brown solid. ¹H NMR (300 MHz, DMSO-d₆): δ=8.48 (d, J=1.4 Hz, 1H), 8.37 (s, 1H), 7.83-7.75 (m, 1H), 7.60-7.56 (m, 1H), 7.56-7.51 (m, 1H), 7.48 (d, J=8.8 Hz, 2H), 7.31-7.21 (m, 2H), 6.95 (t, J=2.6 Hz, 1H), 6.50-6.45 (m, 1H), 4.77 (s, 2H), 4.73 (s, 2H), 4.61 (s, 1H), 3.16 (d, J=6.1 Hz, 2H), 2.11-1.93 (m, 12H), 1.07 (s, 6H). LCMS: 546.32 (M+H). Purity: 98.07% by AUC. RT: 4.70. UV: 240 nm. HPLC: Purity: 96.73% by AUC. RT: 13.09. UV: 250 nm.

### Example 26—Synthesis of 3-fluoro-N-(4-((1r,4r)-4-(4-(2-hydroxyethyl)-1H-1,2,3-triazol-1-yl) cyclohexyl) phenyl)-5,7-dihydro-6H-pyrrolo [3,4-b]pyridine-6-carboxamide (I-31)

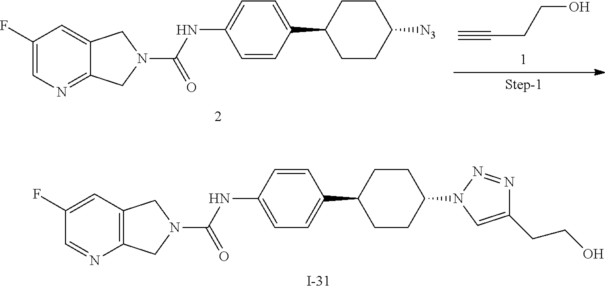

The following LCMS method was used: Column: X Bridge C18 4.6×50 mm×3.5 μm. Mobile Phase A: 10 mm ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Synthesis of 3-fluoro-N-(4-((1r,4r)-4-(4-(2-hydroxyethyl)-1H-1,2,3-triazol-1-yl) cyclohexyl) phenyl)-5,7-dihydro-6H-pyrrolo [3,4-b]pyridine-6-carboxamide (I-31): To a stirred solution of 2 (70 mg, 0.184 mmol) in a mixture of t-BuOH:water (1:1, 6.0 mL) was added sodium ascorbate (18.2 mg, 0.092 mmol), CuSO₄.5H₂O (27.5 mg, 0.092 mmol), and 1 (38.6 mg, 0.552 mmol) at room temperature. The reaction mixture was heated in a microwave at 100° C. for 2 hours. The reaction mixture was monitored by TLC and LCMS. After complete consumption of starting material, the reaction mixture was diluted with water (25 mL) and extracted with 10% MeOH in DCM (2×50 mL). The organic layer was dried over anhydrous Na₂SO₄, filtered, and concentrated under reduced pressure to afford crude product, which was purified by column chromatography (eluent: 1-5% MeOH in DCM) followed by trituration with n-pentane to afford I-31 (40 mg, 49%) as an off-white solid. ¹H NMR (300 MHz, DMSO-d₆): δ=8.52-8.43 (m, 1H), 8.36 (s, 1H), 7.99-7.88 (m, 1H), 7.86-7.73 (m, 1H), 7.55-7.38 (m, 2H), 7.25-7.09 (m, 2H), 4.75 (d, J=14.1 Hz, 5H), 4.60-4.44 (m, 1H), 3.63 (d, J=2.8 Hz, 2H), 2.81-2.76 (m, 2H), 2.15 (d, J=1.1 Hz, 3H), 1.98-1.86 (m, 4H), 1.68 (d, J=8.8 Hz, 2H). LCMS: 451.06 (M+H). Purity: 98.73% by AUC. RT: 4.37. UV: 240 nm. HPLC: Purity: 96.77% by AUC. RT: 11.66. UV: 250 nm.

### Example 27—Synthesis of 3-fluoro-N-(4-((1r,4r)-4-(4-(hydroxymethyl)-1H-1,2,3-triazol-1-yl) cyclohexyl) phenyl)-5,7-dihydro-6H-pyrrolo [3,4-b]pyridine-6-carboxamide (I-33)

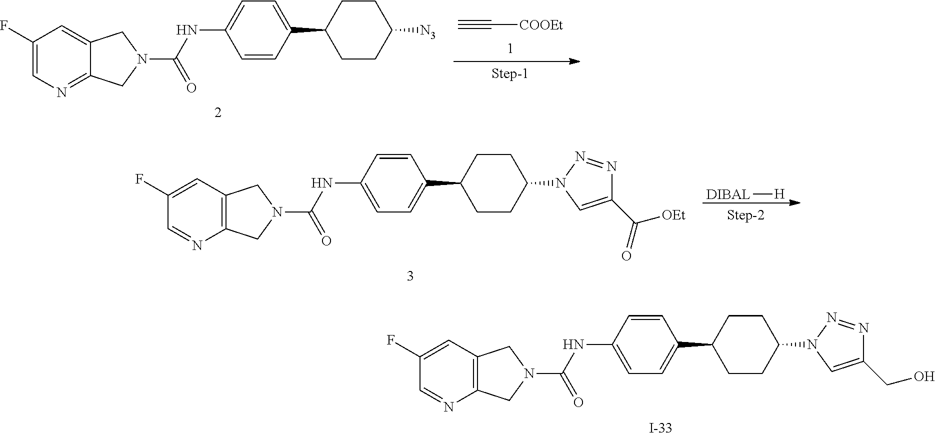

The following LCMS method was used: Column: X Bridge C18 4.6×50 mm×3.5 μm. Mobile Phase A: 10 mm ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: of ethyl 1-((1r,4r)-4-(4-(3-fluoro-6,7-dihydro-5H-pyrrolo [3,4-b]pyridine-6-carboxamido) phenyl) cyclohexyl)-1H-1,2,3-triazole-4-carboxylate (2): To a stirred solution of 2 (230 mg, 0.565 mmol) in a mixture of t-BuOH:water (1:1, 12.0 mL) was added sodium ascorbate (55.44 mg, 0.28 mmol), CuSO₄.5H₂O (84 mg, 0.28 mmol), and 1 (166 mg, 1.695 mmol) at room temperature. The reaction mixture was heated in a microwave at 100° C. for 2 hours. The reaction mixture was monitored by TLC and LCMS. After complete consumption of starting material, the reaction mixture was diluted with water (25 mL). The precipitate thus obtained was filtered and dried to obtain the crude product, which was triturated with MTBE:n-pentane (1:1, 20 mL) to afford 3 (200 mg, 69%) as an off-white solid. 1H NMR (300 MHz, DMSO-d6): δ=8.89 (s, 1H), 8.60-8.19 (m, 2H), 7.94-7.66 (m, 1H), 7.61-7.36 (m, 2H), 7.31-7.04 (m, 2H), 4.87-4.62 (m, 4H), 4.36-4.26 (m, 2H), 4.22-4.00 (m, 1H), 2.71-2.59 (m, 1H), 2.25-2.12 (m, 2H), 2.08-1.86 (m, 4H), 1.77-1.59 (m, 2H), 1.31 (t, J=7.0 Hz, 3H). LCMS: 479.04 (M+H). Purity: 96.99% by AUC. RT: 5.00. UV: 240 nm.

Step-2: Synthesis of 3-fluoro-N-(4-((1r,4r)-4-(4-(hydroxymethyl)-1H-1,2,3-triazol-1-yl) cyclohexyl) phenyl)-5,7-dihydro-6H-pyrrolo [3,4-b]pyridine-6-carboxamide (I-33): To a stirred solution of 3 (100 mg, 0.27 mmol) in THF (5.0 mL) was added DIBAL-H (296 mg, 1.0 M in toluene, 2.08 mmol) slowly at 0° C. The reaction mixture was stirred at room temperature for 3 hours and monitored by TLC and LCMS. After complete consumption of starting material, the reaction mixture was quenched with saturated NH₄Cl and extracted with EtOAc (3×50 mL). The combined organic extracts were dried over Na₂SO₄ to give the crude product, which was purified by column chromatography (eluent: 5% MeOH in DCM) to give I-33 (40 mg, 44% yield) as an off-white solid. ¹H NMR (300 MHz, DMSO-d6): δ=8.48 (s, 1H), 8.37 (s, 1H), 8.05 (s, 1H), 7.87-7.72 (m, 1H), 7.48 (d, J=8.3 Hz, 2H), 7.18 (d, J=8.2 Hz, 2H), 5.17 (t, J=4.7 Hz, 1H), 4.78 (s, 2H), 4.73 (s, 2H), 4.58-4.48 (m, 3H), 2.19-2.12 (m, 2H), 1.98-1.88 (m, 4H), 1.80-1.57 (m, 3H). LCMS: 436.90 (M+H). Purity: 93.49% by AUC. RT: 4.28. UV: 240 nm. HPLC: Purity: 93.56% by AUC. RT: 11.92. UV: 250 nm.

### Example 28—Synthesis of 5-fluoro-N-(4-(4-(4-(hydroxymethyl) thiazol-2-yl) bicyclo [2.2.2]octan-1-yl) phenyl) isoindoline-2-carboxamide (I-34)

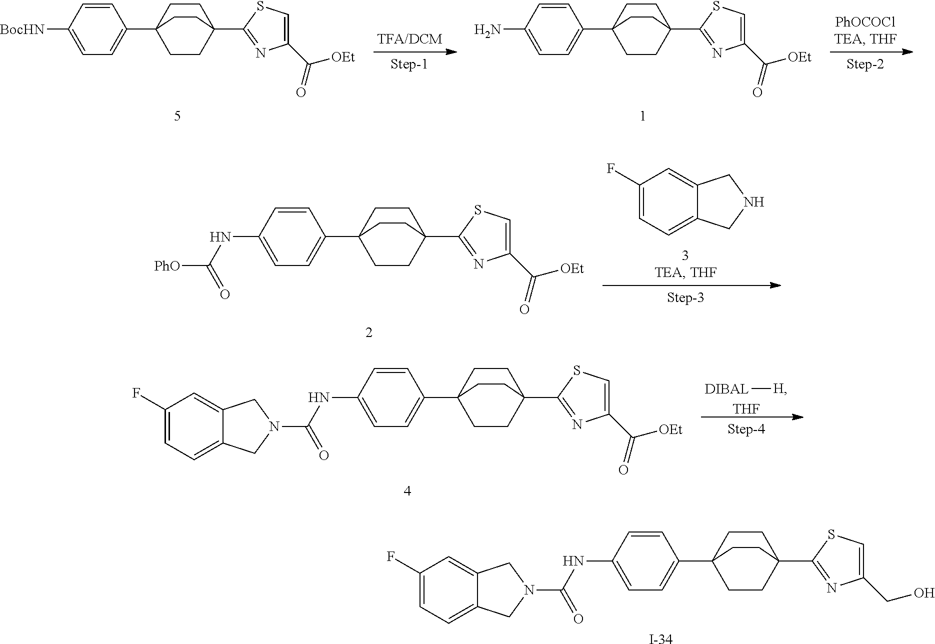

The following LCMS method was used: Column: X Bridge C18 4.6×50 mm×3.5 μm. Mobile Phase A: 10 mM ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of ethyl 2-(4-(4-aminophenyl) bicyclo [2.2.2]octan-1-yl) thiazole-4-carboxylate (1): To a stirred solution of 5 (3.2 g, 7.175 mmol) in DCM (50 mL) was added TFA (1.62 mL, 21.05 mmol), at 0° C. under N₂ atmosphere. The reaction mixture was stirred at room temperature for 2 hours and monitored by TLC and LCMS. After complete consumption of starting material, the volatiles were removed under reduced pressure. The reaction mixture was quenched with NaHCO₃ solution and extracted with DCM (2×150 mL). The combined organic layer was dried over Na₂SO₄ and concentrated under reduced pressure to afford the crude product, which was triturated with n-heptane to give 1 (2.3 g, 92% yield) as a brown solid. ¹H NMR (300 MHz, DMSO-d₆): δ=8.40 (s, 1H), 7.08-6.93 (m, 2H), 6.56-6.46 (m, 2H), 5.21-4.72 (m, 2H), 4.29 (q, J=7.2 Hz, 2H), 2.07-1.75 (m, 12H), 1.29 (t, J=7.1 Hz, 3H). LCMS: 357.16 (M+H). Purity: 91.48% by AUC. RT: 6.73. UV: 240 nm.

Step-2: Synthesis of ethyl 2-(4-(4-((phenoxycarbonyl) amino) phenyl) bicyclo [2.2.2]octan-1-yl) thiazole-4-carboxylate (2): To a stirred solution of 1 (900 mg, 2.54 mmol) and TEA (1.07 mL, 7.63 mmol) in THF (20.0 mL) was added phenyl chloroformate (383 mg, 3.05 mmol) slowly at 0° C. After addition, the reaction mixture was stirred at 0° C. for 30 minutes and at room temperature for 4 hours. The reaction mixture was monitored by TLC and LCMS. After complete consumption of starting material, the reaction mixture was poured into ice-cold water (50 mL). The precipitate was filtered and dried to obtain the crude product, which was triturated with MTBE:pentane (1:1, 20 mL) to afford 2 (900 mg, 76%) as an off-white solid. ¹H NMR (300 MHz, DMSO-d₆): δ=10.22-10.06 (m, 1H), 8.41 (s, 1H), 7.53-7.35 (m, 5H), 7.33-7.19 m, 4H), 4.34-4.23 (m, 2H), 2.07-1.83 (m, 12H), 1.30 (t, J=7.1 Hz, 3H). LCMS: 477.31 (M+H). Purity: 92.89% by AUC. RT: 6.47. UV: 240 nm.

Step-3: Synthesis of ethyl 2-(4-(4-(5-fluoroisoindoline-2-carboxamido) phenyl) bicyclo [2.2.2]octan-1-yl) thiazole-4-carboxylate (4): To a stirred solution of 2 (350 mg, 0.735 mmol) in THF (10 mL) was added triethyl amine (0.31 mL, 2.20 mmol) and 3 (191.4 mg, 1.10 mmol) at room temperature. The reaction mixture was stirred at 60° C. for 16 hours. The reaction mixture was monitored by TLC and LCMS. After complete consumption of starting material, the reaction mixture was cooled to room temperature and poured into ice-cold water (50 mL). The precipitate was filtered and triturated with MTBE:pentane (1:1, 20 mL) to obtain 4 (350 mg, 92% yield) as an off-white solid. ¹H NMR (300 MHz, DMSO-d₆): δ=8.42 (s, 1H), 8.31 (s, 1H), 7.47 (d, J=8.8 Hz, 2H), 7.39 (dd, J=5.2, 8.5 Hz, 1H), 7.29-7.19 (m, 3H), 7.19-7.09 (m, 1H), 4.78-4.67 (m, 4H), 4.29 (d, J=7.2 Hz, 2H), 2.09-1.84 (m, 12H), 1.30 (t, J=7.1 Hz, 3H). LCMS: 520.06 (M+H). Purity: 94.73% by AUC. RT: 6.18. UV: 240 nm.

Step-4: Synthesis of 5-fluoro-N-(4-(4-(4-(hydroxymethyl) thiazol-2-yl) bicyclo [2.2.2]octan-1-yl) phenyl) isoindoline-2-carboxamide (I-34): To a stirred solution of 4 (100 mg, 0.19 mmol) in THF (5.0 mL) was added DIBAL-H (273 mg, 1.0 M in toluene, 1.93 mmol) slowly at 0° C. The reaction mixture was stirred at room temperature for 3 hours and monitored by TLC and LCMS. After complete consumption of starting material, the reaction mixture was quenched with saturated NH₄Cl (25 mL) and extracted with EtOAc (3×50 mL). The combined organic extracts were dried over Na₂SO₄ to give the crude product, which was purified by column chromatography (eluent: 2% MeOH in DCM), which afforded I-34 (45 mg, 49% yield) as an off-white solid. ¹H NMR (300 MHz, DMSO-d₆): δ −7.48 (s, 1H), 7.45 (s, 1H), 7.42-7.35 (m, 1H), 7.25 (s, 4H), 7.23-7.20 (m, 1H), 7.18-7.08 (m, 1H), 5.26 (t, J=5.8 Hz, 1H), 4.73 (d, J=7.8 Hz, 4H), 4.52 (dd, J=0.9, 5.7 Hz, 2H), 2.06-1.74 (m, 12H). LCMS: 478.07 (M+H). Purity: 98.71% by AUC. RT: 5.43. UV: 240 nm. HPLC: Purity—98.10% by AUC. RT: 14.88. UV: 250 nm.

### Example 29—Synthesis of 3-fluoro-N-(4-(1-(1-(2-hydroxy-2-methylpropyl)-1H-1,2,4-triazol-3-yl) piperidin-4-yl) phenyl)-5,7-dihydro-6H-pyrrolo [3,4-b]pyridine-6-carboxamide (I-21)

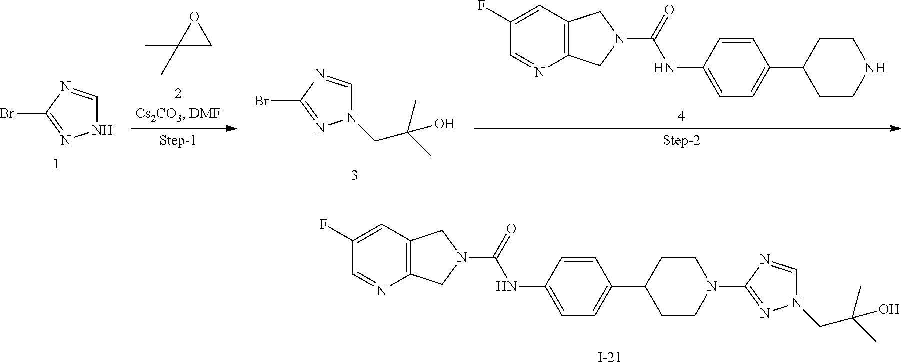

The following LCMS method was used: Column: X Bridge C18 4.6×50 mm×3.5 μm. Mobile Phase A: 10 mM Ammonium Acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of 1-(3-bromo-1H-1,2,4-triazol-1-yl)-2-methylpropan-2-ol (3): To a stirred solution of 1 (50 mg, 0.34 mmol) and Cs₂CO₃ (164 mg, 0.51 mmol) in DMF (3.0 mL) was added 2 (243 mg, 3.38 mmol) at room temperature. The reaction mixture was heated in a microwave at 50° C. for 16 hours. The reaction mixture was monitored by TLC and LCMS. After complete consumption of starting material, the reaction mixture was quenched with water (50 mL) and extracted with EtOAc (2×50 mL). The combined organic extracts were dried over Na₂SO₄ to give the crude product (50 mg), which was used in the next step without purification. ¹H NMR (300 MHz, DMSO-d₆): δ=8.41 (s, 1H), 4.07 (s, 2H), 2.75-2.71 (m, 1H), 1.08 (s, 6H). LCMS: 220.90 (M+2H). Purity: 97.63% by AUC. RT: 3.32. UV: 240 nm.

Step-2: Synthesis of 3-fluoro-N-(4-(1-(1-(2-hydroxy-2-methylpropyl)-1H-1,2,4-triazol-3-yl) piperidin-4-yl) phenyl)-5,7-dihydro-6H-pyrrolo [3,4-b]pyridine-6-carboxamide (I-21): A stirred solution of GPhos-Pd-G3 (79.9 mg, 0.08 mmol) in THF (15.0 mL) was degassed with argon for 5 minutes. To the solution was added 3 (145 mg, 0.66 mmol), 4 (150 mg, 0.44 mmol), and NaOTMS (296 mg, 2.65 mmol), and the mixture was again degassed with argon for 10 minutes. The reaction mixture was heated in a microwave at 100° C. for 2 hours and monitored by TLC and LCMS. After complete consumption of starting material, the reaction mixture was quenched with water (50 mL) and extracted with EtOAc (3×50 mL). The combined organic extracts were dried over Na₂SO₄ to give the crude product, which was purified by column chromatography (eluent: 3% MeOH in DCM) to afford I-21 (52 mg, 24% yield) as an off-white solid. ¹H NMR (300 MHz, DMSO-d₆): δ=8.51-8.44 (m, 1H), 8.37 (s, 1H), 8.02 (s, 1H), 7.83-7.76 (m, 1H), 7.47 (d, J=8.5 Hz, 2H), 7.14 (d, J=8.5 Hz, 2H), 4.80-4.68 (m, 5H), 4.08-3.97 (m, 2H), 3.89 (s, 2H), 2.84-2.72 (m, 2H), 2.66-2.58 (m, 1H), 1.83-1.72 (m, 2H), 1.71-1.56 (m, 2H), 1.09 (s, 6H). LCMS: 478.49 (M−H). Purity: 95.20% by AUC. RT: 4.45. UV: 240 nm. HPLC: Purity: 99.57% by AUC. RT: 12.35. UV: 250 nm.

### Example 30—Synthesis of N-(4-((1r,4r)-4-(4-(2-hydroxy-2-methylpropyl)-1H-1,2,3-triazol-1-yl) cyclohexyl) phenyl)-1,3-dihydro-2H-pyrrolo [3,4-c]pyridine-2-carboxamide (I-20)

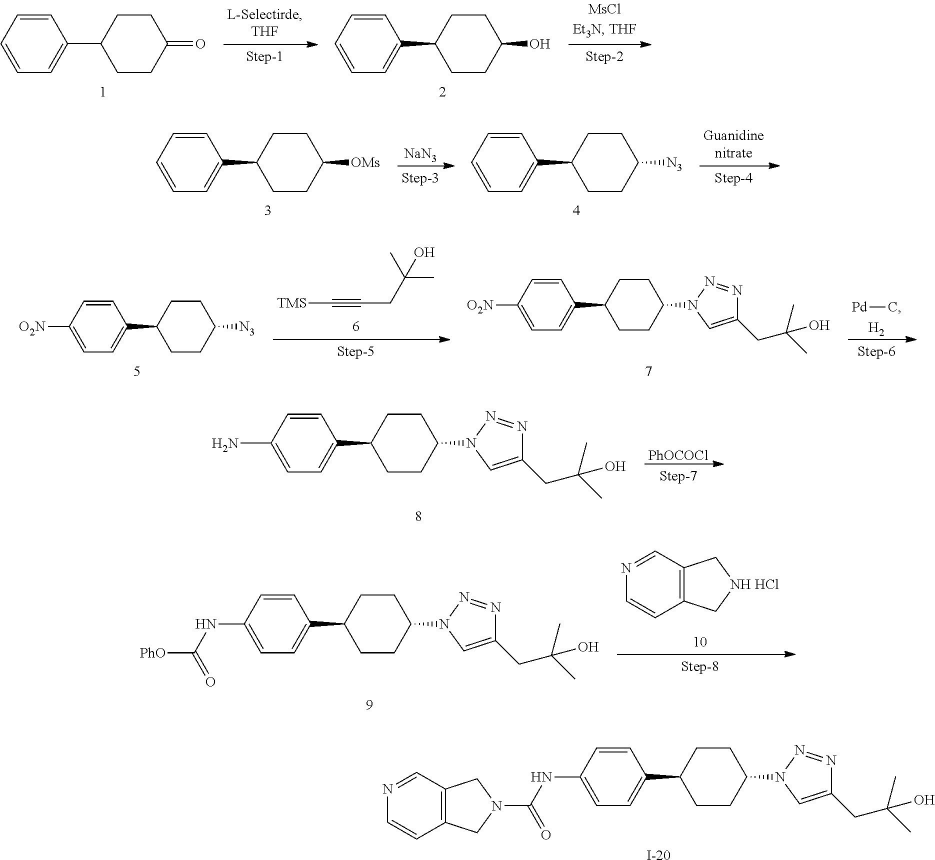

The following LCMS method was used: Column: X Bridge C18 4.6×50 mm×3.5 μm. Mobile Phase A: 10 mM ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of (1s,4s)-4-phenylcyclohexan-1-ol (2): To a stirred solution of 1 (5.0 g, 28.70 mmol) in THF (50.0 mL) was added L-selectride (10.9 g) slowly at 0° C. under N₂ atmosphere. The reaction mixtures was stirred for 2 hours and monitored by TLC and LCMS. After complete consumption of starting material, the reaction mixture was quenched with saturated NH₄Cl and extracted with EtOAc (2×100 mL). The combined organic extracts were dried with Na₂SO₄, filtered, and concentrated to give the crude product, which was purified by column chromatography (eluting with 6% EtOAc-hexanes) to afford 2 (3.5 g, 64% yield) as an off-white solid. 1H NMR (300 MHz, DMSO-d6): δ=7.28-7.20 (m, 5H), 4.31-4.29 (m, 1H), 3.49 (d, J=1.6 Hz, 1H), 1.06-1.06 (m, 1H), 0.88 (d, J=1.3 Hz, 2H), 0.83-0.82 (m, 4H), 0.80 (s, 2H). LCMS: 177.23 (M+H). Purity: 92.78% by AUC. RT: 5.28. UV: 210 nm.

Step-2: Synthesis of (1s,4s)-4-phenylcyclohexyl methane sulfonate (3): To a stirred solution of 2 (3.87 g, 21.98 mmol) and TEA (3.34 g, 32.98 mmol) in THF (40.0 mL) was added mesyl chloride (3.02 g, 26.38 mmol) slowly at 0° C. After addition, the reaction mixture was stirred at 0° C. for 1 hour and monitored by TLC and LCMS. After complete consumption of starting material, the reaction mixture was poured into ice-cold water (50 mL) and extracted with EtOAc (2×50 mL). The combined organic extracts were dried with Na₂SO₄, filtered, and concentrated to give the crude product, which was triturated with n-pentane (30 mL) to obtain 3 (2.6 g, 46% yield) as a gummy liquid. ¹H NMR (300 MHz, CHLOROFORM-d): δ=7.23-7.14 (m, 5H), 4.82-4.67 (m, 1H), 3.95-3.88 (m, 1H), 3.14 (s, 3H), 2.62-2.55 (m, 2H), 2.28 (d, J=7.7 Hz, 2H), 1.91 (d, J=2.4 Hz, 2H), 1.73-1.68 (m, 2H). LCMS: 255.21 (M+H). Purity: 77.02% by AUC. RT: 5.28. UV: 245 nm.

Step-3: Synthesis of ((1r,4r)-4-azidocyclohexyl) benzene (4): To a stirred solution of 3 (300 mg, 1.18 mmol) in DMSO (5.0 mL) was added NaN₃ (174 mg, 2.69 mmol) and tBuNHSO₄ (40.07 mg, 0.18 mmol) slowly at room temperature under N₂ atmosphere. The reaction mixture was stirred at 45° C. for 16 hours and monitored by TLC and LCMS. After complete consumption of starting material, the reaction mixture was cooled to room temperature, quenched with water, and extracted with EtOAc (2×50 mL). The combined organic extracts were dried with Na₂SO₄, filtered, and concentrated to give the crude product, which was purified by column chromatography (eluting with 8% EtOAc-hexanes) to afford 4 (50 g, 21% yield) as an off-white solid. ¹H NMR (300 MHz, DMSO-d₆): δ=7.32-7.14 (m, 5H), 3.49 (t, J=4.1, 11.2 Hz, 1H), 2.57-2.51 (m, 1H), 2.09-1.98 (m, 2H), 1.89-1.77 (m, 2H), 1.66-1.36 (m, 4H).

Step-4: Synthesis of 1-((1r,4r)-4-azidocyclohexyl)-4-nitrobenzene (5): To a stirred solution of 4 (1.0 g, 4.97 mmol) in TFA (10.0 mL) was added guanidine nitrite (606 mg, 4.97 mmol) portion-wise at 0° C. The reaction mixture was stirred at room temperature for 3 hours and monitored by TLC and LCMS. After complete consumption of starting material, the reaction mixture was quenched with water (100 mL) and extracted with EtOAc (2×100 mL). The combined organic extracts were dried over Na₂SO₄, filtered, and concentrated to give the crude product, which was triturated with n-pentane (10 mL) to obtain 5 (1.2 g, 98% yield) as a pale-yellow solid. ¹H NMR (300 MHz, DMSO-d₆): δ=7.09 (s, 4H), 3.59-3.42 (m, 1H), 2.83-2.60 (m, 1H), 2.11-2.00 (m, 2H), 1.91-1.81 (m, 2H), 1.69-1.54 (m, 2H), 1.54-1.37 (m, 2H).

Step-5: Synthesis of 2-methyl-1-(1-((1r,4r)-4-(4-nitrophenyl) cyclohexyl)-1H-1,2,3-triazol-4-yl) propan-2-ol (7): To a stirred solution of 5 (1.2 g, 4.87 mmol) in t-BuOH/H₂O (1:1, 8.0 mL) was added sodium ascorbate (482 mg, 2.43 mmol), CuSO₄.5H₂O (607 mg, 2.43 mmol), and 6 (3.3 g, 19.51 mmol) at room temperature. The reaction mixture was heated in a microwave at 100° C. for 1 hour and monitored by TLC and LCMS. After complete consumption of starting material, the reaction mixture was diluted with water (50 mL) and stirred for 1 hour. The precipitate thus obtained was filtered and dried to give the crude product, which was purified by combi-flash chromatography (eluent: 80% EtOAc-hexanes) to afford 7 (635 mg, 38% yield) as an off-white solid. ¹H NMR (300 MHz, CHLOROFORM-d): δ=8.17-8.14 (m, 2H), 7.7 (s, 1H), 7.39-7.31 (m, 2H), 4.74 (s, 1H), 2.71 (s, 2H), 2.37-2.32 (m, 1H), 2.05 (s, 1H), 1.42-1.39 (m, 2H), 1.30-1.26 (m, 4H), 1.25 (s, 2H), 1.25-1.19 (m, 6H). LCMS: 345.36 (M+H). Purity: 97.67% by AUC. RT: 5.02. UV: 240 nm.

Step-6: Synthesis of 1-(1-((1r,4r)-4-(4-aminophenyl) cyclohexyl)-1H-1,2,3-triazol-4-yl)-2-methylpropan-2-ol (8): To a solution of 7 (635 mg, 1.85 mmol) in MeOH (20 mL) was added Pd—C(200 mg, 10%) at room temperature. The reaction mixture was stirred under hydrogen atmosphere for 3 hours. After complete consumption of starting material, the reaction mixture was filtered through a pad of CELITE® and washed with 10% MeOH-DCM. The combined filtrate was concentrated under reduced pressure to obtain 8 (295 mg, 51% yield) as an off-white solid. ¹H NMR (300 MHz, DMSO-d₆): δ=7.87 (s, 1H), 7.04-6.93 (m, 2H), 6.93-6.84 (m, 2H), 6.65 (s, 2H), 4.87-4.73 (m, 1H), 4.59-4.44 (s, 1H), 2.69 (s, 2H), 2.61 (m, 1H), 2.18-2.08 (m, 2H), 1.99-1.81 (m, 4H), 1.71-1.52 (m, 2H), 1.07 (s, 6H). LC-MS: 315.42 (M+H). Purity: 74.78% by AUC. RT: 4.33. UV: 260 nm.

Step-7: Synthesis of phenyl (4-((1r,4r)-4-(4-(2-hydroxy-2-methylpropyl)-1H-1,2,3-triazol-1-yl) cyclohexyl) phenyl) carbamate (9): To a stirred solution of 7 (300 mg, 0.95 mmol) and DIPEA (246 mg, 1.91 mmol) in THF (10.0 mL) was added phenyl chloroformate (164 mg, 1.05 mmol) slowly at 0° C. After addition, the reaction mixture was stirred at 0° C. for 30 minutes and at room temperature for 2 hours. The reaction mixture was monitored by TLC and LCMS. After complete consumption of starting material, the reaction mixture was poured into ice-cold water (50 mL) and extracted with EtOAc (2×80 mL). The combined organic extracts were dried over Na₂SO₄, filtered, and concentrated to give the crude product, which was triturated with n-heptane (30 mL) to obtain 9 (371 mg, 89% yield) as an off-white solid. ¹H NMR (300 MHz, DMSO-d₆): δ=10.21-10.08 (m, 1H), 9.37-9.28 (m, 1H), 7.46-7.36 (m, 4H), 7.26-7.16 (m, 5H), 4.41 (s, 1H), 2.61 (dd, J=2.9, 8.9, 12.0 Hz, 1H), 2.46-2.38 (m, 1H), 2.19-2.13 (m, 2H), 1.93-1.89 (m, 2H), 1.81-1.57 (m, 4H), 1.39-1.10 (m, 2H), 1.08 (s, 6H). LC-MS: 435.07 (M+H). Purity: 83.37% by AUC. RT: 5.18. UV: 260 nm.

Step-8: Synthesis of N-(4-((1r,4r)-4-(4-(2-hydroxy-2-methylpropyl)-1H-1,2,3-triazol-1-yl) cyclohexyl) phenyl)-1,3-dihydro-2H-pyrrolo [3,4-c]pyridine-2-carboxamide (I-20): To a stirred solution of 9 (100 mg, 0.23 mmol) in DMF (2.0 mL) was added triethyl amine (116.3 mg, 1.15 mmol) and 10 (66.7 mg, 0.34 mmol) at room temperature. The reaction mixture was stirred at 45° C. for 16 hours and monitored by TLC and LCMS. After complete consumption of starting material, the reaction mixture was cooled to room temperature and then poured into ice-cold water (50 mL). The precipitate thus obtained was filtered and dried to obtain the crude product, which was triturated with ACN (5 mL) to afford I-20 (30 mg, 28% yield) as a pale pink solid. ¹H NMR (300 MHz, DMSO-d6): δ=8.61 (s, 1H), 8.50 (d, J=5.1 Hz, 1H), 8.36 (s, 1H), 7.89 (s, 1H), 7.51-7.40 (m, 3H), 7.17 (d, J=8.6 Hz, 2H), 4.80 (d, J=4.2 Hz, 4H), 4.60-4.47 (m, 1H), 4.41 (s, 1H), 2.71 (s, 2H), 2.16 (d, J=10.0 Hz, 2H), 2.00-1.84 (m, 5H), 1.76-1.61 (m, 2H), 1.08 (s, 6H). LCMS: 461.13 (M+H). Purity: 97.09% by AUC. RT: 4.22. UV: 240 nm. HPLC: Purity: 96.25 by AUC. RT: 11.97. UV: 250 nm.

### Example 31—Synthesis of N-(4-(4-(4-(2-hydroxy-2-methylpropyl)-1H-1,2,3-triazol-1-yl) bicyclo [2.2.2]octan-1-yl) phenyl)-1,3-dihydro-2H-pyrrolo [3,4-c]pyridine-2-carboxamide (I-19)

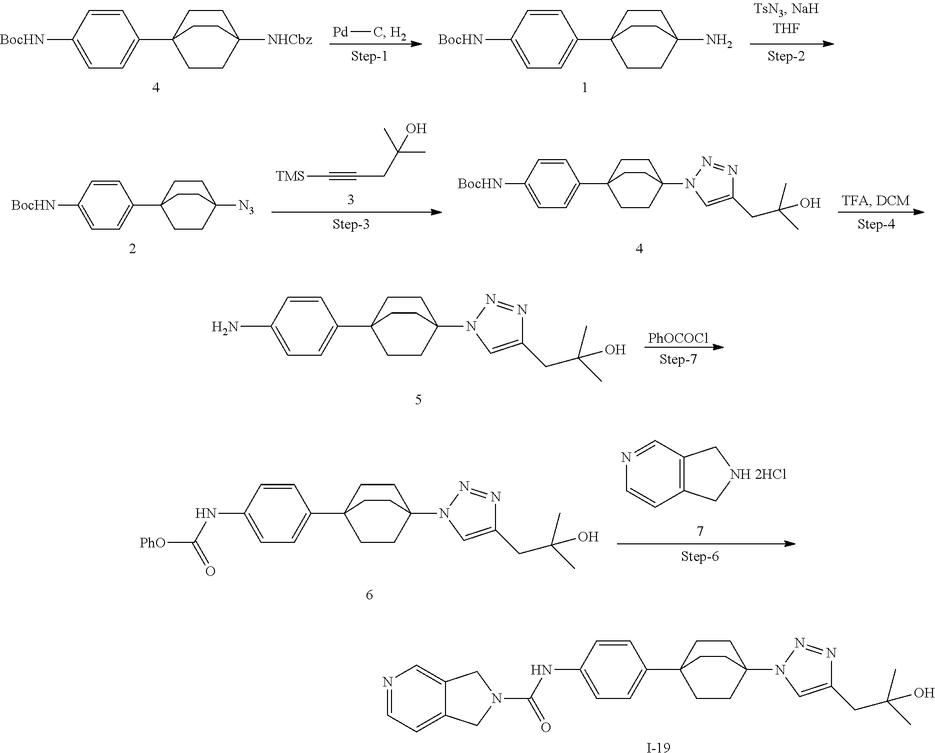

The following LCMS method was used: Column: X Bridge C18 4.6×50 mm×3.5 μm. Mobile Phase A: 10 mM ammonium acetate. Mobile Phase B: 100% ACN. Gradient 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: Methanol (100%).

Step-1: Synthesis of tert-butyl (4-(4-aminobicyclo [2.2.2]octan-1-yl) phenyl) carbamate (1): To a solution of 4 (5.0 g, 11.11 mmol) in THF (175 mL) and EtOH (400 mL) was added Pd—C(1.6 g, 10%) at room temperature. The reaction mixture was stirred under hydrogen atmosphere for 5 hours. After complete consumption of starting material, the reaction mixture was filtered through a CELITE® pad and washed with 10% MeOH in DCM (150 mL). The filtrate was concentrated under reduced pressure to obtain the crude product, which was washed with n-pentane to obtain 1 (3.5 g, 66% yield) as an off-white solid. ¹H NMR (300 MHz, DMSO-d₆): δ=9.20 (s, 1H), 7.32 (d, J=8.6 Hz, 2H), 7.23-7.11 (m, J=8.8 Hz, 2H), 1.81-1.71 (m, 6H), 1.58-1.48 (m, 6H), 1.46 (s, 9H). LC-MS: 317.37 (M+H). Purity: 94.64% by AUC. RT: 4.58. UV: 260 nm.

Step-2: Synthesis of tert-butyl (4-(4-azidobicyclo [2.2.2]octan-1-yl) phenyl) carbamate (2): To a stirred solution of dry NaH (1.6 g, 48.42 mmol) in THF (150 mL) was added 1 (2.55 g, 8.07 mmol) under N₂ atmosphere. The reaction mixture was stirred for 16 hours at room temperature and monitored by TLC and LCMS. To the reaction mixture was added tosyl azide (6.3 g, 32.28 mmol) at room temperature, and the mixture was stirred for another 80 hours with monitoring by TLC and LCMS. After complete consumption of starting material, the reaction mixture was quenched with saturated ammonium chloride solution (100 mL) and extracted with EtOAc (2×100 mL). The combined organic layer was dried over anhydrous Na₂SO₄ and concentrated under reduced pressure to give the crude product, which was purified by combi-flash chromatography (eluent: 2-3% ethyl acetate in hexanes) to afford 2 (2.5 g, 88% yield) as a pale-yellow solid. ¹H NMR (300 MHz, DMSO-d₆): δ=9.23 (s, 1H), 7.34 (d, J=8.7 Hz, 2H), 7.24-7.12 (m, 2H), 1.91-1.83 (m, 6H), 1.83-1.75 (m, 6H), 1.45 (s, 9H). LCMS: 343.36 (M+H). Purity: 93.80% by AUC. RT: 6.98. UV: 245 nm.

Step-3: Synthesis of tert-butyl(4-(4-(4-(2-hydroxy-2-methylpropyl)-1H-1,2,3-triazol-1-yl) bicyclo [2.2.2]octan-1-yl) phenyl) carbamate (4): To a stirred solution of 3 (1.0 g, 5.85 mmol) in t-BuOH/H₂O (1:1, 16.0 mL) was added sodium ascorbate (144 mg, 0.73 mmol), CuSO₄.5H₂O (182 mg, 0.73 mmol), and 2 (500 mg, 1.46 mmol) at room temperature. The reaction mixture was heated in a microwave at 100° C. for 1 hour and monitored by TLC and LCMS. After complete consumption of starting material, the reaction mixture was diluted with water (50 mL) and stirred for 1 hour. The precipitate thus obtained was filtered and dried to give the crude product. Five analogous batches were prepared, and all batches were combined and purified by combi-flash chromatography (eluent: 1-2% MeOH-DCM) to afford 4 (2.8 g, 87% yield) as an off-white solid. ¹H NMR (300 MHz, DMSO-d₆): δ=9.25 (s, 1H), 7.89 (s, 1H), 7.41-7.32 (m, 2H), 7.29-7.19 (m, J=8.8 Hz, 2H), 4.48-4.26 (m, 1H), 2.78-2.61 (m, 2H), 2.23-2.12 (m, 6H), 2.01-1.90 (m, 6H), 1.46 (s, 9H), 1.08 (s, 6H). LCMS: 441.45 (M+H). Purity: 97.75% by AUC. RT: 5.52. UV: 240 nm.

Step-4: Synthesis of 1-(1-(4-(4-aminophenyl) bicyclo [2.2.2]octan-1-yl)-1H-1,2,3-triazol-4-yl)-2-methyl propan-2-ol (5): To a stirred solution of 4 (2.8 g, 6.36 mmol) in DCM (80.0 mL) was added TFA (18.0 mL) at 0° C. The reaction mixture was stirred at room temperature for 16 hours and monitored by TLC and LCMS. After complete consumption of starting material, the volatiles were removed under reduced pressure. The reaction mixture was basified with NaHCO₃ and extracted with EtOAc to afford 5 (2.0 g, 95% yield) as an off-white solid. ¹H NMR (300 MHz, DMSO-d₆): δ=7.86 (s, 1H), 7.01 (d, J=8.5 Hz, 2H), 6.53-6.47 (m, 2H), 4.85 (s, 2H), 4.40 (s, 1H), 2.70 (s, 2H), 2.22-2.11 (m, 6H), 1.96-1.87 (m, 6H), 1.07 (s, 6H). LC-MS: 341.10 (M+H). Purity: 97.47% by AUC. RT: 4.45. UV: 260 nm.

Step-5: Synthesis of phenyl (4-(4-(4-(2-hydroxy-2-methylpropyl)-1H-1,2,3-triazol-1-yl) bicyclo [2.2.2]octan-1-yl) phenyl) carbamate (6): To a stirred solution of 5 (2.0 g, 5.88 mmol) and DIPEA (2.7 mL, 14.70 mmol) in THF (60.0 mL) was added phenyl chloroformate (0.76 mL, 5.88 mmol) slowly at 0° C. After addition, the reaction mixture was stirred at 0° C. for 30 minutes and at room temperature for 2 hours. The reaction mixture was monitored by TLC and LCMS. After complete consumption of starting material, the reaction mixture was poured into ice-cold water (50 mL) and extracted with EtOAc (2×50 mL). The combined organic extracts were dried over Na₂SO₄, filtered, and concentrated to give the crude product, which was triturated with EtOAc (30 mL) to give 6 (700 mg, 25% yield) as an off-white solid. ¹H NMR (300 MHz, DMSO-d₆): δ=10.21-10.09 (m, 1H), 7.87 (s, 1H), 7.48-7.37 (m, 3H), 7.37-7.30 (m, 2H), 7.28-7.11 (m, 3H), 6.78-6.71 (m, 1H), 4.40 (s, 1H), 2.70 (s, 2H), 2.25-2.13 (m, 6H), 2.03-1.90 (m, 6H), 1.07 (s, 6H). LC-MS: 461.16 (M+H). Purity: 98.50% by AUC. RT: 5.33. UV: 260 nm.

Step-6: Synthesis of N-(4-(4-(4-(2-hydroxy-2-methylpropyl)-1H-1,2,3-triazol-1-yl) bicyclo [2.2.2]octan-1-yl) phenyl)-1,3-dihydro-2H-pyrrolo [3,4-c]pyridine-2-carboxamide (I-19): To a stirred solution of 6 (300 mg, 0.65 mmol) in DMF (10 mL) was added triethyl amine (1.36 mL, 9.78 mmol) and 7 (189 mg, 0.97 mmol) at room temperature. The reaction mixture was stirred at 45° C. for 16 hours. The reaction mixture was monitored by TLC and LCMS. After complete consumption of starting material, the reaction mixture was cooled to room temperature and poured into ice-cold water (50 mL). The solid thus obtained was filtered and dried to obtain the crude product, which was triturated with EtOH (20 mL) and MTBE (10 mL) to afford I-19 (210 mg, 66% yield) as an off-white solid. ¹H NMR (300 MHz, DMSO-d₆): δ=8.61 (s, 1H), 8.50 (d, J=5.0 Hz, 1H), 8.37 (s, 1H), 7.87 (s, 1H), 7.52-7.40 (m, 3H), 7.31-7.23 (m, 2H), 4.79 (d, J=4.0 Hz, 4H), 4.39 (s, 1H), 2.70 (s, 2H), 2.24-2.14 (m, 6H), 2.07-1.93 (m, 6H), 1.07 (s, 6H). LCMS: 487.17 (M+H). Purity: 97.09% by AUC. RT: 4.33. UV: 240 nm. HPLC: Purity: 97.39 by AUC. RT: 12.32. UV: 250 nm.

### Example 32—Synthesis of 5-fluoro-N-(4-((1r,4r)-4-(5-(2-((2-hydroxy-2-methylpropyl) amino)-2-oxoethyl)-1,3,4-oxadiazol-2-yl)cyclohexyl)phenyl)isoindoline-2-carboxamide (II-2)

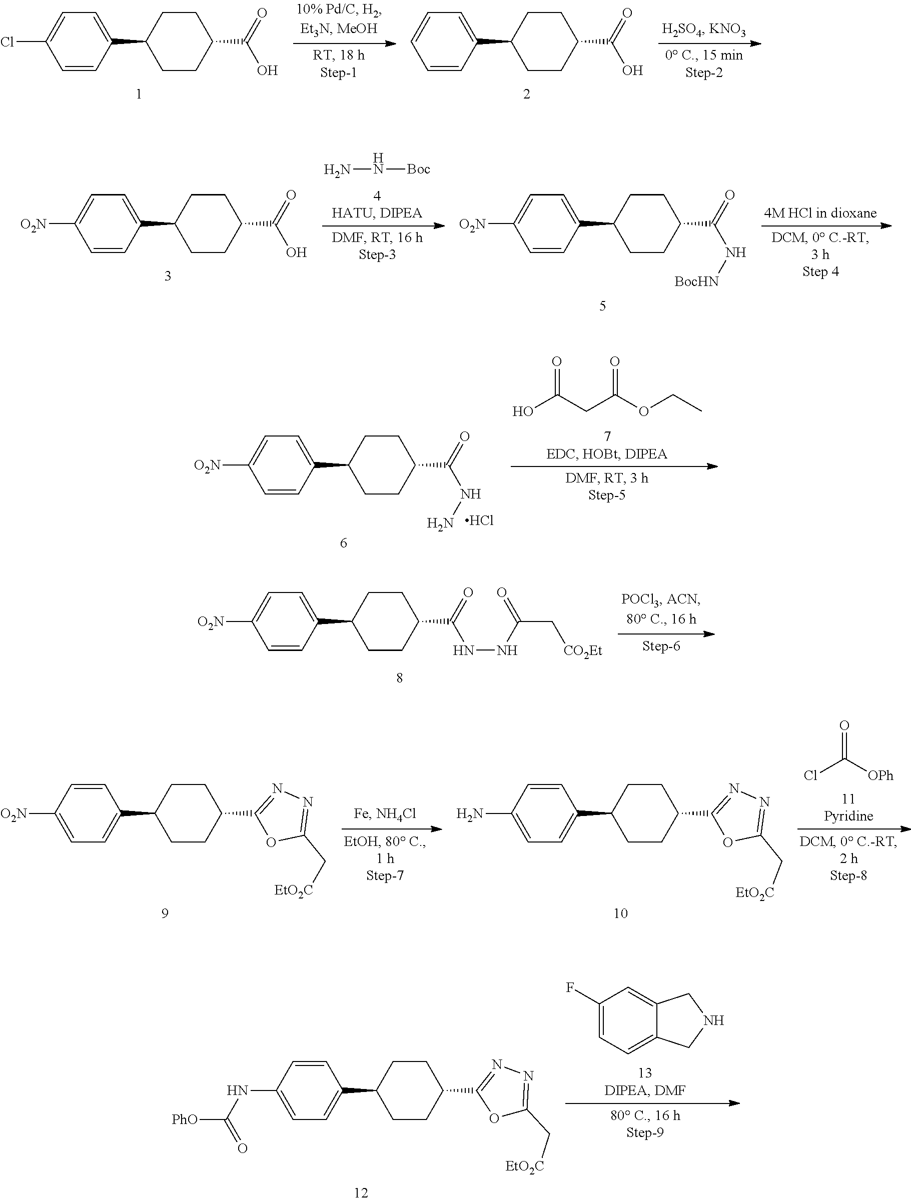

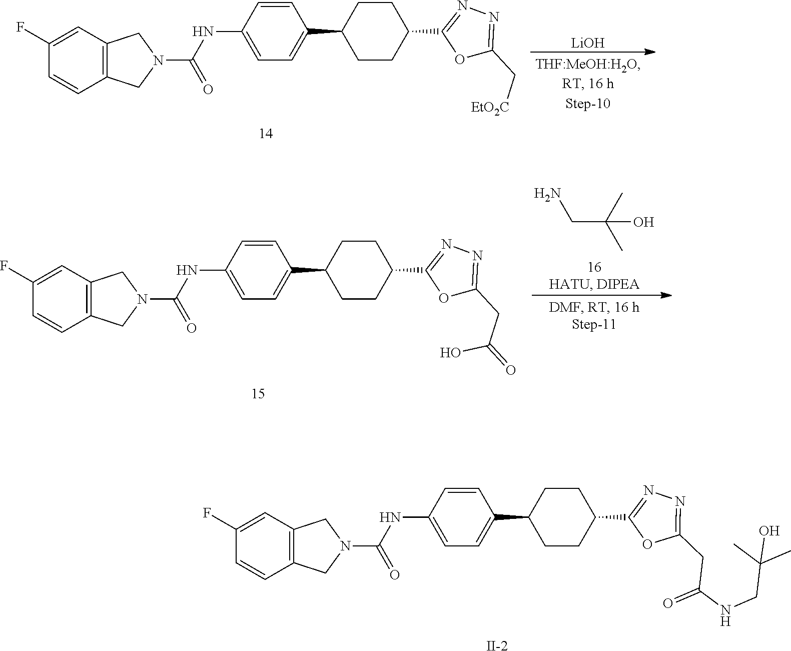

Step-1: Synthesis of 4-phenylcyclohexanecarboxylic acid (2): To a stirred solution of compound 1 (30 g, 15.7 mmol) in methanol (300 mL) was added TEA (18 mL, 128 mmol) and Pd—C (50% wet, 9 g) at room temperature. The reaction mixture was stirred for 16 hours at 50° C. under H₂ conditions. After completion of the reaction (monitored by TLC), the reaction mixture was diluted with methanol and filtered through a CELITE® pad. The filtrate was concentrated under reduced pressure to give crude product, which was triturated with n-heptane and dried to afford the title compound 2 (24 g, 117.6 mmol, 93.38% yield) as a white solid. LC-MS: Ret. Time: 2.00 min. MS calcd. for chemical formula C₁₃H₁₆O₂, 204.3; Found: 205.3[M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-2: Synthesis of 4-(4-nitrophenyl) cyclohexane carboxylic acid (3): To a stirred solution of KNO₃ (1.75 g, 17.13 mmol) in H₂SO₄ (100 mL) was added compound 2 (10 g, 48.95 mmol) at 0° C. The reaction mixture was stirred at room temperature for 1 hour. After completion of the reaction (monitored by TLC), ice-cooled water was added to reaction mixture to form a solid, which was filtered and dried to afford the title compound 3 (4 g, 15.24 mmol, 31.14% yield) as a white solid. LC-MS: Ret. Time: 1.14 min. MS calcd. for chemical formula: C₁₃H₁₅NO₄: 249.3; Found: 232(M−18)⁺. Method: FA BEH 4.2 MINS Column: Acuity UPLC BEH C18 (2.1×50) mm, 1.7 μm. Flow rate: 0.5 mL/min. Mobile Phase A: 0.05% formic acid in water. Mobile Phase B: 0.05% formic acid in acetonitrile. Column Temp.: 40° C. Gradient Program Time/B: 0.0/2, 0.3/2, 2.0/98, 3.5/98, 3.6/2, 4.2/2.

Step-3: Synthesis of tert-butyl 2-((1r,4r)-4-(4-nitrophenyl)cyclohexane-1-carbonyl) hydrazine-1-carboxylate (5): To a stirred solution of compound 3 (3.6 g, 14 mmol) in DMF (36 mL) was added HATU (8.5 g, 22 mmol) and DIPEA (7.6 mL, 43 mmol) followed by compound 4 (2.3 g, 17 mmol) at room temperature. The reaction mixture was stirred at room temperature for 16 hours under nitrogen atmosphere. After completion of the reaction (monitored by TLC), ice-cooled water was added to the reaction mixture to form a precipitate. The precipitate was filtered and dried to afford the title compound 5 (3.5 g, 9.631 mmol, 66.79% yield) as an off-white solid. LC-MS: Ret. Time: 1.17 min, MS calcd. for chemical formula C₁₈H₂₅N₃O₅: 363.4; Found: 264 [M−10]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-4: Synthesis of 4-(4-nitrophenyl) cyclohexane carbohydrazide (6): To a stirred solution of compound 5 (3.5 g, 9.631 mmol) in DCM (35 mL) was added HCl (4 M) in 1, 4-dioxane (5 mL) at 0° C. The reaction mixture was stirred at room temperature for 2 hours. After completion of the reaction (monitored by TLC), the reaction solvent was evaporated to give a residue, which was washed with sodium bicarbonate solution. The organic compound was extracted with ethyl acetate, and the combined organic layer was dried over anhydrous sodium sulphate. The solvent was removed under reduced pressure to afford the desired compound 6 (2.0 g, 7.595 mmol, 54% yield) as an off-white solid. LC-MS: Ret. Time: 0.92 min. MS calcd. for chemical formula: C₁₃H₁₇N₃O₃: 263.3; Found: 264.21[M+H]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-5: Synthesis of ethyl 3-[2-[4-(4-nitrophenyl)cyclohexanecarbonyl]hydrazino]-3-oxo-propanoate (8): To a stirred solution of compound 6 (2.0 g, 5.393 mmol) in DMF (5 mL) was added EDC.HCl (2.17 g, 11.393 mmol), HOBt (1.5 g, 11.393 mmol), and DIPEA (1.6 mL, 15.18 mmol) followed by compound 7 (1.2 g, 9.115 mmol) at room temperature. The reaction mixture was stirred at room temperature for 16 hours under nitrogen atmosphere. After completion of the reaction (monitored by TLC), ice-cooled water was added to the reaction mixture to form a precipitate. The precipitate was filtered and dried to afford the title compound 8 (2.2 g, 5.829 mmol, 76.92% yield) as an off-white solid. LC-MS: Ret. Time: 1.07 min. MS calcd. for chemical formula: C₁₈H₂₃N₃O₆ 377.4; Found: 378.31 [M+H]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-6: Synthesis of ethyl 2-[5-[4-(4-nitrophenyl)cyclohexyl]-1,3,4-oxadiazol-2-yl]acetate (9): To a stirred solution of compound 8 (2 g, 5.285 mmol) in ACN (20 mL) was added POCl₃ (1.4 mL, 15.856 mmol), at 0° C. The reaction mixture was refluxed at 80° C. for 16 hours. After completion of the reaction (monitored by TLC), the reaction mixture was cooled to room temperature, washed with sodium bicarbonate solution, and extracted with ethyl acetate. The combined organic layer was dried over anhydrous sodium sulphate, and the solvent was removed under reduced pressure to give a crude product, which was purified by flash column chromatography (100-200 silica; 50% ethyl acetate-hexane eluent) to afford the desired compound 9 (1.2 g, 2.3 mmol, 63.15% yield) as an off-white sold. LC-MS: Ret. Time: 1.28 min. MS calcd. for chemical formula: C₁₈H₂₁N₃O₅ 359.4; Found: 360.29 [M+H]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-7: Synthesis of ethyl 2-[5-[4-(4-aminophenyl)cyclohexyl]-1,3,4-oxadiazol-2-yl]acetate (10): To a stirred solution of compound 9 (0.7 g, 1.948 mmol) in ethanol (10 ml) and water (2 mL) was added Fe (0.89 g, 13.63 mmol) and ammonium chloride (0.729 g, 13.63 mmol) at room temperature. The reaction mixture was stirred for 2 hours at 80° C. After completion of the reaction (monitored by TLC), the reaction mixture was diluted with methanol and filtered through a CELITE® pad. The filtrate was concentrated under reduced pressure to afford the title compound 10 (0.4 g, 0.432 mmol, 22.44% yield) as a white solid. LC-MS: Ret. Time: 0.86 min, MS calcd. for chemical formula: C₁₈H₂₃N₃O₃ 329.4; Found: 330.29 [M+H]⁺. Method: FA BEH 2.5 min, Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-8: Synthesis of ethyl 2-[5-[4-[4-(phenoxycarbonylamino)phenyl]cyclohexyl]-1,3,4-oxadiazol-2-yl]acetate (12): To a stirred solution of compound 10 (0.4 g, 1.121 mmol) in DCM (10 mL) were added pyridine (0.46 mL, 3.636 mmol) and compound 11 (0.2 mL, 1.45 mmol) at 0° C. The reaction mixture was stirred at room temperature for 1 hour under nitrogen atmosphere. After completion of the reaction (monitored by TLC), the reaction mixture was diluted with water and extracted with EtOAc. The combined organic layers were concentrated under reduced pressure to give a crude product, which was purified by flash column chromatography (100-200 silica; 5% methanol-DCM as eluent) to afford the title compound 12 (0.6 g, 0.760 mmol, 47.69% yield) as a white solid. LC-MS: Ret. Time: 1.32 min. MS calcd. for chemical formula: C₂₅H₂₇N₃O₅ 449.5; Found: 450.27 [M+H]⁺. Method: FA BEH 2.5 min Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-9: Synthesis of ethyl 2-[5-[4-[4-[(5-fluoroisoindoline-2-carbonyl)amino]phenyl]cyclohexyl]-1,3,4-oxadiazol-2-yl]acetate (14): To a stirred solution of compound 12 (0.5 g, 0.344 mmol) in DMF (5 mL) were added DIPEA (0.58 mL, 1.112 mmol) and compound 13 (0.183 g, 1.335 mmol) at 0° C. The reaction mixture was stirred at 80° C. for 3 hours under nitrogen atmosphere. After completion of the reaction (monitored by TLC), the reaction mixture was cooled to room temperature and ice-cooled water was added to the reaction mixture to precipitate a solid. The precipitate was filtered and dried to give a crude product, which was purified by flash column chromatography (230-400 silica; 5% methanol-DCM as eluent) to afford 14 (0.04 g, 0.657 mmol, 59.14% yield) as a white solid. LC-MS: Ret. Time: 1.27 min. MS calcd. for chemical formula: C₂₇H₂₉FN₄O₄ 492.5; Found: 493.33 [M+H]⁺. Method: TFA BEH 2.5 MIN Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-10: Synthesis of 2-[5-[4-[4-[(5-fluoroisoindoline-2-carbonyl)amino]phenyl]cyclohexyl]-1,3,4-oxadiazol-2-yl]acetic acid: To a stirred solution of compound 14 (0.15 g, 0.3046 mmol) in MeOH (2 mL), THF (2 mL), and water (2 mL) was added LiOH (0.02 mg, 0.913 mmol) at 0° C. The reaction mixture was stirred at room temperature for 2 hours under nitrogen atmosphere. After completion of the reaction (monitored by TLC), the solvent was evaporated under reduced pressure to give a residue, which was diluted with water and washed with ether. The aqueous layer was acidified with saturated citric acid solution to give a crude product, which was filtered and dried to afford compound 15 (0.08 g, 0.1412 mmol, 46.37% yield) as a white solid. LC-MS: Ret. Time: 1.21 min. MS calcd. for chemical formula: C₂₅H₂₅FN₄O₄ 464.5; Found: 465.32 [M+H]⁺. Method: TFA BEH 2.5 MIN Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-11: Synthesis of 5-fluoro-N-(4-((1r,4r)-4-(5-(2-((2-hydroxy-2-methylpropyl) amino)-2-oxoethyl)-1,3,4-oxadiazol-2-yl)cyclohexyl)phenyl)isoindoline-2-carboxamide (II-2): To a stirred solution of compound 15 (0.08 g, 0.172 mmol) in DMF (5 mL) was added HATU (0.08 g, 0.258 mmol), DIPEA (0.1 mL, 0.516 mmol)), and compound 16 (0.018 g, 0.206 mmol) at room temperature. The reaction mixture was stirred at room temperature for 16 hours under nitrogen atmosphere. After completion of the reaction (monitored by TLC), ice-cooled water was added to the reaction mixture and a solid was precipitated. The solid was filtered and dried to give crude product, which was purified by Prep-HPLC to afford the title compound II-2 (0.006 g, 0.0112 mmol, 6.52% yield) as an off-white solid. Purification method: Column: Xtimate C18 (250×21.2) mm, 5 μm. Mobile phase A: 0.1% FA in water. Mobile phase B: 100% ACN. Flow rate: 18 mL/min. Instrument ID: PREP-32, Gradient (Time/% B): 0/10, 3/10, 8/55, 22/80, 23/98. LC-MS: Ret. Time: 1.11 min. MS calcd. for chemical formula: C₂₉H₃₄FN₅O₄ 535.6; Found: 536.41 [M+H]⁺. LC-MS Method: FA BEH 2.5 MIN Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3. HPLC: Ret. Time: 7.100 min. HPLC Method: Column: X-Select CSH C18 (4.6×150) mm, 5.0 μm. Mobile Phase A: 0.1% FA in ACN:H₂O (5:95 (v/v)). Mobile Phase B: acetonitrile (100%). Colum Temperature: 30° C. Flow Rate: 1.0 mL/min. Gradient: 0.00/5, 1.00/5, 8.00/100, 12.00/100, 14.00/5, 18.00/5. Diluent: ACN:water (80:20). Instrument ID: AMC-HPLC-25. ¹H NMR (400 MHz, DMSO-d₆): δ: 8.28 (s, 1H), 8.19-8.13 (m, 1H), 7.46 (d, J=8.6 Hz, 2H), 7.41-7.36 (m, 1H), 7.25-7.21 (m, 1H), 7.17-7.11 (m, 3H), 4.73 (br d, J=11.6 Hz, 4H), 4.47 (s, 1H), 3.88 (s, 2H), 3.06 (d, J=6.0 Hz, 2H), 2.98 (ddd, J=11.5, 8.2, 3.4 Hz, 1H), 2.57-2.54 (m, 1H), 2.17-2.10 (m, 2H), 1.93-1.85 (m, 2H), 1.69-1.53 (m, 4H), 1.07 (s, 6H).

### Example 33—Synthesis of 5-fluoro-N-[4-[1-[1-(2-hydroxy-2-methyl-propyl) triazol-4-yl]-4-piperidyl]phenyl]isoindoline-2-carboxamide (I-23)

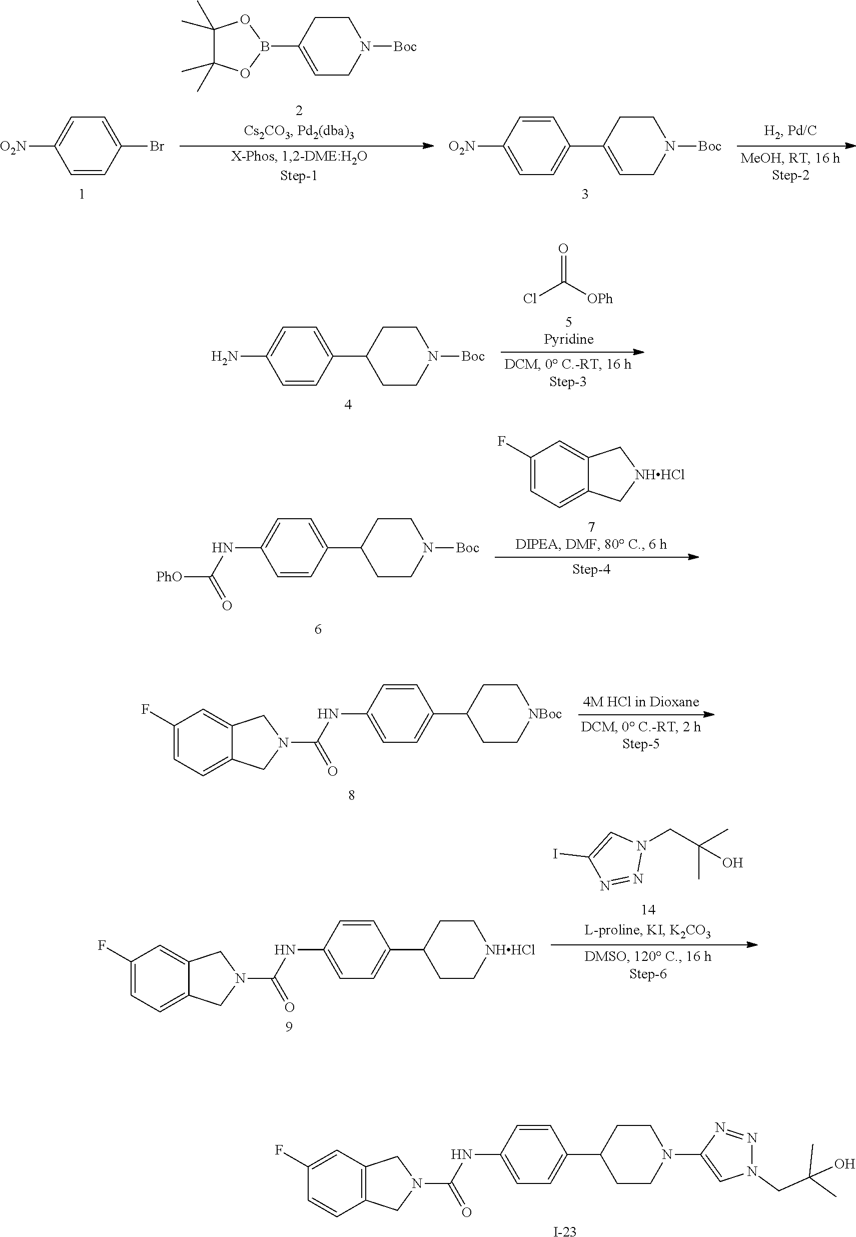

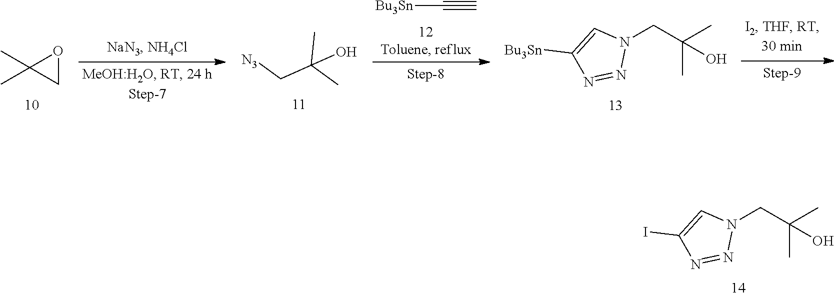

Step-1: Synthesis of tert-butyl 4-(4-nitrophenyl)-3,6-dihydropyridine-1(2H)-carboxylate (3): To a stirred solution of compound 1 (10 g, 49.505 mmol) in 1,4-dioxane:H₂O (10:2, 120 mL) was added compound 2 (18.93 g, 59.406 mmol) and Cs₂CO₃ (48.4 g, 148.51 mmol) at room temperature. The reaction mixture was degassed with argon for 5 minutes. Pd₂dba₃ (2.33 g, 2.457 mmol) and X-phos (2.40 g, 4.95 mmol) were added to the mixture at room temperature. The reaction mixture was stirred for 15 minutes while being degassed with argon, and then the reaction mixture was stirred at 90° C. for 16 hours. After completion of the reaction (monitored by TLC), the reaction mixture was cooled to room temperature, diluted with ethyl acetate, and filtered through a CELITE® pad. The filtrate was washed with water followed by brine and extracted with ethyl acetate. The organic layer was dried over anhydrous Na₂SO₄ and concentrated under reduced pressure to give a crude product, which was triturated with diethyl ether to afford the title compound 3 (12 g, 39.04 mmol, 78.86% yield) as a white solid. LC-MS: Ret. Time: 1.41 min. MS calcd. for chemical formula C₁₆H₂₀N₂O₄, 304; Found: 249 [M−56]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-2: Synthesis of 4-(piperidin-4yl) aniline (4): To a stirred solution of compound 3 (12 g, 39.603 mmol) in MeOH (120 mL) was added Pd—C(50% wet, 4.8 g) at room temperature. The reaction mixture stirred for 16 hours at room temperature under H₂ conditions (100 psi). After completion of the reaction (monitored by TLC and LCMS), the reaction mixture was filtered through a CELITE® pad, and the filtrate was and concentrated under reduced pressure to afford the title compound 4 (9.5 g, 32.608 mmol, 87% yield) as a white solid. LC-MS: Ret. Time: 1.95 min. MS calcd. for chemical formula C₁₆H₂₄N₂O₂:277; Found: 221(M−56)⁺. Method: FA Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-3: Synthesis of tert-butyl 4-(4-((phenoxycarbonyl)amino)phenyl)piperidine-1-carboxylate (6): To a stirred solution of compound 4 (5 g, 18.09 mmol) in DCM (50 mL) were added pyridine (4.41 mL, 54.27 mmol) and compound 5 (2.75 mL, 21.71 mmol) at 0° C. The mixture was stirred at room temperature for 1 hour under nitrogen atmosphere. After completion of the reaction (monitored by TLC), the reaction mixture was diluted with water and extracted with EtOAc. The combined organic layers were concentrated under reduced pressure to give the crude compound, which was triturated with diethyl ether, filtered, and dried under reduced pressure to afford the title compound 5 (8 g, 20.20 mmol, 73% yield) as a white solid. LC-MS: Ret. Time: 1.68 min. MS calcd. for chemical formula: C₂₃H₂₈N₂O₄: 396.47; Found: 297.25 [(M−100]⁺. Method: TFA CORTECS 2.5 MIN Column: CORTECS C18 (3.0×30) mm, 1.6 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-4: Synthesis of tert-butyl 4-(4((5-fluoroisoindoline-2-carbonylamino) phenyl) piperidine-1-carboxylate (8): To a stirred solution of compound 6 (3.8 g, 9.6 mmol) in DMF (38 mL) were added DIPEA (5 mL, 29 mmol) and compound 7 (1.5 g, 8.6 mmol) at 0° C. The reaction mixture was stirred at 80° C. for 3 hours under nitrogen atmosphere. After completion of the reaction (monitored by TLC), the reaction mixture was diluted with ice-cold water. The precipitated solid was filtered and dried under reduced pressure to give the crude compound, which was triturated with diethyl ether, filtered, and dried under reduced pressure to afford the title compound 8 (4.1 g, 9.3 mmol, 97% yield) as a white solid. LC-MS: Ret. Time: 2.206 min. MS calcd. for chemical formula C₂₅H₃₀FN₃O₃: 439.5; Found: 384.3 [M−56]⁺. Method: column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-5: Synthesis of 5-fluoro-N-(4-(piperidin-4-yl)phenyl) isoindoline-2-carboxamide hydrochloride (9): To a stirred solution of compound 8 (4 g, 9.101 mmol) in DCM (40 mL) was added HCl (4 M) in 1,4-dioxane (10 mL) at 0° C. The reaction mixture was stirred at room temperature for 2 hours. After completion of the reaction (monitored by TLC), volatiles were removed under reduced pressure to give a residue, which was stirred in diethyl ether (30 mL) and then pentane (5 mL×2). The solid compound thus obtained was filtered and dried under reduced pressure to afford the desired compound 9 (3 g, 21.33 mmol, 88.23% yield) as a brown solid. The resulting crude thus obtained was used in the next step without purification as a HCl salt. LC-MS: Ret. Time: 1.44 min. MS calcd. for chemical formula: C₂₀H₂₂FN₃O: 339.4; Found: 340.4[M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-6: Synthesis of 5-fluoro-N-[4-[1-[1-(2-hydroxy-2-methyl-propyl) triazol-4-yl]-4-piperidyl]phenyl]isoindoline-2-carboxamide (I-23): To a stirred solution of compound 9 (0.4 g, 1.179 mmol) in DMSO (5 mL) were added compound 14 (0.314 g, 1.179 mmol), L-Proline (0.027 g, 0.23 mmol), CuI (0.44 g, 0.23 mmol), and K₂CO₃ (0.488 g, 3.53 mmol) at room temperature. The reaction mixture was stirred at 120° C. for 16 hours. After completion of the reaction (monitored by TLC), the reaction mixture was cooled to room temperature, diluted with ice-cooled water, and extracted with ethyl acetate. The combined organic layer was dried over anhydrous sodium sulphate, and the solvent was removed under reduced pressure to give the crude product, which was purified by SFC-achiral to afford I-23 (free base, 0.005 g, 0.0104 mmol) as an off-white solid. SFC achiral purification method: Column: IC (20×250×nm, 5 μm). Mobile phase A: hexane. Mobile phase B: EtOH:MeOH (1:1), Eluent: A:B (30:70). Flow rate: 20 mL/min. No. of injections: 4 inj (5.5 mg/inj/15 min). Instrument ID: Prep-7. LC-MS: Ret. Time: 1.21 min. Purity: 98.03. MS calcd. for chemical formula: C₂₆H₃₁FN₆O 478.6; Found: 479.30 [M+H]⁺. Method: TFA BEH 2.5 MIN Column: XBridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

¹H NMR (400 MHz, DMSO-d₆): δ: 8.30 (s, 1H), 7.47 (d, J=8.5 Hz, 2H), 7.42-7.36 (m, 1H), 7.34 (s, 1H), 7.26-7.21 (m, 1H), 7.15 (d, J=8.6 Hz, 3H), 4.80 (s, 1H), 4.77-4.70 (m, 4H), 4.14 (s, 2H), 3.74 (br d, J=11.9 Hz, 2H), 2.73-2.66 (m, 2H), 2.63-2.58 (m, 1H), 1.85-1.68 (m, 4H), 1.08 (s, 6H). Purification Method: Column: Azzota pack C8 (200×30 nm) 10 μm. Mobile phase A: 0.1% TFA in water. Mobile phase B: 100% ACN. Flow rate: 25 mL/min. Instrument ID: PREP-28. Gradient (Time/% B): 0/10, 3/10, 8/40, 20/56, 21/98, 25/98, 26/10, 30/10. HPLC: Ret time: 7.39 min. Purity: 99.16%. Column: X-Select CSH C18 5 μm (4.6×150) mm. Mobile Phase A: 5 mM AA in water. Mobile Phase B: 100% acetonitrile. Column Temperature: 30° C. Flow Rate: 1.0 mL/min. Gradient: 0.0/5, 1.0/5, 8.0/95, 12.0/95, 14.0/5, 18.0/5.

Step-7: Synthesis of 2-hydroxy-2-methyl-propane-1-diazonium (11): To a stirred solution of compound 10 (3 g, 41.60 mmol) in methanol (90 mL) and water (9.6 mL) was added NaN₃ (6.83 g, 104.0 mmol) at room temperature. The reaction mixture was stirred for 16 hours at the same temperature. After completion of the reaction (monitored by TLC, KMnO₄ active), the reaction mixture was washed with water and extracted with diethyl ether. The combined organic layer was dried over anhydrous sodium sulphate, and the solvent was removed under reduced pressure to afford the desired compound 11 as a colorless liquid. ¹H NMR (400 MHz, CHLOROFORM-d): δ: 3.26 (s, 2H), 1.81 (s, 1H), 1.26 (s, 6H).

Step-8: Synthesis of 2-methyl-1-(4-tributylstannyltriazol-1-yl) propan-2-ol (12): To a stirred solution of compound 11 (1.7 g, 15 mmol) in toluene (17 mL) was added tributyl(ethynyl)stannane (4.7 g, 15 mmol) at room temperature. The reaction mixture was stirred at 100° C. for 16 hours. After completion of the reaction (monitored by TLC), the reaction mixture was cooled to room temperature, diluted with water, and extracted with ethyl acetate. The combined organic layer was dried over anhydrous sodium sulphate, and the solvent was removed under reduced pressure to give the crude product, which was purified by flash column chromatography (100-200 silica, 50% ethyl acetate-hexane as eluent) to afford compound 12 (1.7 g, 3.95 mmol, 26.98% Yield) as a colourless liquid. LC-MS: Ret. Time: 2.97 min. MS calcd. for chemical formula C₁₈H₃₇N₃O₅: 430.2; Found: 430.77 [M+H]⁺. Method: FA BEH 4.2 MIN Column: X Bridge BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 45° C. Gradient Program Time/B: 0.0/2, 0.3/2, 2.0/98, 3.50/98, 3.60/2, 4.2/2.

Step-9: Synthesis of 1-(4-iodotriazol-1-yl)-2-methyl-propan-2-ol (14): To a stirred solution of compound 13 (1.7 g, 3.95 mmol) in THF (17 mL) was added I₂ (0.4 g, 1.58 mmol) at room temperature. The reaction mixture was stirred at the same temperature for 2 hours. After completion of the reaction (monitored by TLC), the reaction mixture was diluted with water and extracted with ethyl acetate. The combined organic layer was dried over anhydrous sodium sulphate, and the solvent was removed under reduced pressure to give crude product, which was purified by flash column chromatography (100-200 silica, 80% ethyl acetate-hexane as eluent) to afford compound 14 (1.7 g, 3.95 mmol, 26.98% Yield) as a pale-yellow, gummy liquid. LC-MS: Ret. Time: 0.70 min. MS calcd. for chemical formula: C₆H₁₀IN₃O: 267.07; Found: 268.06 [M+H]⁺. Method: TFA 10 MINS Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 40° C. Gradient Program Time/B: 0.0/2, 1.5/2, 6/98, 8/98, 9.0/2, 10/2.

### Example 34—Synthesis of 3-fluoro-N-[4-[4-[5-(2-hydroxy-2-methyl-propyl)-1,3,4-oxadiazol-2-yl]cyclohexyl]phenyl]-5,7-dihydropyrrolo[3,4-b]pyridine-6-carboxamide (I-22)

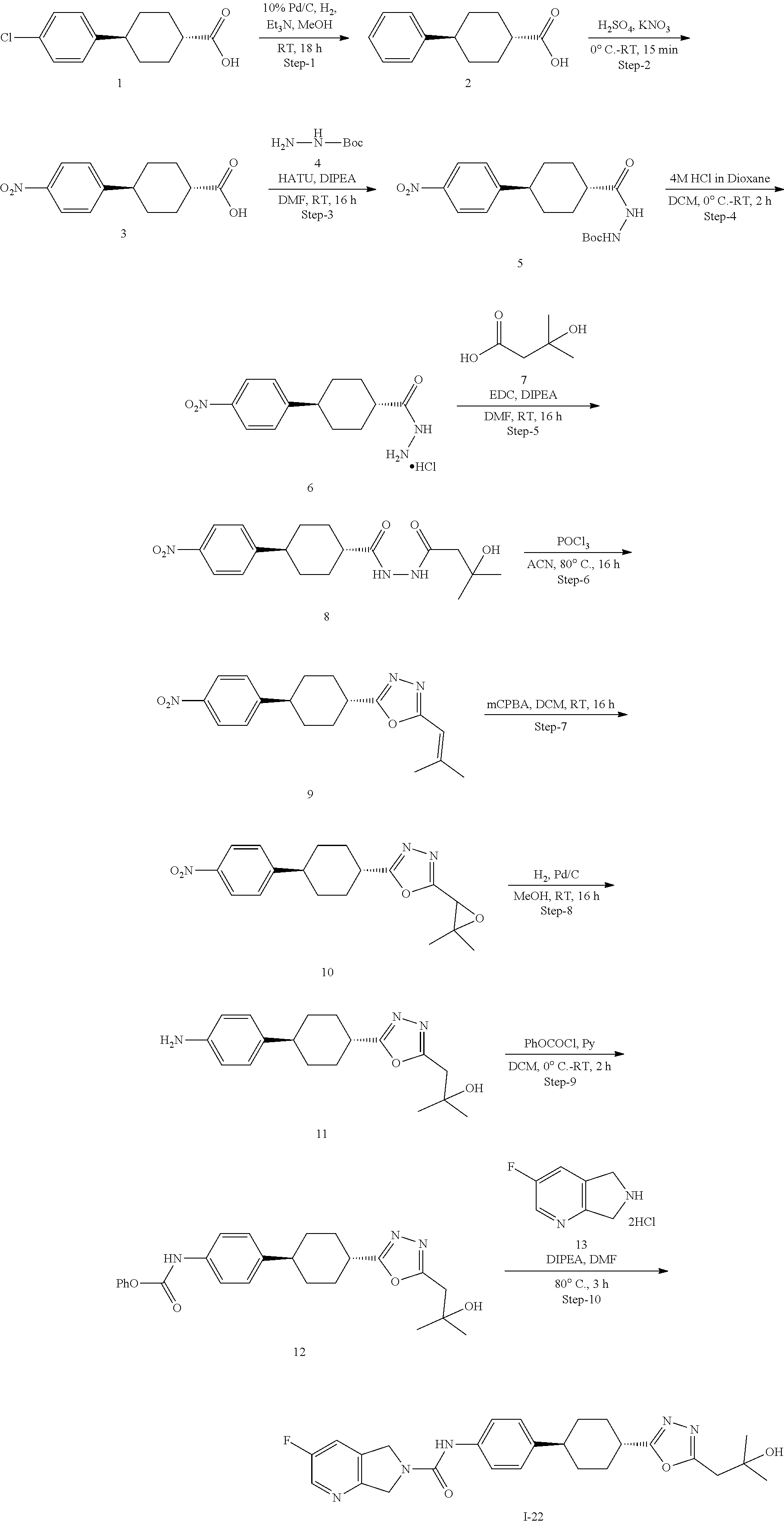

Step-1: Synthesis of 4-phenylcyclohexanecarboxylic acid (2): To a stirred solution of compound 1 (30 g, 15.7 mmol) in MeOH (300 ml) was added TEA (18 ml, 128 mmol) and Pd—C(50% wet, 9 g) at room temperature. The reaction mixture was stirred for 16 hours at 50° C. under H₂ conditions. After completion of the reaction (monitored by TLC), the reaction mixture was diluted with MeOH and filtered through a CELITE® pad. The filtrate was concentrated under reduced pressure to give the crude product, which was triturated with n-heptane and dried to afford the title compound 2 (24 g, 117.6 mmol, 93.38% yield) as a white solid. LC-MS: Ret. Time: 2.00 min. MS calcd. for chemical formula C₁₃H₁₆O₂, 204.3; Found: 205.3[M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-2: Synthesis of 4-(4-nitrophenyl) cyclohexane carboxylic acid (3): To a stirred solution of KNO₃ (3.5 g, 34.26 mmol) in H₂SO₄ (200 mL) was added compound 2 (20 g, 97.90 mmol) at 0° C. The reaction mixture was stirred at room temperature for 1 hour. After completion of the reaction (monitored by TLC), ice-cooled water was added to the reaction mixture. The solid thus precipitated was filtered and dried to afford the title compound 3 (9 g, 34.30 mmol, 35.03% yield) as a white solid. LC-MS: Ret. Time: 2.26 min. MS calcd. for chemical formula: C₁₃H₁₅NO₄: 249.3; Found: 232(M−18)⁺. Method A: FA BEH 4.2 MINS Column: Acuity UPLC BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 40° C. Gradient Program Time/B: 0.0/2, 0.3/2, 2.0/98, 3.5/98, 3.6/2, 4.2/2.

Step-3: Synthesis of tert-butyl 2-((1r,4r)-4-(4-nitrophenyl)cyclohexane-1-carbonyl) hydrazine-1-carboxylate (5): To a stirred solution of compound 3 (9 g, 36.10 mmol) in DMF (90 mL) was added tert-butyl carbazate (5.48 g, 43.32 mmol), DIPEA (18.9 mL, 108.3 mmol), and compound 4 (2.95 g, 18.84 mmol) at room temperature. The reaction mixture was stirred at room temperature for 16 hours under nitrogen atmosphere. After completion of the reaction (monitored by TLC), ice-cooled water was added to the reaction mixture. The solid thus precipitated was filtered and dried to afford the title compound 5 (4 g, 8.80 mmol, 24.39% yield) as an off-white solid. LC-MS: Ret. Time: 1.22 min. MS calcd. for chemical formula C₁₈H₂₅N₃O₅: 363.4; Found: 264 [M−10]⁺. Method G: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-4: Synthesis of 4-(4-nitrophenyl) cyclohexane carbohydrazide (6): To a stirred solution of compound 5 (4 g, 11.01 mmol) in DCM (40 mL) was added HCl (4 M) in 1,4-dioxane (5 mL) at 0° C. The mixture was stirred at room temperature for 2 hours. After completion of the reaction (monitored by TLC), the solvent was evaporated to give a residue, which was washed with sodium bicarbonate solution and extracted with ethyl acetate. The combined organic layer was dried over anhydrous sodium sulphate, and the solvent was removed under reduced pressure to afford the desired compound 6 (2.63 g, 9.5 mmol, 90.68% yield) as an off-white solid. LC-MS: Ret. Time: 0.92 min. MS calcd. for chemical formula: C₁₃H₁₇N₃O₃: 263.3; Found: 264.21[M+H]⁺. Method G: FA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-5: Synthesis of (1r,4r)-N′-(3-hydroxy-3-methylbutanoyl)-4-(4-nitrophenyl) cyclohexane-1-carbohydrazide (8): To a stirred solution of compound 6 (0.5 g, 1.899 mmol) in DMF (5 mL) was added EDC.HCl (0.568 g, 2.84 mmol), HOBt (0.40 g, 2.84 mmol), DIPEA (0.99 mL, 5.697 mmol), and compound 7 (0.269 g, 2.279 mmol) at room temperature. The reaction mixture was stirred at room temperature for 16 hours under nitrogen atmosphere. After completion of the reaction (monitored by TLC), ice-cooled water was added to the reaction mixture. The solid thus precipitated was filtered and dried to afford the title compound 7 (0.6 g, 1.403 mmol, 73.90% yield) as an off-white solid. LC-MS: Ret. Time: 1.65 min. MS calcd. for chemical formula: C₁₈H₂₅N₃O₅ 363.4; Found: 364.1 [M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-6: Synthesis of 2-(2-methylprop-1-enyl)-5-[4-(4-nitrophenyl) cyclohexyl]-1, 3, 4-oxadiazole (9): To a stirred solution of compound 8 (0.6 g, 1.65 mmol) in ACN (20 mL) was added POCl₃ (0.46 mL, 4.95 mmol) at 0° C. The reaction mixture was refluxed at 80° C. for 16 hours. After completion of the reaction (monitored by TLC), the reaction mixture was cooled to room temperature, washed with sodium bicarbonate solution, and extracted with ethyl acetate. The combined organic layer was dried over anhydrous sodium sulphate, and the solvent was removed under reduced pressure to give the crude product, which was purified by flash column chromatography (100-200 silica, 50% ethyl acetate-hexane as eluent) to afford the desired compound 9 (0.4 g, 1.22 mmol 74% yield) as an off-white sold. LC-MS: Ret. Time: 3.21 min. MS calcd. for chemical formula: C₁₈H₂₁N₃O₃ 327.4; Found: 328.4 [M+H]⁺. Method: Column: XbridgeC18 (4.6×50 mm, 3.5 μm). Mobile Phase A: 10 mM NH₄HCO₃ in water. Mobile Phase: B: ACN. Gradient (T/B %): 0.0/2, 0.3/2, 2.5/98, 4.0/98, 4.2/2, 5/2. Flow rate: 1 mL/min. Column Temp.: 40° C.

Step-7: Synthesis of 2-[(2-methyloxiran-2-yl) methyl]-5-[4-(4-nitrophenyl) cyclohexyl]-1,3,4-oxadiazole (10): To a stirred solution of compound 9 (0.4 g, 1.22 mmol) in DCM (20 mL) was added mCPBA (1.1 g, 6.11 mmol), at 0° C. The reaction mixture was stirred at room temperature for 16 hours. After completion of the reaction (monitored by TLC), the reaction mixture was washed with sodium bicarbonate solution and extracted with ethyl acetate. The combined organic layer was dried over anhydrous sodium sulphate, and the solvent was removed under reduced pressure to give the crude product, which was purified by flash column chromatography (100-200 silica, 50% ethyl acetate-hexane as eluent) to afford the desired compound 10 (0.35 g, 1.02 mmol, 85% yield) as an off-white sold. LC-MS: Ret. Time: 2.03 min. MS calcd. for chemical formula: C₁₈H₂₁N₃O₄ 343.4; Found: 343.9 [M+H]⁺. Method: Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-8: Synthesis of 1-[5-[4-(4-aminophenyl) cyclohexyl]-1, 3, 4-oxadiazol-2-yl]-2-methyl-propan-2-ol (11): To a stirred solution of compound 9 (0.28 g, 0.8154 mmol) in methanol (10 mL) was added Pd—C(50% wet, 50 mg) at room temperature. The reaction mixture was stirred for 16 hours at room temperature under H₂ conditions. After completion of the reaction (monitored by TLC), the reaction mixture was diluted with methanol and filtered through a CELITE® pad. The filtrate was concentrated under reduced pressure to afford the title compound 10 (0.2 g, 0.570 mmol, 69.99% yield) as a white solid. LC-MS: Ret. Time: 1.85 min. MS calcd. for chemical formula: C₁₈H₂₅N₃O₂ 315.4; Found: 316.14 [M+H]⁺. Method A: FA BEH 4.2 MIN Column: X Bridge BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 45° C. Gradient Program Time/B: 0.0/2, 0.3/2, 2.0/98, 3.50/98, 3.60/2, 4.2/2.

Step-9: Synthesis of phenyl (4-((1r,4r)-4-(5-(2-hydroxy-2-methylpropyl)-1,3,4-oxadiazol-2-yl)cyclohexyl)phenyl)carbamate (12): To a stirred solution of compound 11 (0.2 g, 0.6341 mmol) in DCM (10 mL) were added pyridine (0.15 mL, 1.902 mmol) and phenyl chloroformate (0.1 mL, 0.760 mmol) at 0° C. The reaction mixture was stirred at room temperature for 2 hours under nitrogen atmosphere. After completion of the reaction (monitored by TLC), the reaction mixture was diluted with water and extracted with EtOAc. The combined organic layers were concentrated under reduced pressure to give a crude product, which was purified by flash column chromatography (100-200 silica, 5% methanol-DCM as eluent) to afford the title compound 12 (0.15 g, 0.344 mmol, 54% yield) as a white solid. LC-MS: Ret. Time: 1.39 min. MS calcd. for chemical formula: C₂₅H₂₉N₃O₄ 435.5; Found: 436.38 [M+H]⁺. Method X: FA CORTECS 2.5 MIN Column: CORTECS C18 (3.0×30) mm, 1.6 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-10: Synthesis of 3-fluoro-N-[4-[4-[5-(2-hydroxy-2-methyl-propyl)-1,3,4-oxadiazol-2-yl]cyclohexyl]phenyl]-5,7-dihydropyrrolo[3,4-b]pyridine-6-carboxamide (I-22): To a stirred solution of compound 12 (0.15 g, 0.344 mmol) in DMF (5 mL) were added DIPEA (0.3 mL, 1.724 mmol) and compound 13 (0.057 g, 0.413 mmol) at 0° C. The reaction mixture was stirred at 80° C. for 3 hours under nitrogen atmosphere. After completion of the reaction (monitored by TLC), the reaction mixture was cooled to room temperature. Ice-cooled water was added to the reaction mixture. The precipitate thus formed was filtered and dried under reduced pressure to give crude product, which was purified by Flash column chromatography (230-400 silica, 5% methanol-DCM as eluent) to afford I-22 (0.065 g, 0.1313 mmol 28.63% yield) as a white solid. LC-MS: Ret. Time: 1.18 min. Purity: 94%. MS calcd. for chemical formula: C₂₆H₃₀FN₅O₃ 479.6; Found: 480.38 [M+H]⁺. Method: Method Details: TFA CORTECS 2.5 MIN Column: CORTECS C18 (3.0×30) mm, 1.6 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3. HPLC Method: RT time 6.77 min. Purity: 95.06%. Column: X-Select CSH C18 5 μm (4.6×150) mm. Mobile Phase A: 2.5 mM ammonium bicarbonate in water. Mobile Phase B: acetonitrile (100%). Colum Temperature: 45° C. Flow Rate: 1.2 mL/min. Gradient: 0.0/5, 1.0/5, 8.0/95, 12.0/95, 14.0/5, 18.0/5. Diluent: ACN:water (80:20). Instrument ID: AMC-HPLC-31. ¹H NMR (400 MHz, DMSO-d₆): δ: 8.50-8.46 (m, 1H), 8.35 (s, 1H), 7.81-7.78 (m, 1H), 7.47 (d, J=8.5 Hz, 2H), 7.15 (d, J=8.6 Hz, 2H), 4.78 (br s, 2H), 4.75-4.70 (m, 3H), 3.01-2.92 (m, 1H), 2.90 (s, 2H), 2.54-2.52 (m, 1H), 2.18-2.11 (m, 2H), 1.95-1.85 (m, 2H), 1.61 (br d, J=10.0 Hz, 4H), 1.19 (s, 6H).

### Example 35—Synthesis of 5-fluoro-N-[4-[1-[1-(2-hydroxy-2-methyl-propyl) triazol-4-yl]-4-piperidyl]phenyl]isoindoline-2-carboxamide (I-23)

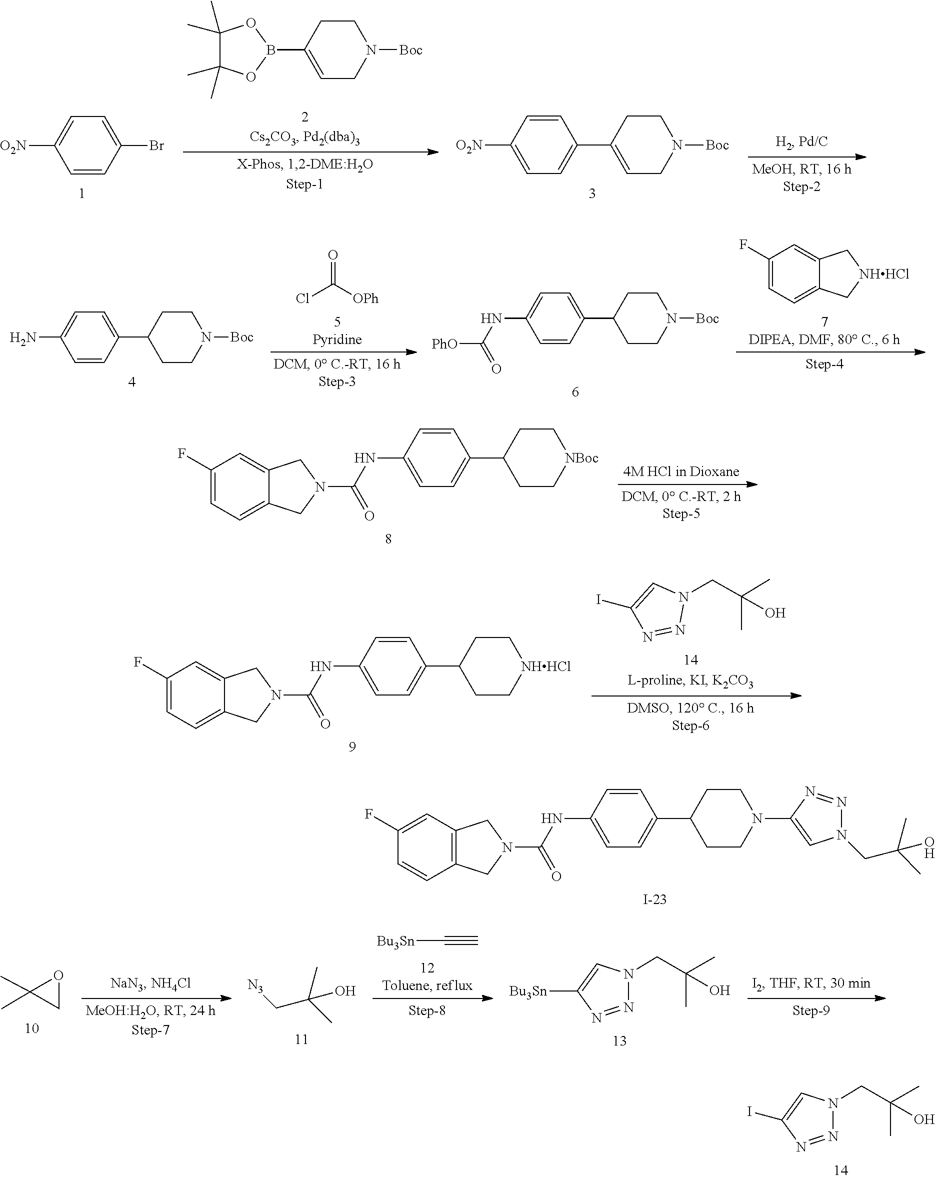

Step-1: Synthesis of tert-butyl 4-(4-nitrophenyl)-3,6-dihydropyridine-1(2H)-carboxylate (3): To a stirred solution of compound 1 (10 g, 49.505 mmol) in 1,4-dioxane:H₂O (10:2, 120 mL) was added compound 2 (18.93 g, 59.406 mmol) and Cs₂CO₃ (48.4 g, 148.51 mmol) at room temperature. The reaction mixture was degassed with argon for 5 minutes. Pd₂dba₃ (2.33 g, 2.457 mmol) and X-phos (2.40 g, 4.95 mmol) were added to the reaction mixture at room temperature, and the reaction mixture was stirred for 15 minutes while being degassed with argon. The reaction mixture was stirred at 90° C. for 16 hours. After completion of the reaction (monitored by TLC), the reaction mixture was cooled to room temperature, diluted with ethyl acetate, and filtered through a CELITE® pad. The filtrate was washed with water followed by brine and then extracted with ethyl acetate. The organic layer was dried over anhydrous Na₂SO₄ and concentrated under reduced pressure to give the crude product, which was triturated with diethyl ether to afford the title compound 3 (12 g, 39.04 mmol, 78.86% yield) as a white solid. LC-MS: Ret. Time: 1.41 min. MS calcd. for chemical formula C₁₆H₂₀N₂O₄, 304; Found: 249 [M−56]⁺. Method: TFA BEH 2.5 MIN Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-2: Synthesis of 4-(piperidin-4yl) aniline (4): To a stirred solution of compound 3 (12 g, 39.603 mmol) in MeOH (120 mL) was added Pd—C(50% wet, 4.8 g), at room temperature. The reaction mixture was stirred for 16 hours at room temperature under H₂ conditions (100 psi). After completion of the reaction (monitored by TLC and LCMS), the reaction mixture was filtered through a CELITE® pad, and the filtrate was concentrated under reduced pressure to afford the title compound 4 (9.5 g, 32.608 mmol, 87% yield) as a white solid. LC-MS: Ret. Time: 1.95 min. MS CALCD. for chemical formula C₁₆H₂₄N₂O₂, 277; Found: 221(M−56)⁺. Method: FA Column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-3: Synthesis of tert-butyl 4(-4((phenoxycorbonlyl) amino) phenyl) piperidine-1-carboxylate (6): To a stirred solution of compound 4 (5 g, 18.09 mmol) in DCM (50 mL) were added pyridine (4.41 mL, 54.27 mmol) and compound 5 (2.75 mL, 21.71 mmol) at 0° C. The reaction mixture was stirred at room temperature for 1 hour under nitrogen atmosphere. After completion of the reaction (monitored by TLC), the reaction mixture was diluted with water and extracted with EtOAc. The combined organic layers were concentrated under reduced pressure to give the crude product, which was triturated with diethyl ether, filtered, and dried under reduced pressure to afford the title compound 5 (8 g, 20.20 mmol, 73% yield) as a white solid. LC-MS: Ret. Time: 1.68 min. MS calcd. for chemical formula: C₂₃H₂₈N₂O₄: 396.47; Found: 297.25 [(M−100]⁺. Method: TFA CORTECS 2.5 MIN Column: CORTECS C18 (3.0×30) mm, 1.6 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/3.

Step-4: Synthesis of tert-butyl 4-(4-(5-fluoroisoindoline-2-carboxamido)phenyl) piperidine-1-carboxylate (8): To a stirred solution of compound 6 (3.8 g, 9.6 mmol) in DMF (38 mL) were added DIPEA (5 mL, 29 mmol) and compound 7 (1.5 g, 8.6 mmol) at 0° C. The reaction mixture was stirred at 80° C. for 3 hours under nitrogen atmosphere. After completion of the reaction (monitored by TLC), the reaction mixture was diluted with ice-cold water. The precipitate thus obtained was filtered and dried under reduced pressure to give the crude product, which was triturated with diethyl ether, filtered, and dried under reduced pressure to afford the title compound 8 (4.1 g, 9.3 mmol, 97% yield) as a white solid. LC-MS: Ret. Time: 2.206 min. MS calcd. for chemical formula C₂₅H₃₀FN₃O₃: 439.5; Found: 384.3 [M−56]⁺. Method: column: X-Select CSH C18, (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-5: Synthesis of 5-fluoro-N-(4-(4piperidyl) phenyl) isoindoline-2-carboxamide hydrochloride (9): To a stirred solution of compound 8 (4 g, 9.101 mmol) in DCM (40 mL) was added HCl (4 M) in 1,4-dioxane (10 mL) at 0° C. The reaction mixture was stirred at room temperature for 2 hours. After completion of the reaction (monitored by TLC), volatiles were removed under reduced pressure. The residue was stirred in diethyl ether (30 mL) and then with pentane (5 mL×2). The solid thus obtained was filtered and dried under reduced pressure to afford the desired compound 9 (3 g, 21.33 mmol, 88.23% yield) as a brown solid. The crude product thus obtained was used in the next step without purification as an HCl salt. LC-MS: Ret. Time: 1.44 min. MS calcd. for chemical formula: C₂₀H₂₂FN₃O: 339.4; Found: 340.4[M+H]⁺. Method: Column: X-Select CSH C18 (50 mm×3.0 mm, 2.5 μm). Mobile Phase A: 0.05% formic acid in water. Mobile Phase B: 0.05% formic acid in acetonitrile. Flow rate: 1.0 mL/min. Column temperature: 40° C. Gradient Program (B %): 0.01/2, 0.3/2, 2.0/98, 2.8/98, 3.0/2, 3.7/2.

Step-6: Synthesis of 5-fluoro-N-[4-[1-[1-(2-hydroxy-2-methyl-propyl) triazol-4-yl]-4-piperidyl]phenyl]isoindoline-2-carboxamide (I-23): To a stirred solution of compound 9 (0.4 g, 1.179 mmol) in DMSO (5 mL) were added compound 14 (0.314 g, 1.179 mmol), L-Proline (0.027 g, 0.23 mmol), CuI (0.44 g, 0.23 mmol), and K₂CO₃ (0.488 g, 3.53 mmol) at room temperature. The reaction mixture was stirred at 120° C. for 16 hours. After completion of the reaction (monitored by TLC), the reaction mixture was cooled room temperature, diluted with ice-cooled water, and extracted with ethyl acetate. The combined organic layer was dried over anhydrous sodium sulphate, and the solvent was removed under reduced pressure to give the crude product, which was purified by Prep-HPLC to afford I-23 (TFA salt, 0.025 g, 0.052 mmol) as an off-white solid. LC-MS: Ret. Time: 1.25 min. Purity: 99.61%. MS calcd. for chemical formula C₂₆H₃₁FN₆O₂: 478.6; Found: 479.30 [M+H]⁺. Method: TFA CORTECS 2.5 MIN Column: CORTECS C18 (3.0×30) mm, 1.6 μm. Flow rate: 0.85 mL/min. Mobile Phase A: 0.05% TFA in water. Mobile Phase B: 0.05% TFA in acetonitrile. Column Temp.: 45° C. Gradient Program Time/B: 0.0/3, 0.1/3, 1.4/97, 2.0/97, 2.05/3, 2.5/. HPLC: Ret time: 7.540 min. Purity: 99.3%. Column: X-Select CSH C18 5 μm (4.6×150) mm. Mobile Phase A: 0.05% TFA in water. Mobile Phase B: 100% acetonitrile. Colum Temperature: 30° C. Flow: 1.0 mL/min. Gradient: 0.0/5, 1.0/5, 8.0/95, 12.0/95, 14.0/5, 18.0/5. Diluent: ACN:water (80:20). Instrument ID: AMC-HPLC-32. ¹H NMR (400 MHz, DMSO-d₆): δ ¹H NMR (400 MHz, DMSO-d₆) δ 8.30 (s, 1H), 7.47 (d, J=8.5 Hz, 2H), 7.39 (dd, J=8.4, 5.2 Hz, 1H), 7.35 (s, 1H), 7.25-7.21 (m, 1H), 7.20-7.10 (m, 3H), 4.73 (br d, J=11.6 Hz, 4H), 4.14 (s, 2H), 3.74 (br d, J=12.0 Hz, 2H), 2.75-2.65 (m, 2H), 2.62-2.56 (m, 1H), 1.86-1.67 (m, 4H), 1.08 (s, 6H). Prep-HPLC purification method: Column AZZOTA Pack C8 (200×30 mm) 10 μm. Mobile Phase A: 0.1% TFA in water. Mobile Phase B: 100% ACN. Flow rate: 25 mL/min. Instrument ID: PREP-28 Gradient (Time/% B): 0/10, 3/10, 8/40, 20/56, 21/98, 25/98, 26/10, 30/10.

Step-7: Synthesis of 2-hydroxy-2-methyl-propane-1-diazonium (11): To a stirred solution of compound 10 (3 g, 41.60 mmol) in methanol (90 mL) and water (9.6 mL) was added NaN₃ (6.83 g, 104.0 mmol) at room temperature. The reaction mixture was stirred for 16 hours at the same temperature. After completion of the reaction (monitored by TLC, KMnO₄ active), the reaction mixture was washed with water and extracted with diethyl ether. The combined organic layer was dried over anhydrous sodium sulphate, and the solvent was removed under reduced pressure to afford the desired compound 11 as a colorless liquid. ¹H NMR (400 MHz, CHLOROFORM-d): δ 3.26 (s, 2H), 1.81 (s, 1H), 1.26 (s, 6H).

Step-8: Synthesis of 2-methyl-1-(4-tributylstannyltriazol-1-yl) propan-2-ol (12): To a stirred solution of compound 11 (1.7 g, 15 mmol) in toluene (17 mL) was added tributyl(ethynyl)stannane (4.7 g, 15 mmol) at room temperature. The reaction mixture was stirred at 100° C. for 16 hours. After completion of the reaction (monitored by TLC), the reaction mixture was cooled to room temperature, diluted with water, and extracted with ethyl acetate. The combined organic layer was dried over anhydrous sodium sulphate, and the solvent was removed under reduced pressure to give the crude product, which was purified by flash column chromatography (100-200 silica, 50% ethyl acetate-hexane as eluent) to afford compound 12 (1.7 g, 3.95 mmol, 26.98% Yield) as a colourless liquid. LC-MS: Ret. Time: 2.97 min. MS calcd. for chemical formula C₁₈H₃₇N₃O₅: 430.2; Found: 430.77 [M+H]⁺. Method: FA BEH 4.2 MIN Column: X Bridge BEH C18 (2.1×50) mm, 1.7 μm. Flow rate: 0.5 mL/min. Mobile Phase A: 0.05% FA in water. Mobile Phase B: 0.05% FA in acetonitrile. Column Temp.: 45° C. Gradient Program Time/B: 0.0/2, 0.3/2, 2.0/98, 3.50/98, 3.60/2, 4.2/2.

Step-9: Synthesis of 1-(4-iodotriazol-1-yl)-2-methyl-propan-2-ol (14): To a stirred solution of compound 13 (1.7 g, 3.95 mmol) in THF (17 mL) was added 12 (0.4 g, 1.58 mmol) at room temperature. The reaction mixture was stirred at the same temperature for 2 hours. After completion of the reaction (monitored by TLC), the reaction mixture was diluted with water and extracted with ethyl acetate. The combined organic layer was dried over anhydrous sodium sulphate, and the solvent was removed under reduced pressure to give the crude product, which was purified by flash column chromatography (100-200 silica, 80% ethyl acetate-hexane as eluent) to afford compound 14 (1.7 g, 3.95 mmol, 26.98% Yield) as a pale-yellow gummy liquid. LC-MS: Ret. Time: 0.70 min. MS calcd. for chemical formula: C₆H₁₀IN₃O: 267.07; Found: 268.06 [M+H]⁺. Method: TFA 10 MINS Column: X Bridge BEH C18 (2.1×30) mm, 2.5 μm. Flow rate: 0.85 mL/min. Mobile Phase A: 0.05% TFA in water. Mobile Phase B: 0.05% TFA in acetonitrile. Column Temp.: 40° C. Gradient Program Time/B: 0.0/2, 1.5/2, 6/98, 8/98, 9.0/2, 10/2.

### Example 36—Synthesis of 3-fluoro-N-(4-(4-(5-(2-hydroxyethyl)-1,3,4-oxadiazol-2-yl) bicyclo [2.2.2]octan-1-yl) phenyl)-5,7-dihydro-6H-pyrrolo [3,4-b]pyridine-6-carboxamide (I-38)

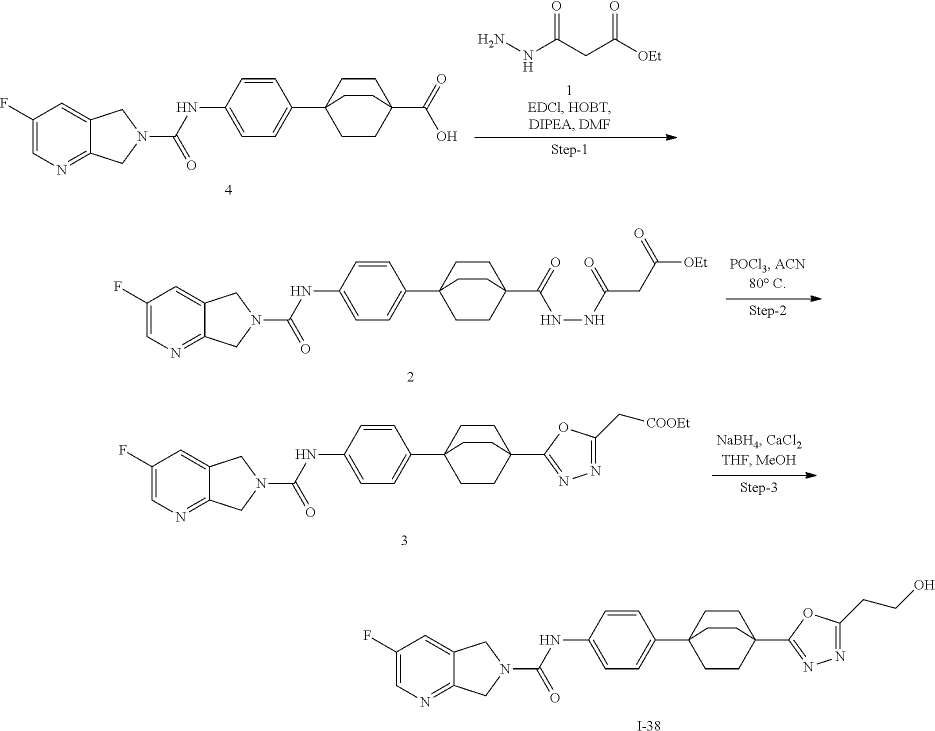

The following LCMS method was used: Column: X Bridge C18 4.6×50 mm×3.5 μm. Mobile Phase A: 10 mM ammonium acetate. Mobile Phase B: 100% ACN. Gradient: 0.0/10, 0.5/10, 4.0/90, 8.0/90, 8.1/10, 10.0/10. Flow rate: 0.6 mL/min. The following HPLC method was used: Column: X Bridge C18 4.6×250 mm×5.0 μm. Mobile Phase: Solvent A: 10 mM ammonium acetate in water. Solvent B: CH₃CN. Flow rate: 1.2 mL/min. Diluent: methanol (100%).

Preparation of ethyl 3-(2-(4-(4-(3-fluoro-6,7-dihydro-5H-pyrrolo [3,4-b]pyridine-6-carboxamido) phenyl) bicyclo [2.2.2]octane-1-carbonyl) hydrazineyl)-3-oxopropanoate (2): To a stirred solution of 4 (1.0 g, 2.44 mmol) in DMF (10 mL) was added 1 (530 mg, 3.66 mmol), EDCI (2.1 g, 10.99 mmol), HOBt (1.48 g, 10.99 mmol), and DIPEA (3.6 mL, 19.55 mmol) at room temperature. The reaction mixture was stirred at room temperature for 48 hours, and the progress of the reaction was monitored by TLC. After complete consumption of starting material, the reaction mixture was diluted with ice-cold water to form a precipitate. The precipitate was filtered, washed with water, and dried to give a residue, which was triturated with MTBE and n-pentane (20 mL) to obtain 2 (900 mg, 68% yield) as an off-white solid. ¹H NMR (300 MHz, DMSO-d₆): δ=9.70 (s, 2H), 8.47 (s, 1H), 8.35 (s, 1H), 7.79 (dd, J=2.3, 9.0 Hz, 1H), 7.45 (d, J=8.7 Hz, 2H), 7.26-7.16 (m, J=8.6 Hz, 2H), 4.77 (s, 2H), 4.72 (s, 2H), 4.13-4.01 (m, 2H), 3.25 (s, 2H), 1.79 (d, J=3.7 Hz, 12H), 1.23-1.15 (m, 3H). LCMS: 538.14 (M+H). Purity: 61.96% by AUC. RT: 4.52. UV: 240 nm.

Preparation of ethyl 2-(5-(4-(4-(3-fluoro-6,7-dihydro-5H-pyrrolo [3,4-b]pyridine-6-carboxamido) phenyl) bicyclo [2.2.2]octan-1-yl)-1,3,4-oxadiazol-2-yl) acetate (3): To a stirred solution of 2 (300 mg, 0.54 mmol) in ACN (10.0 mL) was added POCl₃ (268 mg, 1.65 mmol) slowly at 0° C. The reaction mixture was stirred at 80° C. for 8 hours and monitored by TLC and LCMS. After complete consumption of starting material, the volatiles were removed under reduced pressure to obtain a residue, which was diluted with water (30 mL) and basified to pH ˜8-9 by addition of saturated aqueous NaHCO₃ solution. The reaction mixture was extracted with EtOAc (2×30 mL). The combined organic layers were dried over Na₂SO₄ and concentrated under reduced pressure to afford the crude product, which was purified by column chromatography (eluent: 70% EtOAc in hexane) to afford 3 (100 mg, 38% yield) as an off-white solid. ¹H NMR (300 MHz, DMSO-d₆): δ=8.48 (d, J=1.4 Hz, 1H), 8.36 (s, 1H), 7.80 (dd, J=2.7, 9.0 Hz, 1H), 7.48 (d, J=8.8 Hz, 2H), 7.25 (d, J=8.8 Hz, 2H), 4.77 (s, 2H), 4.72 (s, 2H), 4.18-4.10 (m, 4H), 2.03-1.93 (m, 6H), 1.92-1.82 (m, 6H), 1.19 (t, J=7.1 Hz, 3H). LCMS: 520.14 (M+H). Purity: 91.96% by AUC. RT: 5.08. UV: 240 nm.

Preparation of 3-fluoro-N-(4-(4-(5-(2-hydroxyethyl)-1,3,4-oxadiazol-2-yl) bicyclo [2.2.2]octan-1-yl) phenyl)-5,7-dihydro-6H-pyrrolo [3,4-b]pyridine-6-carboxamide (I-38): To a stirred solution of 3 (100 mg, 0.19 mmol) in a mixture of THF:MeOH (2:1, 7.0 mL) was added CaCl₂ (63.5 mg, 0.57 mmol) followed by NaBH₄ (43.8 mg, 1.15 mmol) portion-wise at 0° C. The reaction mixture was stirred at room temperature for 1 hour and monitored by TLC and LCMS. After complete consumption of starting material, the reaction mixture was quenched with saturated aqueous NH₄Cl solution (25 mL) and basified to pH ˜8-9 by addition of saturated aqueous Na₂CO₃ solution. The reaction mixture was extracted with EtOAc (2×30 mL). The combined organic layers were dried over Na₂SO₄ and concentrated under reduced pressure to afford the crude product, which was purified by column chromatography (eluent: 2% MeOH in DCM) to afford I-38 (55 mg, 60% yield) as an off-white solid. ¹H NMR (300 MHz, DMSO-d₆): δ=8.50-8.46 (m, 1H), 8.36 (s, 1H), 7.82-7.78 (m, 1H), 7.47 (d, J=8.8 Hz, 2H), 7.25 (d, J=8.7 Hz, 2H), 4.94-4.88 (m, 1H), 4.77 (s, 2H), 4.73 (s, 2H), 3.75 (q, J=6.3 Hz, 2H), 2.94 (t, J=6.4 Hz, 2H), 2.02-1.92 (m, 6H), 1.92-1.81 (m, 6H). LCMS: 478.04 (M+H). Purity: 98.85% by AUC. RT: 4.47. UV: 240 nm. HPLC: Purity: 97.71% by AUC. RT: 12.52. UV: 245 nm.

### Example 37—Synthesis of 3-fluoro-N-(4-(4-(5-(2-hydroxy-2-methylpropyl)-1,3,4-oxadiazol-2-yl) bicyclo [2.2.2]octan-1-yl)phenyl)-5,7-dihydro-6H-pyrrolo[3,4-b]pyridine-6-carboxamide

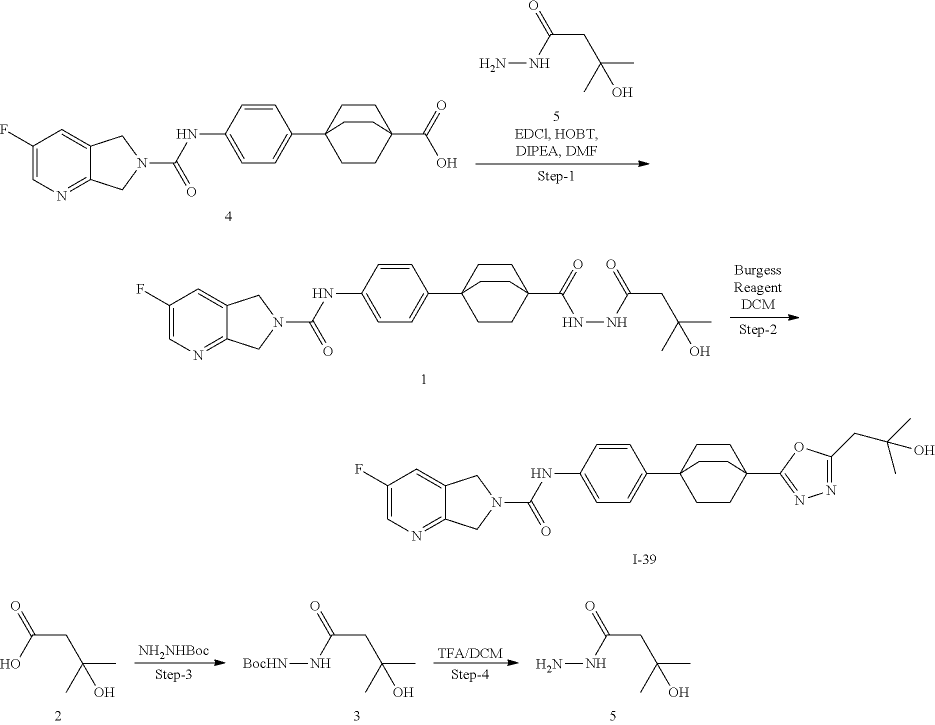

Preparation of 3-fluoro-N-(4-(4-(2-(3-hydroxy-3-methylbutanoyl)hydrazine-1-carbonyl) bicyclo [2.2.2]octan-1-yl)phenyl)-5,7-dihydro-6H-pyrrolo[3,4-b]pyridine-6-carboxamide (1): To a stirred solution of 4 (780 mg, 1.91 mmol) in DMF (20 mL) was added 5 (377 mg, 2.86 mmol), EDCI (1.64 g, 8.58 mmol), HOBt (1.16 g, 8.58 mmol), and DIPEA (2.81 mL, 15.26 mmol) at room temperature. The reaction mixture was stirred at room temperature for 16 hours, and the progress of the reaction was monitored by TLC. After complete consumption of starting material, the reaction mixture was diluted with ice-cold water (100 mL) to form a precipitate. The precipitate was filtered, washed with water, and dried to obtain the crude product, which was purified by column chromatography (eluent: 1 to 10% MeOH in DCM) to afford 1 (250 mg, 25% yield) as an off-white solid. ¹H NMR (300 MHz, DMSO-d₆): δ 9.53 (s, 1H), 9.31 (s, 1H), 8.48 (d, J=1.3 Hz, 1H), 8.34 (s, 1H), 7.79 (dd, J=2.7, 9.0 Hz, 1H), 7.49-7.42 (m, 2H), 7.22 (d, J=8.8 Hz, 2H), 4.75 (d, J=14.3 Hz, 4H), 4.67-4.65 (m, 1H), 2.23 (s, 2H), 1.88-1.69 (m, 12H), 1.25-1.15 (m, 6H). LCMS: 524.14 (M+H). Purity: 84.97% by AUC. RT: 4.30. UV: 235 nm.

Preparation of 3-fluoro-N-(4-(4-(5-(2-hydroxy-2-methylpropyl)-1,3,4-oxadiazol-2-yl) bicyclo [2.2.2]octan-1-yl)phenyl)-5,7-dihydro-6H-pyrrolo[3,4-b]pyridine-6-carboxamide (I-39): To a stirred solution of 1 (200 mg, 0.38 mmol) in DCM (5 mL) was added Burgess reagent (136 mg, 0.57 mmol) at room temperature. The reaction mixture was stirred at room temperature for 16 hours. After 16 hours, a non-polar spot along with unreacted starting material was observed via thin-layer chromatography (TLC). The reaction mixture was diluted with DCM (15 mL) and washed with water (25 mL). The organic layer was separated, dried over anhydrous Na₂SO₄, filtered, and concentrated to obtain the crude product, which was purified by column chromatography (eluent: 1 to 5% MeOH in DCM) to afford I-39 (22 mg, 11% yield) as an off-white solid. ¹H NMR (300 MHz, DMSO-d₆): δ 8.48 (d, J=1.2 Hz, 1H), 8.36 (s, 1H), 7.79 (dd, J=2.7, 9.0 Hz, 1H), 7.47 (d, J=8.7 Hz, 2H), 7.25 (d, J=8.8 Hz, 2H), 4.79-4.70 (m, 5H), 2.90 (s, 2H), 2.03-1.82 (m, 12H), 1.18 (s, 6H). LCMS: 506.07 (M+H). Purity: 98.27% by AUC. RT: 4.65. UV: 245 nm. HPLC: Purity: 96.32% by AUC. RT: 13.09. UV: 240 nm.

Preparation of tert-butyl 2-(3-hydroxy-3-methylbutanoyl)hydrazine-1-carboxylate (3): To a stirred solution of 2 (5.0 g, 42.37 mmol) in DMF (50 mL) was added tert-butyl hydrazinecarboxylate (6.15 g, 46.61 mmol), EDCI (12.1 g, 63.55 mmol), HOBt (8.5 g, 63.55 mmol), and DIPEA (23.4 mL, 127 mmol) at room temperature. The reaction mixture was stirred at room temperature for 16 hours, and the progress of the reaction was monitored by TLC. After complete consumption of starting material, the reaction mixture was diluted with ice-cold water (100 mL) and extracted with EtOAc (2×100 mL). The organic layer was separated, dried over anhydrous Na₂SO₄, filtered, and concentrated to obtain the crude product, which was purified by column chromatography (eluent: 0 to 10% MeOH in DCM) to afford 3 (4.0 g, 40% yield) as a white solid. ¹H NMR (300 MHz, DMSO-d₆): δ 9.47 (s, 1H), 8.71 (s, 1H), 4.62 (s, 1H), 2.19 (s, 2H), 1.39 (s, 9H), 1.17 (s, 6H).

Preparation of 3-hydroxy-3-methylbutanehydrazide (5): To stirred solution of 3 (4.0 g, 17.24 mmol) in DCM (40 mL) was added TFA (13.19 mL, 172 mmol) at room temperature. The reaction mixture was stirred at room temperature for 16 hours, and the progress of the reaction was monitored by TLC. After complete consumption of starting material, the volatiles were removed under reduced pressure to obtain the crude product, which was basified to pH ˜8-9 by addition of saturated NaHCO₃ solution. The aqueous layer was extracted with 30% IPA-DCM (100 mL), filtered, and concentrated to obtain the crude product 5 (1.8 g, 82% yield) as a colorless syrup. The crude product was used directly in the next step without purification. ¹H NMR (300 MHz, DMSO-d₆): δ 9.29-8.55 (m, 1H), 7.59-6.90 (m, 2H), 4.82 (br s, 1H), 2.16-2.13 (m, 2H), 1.14-1.10 (m, 6H).

### Example 38—Synthesis of 5-fluoro-N-(4-(4-(5-(2-hydroxy-2-methylpropyl)-1,3,4-oxadiazol-2-yl)bicyclo[2.2.2]octan-1-yl)phenyl)isoindoline-2-carboxamide (I-40)

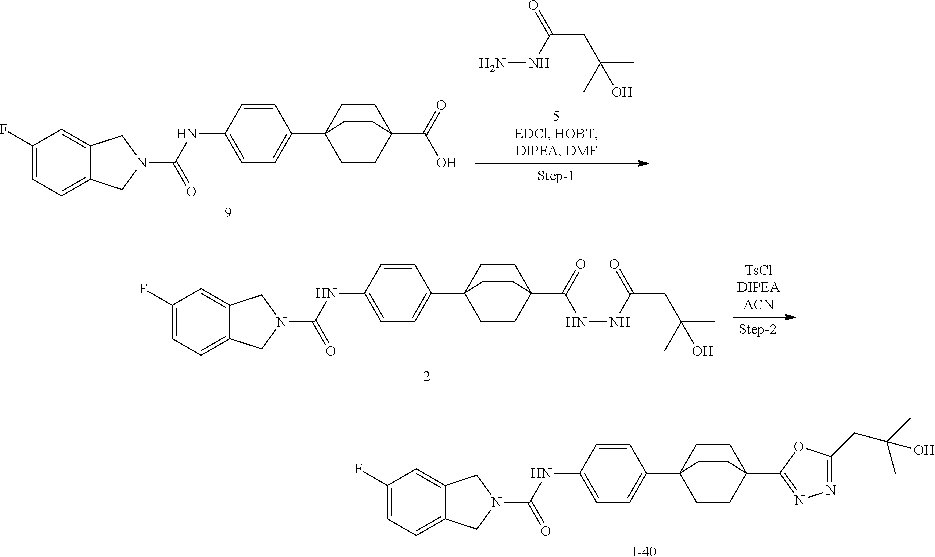

Preparation of 5-fluoro-N-(4-(4-(2-(3-hydroxy-3-methylbutanoyl)hydrazine-1-carbonyl)bicyclo [2.2.2]octan-1-yl)phenyl)isoindoline-2-carboxamide (3): To a stirred solution of 9 (1.0 g, 2.45 mmol) in DMF (20 mL) was added 5 (485 mg, 3.67 mmol), EDCI (2.1 g, 11.02 mmol), HOBt (1.48 g, 11.02 mmol), and DIPEA (3.61 mL, 19.60 mmol) at room temperature. The reaction mixture was stirred at room temperature for 16 hours, and the progress of the reaction was monitored by TLC. After complete consumption of starting material, the reaction mixture was diluted with ice-cold water to form a precipitate. The precipitate was filtered, washed with water, and dried to obtain the crude product, which was purified by column chromatography (eluent: 0 to 10% MeOH in DCM) to afford 2 (0.5 g, 39% yield) as an off-white solid. ¹H NMR (300 MHz, DMSO-d₆): δ 9.53 (s, 1H), 9.32 (s, 1H), 8.28 (s, 1H), 7.48-7.35 (m, 4H), 7.28-7.08 (m, 6H), 4.72 (br d, J=8.3 Hz, 4H), 4.66 (s, 1H), 2.23 (s, 2H), 1.91-1.70 (m, 12H), 1.21-1.16 (m, 6H). LCMS: 523.05 (M+H). Purity: 99.10% by AUC. RT: 4.77. UV: 254 nm.

Preparation of 5-fluoro-N-(4-(4-(5-(2-hydroxy-2-methylpropyl)-1,3,4-oxadiazol-2-yl)bicyclo[2.2.2]octan-1-yl)phenyl)isoindoline-2-carboxamide (I-40): In a sealed tube, to a stirred solution of 2 (500 mg, 0.96 mmol) in ACN (10 mL), was added TsCl (636 mg, 3.35 mmol) and DIPEA (0.8 mL, 4.78 mmol) at room temperature. The reaction mixture was stirred at 60° C. for 8 hours, and the progress of the reaction was monitored by TLC. The reaction mixture was concentrated under reduced pressure to give a residue, which was purified by flash column chromatography (eluent: 0 to 5% MeOH in DCM) to afford I-40 (80 mg, 16% yield) as a pale-yellow solid. ¹H NMR (300 MHz, DMSO-d₆): δ 8.30 (s, 1H), 7.47 (d, J=8.8 Hz, 2H), 7.39 (dd, J=5.2, 8.5 Hz, 1H), 7.27-7.20 (m, 3H), 7.18-7.10 (m, 1H), 4.77-4.69 (m, 5H), 2.89 (s, 2H), 2.02-1.82 (m, 12H), 1.18 (s, 6H). LCMS: 505.12 (M+H). Purity: 99.70% by AUC. RT: 5.18. UV: 210 nm. HPLC: Purity: 98.37% by AUC. RT: 14.39. UV: 245 nm.

### Example 39—Biochemical and Cell-Based Biological Assays

Exemplary compounds were evaluated for biological activity in the assays described below.

### Part I—Biochemical Assay

Experimental Procedure for Biochemical Assay: Human recombinant enzyme (Uniprot P43490) was expressed with an N-terminal histidine tag (synthetic construct synthesized by ATUM) in E. coli BL21(DE3) using the procedure described by Burgos E S [Biochemistry 2008, 47(42), 11086; PMID: 18823127]. The enzyme was purified using nickel capture resin (IMAC procedure, same reference).

With a commercial kit (BPS Bioscience #71276-1), the two-step enzyme-coupled assay converts NMN into NAD+(the reactions consumes ATP and is catalyzed by nicotinamide nucleotide adenylyltransferase, NMNAT). The intermediate is further converted into fluorescent NADH by alcohol dehydrogenase (ADH) in the presence of ethanol and semi-carbazide [Burgos E S and Schramm V L Biochemistry 2008, 47(42), 11086; PMID: 18823127]. The procedure follows the manufacturer's recommendations with a few modifications (human N-terminal histidine-tagged NAMPT was used at a final concentration of 10 nM, ATP concentration was increased to 2.5 mM, and MgCl₂ was added to a final concentration of 5 mM). Inhibitor concentrations ranged from 0.625 to 625 nM (four-point dose-response), and the final DMSO concentration was 1% v/v. The reactions (background, positive, negative controls, and NADH calibration standards, total volume=100 mL) were monitored continuously for 2.5 hours. Slopes were established (RFU/h), corrected for background signal (enzyme without NAM and without inhibitor), and normalized to vehicle-treated wells (human enzyme with substrates and without inhibitor).

### Part II—Cell-Based Assay

Experimental Procedure for Cell-Based Assay: 96-well plates (VWR #10861-666) were seeded with HCT116 cells (ATCC #CCL-247; 3000 cells per well, 100 mL media). DMEM (Gibco #12430-054) with 10% FBS (ATCC #30-2020) was used for culture. Cells were exposed to the inhibitors for 96 hours (dose-response: either 0.125-1250 nM or 0.012-125 nM; final DMSO concentration is 1%). After four days at 37° C. under 5% CO₂ atmosphere, CellTiter 96 AQueous One Solution (Promega #G3581; 25 mL per well) was added to cells and media only wells (background signal). After 1 hour incubation at 37° C., the signal was stabilized by the addition of an SDS solution (10% w/v; 25 mL) and absorbance was measured at 490 nm. The signal was corrected for background signal and normalized to vehicle-treated wells (DMSO 1%).

### Part III—Results

The results are reported in the table below. For each assay, compounds with an EC₅₀ less than or equal to 5 nM are designated as “A”. Compounds with an EC₅₀ greater than 5 nM and less than or equal to 10 nM are designated as “B”. Compounds with an EC₅₀ greater than 10 nM and less than or equal to 25 nM are designated as “C”.

For the biochemical assay, compounds with an EC₅₀ greater than 25 nM are designated as “D”. For the cell-based assay, compounds with an EC₅₀ greater than 25 nM and less than or equal to 500 nM are designated as “D”.

TABLE 3
Biochemical and Cellular EC₅₀ Data

| Compound | Biochemical | HCT116 Cell Based |
|---|---|---|
| No. | Assay EC₅₀ | Assay EC₅₀ |
| I-1 | C | C |
| I-2 | C | B |
| I-3 | D | D |
| I-4 | C | B |
| I-5 | C | C |
| I-6 | C | D |
| I-7 | C | C |
| I-8 | C | C |
| I-9 | C | A |
| I-10 | C | C |
| I-11 | B | A |
| I-12 | C | A |
| I-13 | B | B |
| I-14 | A | B |
| I-15 | A | A |
| I-16 | A | A |
| I-17 | C | B |
| I-26 | C | C |
| I-27 | C | B |
| I-28 | D | B |
| I-29 | D | D |
| I-30 | D | D |
| I-31 | D | D |
| I-32 | D | D |
| I-33 | C | D |
| I-34 | D | C |
| I-35 | C | C |
| I-36 | D | D |
| I-37 | D | D |
| II-1 | B | D |
| II-2 | C | A |
| II-3 | C | D |
| II-4 | D | D |
| II-5 | D | D |
| II-6 | D | D |

### Example 40—Additional Biochemical and Cell-Based Biological Assays

Exemplary compounds were evaluated for biological activity in the assays described below.

### Part I—Biochemical Assay

Experimental Procedure for Biochemical Assay: Human recombinant NAMPT enzyme was from BPS (Cat #91004). Alcohol Dehydrogenase (Cat #A3263), nicotinamide (Cat #72340) and phosphoribosyl pyrophosphate (Cat #P8296) were purchased from Sigma-Aldrich. FK-866 was purchased from Cayman Chemical (Cat #13287). The compounds were dissolved in DMSO. The serial dilution of the compounds was first performed in 100% DMSO with the highest concentration at 1.25 mM. Each intermediate compound dilution (in 100% DMSO) will then give directly diluted 10× fold into assay buffer for an intermediate dilution of 10% DMSO in NAMPT assay buffer and 5 μl of the dilution was added to a 50 μL reaction so that the final concentration of DMSO is 1% in all of reactions. For NAMPT, the compounds were preincubated with NAMPT enzyme (100 nM) for 30 minutes. All of the enzymatic reactions were conducted in triplicate at 30° C. for 120 minutes by adding the substrate mixture containing in a 50 μl mixture containing 50 mM Tris-HCl, pH 8.0, 12.5 mM MgCl₂, 20 μM nicotinamide, 40 μM PRPP, 20 μM ATP, 30 μg/mL of alcohol dehydrogenase, 10 μg/mL of NMNAT, 1.5% alcohol, 1 mM DTT, 0.02% BSA, 0.01% Tween 20. The final concentration of DMSO in all reactions was 1%. Fluorescence intensity was measured at an excitation of 340 nm and an emission of 460 nm using a Tecan Infinite M1000 microplate reader.

### Part II—Cell-Based Assay

Experimental Procedure for Cell-Based Assay: HCT116 cells (Cat #CCL-247), DMEM (Cat #30-2002), FBS (Cat #30-2020) were purchased from ATCC. HCT116 cells were plated (3000 cells/well) on white 96-well tissue culture plate (Corning, 70-200-628) and incubated for 18 hr. Compounds were dissolved in 100% DMSO with the highest concentration at 1.25 mM. Cells were incubated with inhibitors (dose response; ⅓-fold dilution, 0.57 nM˜1250 nM; final DMSO concentration as 1%). After 96-hr incubation, CellTiterGlo 2.0 reagent (Promega, G9243; 100 μL per well) was added to each well. Mix the contents on an orbital shaker in the dark for 2-5 minutes. After 10 minutes of incubation, the luminescent signals were measured using Envision microplate reader. The signal was normalized to vehicle-treated wells (1% DMSO).

### Part III—Results

The results are reported in the table below. For each assay, compounds with an EC₅₀ less than or equal to 5 nM are designated as “A”. Compounds with an EC₅₀ greater than 5 nM and less than or equal to 10 nM are designated as “B”. Compounds with an EC₅₀ greater than 10 nM and less than or equal to 25 nM are designated as “C”. N/A indicates no results available.

For the biochemical assay, compounds with an EC₅₀ greater than 25 nM are designated as “D”. For the cell-based assay, compounds with an EC₅₀ greater than 25 nM and less than or equal to 500 nM are designated as “D”.

TABLE 4
Biochemical and Cellular EC₅₀ Data

| Compound | Biochemical | HCT116 Cell Based |
|---|---|---|
| No. | Assay EC₅₀ | Assay EC₅₀ |
| I-18 | D | C |
| I-19 | D | A |
| I-20 | D | A |
| I-21 | D | C |
| I-22 | D | C |
| I-23 | D | A |
| I-24 | D | D |
| I-25 | D | D |
| I-38 | D | D |
| I-39 | N/A | C |
| I-40 | N/A | A |

### Example 41—Hepatocyte Assays

Certain compounds described herein were evaluated for biological activity in the hepatocyte assays described below.

### Part I—Human Hepatocyte Assay

Experimental Procedure for Assay: 100 μL of 1×10⁶ cells/mL human hepatocytes (Cat #X008005, BIO-IVT, USA) in INVITROGRO KHB Buffer (BIO-IVT, USA) were aliquoted into each well of 1.0 mL glass, flat-bottom inserts (Cat #96F610, Analytical Sales and Services, USA) placed in aluminum base plates (Cat #96AB11, Analytical Sales and Services, USA). The plates were placed in a CO₂ incubator on an orbital shaker to allow the hepatocytes to warm for 10 minutes (min). Next, 0.1 μL of test compound (0.1 mM in DMSO) and assay controls (1 mM verapamil in DMSO; Cat #V4629, Sigma, Germany) were added to the respective wells of the glass, flat-bottom inserts to start the reaction using i.DOT liquid handler (DISPENDIX, Germany). The final concentrations of the test compounds and assay controls were 0.1 μM and 1 μM, respectively. The plates were returned to the incubator and placed on an orbital shaker. Each glass tube with 100 μL hepatocytes was mixed with 3 volumes (300 L) of acetonitrile containing internal standards, which were 100 ng/mL each of cetirizine (Cat #C3618, Sigma, Germany), tolbutamide (Cat #T3690, TCI, Japan), telmisartan (Cat #T2861, TCI, Japan), and bicalutamide (Cat #PHR1678, Sigma, Germany) to terminate the reaction at the following time points: 0.5, 5, 10, 15, 30, 60, 90 and 120 min. The samples were vortex-mixed for 5 min and centrifuged at 4,000 rpm for 10 min. After centrifugation, 100 μL of the supernatant was transferred to a 96-well loading plate and mixed with 100 μL of ultra-pure water, and then used for LC-MS/MS analysis. Cell viability was determined after completion of the experiment.

Data Analysis: Peak area ratios (analyte to internal standard) were considered for calculation of percent parent compound remaining (% PCR). The time point at 0.5 min was considered as 100% PCR; for other time points, % PCR was calculated against 0.5 min. The slope (k) of the linear range of the natural logarithmic (ln) curve of % PCR versus time was used for calculation of half-life (t_{1/2}).

The half-life and intrinsic clearance of compounds in hepatocytes were calculated using the below formulae:

$$Half - life ( {t}_{1 / 2} ) ( min ) = - 1 * 0.693 / k$$

$$V ( μ L / {10}^{6} cells ) = 1000 / number of cells incubated in million per mL$$

$$Intrinsic clearance ( μ L / min / million cells ) = V \times 0.693 / {t}_{1 / 2}$$

The in vitro intrinsic clearance (CLᵢₙₜ) (mL/min/kg) was calculated using the following formula: CLᵢₙₜ=(k/cells in incubation)×(hepatocellularity/1 g of liver weight)×(liver weight (g)/1 kg of body weight)

The scaled hepatic clearance CL_{hb} (mL/min/kg) was calculated using the below formula without correcting to fraction unbound in hepatocyte and plasma:
Qh: Liver blood flow (mL/min/kg);
CLᵢₙₜ: Scaled in vitro intrinsic clearance (mL/min/kg);

$${CL}_{blood} = ( Q \times {CL}_{int} ) / ( Q + {CL}_{int} )$$

$$\% Qh = {CL}_{hb} * 100 / hepatic blood flow$$

$$Extraction ratio = \% Qh / 100$$

Scaling factors: cells (×10⁶ cells/g liver)=120; liver body weight (gm/kg body weight)=20; hepatic blood flow (mL/min/kg)=21

### Part II—Dog Hepatocyte Assay

Experimental Procedure for Assay: The assay was performed as described in Part I for the human hepatocyte assay using dog hepatocytes (Cat #M00205, BIO-IVT, USA). The assay was quenched using 50 ng/mL each of cetirizine, tolbutamide, telmisartan, and bicalutamide in place of the 100 ng/mL concentrations used for the human hepatocyte assay.

Data Analysis: Data analysis was performed as described in Part I of the human hepatocyte assay using the following modified scaling factors: cells (×10⁶ cells/g liver)=240; liver body weight (gm/kg body weight)=32; hepatic blood flow (mL/min/kg)=35.

### Part III—Monkey Hepatocyte Assay

Experimental Procedure for Assay: The assay was performed as described in Part I for the human hepatocyte assay using monkey hepatoctyes (Cat #M00305, BIO-IVT, USA). The assay was quenched using 50 ng/mL each of cetirizine, tolbutamide, telmisartan, and bicalutamide in place of the 100 ng/mL concentrations used for the human hepatocyte assay.

Data Analysis: Data analysis was performed as described in Part I of the human hepatocyte assay using the following modified scaling factors: cells (×10⁶ cells/g liver)=120; liver body weight (gm/kg body weight)=32; hepatic blood flow (mL/min/kg)=44.

### Part IV—Rat Hepatocyte Assay

Experimental Procedure for Assay: The assay was performed as described in Part I for the human hepatocyte assay using rat hepatoctyes (Cat #M00005, BIO-IVT, USA). The assay was quenched using 50 ng/mL each of cetirizine, tolbutamide, telmisartan, and bicalutamide in place of the 100 ng/mL concentrations used for the human hepatocyte assay.

Data Analysis: Data analysis was performed as described in Part I of the human hepatocyte assay using the following modified scaling factors: cells (×10⁶ cells/g liver)=120; liver body weight (gm/kg body weight)=40; hepatic blood flow (mL/min/kg)=55.

### Part V—Results

The hepatocyte assays were used to assess metabolic stability (e.g., half-life and clearance) of the tested compounds in different species. The assays were useful for identifying compounds having similar metabolic profiles in humans and relevant preclinical species.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 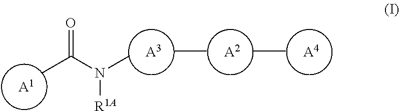
or a pharmaceutically acceptable salt thereof; wherein:
A¹ is 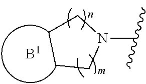
substituted with p occurrences of R², wherein B¹ is a 6-membered aryl ring or a 5-6 membered heteroaryl ring containing 1 or 2 heteroatoms independently selected from nitrogen and sulfur;
A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³;
A³ is phenylene substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1A} and R^{1B} each represent independently for each occurrence hydrogen or C₁₋₄ alkyl;
R² represents independently for each occurrence halo, C₁₋₄ haloalkyl, C₁₋₄ alkoxyl, C₁₋₄ alkyl, or C₁₋₄ hydroxyalkyl;
R³ represents independently for each occurrence halo or C₁₋₄ alkyl;
R⁴ represents independently for each occurrence C₁₋₆ hydroxyalkyl, C₁₋₆ alkyl, —(C₁₋₆ alkylene substituted with 1 occurrence of —N(R^{1B})—C(O)R⁵ or —N(R^{1B})—SO₂R₅), C₁₋₆ hydroxyhalo-alkyl, C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur);
R⁵ represents independently for each occurrence C₁₋₄ alkyl or —(C₀₋₃ alkylene)-(C₃₋₇ cycloalkyl);
m and n are independently 1 or 2;
p is 0, 1, 2, 3, or 4; and
x is 0, 1, or 2.

2. (canceled)

3. The compound of claim 1, or a pharmaceutically acceptable salt thereof, wherein A¹ is 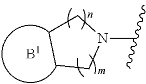
substituted with p occurrences of R², wherein B¹ is a 6-membered aryl ring or a 6-membered heteroaryl ring containing 1 or 2 heteroatoms that are nitrogen; and m and n are 1.

4. (canceled)

5. The compound of claim 1, or a pharmaceutically acceptable salt thereof, wherein A² is a 5-8 membered saturated bridged bicyclic carbocyclylene, a 6-membered saturated or partially unsaturated monocyclic carbocyclylene, or a 6-membered saturated or partially unsaturated monocyclic heterocyclylene containing 1 heteroatom that is nitrogen; each of which is substituted with 0 or 1 occurrence of R³.

6. (canceled)

7. The compound of claim 1, wherein the compound is represented by Formula I-A: 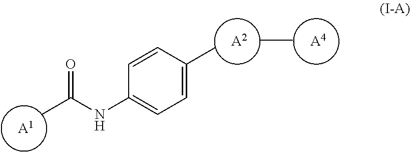
or a pharmaceutically acceptable salt thereof; wherein:
A¹ is 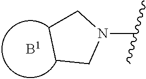 [SMILES: *N1CCCC1]
substituted with p occurrences of R², wherein B¹ is a 6-membered aryl ring or a 6-membered heteroaryl ring containing 1 or 2 heteroatoms that are nitrogen;
A² is a 5-8 membered saturated bridged bicyclic carbocyclylene or a 6-membered saturated or partially unsaturated monocyclic carbocyclylene; each of which is substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1B} represents independently for each occurrence hydrogen or C₁₋₄ alkyl;
R² represents independently for each occurrence halo or C₁₋₄ haloalkyl;
R³ is halo or C₁₋₄ alkyl;
R⁴ represents independently for each occurrence C₁₋₆ hydroxyalkyl, C₁₋₆ hydroxyhalo-alkyl, C₁₋₆ alkyl, —(C₁₋₆ alkylene substituted with 1 occurrence of —N(R^{1B})—C(O)R⁵ or —N(R^{1B})—SO₂R⁵), C₁₋₆ haloalkyl, C₁₋₄ alkoxyl, or —(C₀₋₃ alkylene)-(a monovalent 5-7 membered ring containing 0, 1, or 2 heteroatoms independently selected from nitrogen, oxygen, and sulfur);
R⁵ represents independently for each occurrence C₁₋₄ alkyl or —(C₀₋₃ alkylene)-(C₃₋₇ cycloalkyl);
p is 1 or 2; and
x is 0, 1, or 2.

8-9. (canceled)

10. The compound of claim 1, or a pharmaceutically acceptable salt thereof, wherein A² is (i) 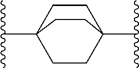 [SMILES: *C12CCC(*)(CC1)CC2]
substituted with 0 or 1 occurrence of R³ or (ii) 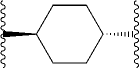 [SMILES: *[C@H]1CC[C@H](*)CC1]
substituted with 0 or 1 occurrence of R³.

11-12. (canceled)

13. The compound of claim 1, or a pharmaceutically acceptable salt thereof, wherein A¹ is 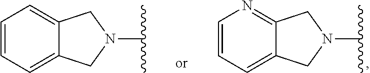 [SMILES: *N1Cc2ccccc2C1 ; *N1Cc2cccnc2C1]
each of which is substituted with p occurrences of R², wherein p is 0 or 1.

14-17. (canceled)

18. The compound of claim 1, or a pharmaceutically acceptable salt thereof, wherein A¹ is

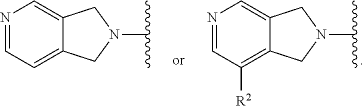

19. (canceled)

20. The compound of claim 1, or a pharmaceutically acceptable salt thereof, wherein R² is fluoro.

21. The compound of claim 1, or a pharmaceutically acceptable salt thereof, wherein A⁴ is a 5-membered heteroaryl containing 1, 2, 3, or 4 heteroatoms independently selected from nitrogen, oxygen, and sulfur, wherein the heteroaryl is substituted with x occurrences of R⁴.

22. (canceled)

23. The compound of claim 1, or a pharmaceutically acceptable salt thereof, wherein A⁴ is pyrazolyl, triazolyl, oxadiazolyl, or thiadiazolyl, each of which is substituted with x occurrences of R⁴.

24-26. (canceled)

27. The compound of claim 1, or a pharmaceutically acceptable salt thereof, wherein R⁴ represents independently for each occurrence C₁₋₆ hydroxyalkyl.

28-33. (canceled)

34. The compound of claim 1, or a pharmaceutically acceptable salt thereof, wherein R⁴ represents independently for each occurrence 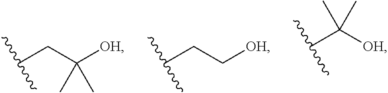
methyl, or

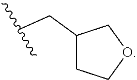 [SMILES: *CC1CCOC1]

35. The compound of claim 1, or a pharmaceutically acceptable salt thereof, wherein x is 1.

36. A compound selected from: 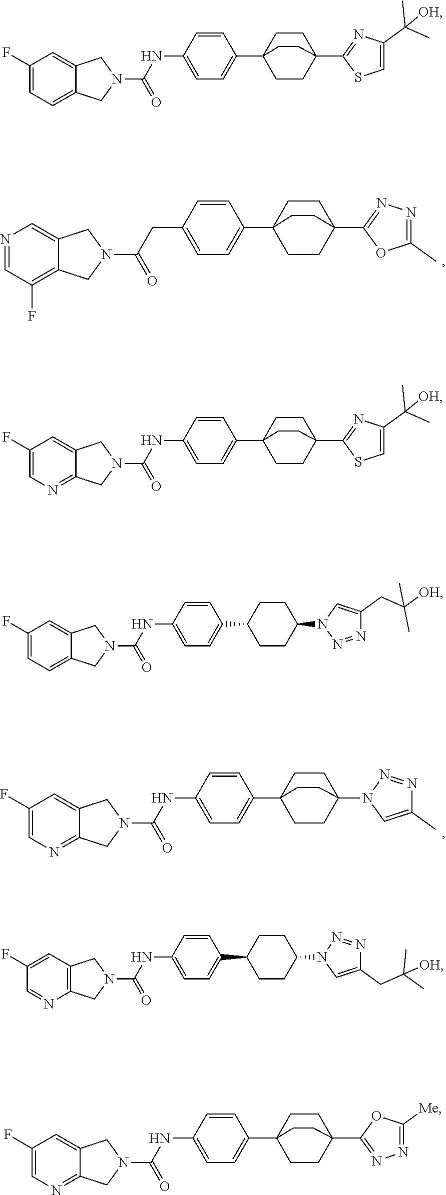 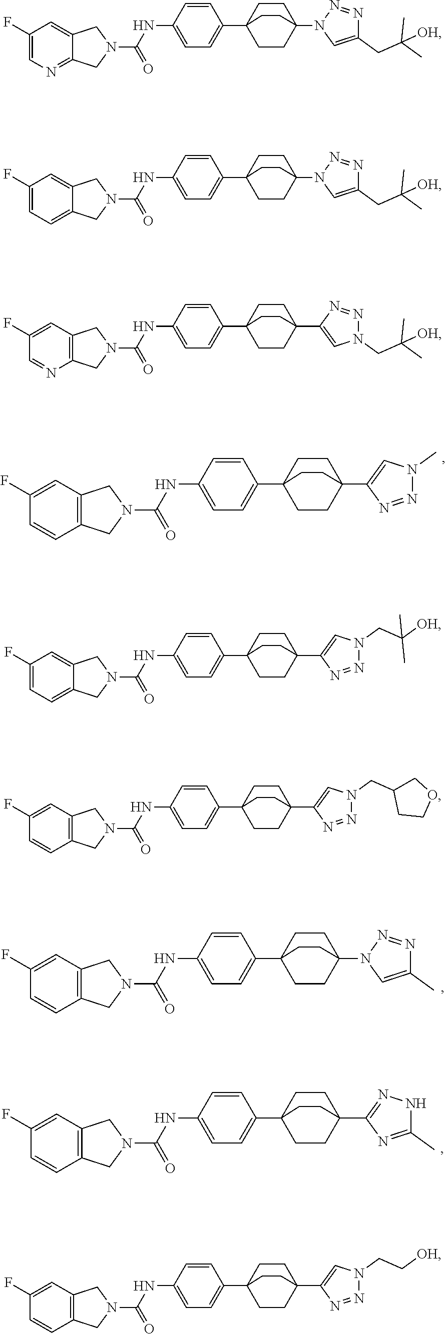 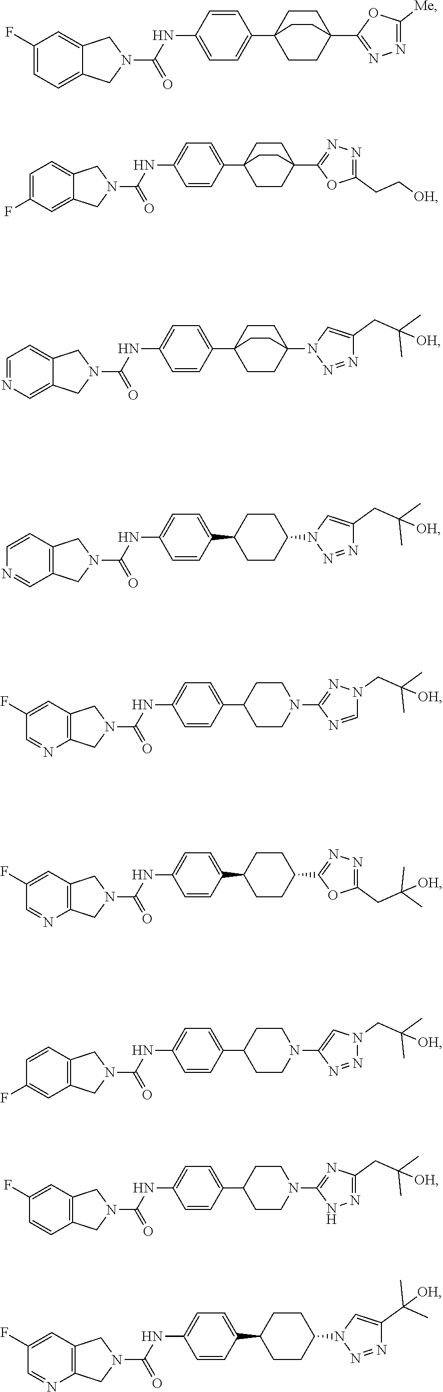 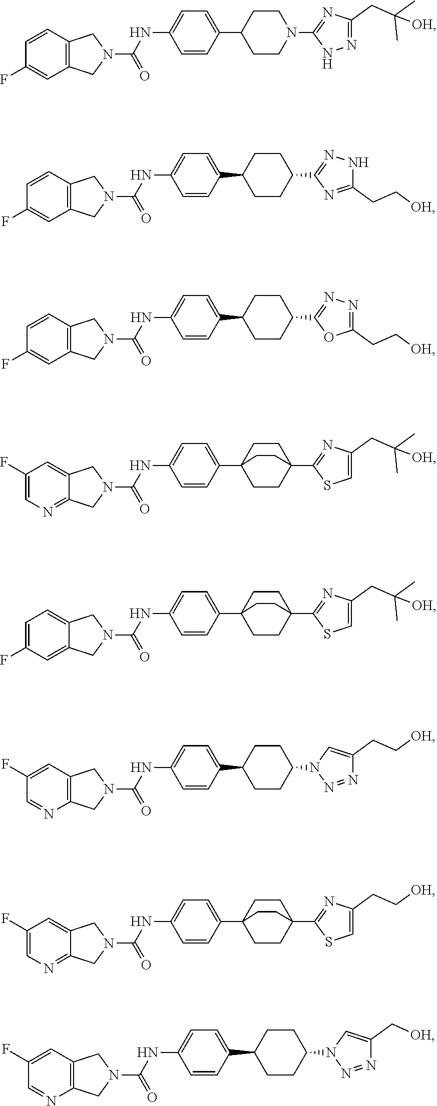 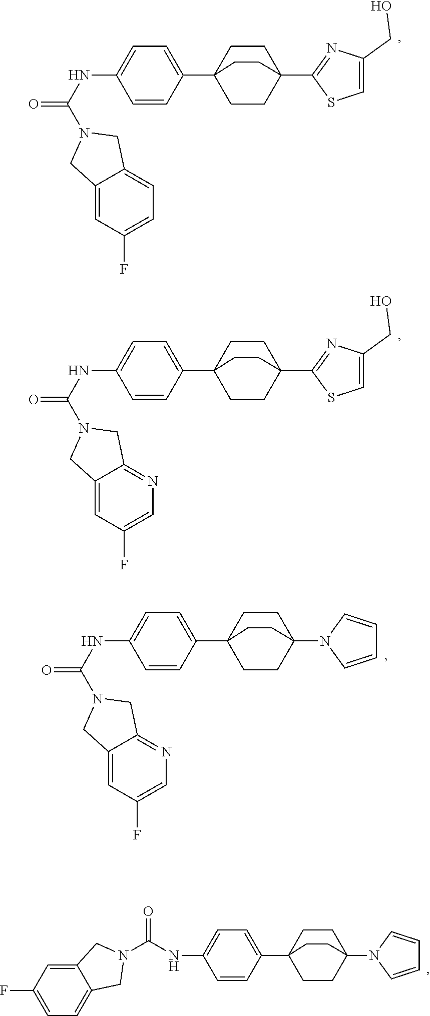 [SMILES: O=C(Nc1ccc(C23CCC(c4nc(CO)cs4)(CC2)CC3)cc1)N1Cc2ccc(F)cc2C1 ; O=C(Nc1ccc(C23CCC(c4nc(CO)cs4)(CC2)CC3)cc1)N1Cc2cc(F)cnc2C1 ; O=C(Nc1ccc(C23CCC(n4cccc4)(CC2)CC3)cc1)N1Cc2cc(F)cnc2C1 ; O=C(Nc1ccc(C23CCC(n4cccc4)(CC2)CC3)cc1)N1Cc2ccc(F)cc2C1] 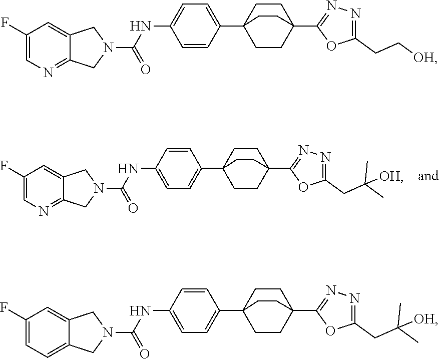 [SMILES: O=C(Nc1ccc(C23CCC(c4nnc(CCO)o4)(CC2)CC3)cc1)N1Cc2cc(F)cnc2C1 ; CC(C)(O)Cc1nnc(C23CCC(c4ccc(NC(=O)N5Cc6cc(F)cnc6C5)cc4)(CC2)CC3)o1 ; CC(C)(O)Cc1nnc(C23CCC(c4ccc(NC(=O)N5Cc6ccc(F)cc6C5)cc4)(CC2)CC3)o1]
or a pharmaceutically acceptable salt thereof.

37. (canceled)

38. A pharmaceutical composition comprising the compound of claim 1, or a pharmaceutically acceptable salt thereof, and a pharmaceutically acceptable carrier.

39. A compound represented by Formula II: 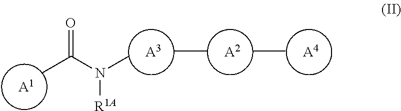
or a pharmaceutically acceptable salt thereof; wherein:
A¹ is 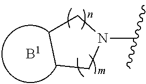
substituted with p occurrences of R², wherein B¹ is a 6-membered aryl ring or a 5-6 membered heteroaryl ring containing 1 or 2 heteroatoms independently selected from nitrogen and sulfur;
A² is a 5-8 membered saturated bicyclic carbocyclylene, a 6-membered saturated or partially unsaturated monocyclic carbocyclylene, or a 4-8 membered saturated or partially unsaturated heterocyclylene containing 1 heteroatom selected from nitrogen and oxygen; each of which is substituted with 0 or 1 occurrence of R³;
A³ is phenylene substituted with 0 or 1 occurrence of R³;
A⁴ is a 5-6 membered heteroaryl containing 1, 2, 3, or 4 heteroatoms independently selected from nitrogen, oxygen, and sulfur, wherein the heteroaryl is substituted with x occurrences of R⁴;
R^{1A} and R^{1B} each represent independently for each occurrence hydrogen or C₁₋₄ alkyl;
R² represents independently for each occurrence halo, C₁₋₄ haloalkyl, C₁₋₄ alkoxyl, C₁₋₄ alkyl, or C₁₋₄ hydroxyalkyl;
R³ represents independently for each occurrence halo or C₁₋₄ alkyl;
R⁴ represents independently for each occurrence —(C₀₋₆ alkylene)-CO₂R⁶; —(C₀₋₆ alkylene)-C(O)N(R^{1B})—(C₁₋₆ hydroxyalkyl), or —(C₀₋₄ alkylene)-COR⁵;
R⁵ represents independently for each occurrence C₁₋₄ alkyl or —(C₀₋₃ alkylene)-(C₃₋₇ cycloalkyl);
R⁶ represents independently for each occurrence hydrogen, C₁₋₆ alkyl, or —(C₀₋₃ alkylene)-(C₃₋₇ cycloalkyl);
m and n are independently 1 or 2;
p is 0, 1, 2, 3, or 4; and
x is 0, 1, or 2.

40-71. (canceled)

72. A method for treating a disease or condition mediated by NAMPT in a subject in need thereof, comprising administering to the subject a therapeutically effective amount of the compound of claim 1 or a pharmaceutically acceptable salt thereof.

73-86. (canceled)

87. A method of inhibiting the activity of NAMPT, comprising contacting a NAMPT with an effective amount of the compound of claim 1, or a pharmaceutically acceptable salt thereof, to inhibit the activity of said NAMPT.

88-89. (canceled)

90. The compound of claim 36, which is: 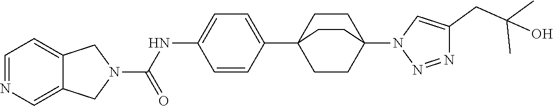 [SMILES: CC(C)(O)Cc1cn(C23CCC(c4ccc(NC(=O)N5Cc6ccncc6C5)cc4)(CC2)CC3)nn1]
or a pharmaceutically acceptable salt thereof.

91. The compound of claim 36, which is: 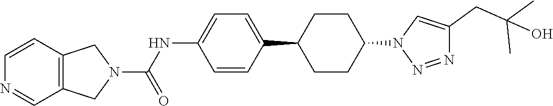 [SMILES: CC(C)(O)Cc1cn([C@H]2CC[C@H](c3ccc(NC(=O)N4Cc5ccncc5C4)cc3)CC2)nn1]
or a pharmaceutically acceptable salt thereof.
